(19) United States
(12) Patent Application Publication
(10) Pub. No.: US 2022/0281911 A1
(43) Pub. Date: Sep. 8, 2022

(54) OLIGONUCLEOTIDE CONJUGATE COMPOSITIONS AND METHODS OF USE

(71) Applicants: MiNA THERAPEUTICS LIMITED, London (GB); LGC GENOMICS LTD., Teddington Middlesex (GB)

(72) Inventors: Alexandre Debacker, London (GB); Jon Voutila, London (GB); Lee Mitchell, Riddrie (GB); Catherine M. McKeen, Livingston (GB)

(21) Appl. No.: 17/636,437

(22) PCT Filed: Aug. 19, 2020
(86) PCT No.: PCT/EP2020/073187

§ 371 Date: Feb. 18, 2022

Related U.S. Application Data
(60) Provisional application No. 62/888,748, filed on Aug. 19, 2019.
(60) Provisional application No. 63/064,114, filed on Aug. 11, 2020.

(51) Int. Cl. C07H 21/04; C07H 21/02

(52) U.S. Cl. CPC C07H 21/04; C07H 21/02; C12N 15/10

(57) ABSTRACT

The disclosure relates to GalNAc moieties comprising at least one GalNAc monomer. The disclosure also relates to GalNAc-oligonucleotide conjugates comprising GalNAc moieties and oligonucleotides, e.g., saRNAs or siRNAs useful in regulating the expression of a target gene. Methods of using the GalNAc-oligonucleotide conjugates are also provided.

### CROSS-REFERENCE TO RELATED APPLICATIONS

This application claims priority to U.S. Provisional Application No. 62/888,748 filed Aug. 19, 2019, and U.S. Provisional Application No. 63/064,114 filed Aug. 11, 2020, the contents of each of which are incorporated herein by reference in their entirety.

### REFERENCE TO SEQUENCE LISTING

The present application is being filed along with a Sequence Listing in electronic format. The sequence listing filed, entitled 2058-1026USPCT SL.txt, was created on Aug. 17, 2020 and is 44,351 bytes in size. The information in electronic format of the Sequence Listing is incorporated herein by reference in its entirety.

### FIELD OF THE DISCLOSURE

The disclosure relates to GalNAc moieties comprising at least one GalNAc monomer. The disclosure also relates to GalNAc-oligonucleotide conjugates comprising GalNAc moieties and oligonucleotides, e.g., small activating RNA (saRNAs) or small inhibiting (siRNAs).

### BACKGROUND

CCAAT/enhancer-binding protein a (C/EBPα, C/EBP alpha, C/EBPA or CEBPA) is a leucine zipper protein that is conserved across humans and rodents. This nuclear transcription factor is enriched in hepatocytes, myelomonocytes, adipocytes, as well as other types of mammary epithelial cells [Lekstrom-Himes et al., J. Bio. Chem, vol. 273, 28545-28548 (1998)]. It is composed of two transactivation domains in the N-terminal part, and a leucine zipper region mediating dimerization with other C/EBP family members and a DNA-binding domain in the C-terminal part. The binding sites for the family of C/EBP transcription factors are present in the promoter regions of numerous genes that are involved in the maintenance of normal hepatocyte function and response to injury. C/EBPα has a pleiotropic effect on the transcription of several liver-specific genes implicated in the immune and inflammatory responses, development, cell proliferation, anti-apoptosis, and several metabolic pathways [Darlington et al., Current Opinion of Genetic Development, vol. 5(5), 565-570 (1995)]. It is essential for maintaining the differentiated state of hepatocytes. It activates albumin transcription and coordinates the expression of genes encoding multiple ornithine cycle enzymes involved in urea production, therefore playing an important role in normal liver function.

There is a need for targeted modulation of CEBPA for therapeutic purposes with saRNA.

### BRIEF DESCRIPTION OF THE DRAWINGS

The foregoing and other objects, features and advantages will be apparent from the following description of particular embodiments of the invention, as illustrated in the accompanying drawings in which like reference characters refer to the same parts throughout the different views. The drawings are not necessarily to scale, emphasis instead being placed upon illustrating the principles of various embodiments of the invention.

FIG. 1 shows CEBPA mRNA level after saRNA passive delivery in primary rat hepatocyte at 500 nM.

FIG. 2 shows Albumin mRNA level after saRNA passive delivery in primary rat hepatocyte at 500 nM.

FIG. 3 shows CEBPA mRNA level after saRNA passive delivery in primary rat hepatocyte at 1 μM.

FIG. 4 shows Albumin mRNA level after saRNA passive delivery in primary rat hepatocyte at 1 μM.

FIG. 5 shows the level of CEBPA mRNA after injection of normal mice at 40 mg/Kg on day 1 and day 3 and killed at day 5. CEBPA is normalized to PBS with B2M as Housekeeping. RNA was extracted from frozen liver sample and mRNA level was measured by qPCR.

FIG. 6 shows the level of CEBPA mRNA after injection of normal mice at 40 mg/Kg on day 1 and day 3 and killed at day 5. CEBPA is normalized to PBS with B2M as Housekeeping. RNA extracted from frozen liver sample and mRNA level measured by qPCR.

FIG. 7 shows the level of Albumin mRNA after injection of normal mice at 40 mg/Kg on day 1 and day 3 and killed at day 5. Albumin is normalized to PBS with B2M as Housekeeping. RNA was extracted from frozen liver sample and mRNA level was measured by qPCR.

FIG. 8 shows the level of CEBPA mRNA in liver after SC injection of GalNAc saRNA conjugates in normal mice 30 mg/kg on day 1 and day 3 and killed at day 5.

FIG. 9 shows in-vitro dose response of CEBPa-saRNA-GalNAc conjugates L80 (XD-14369K1 conjugated to GalNac cluster G7) and L81 (XD-14369K1 conjugated to GalNac cluster G8).

FIG. 10 shows C5 mRNA levels after C5-siRNA-GalNAc conjugates were transfected.

### SUMMARY OF THE DISCLOSURE

The present invention provides compositions, methods and kits for the design, preparation, manufacture, formulation and/or use of short (or small) activating RNA (saRNA), whether modified or not, that modulate target gene expression and/or function for therapeutic purposes, including diagnosing and prognosis. The terms “modified” or, as appropriate, “modification” refer to structural and/or chemical modifications with respect to any one or more of the components of a nucleotide (sugar, base or backbone). In the case of the base, any of the standard nucleobases: A, G, U or C ribonucleobases may be modified. Nucleotides in the saRNAs of the present invention may comprise non-standard nucleotides, such as non-naturally occurring nucleotides or chemically synthesized nucleotides or deoxynucleotides.

One aspect of the invention provides a synthetic isolated small activating RNA (saRNA) which up-regulates the expression of a target gene, wherein the saRNA comprises at least one modification to at least one of the base, sugar or backbone of the polynucleotide comprising the saRNA.

Another aspect of the invention provides a N-Acetyl-Galactosamine (GalNAc) monomer comprising a structure selected from the group consisting of

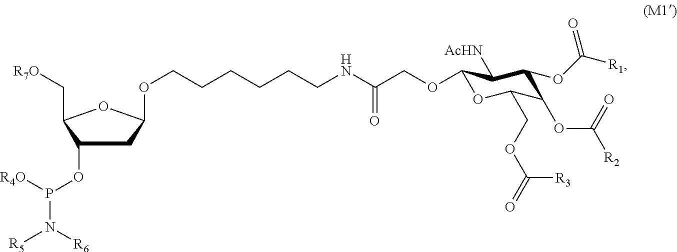

wherein R1, R2, and R3 can be the same or different, and wherein R1, R2, and R3 are independently selected from an alkyl, aryl, and alkenyl group, wherein R4 is a suitable protecting group or a C1-6 straight or branched alkyl group, wherein R5 and R6 are each independently a C1-6 straight or branched alkyl group, and wherein R7 is a suitable protecting group;

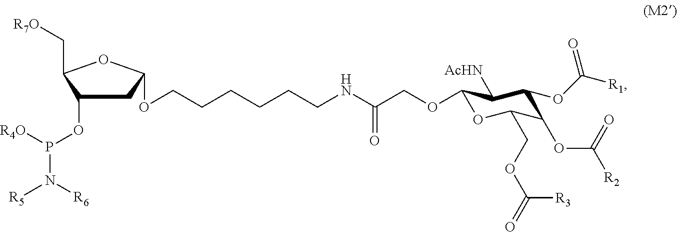

wherein R1, R2, and R3 can be the same or different, and wherein R1, R2, and R3 are independently selected from an alkyl, aryl, and alkenyl group; wherein R4 is a protecting group or a C1-6 straight or branched alkyl group, wherein R5 and R6 are each independently C1-6 straight or branched alkyl; and wherein R7 is a suitable protecting group;

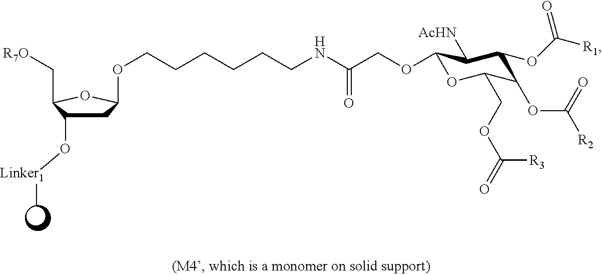

wherein R1, R2, and R3 can be the same or different, and wherein R1, R2, and R3 are independently selected from an alkyl, aryl, and alkenyl group, wherein R7 is a suitable protecting group, and wherein Linker1 is a cleavable linker; and

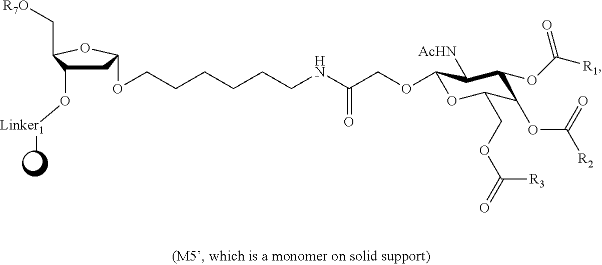

wherein R1, R2, and R3 can be the same or different, and wherein R1, R2, and R3 are independently selected from the group consisting of an alkyl, aryl, and alkenyl group, wherein R7 is a suitable protecting group, and wherein Linker1 is a cleavable linker.

Another aspect of the invention GalNAc moiety comprising at least one GalNAc monomer, wherein the GalNAc monomers are selected from the group consisting of

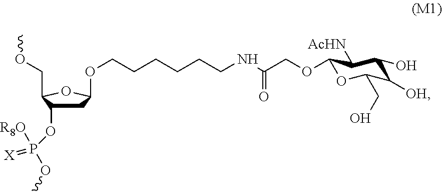

wherein R₈ is —H or a C1-6 straight or branched alkyl group;

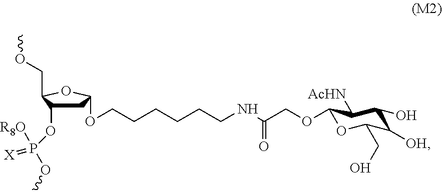

wherein R₈ is —H or a C1-6 straight or branched alkyl group, and wherein X is O or S;

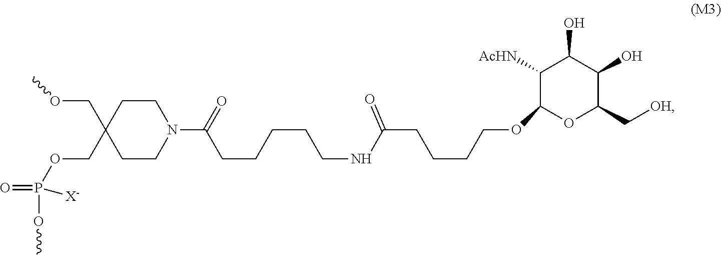

wherein X is O or S;

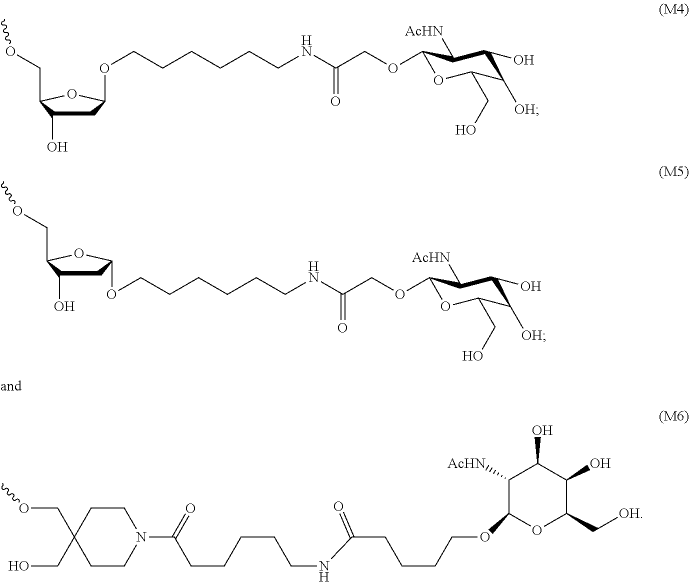

Another aspect of the invention provides a conjugate comprising an oligonucleotide connected to a carbohydrate moiety (such as an N-acetyl-galactosamine (GalNAc) moiety) via a linker. In the context of the present application, the term “moiety” means one unit or component in a whole compound or conjugate. For example, a conjugate can have a GalNAc moiety, a linker moiety, and a saRNA moiety. A GalNAc moiety can comprise one or more GalNAc monomers together. The term “GalNAc cluster” or “GalNAc multimer” means two or more GalNAc monomers together. Therefore, in some situations (i.e. where there are two or more molecules together), the terms “GalNAc moiety”, “GalNAc cluster” and “GalNAc multimer” may be synonymous. The oligonucleotide may be antisense oligonucleotides (ASO), small activating RNAs (saRNAs), small inhibiting RNAs (siRNAs), microRNAs (miRNAs), modified mRNAs, self-amplifying RNAs, circular RNAs, aptamer RNAs, ribozymes, plasmids, and immune stimulating nucleic acids. The oligonucleotide may be single-stranded or double-stranded. The oligonucleotide may comprise naturally-occurring nucleotides, synthetic nucleotides, and/or modified nucleotides. The terms “small activating RNA”, “short activating RNA”, or “saRNA” in the context of the present invention means a single-stranded or double-stranded RNA that upregulates or has a positive effect on the expression of a specific gene. The gene is the target gene of the saRNA. The terms “small interfering RNA,” “small inhibiting RNA,” or “siRNA” in the context mean a double-stranded RNA involved in the RNA interference (RNAi) pathway and interfering with or inhibiting the expression of a specific gene. The gene is the target gene of the siRNA.

Another aspect of the invention provides a pharmaceutical composition comprising a modified saRNA or a conjugate comprising an saRNA connected to a carbohydrate moiety (such as a GalNAc moiety) and at least one pharmaceutically acceptable excipient.

Another aspect of the invention provides a method of delivering an saRNA to cells comprising administering a conjugate comprising an saRNA connected to a carbohydrate moiety (such as a GalNAc moiety).

Another aspect of the invention provides a method of up-regulating the expression of a target gene comprising administering a modified saRNA or a conjugate comprising an saRNA connected to a carbohydrate moiety (such as a GalNAc moiety).

Another aspect of the invention provides treating or preventing a disease comprising administering a modified saRNA or a conjugate comprising an saRNA connected to a carbohydrate moiety (such as a GalNAc moiety), wherein the saRNA up-regulates the expression of a target gene, and wherein the target gene is associated with the disease.

Another aspect of the invention provides a pharmaceutical composition comprising a modified siRNA or a conjugate comprising an siRNA connected to a carbohydrate moiety (such as a GalNAc moiety) and at least one pharmaceutically acceptable excipient. The siRNA may down-regulate the expression of target genes such as but not limited to complement C5 (C5) or transthyretin (TTR).

Another aspect of the invention provides a method of delivering an siRNA to cells comprising administering a conjugate comprising an siRNA connected to a carbohydrate moiety (such as a GalNAc moiety).

Another aspect of the invention provides a method of down-regulating the expression of a target gene comprising administering a modified siRNA or a conjugate comprising an siRNA connected to a carbohydrate moiety (such as a GalNAc moiety).

Another aspect of the invention provides treating or preventing a disease comprising administering a modified siRNA or a conjugate comprising an siRNA connected to a carbohydrate moiety (such as a GalNAc moiety), wherein the siRNA down-regulates the expression of a target gene, and wherein the target gene is associated with the disease.

The details of various embodiments of the invention are set forth in the description below. Other features, objects, and advantages of the invention will be apparent from the description and the drawings, and from the claims.

### DETAILED DESCRIPTION

The present invention provides compositions, methods and kits for modulating target gene expression and/or function for therapeutic purposes. These compositions, methods and kits comprise at least one saRNA that upregulates the expression of a target gene, wherein the saRNA comprises at least one chemical modification.

I. Design and Synthesis of saRNA

The terms “small activating RNA”, “short activating RNA”, or “saRNA” in the context of the present invention means a single-stranded or double-stranded RNA that upregulates or has a positive effect on the expression of a specific gene. The saRNA may be single-stranded of 14 to 30 nucleotides. The saRNA may also be double-stranded, each strand comprising 14 to 30 nucleotides. The gene is called the target gene of the saRNA. As used herein, the target gene is a double-stranded DNA comprising a coding strand and a template strand. For example, an saRNA that upregulates the expression of the CEBPA gene is called an “CEBPA-saRNA” and the CEBPA gene is the target gene of the CEBPA-saRNA. A target gene may be any gene of interest. In some embodiments, a target gene has a promoter region on the template strand.

By “upregulation” or “activation” of a gene or an mRNA is meant an increase in the level of expression of a gene or mRNA, or levels of the polypeptide(s) encoded by the mRNA or the activity thereof. The saRNA of the present invention may have a direct upregulating effect on the expression of the target gene.

The saRNAs of the present invention may have an indirect upregulating effect on the RNA transcript(s) transcribed from the template strand of the target gene and/or the polypeptide(s) encoded by the target gene or mRNA. The RNA transcript transcribed from the target gene is referred to thereafter as the target transcript. The target transcript may be an mRNA of the target gene. The target, transcript may exist in the mitochondria. The saRNAs of the present invention may have a downstream effect on a biological process or activity. In such embodiments, an saRNA targeting a first transcript may have an effect (either upregulating or downregulating) on a second, non-target transcript.

In one embodiment, the saRNA of the present invention may show efficacy in proliferating cells. As used herein with respect to cells, “proliferating” means cells which are growing and/or reproducing rapidly.

### Target Antisense RNA Transcript of a Target Gene

In one embodiment, the saRNAs of the present invention is designed to be complementary to a target antisense RNA transcript of a target gene, and it may exert its effect on the target gene expression and/or function by down-regulating the target antisense RNA transcript. The target antisense RNA transcript is transcribed from the coding strand of the target gene and may exist in the nucleus of a cell.

The term “complementary to” in the context means being able to hybridize with the target antisense RNA transcript under stringent conditions.

The term “antisense” when used to describe a target antisense RNA transcript in the context of the present invention means that the sequence is complementary to a sequence on the coding strand of a gene.

It is to be understood that thymidine of the DNA is replaced by uridine in RNA and that this difference does not alter the understanding of the terms “antisense” or “complementarity”.

The target antisense RNA transcript may be transcribed from a locus on the coding strand between up to 100, 80, 60, 40, 20 or 10 kb upstream of a location corresponding to the target gene's transcription start site (TSS) and up to 100, 80, 60, 40, 20 or 10 kb downstream of a location corresponding to the target gene's transcription stop site.

In one embodiment, the target antisense RNA transcript is transcribed from a locus on the coding strand located within +/−1 kb of the target gene's transcription start site.

In another embodiment, the target antisense RNA transcript is transcribed from a locus on the coding strand located within +/−500 nt, +/−250 nt, +/−100 nt, +/−10 nt, +/−5 nt or +/−1 nt of the target gene's transcription start site.

In another embodiment, the target antisense RNA transcript is transcribed from a locus on the coding strand located +/−2000 nucleotides of the target gene's transcription start site.

In another embodiment, the locus on the coding strand is no more than 1000 nucleotides upstream or downstream from a location corresponding to the target gene's transcription start site.

In another embodiment, the locus on the coding strand is no more than 500 nucleotides upstream or downstream from a location corresponding to the target gene's transcription start site.

The term “transcription start site” (TSS) as used herein means a nucleotide on the template strand of a gene corresponding to or marking the location of the start of transcription. The TSS may be located within the promoter region on the template strand of the gene.

The term “transcription stop site” as used herein means a region, which can be one or more nucleotides, on the template strand of a gene, which has at least one feature such as, but not limited to, a region which encodes at least one stop codon of the target transcript, a region encoding a sequence preceding the 3′UTR of the target transcript, a region where the RNA polymerase releases the gene, a region encoding a splice site or an area before a splice site and a region on the template strand where transcription of the target transcript terminates.

The phrase “is transcribed from a particular locus” in the context of the target antisense RNA transcript of the invention means the transcription of the target antisense RNA transcript starts at the particular locus.

The target antisense RNA transcript is complementary to the coding strand of the genomic sequence of the target gene, and any reference herein to “genomic sequence” is shorthand for “coding strand of the genomic sequence”.

The “coding strand” of a gene has the same base sequence as the mRNA produced, except T is replaced by U in the mRNA. The “template strand” of a gene is therefore complementary and antiparallel to the mRNA produced.

Thus, the target antisense RNA transcript may comprise a sequence which is complementary to a genomic sequence located between 100, 80, 60, 40, 20 or 10 kb upstream of the target gene's transcription start site and 100, 80, 60, 40, 20 or 10 kb downstream of the target gene's transcription stop site.

In one embodiment, the target antisense RNA transcript comprises a sequence which is complementary to a genomic sequence located between 1 kb upstream of the target gene's transcription start site and 1 kb downstream of the target gene's transcription stop site.

In another embodiment, the target antisense RNA transcript comprises a sequence which is complementary to a genomic sequence located between 500, 250, 100, 10, 5 or 1 nucleotide upstream of the target gene's transcription start site and ending 500, 250, 100, 10, 5 or 1 nucleotide downstream of the target gene's transcription stop site.

The target antisense RNA transcript may comprise a sequence which is complementary to a genomic sequence which includes the coding region of the target gene. The target antisense RNA transcript may comprise a sequence which is complementary to a genomic sequence that aligns with the target gene's promoter region on the template strand. Genes may possess a plurality of promoter regions, in which case the target antisense RNA transcript may align with one, two or more of the promoter regions. An online database of annotated gene loci may be used to identify the promoter regions of genes. The terms ‘align’ and ‘alignment’ when used in the context of a pair of nucleotide sequences mean the pair of nucleotide sequences are complementary to each other or have sequence identity with each other.

The region of alignment between the target antisense RNA transcript and the promoter region of the target gene may be partial and may be as short as a single nucleotide in length, although it may be at least 15 or at least 20 nucleotides in length, or at least 25 nucleotides in length, or at least 30, 35, 40, 45 or 50 nucleotides in length, or at least 55, 60, 65, 70 or 75 nucleotides in length, or at least 100 nucleotides in length. Each of the following specific arrangements is intended to fall within the scope of the term “alignment”:

a) The target antisense RNA transcript and the target gene's promoter region are identical in length and they align (i.e. they align over their entire lengths).

b) The target antisense RNA transcript is shorter than the target gene's promoter region and aligns over its entire length with the target gene's promoter region (i.e. it aligns over its entire length to a sequence within the target gene's promoter region).

c) The target antisense RNA transcript is longer than the target gene's promoter region and the target gene's promoter region is aligned fully by it (i.e. the target gene's promoter region is aligned over its entire length to a sequence within the target antisense RNA transcript).

d) The target antisense RNA transcript and the target gene's promoter region are of the same or different lengths and the region of alignment is shorter than both the length of the target antisense RNA transcript and the length of the target gene's promoter region.

The above definition of “align” and “alignment” applies mutatis mutandis to the description of other overlapping, e.g., aligned sequences throughout the description. Clearly, if a target antisense RNA transcript is described as aligning with a region of the target gene other than the promoter region then the sequence of the target antisense RNA transcript aligns with a sequence within the noted region rather than within the promoter region of the target gene.

In one embodiment, the target antisense RNA transcript is at least 1 kb, or at least 2, 3, 4, 5, 6, 7, 8, 9 or 10, e.g., 20, 25, 30, 35 or 40 kb long.

In one embodiment, the target antisense RNA transcript comprises a sequence which is at least 75%, or at least 85%, or at least 90%, or at least 95% complementary along its full length to a sequence on the coding strand of the target gene.

The present invention provides saRNAs targeting the target antisense RNA transcript and may effectively and specifically down-regulate such target antisense RNA transcripts. This can be achieved by saRNA having a high degree of complementarity to a region within the target antisense RNA transcript. The saRNA will have no more than 5, or no more than 4 or 3, or no more than 2, or no more than 1, or no mismatches with the region within the target antisense RNA transcript to be targeted.

As the target antisense RNA transcript has sequence identity with a region of the template strand of the target gene, the target antisense RNA transcript will be in part identical to a region within the template strand of the target gene allowing reference to be made either to the template strand of the gene or to a target antisense RNA transcript. The location at which the saRNA hybridizes or binds to the target antisense RNA transcript (and hence the same location on the template strand) is referred to as the “targeted sequence” or “target site”.

The guide or antisense strand of the saRNA (whether single- or double-stranded) may be at least 80%, 90%, 95%, 98%, 99% or 100% identical with the reverse complement of the targeted sequence on the template strand of the target gene. In another word, the guide or antisense strand of the saRNA may be at least 80%, 90%, 95%, 98%, 99% or 100% complementary to the targeted sequence. Thus, the reverse complement of the guide or antisense strand of the saRNA has a high degree of sequence identity with the targeted sequence. The targeted sequence may have the same length, i.e., the same number of nucleotides, as the saRNA and/or the reverse complement of the saRNA.

In some embodiments, the targeted sequence comprises at least 14 and less than 30 nucleotides.

In some embodiments, the targeted sequence has 17, 18, 19, 20, 21, 22, or 23 nucleotides.

In some embodiments, the location of the targeted sequence is situated within a promoter area of the template strand.

In some embodiments, the targeted sequence is located within a TSS (transcription start site) core of the template stand. A “TSS core” or “TSS core sequence” as used herein, refers to a region between 2000 nucleotides upstream and 2000 nucleotides downstream of the TSS (transcription start site). Therefore, the TSS core comprises 4001 nucleotides and the TSS is located at position 2001 from the 5′ end of the TSS core sequence.

In some embodiments, the targeted sequence is located between 1000 nucleotides upstream and 1000 nucleotides downstream of the TSS.

In some embodiments, the targeted sequence is located between 500 nucleotides upstream and 500 nucleotides downstream of the TSS.

In some embodiments, the targeted sequence is located between 250 nucleotides upstream and 250 nucleotides downstream of the TSS.

In some embodiments, the targeted sequence is located between 100 nucleotides upstream and 100 nucleotides downstream of the TSS.

In some embodiments, the targeted sequence is located between 10 nucleotides upstream and 10 nucleotides downstream of the TSS.

In some embodiments, the targeted sequence is located between 5 nucleotides upstream and 5 nucleotides downstream of the TSS.

In some embodiments, the targeted sequence is located between 1 nucleotide upstream and 1 nucleotide downstream of the TSS.

In some embodiments, the targeted sequence is located upstream of the TSS in the TSS core. The targeted sequence may be less than 2000, less than 1000, less than 500, less than 250, less than 100, less than 10 or less than 5 nucleotides upstream of the TSS.

In some embodiments, the targeted sequence is located downstream of the TSS in the TSS core. The targeted sequence may be less than 2000, less than 1000, less than 500, less than 250, less than 100, less than 10 or less than 5 nucleotides downstream of the TSS.

In some embodiments, the targeted sequence is located +/−50 nucleotides surrounding the TSS of the TSS core. In some embodiments, the targeted sequence substantially overlaps the TSS of the TSS core. In some embodiments, the targeted sequence overlap begins or ends at the TSS of the TSS core. In some embodiments, the targeted sequence overlaps the TSS of the TSS core by 1, 2, 3, 4, 5, 6, 7, 8, 9, 10, 11, 12, 13, 14, 15, 16, 17, 18 or 19 nucleotides in either the upstream or downstream direction.

The location of the targeted sequence on the template strand is defined by the location of the 5′ end of the targeted sequence. The 5′ end of the targeted sequence may be at any position of the TSS core and the targeted sequence may start at any position selected from position 1 to position 4001 of the TSS core. For reference herein, when the 5′ most end of the targeted sequence from position 1 to position 2000 of the TSS core, the targeted sequence is considered upstream of the TSS and when the 5′ most end of the targeted sequence is from position 2002 to 4001, the targeted sequence is considered downstream of the TSS. When the 5′ most end of the targeted sequence is at nucleotide 2001, the targeted sequence is considered to be a TSS centric sequence and is neither upstream nor downstream of the TSS.

For further reference, for example, when the 5′ end of the targeted sequence is at position 1600 of the TSS core, i.e., it is the 1600^{th} nucleotide of the TSS core, the targeted sequence starts at position 1600 of the TSS core and is considered to be upstream of the TSS.

In some embodiments, the TSS core is a sequence for the target gene as described in Tables 1 and 2 of WO2016170348, the contents of which are incorporated herein by reference in their entirety.

In one embodiment, the TSS core is a sequence such as, but not limited to, SEQ ID NO: 1-4047, 315236-318726, 584785-589061, 913310-917531, 1241080-1245401, 1559932-1564372 and 1879189-1889207 of WO2016170348, the contents of which are incorporated herein by reference in their entirety.

In one non-limiting example, the target gene is CCAAT/enhancer-binding protein a (C/EBPα, C/EBP alpha, C/EBPA or CEBPA). CEBPA-saRNAs are provided in the present application to up-regulate CEBPA expression. CEBPA is an intronless gene 2591 nucleotides long with a single TSS. CEBPA TSS core sequence is shown in Table 1.

TABLE 1
CEBPA mRNA and TSS core sequences

| CEBPA mRNA | Protein encoded | CEBPA TSS | SEQ ID of |
|---|---|---|---|
| (target transcript) | by target | core genomic | CEBPA |
| REF. ID No. | transcript | location | TSS core |
| NM_001285829 | NP_001272758 | chr19: 33302564 | 1 |
| | | minus strand | |
| NM_001287424 | NP_001274353 | | |
| NM_001287435 | NP_001274364 | | |
| NM_004364 | NP_004355 | | |

In one embodiment, the saRNA of the present invention may have two strands that form a duplex, one strand being a guide strand. The saRNA duplex is also called a double-stranded saRNA. A double-stranded saRNA or saRNA duplex, as used herein, is an saRNA that includes more than one, and preferably, two, strands in which interstrand hybridization can form a region of duplex structure. The two strands of a double-stranded saRNA are referred to as an antisense strand or a guide strand, and a sense strand or a passenger strand.

The antisense strand of an saRNA duplex, used interchangeably with guide strand of an saRNA, antisense strand saRNA, or antisense saRNA, has a high degree of complementarity to a region within the target antisense RNA transcript. The antisense strand may have no more than 5, or no more than 4 or 3, or no more than 2, or no more than 1, or no mismatches with the region within the target antisense RNA transcript or targeted sequence. Therefore, the antisense strand has a high degree of complementary to the targeted sequence on the template strand. The sense strand of the saRNA duplex, used interchangeably with sense strand saRNA or sense saRNA, has a high degree of sequence identity with the targeted sequence on the template strand. In some embodiments, the targeted sequence is located within the promoter area of the template strand. In some embodiments, the targeted sequence is located within the TSS core of the template stand.

The location of the antisense strand and/or sense strand of the saRNA duplex, relative to the targeted sequence is defined by making reference to the TSS core sequence. For example, when the targeted sequence is downstream of the TSS, the antisense saRNA and the sense saRNA start downstream of the TSS. In another example, when the targeted sequence starts at position 200 of the TSS core, the antisense saRNA and the sense saRNA start upstream of the TSS.

A “strand” in the context of the present invention means a contiguous sequence of nucleotides, including non-naturally occurring or modified nucleotides. Two or more strands may be, or each form a part of, separate molecules, or they may be connected covalently, e.g., by a spacer such as a polyethyleneglycol linker. At least one strand of an saRNA may comprise a region that is complementary to a target antisense RNA. Such a strand is called an antisense or guide strand of the saRNA duplex. A second strand of an saRNA that comprises a region complementary to the antisense strand of the saRNA is called a sense or passenger strand.

An saRNA duplex may also be formed from a single molecule that is at least partly self-complementary forming a hairpin structure, including a duplex region. In such case, the term “strand” refers to one of the regions of the saRNA that is complementary to another internal region of the saRNA. The guide strand of the saRNA will have no more than 5, or no more than 4 or 3, or no more than 2, or no more than 1, or no mismatches with the sequence within the target antisense RNA transcript.

In some embodiments, the passenger strand of an saRNA may comprise at least one nucleotide that is not complementary to the corresponding nucleotide on the guide strand, called a mismatch with the guide strand. The mismatch with the guide strand may encourage preferential loading of the guide strand (Wu et al., PLoS ONE, vol.6 (12):e28580 (2011), the contents of which are incorporated herein by reference in their entirety). In one embodiment, the at least one mismatch with the guide strand may be at 3′ end of the passenger strand. In one embodiment, the 3′ end of the passenger strand may comprise 1-5 mismatches with the guide strand. In one embodiment, the 3′ end of the passenger strand may comprise 2-3 mismatches with the guide strand. In one embodiment, the 3′ end of the passenger strand may comprise 6-10 mismatches with the guide strand.

In one embodiment, an saRNA duplex may show efficacy in proliferating cells.

An saRNA duplex may have siRNA-like complementarity to a region of a target antisense RNA transcript; that is, 100% complementarity between nucleotides 2-6 from the 5′ end of the guide strand in the saRNA duplex and a region of the target antisense RNA transcript. Other nucleotides of the saRNA may, in addition, have at least 80%, 90%, 95%, 98%, 99% or 100% complementarity to a region of the target antisense RNA transcript. For example, nucleotides 7 (counted from the 5′ end) until the 3′ end of the saRNA may have least 80%, 90%, 95%, 98%, 99% or 100% complementarity to a region of the target antisense RNA transcript.

The terms “small interfering RNA” or “siRNA” in the context mean a double-stranded RNA typically 20-25 nucleotides long involved in the RNA interference (RNAi) pathway and interfering with or inhibiting the expression of a specific gene. The gene is the target gene of the siRNA. For example, siRNA that interferes the expression of A3GALT2 gene is called “A3GALT2-siRNA” and the A3GALT2 gene is the target gene. An siRNA is usually about 21 nucleotides long, with 3′ overhangs (e.g., 2 nucleotides) at each end of the two strands.

An siRNA inhibits target gene expression by binding to and promoting the cleavage of one or more RNA transcripts of the target gene at specific sequences. Typically in RNAi the RNA transcripts are mRNA, so cleavage of mRNA results in the down-regulation of gene expression. In the present invention, not willing to be bound with any theory, one of the possible mechanisms is that saRNA of the present invention may modulate the target gene expression by binding to the target antisense RNA transcript. The target antisense RNA transcript may or may not be cleaved.

A double-stranded saRNA may include one or more single-stranded nucleotide overhangs. The term “overhang” or “tail” in the context of double-stranded saRNA and siRNA refers to at least one unpaired nucleotide that protrudes from the duplex structure of saRNA or siRNA. For example, when a 3′-end of one strand of an saRNA extends beyond the 5′-end of the other strand, or vice versa, there is a nucleotide overhang. An saRNA may comprise an overhang of at least one nucleotide; alternatively, the overhang may comprise at least two nucleotides, at least three nucleotides, at least four nucleotides, at least five nucleotides or more. A nucleotide overhang may comprise of consist of a nucleotide/nucleoside analog, including a deoxynucleotide/nucleoside. The overhang(s) may be on the sense strand, the antisense strand or any combination thereof. Furthermore, the nucleotide(s) of an overhang can be present on the 5′ end, 3′ end or both ends of either an antisense or sense strand of an saRNA. Where two oligonucleotides are designed to form, upon hybridization, one or more single-stranded overhangs, and such overhangs shall not be regarded as mismatches with regard to the determination of complementarity. For example, an saRNA comprising one oligonucleotide 19 nucleotides in length and another oligonucleotide 21 nucleotides in length, wherein the longer oligonucleotide comprises a sequence of 19 nucleotides that is fully complementary to the shorter oligonucleotide, can yet be referred to as “fully complementary” for the purposes described herein. The overhang nucleotide may be a natural or a non-natural nucleotide. The overhang may be a modified nucleotide as defined herein.

In one embodiment, the antisense strand of a double-stranded saRNA has a 1-10 nucleotide overhang at the 3′ end and/or the 5′ end. In one embodiment, the antisense strand of a double-stranded saRNA has 1-4 nucleotide overhang at its 3′ end, or 1-2 nucleotide overhang at its 3′ end. In one embodiment, the sense strand of a double-stranded saRNA has a 1-10 nucleotide overhang at the 3′ end and/or the 5′ end. In one embodiment, the sense strand of a double-stranded saRNA has 1-4 nucleotide overhang at its 3′ end, or 1-2 nucleotide overhang at its 3′ end. In one embodiment, both the sense strand and the antisense strand of a double-stranded saRNA have 3′ overhangs. The 3′ overhangs may comprise one or more uracils, e.g., the sequences UU or UUU. In one embodiment, one or more of the nucleotides in the overhang is replaced with a nucleoside thiophosphate, wherein the internucleoside linkage is thiophosphate. In one embodiment, the overhang comprises one or more deoxyribonucleoside, e.g., the sequence dTdT or dTdTdT. In one embodiment, the overhang comprises the sequence dT*dT, wherein ‘*’ is a thiophosphate internucleoside linkage (sometimes referred to as ‘s’). In one embodiment, the overhang comprises at least one 2′-OMe modified U (referred to as u). In one embodiment, the overhang comprises u*u (also referred to as usu). In one embodiment, the overhang comprises uu. In one embodiment, the overhang comprises an inverted nucleotide or nucleoside, which is connected to a strand with reversed linkage (3′-3′ or 5′-5′ linkage). For example, the overhang may comprise an inverted dT, or an inverted abasic nucleoside. An inverted abasic nucleoside does not have a base moiety.

The skilled person will appreciate that it is convenient to define the saRNA of the present invention by reference to the target antisense RNA transcript or the targeted sequence, regardless of the mechanism by which the saRNA modulates the target gene expression. However, the saRNA of the present invention may alternatively be defined by reference to the target gene. The target antisense RNA transcript is complementary to a genomic region on the coding strand of the target gene, and the saRNA of the present invention is in turn complementary to a region of the target antisense RNA transcript, so the saRNA of the present invention may be defined as having sequence identity to a region on the coding strand of the target gene. All of the features discussed herein with respect to the definition of the saRNA of the present invention by reference to the target antisense RNA transcript apply mutatis mutandis to the definition of the saRNA of the present invention by reference to the target gene so any discussion of complementarity to the target antisense RNA transcript should be understood to include identity to the genomic sequence of the target gene. Thus, the saRNA of the present invention may have a high percent identity, e.g. at least 80%, 90%, 95%, 98% or 99%, or 100% identity, to a genomic sequence on the target gene. The genomic sequence may be up to 2000, 1000, 500, 250, or 100 nucleotides upstream or downstream of the target gene's transcription start site. It may align with the target gene's promoter region. Thus, the saRNA may have sequence identity to a sequence that aligns with the promoter region of the target gene.

In one embodiment, the existence of the target antisense RNA transcript does not need to be determined to design the saRNA of the present invention. In another word, the design of the saRNA does not require the identification of the target antisense RNA transcript. For example, the nucleotide sequence of the TSS core, the sequence in the region 2000 nucleotides upstream of the target gene's transcription start site to 2000 nucleotides downstream of the target gene's transcription start may be obtained by the genomic sequence of the coding strand of the target gene, by sequencing or by searching in a database. Targeted sequence within the TSS core starting at any position from position I to position 4001 of the TSS core on the template strand can be selected and can then be used to design saRNA sequences. As discussed above, the saRNA has a high degree of sequence identity with the reverse complement of the targeted sequence.

The saRNA sequence's off-target hit number in the whole genome, 0 mismatch (0 mm) hit number, and 1 mismatch (1 mm) hit number are then determined. The term “off-target hit number” refers to the number of other sites in the whole genome that are identical to the saRNA's targeted sequence on the template strand of the target gene. The term “0 mm hit number” refers to the number of known protein coding transcript other than the target transcript of the saRNA, the complement of which the saRNA may hybridize with or bind to with 0 mismatch. In another word, “0 mm hit number” counts the number of known protein coding transcript, other than the target transcript of the saRNA that comprises a region completely identical with the saRNA sequence. The term “1 mm hit number” refers to the number of known protein coding transcript other than the target transcript of the saRNA, the complement of which the saRNA may hybridize with or bind to with 1 mismatch. In another word, “1 mm hit number” counts the number of known protein coding transcript, other than the target transcript of the saRNA that comprises a region identical with the saRNA sequence with only 1 mismatch. In one embodiment, only saRNA sequences that have no off-target hit, no 0 mm hit and no 1 mm hit are selected. For those saRNA sequences disclosed in the present application, each has no off-target hit, no 0 mm hit and no 1 mm hit.

The method disclosed in US 2013/0164846 filed Jun. 23, 2011 (saRNA algorithm), the contents of which are incorporated herein by reference in their entirety, may also be used to design saRNA. The design of saRNA is also disclosed in U.S. Pat. No. 8,324,181 and U.S. Pat. No. 7,709,566 to Corey et al., US Pat. Pub. No. 2010/0210707 to Li et al., and Voutila et al., Mol Ther Nucleic Acids, vol. 1, e35 (2012), the contents of each of which are incorporated herein by reference in their entirety.

“Determination of existence” means either searching databases of ESTs and/or antisense RNA transcripts around the locus of the target gene to identify a suitable target antisense RNA transcript, or using RT PCR or any other known technique to confirm the physical presence of a target antisense RNA transcript in a cell.

In some embodiments, the saRNA of the present invention may be single or, double-stranded. Double-stranded molecules comprise a first strand and a second strand. If double-stranded, each strand of the duplex may be at least 14, or at least 18, e.g. 19, 20, 21 or 22 nucleotides in length. The duplex may be hybridized over a length of at least 12, or at least 15, or at least 17, or at least 19 nucleotides. Each strand may be exactly 19 nucleotides in length. Preferably, the length of the saRNA is less than 30 nucleotides since oligonucleotide duplex exceeding this length may have an increased risk of inducing the interferon response. In one embodiment, the length of the saRNA is 19 to 25 nucleotides. The strands forming the saRNA duplex may be of equal or unequal lengths.

In one embodiment, the saRNAs of the present invention comprise a sequence of at least 14 nucleotides and less than 30 nucleotides which has at least 80%, 90%, 95%, 98%, 99% or 100% complementarity to the targeted sequence. In one embodiment, the sequence which has at least 80%, 90%, 95%, 98%, 99% or 100% complementarity to the targeted sequence is at least 15, 16, 17, 18 or 19 nucleotides in length, or 18-22 or 19 to 21, or exactly 19.

The saRNA of the present invention may include a short 3′ or 5′ sequence which is not complementary to the target antisense RNA transcript. In one embodiment, such a sequence is at 3′ end of the strand. The sequence may be 1 -5 nucleotides in length, or 2 or 3. The sequence may comprise uracil, so it may be a 3′ stretch of 2 or 3 uracils. The sequence may comprise one or more deoxyribonucleoside, such as dT. In one embodiment, one or more of the nucleotides in the sequence is replaced with a nucleoside thiophosphate, wherein the internucleoside linkage is thiophosphate. As a non-limiting example, the sequence comprises the sequence dT*dT, wherein * is a thiophosphate internucleoside linkage. This non-complementary sequence may be referred to as “tail”. If a 3′ tail is present, the strand may be longer, e.g., 19 nucleotides plus a 3′ tail, which may be UU or UUU. Such a 3′ tail shall not be regarded as mismatches with regard to determine complementarity between the saRNA and the target antisense RNA transcript.

Thus, the saRNA of the present invention may consist of (i) a sequence having at least 80% complementarity to a region of the target antisense RNA transcript; and (ii) a 3′ tail of 1 -5 nucleotides, which may comprise or consist of uracil residues. The saRNA will thus typically have complementarity to a region of the target antisense RNA transcript over its whole length, except for the 3′ tail, if present. Any of the saRNA sequences disclosed in the present application may optionally include such a 3′ tail. Thus, any of the saRNA sequences disclosed in the saRNA Tables and Sequence Listing may optionally include such a 3′ tail. The saRNA of the present invention may further comprise Dicer or Drosha substrate sequences.

The saRNA of the present invention may contain a flanking sequence. The flanking sequence may be inserted in the 3′ end or 5′ end of the saRNA of the present invention. In one embodiment, the flanking sequence is the sequence of a miRNA, rendering the saRNA to have miRNA configuration and may be processed with Drosha and Dicer. In a non-limiting example, the saRNA of the present invention has two strands and is cloned into a microRNA precursor, e.g., miR-30 backbone flanking sequence.

The saRNA of the present invention may comprise a restriction enzyme substrate or recognition sequence. The restriction enzyme recognition sequence may be at the 3′ end or 5′ end of the saRNA of the present invention. Non-limiting examples of restriction enzymes include NotI and AscI.

In one embodiment, the saRNA of the present invention consists of two strands stably base-paired together. In some embodiments, the passenger strand may comprise at least one nucleotide that is not complementary to the corresponding nucleotide on the guide strand, called a mismatch with the guide strand. In one embodiment, the at least one mismatch with the guide strand may be at 3′ end of the passenger strand. In one embodiment, the 3′ end of the passenger strand may comprise 1-5 mismatches with the guide strand. In one embodiment, the 3′ end of the passenger strand may comprise 2-3 mismatches with the guide strand. In one embodiment, the 3′ end of the passenger strand may comprise 6-10 mismatches with the guide strand.

In some embodiments, the double-stranded saRNA may comprise a number of unpaired nucleotides at the 3′ end of each strand forming 3′ overhangs. The number of unpaired nucleotides forming the 3′ overhang of each strand may be in the range of 1 to 5 nucleotides, or 1 to 3 nucleotides, or 2 nucleotides. The 3′ overhang may be formed on the 3′ tail mentioned above, so the 3′ tail may be the 3′ overhang of a double-stranded saRNA.

Thus, the saRNA of the present invention may be single-stranded and consists of (i) a sequence having at least 80% complementarity to a region of the target antisense RNA transcript; and (ii) a 3′ tail of 1 -5 nucleotides, which may comprise uracil residues. The saRNA of the present invention may have complementarity to a region of the target antisense RNA transcript over its whole length, except for the 3′ tail, if present. As mentioned above, instead of “complementary to the target antisense RNA transcript” the saRNA of the present invention may also be defined as having “identity” to the coding strand of the target gene. The saRNA of the present invention may be double-stranded and consists of a first strand comprising (i) a first sequence having at least 80% complementarity to a region of the target antisense RNA transcript and (ii) a 3′ overhang of 1 -5 nucleotides; and a second strand comprising (i) a second sequence that forms a duplex with the first sequence and (ii) a 3′ overhang of 1-5 nucleotides.

As described herein, the genomic sequence of the target gene may be used to design saRNA of the target gene. The sequence of a target antisense RNA transcript may be determined from the sequence of the target gene for designing saRNA of the target gene. However, the existence of such a target antisense RNA transcript does not need to be determined.

One aspect of the present invention provides an saRNA that modulates the expression of a target gene. Also provided is an saRNA that modulates the level of a target transcript. In some embodiments, the target transcript is a coding transcript, e.g., mRNA. Another aspect of the present invention provides an saRNA that modulates the level of a protein encoded by the coding target transcript. In one embodiment, the expression of target gene is increased by at least 20, 30, 40%, or at least 45, 50, 55, 60, 65, 70, 75%, or at least 80% in the presence of the saRNA of the present invention compared to the expression of target gene in the absence of the saRNA of the present invention. In a further embodiment, the expression of target gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the saRNA of the present invention compared to the expression of target gene in the absence of the saRNA of the present invention. The modulation of the expression of target gene may be reflected or determined by the change of mRNA levels encoding the target gene.

The saRNA of the present invention may be produced by any suitable method, for example synthetically or by expression in cells using standard molecular biology techniques which are well-known to a person of ordinary skill in the art. For example, the saRNA of the present invention may be chemically synthesized or recombinantly produced using methods known in the art.

The saRNAs of the present invention may be single-stranded and comprise 14-30 nucleotides. The sequence of a single-stranded saRNA may have at least 60%, 70%, 80% or 90% identity with a sequence such as, but not limited to, SEQ ID NOs: 4048-315235, 318727-584784, 589062-913309, 917532-1241079, 1245402-1559931, 1564373-1879188, and 1889208-2585259 of WO2016170348, the contents of which are incorporated herein by reference in their entirety.

In one embodiment, the single-stranded saRNA comprises a sequence such as, but not limited to, SEQ ID NOs: 4048-315235, 318727-584784, 589062-913309, 917532-1241079, 1245402-1559931, 1564373-1879188, and 1889208-2585259 of WO2016170348, the contents of which are incorporated herein by reference in their entirety.

In one embodiment, the saRNA is a single-stranded saRNA which comprises an antisense sequence such as, but not limited to any of the antisense sequences described in the sequence listing referenced at the beginning of this application.

In one embodiment, the saRNA is a single-stranded saRNA which comprises an antisense sequence such as, but not limited to any of the sense sequences described in the sequence listing referenced at the beginning of this application.

The single stranded saRNAs of the present invention may be modified or unmodified.

In one embodiment, the single-stranded saRNA may have a 3′ tail.

In one embodiment, the saRNAs may be double-stranded. The two strands form a duplex, also known as an saRNA duplex, and each strand comprises 14-30 nucleotides. The first strand of a double-stranded saRNA may have at least 60%, 70%, 80% or 90% identity with a sequence such as, but not limited to, SEQ ID NOs: 4048-315235, 318727-584784, 589062-913309, 917532-1241079, 1245402-1559931, 1564373-1879188, and 1889208-2585259 of WO2016170348, the contents of which are incorporated herein by reference in their entirety. In one embodiment, the first strand of the double-stranded saRNA comprises a sequence such as, but not limited to, SEQ ID NOs: 4048-315235, 318727-584784, 589062-913309, 917532-1241079, 1245402-1559931, 1564373-1879188, and 1889208-2585259. The second strand of a double-stranded saRNA may have at least 60%, 70%, 80% or 90% identity with a sequence such as, but not limited to, SEQ ID NOs: 4048-315235, 318727-584784, 589062-913309, 917532-1241079, 1245402-1559931, 1564373-1879188, and 1889208-2585259 of WO2016170348, the contents of which are incorporated herein by reference in their entirety. In one embodiment, the second strand of the double-stranded saRNA comprises a sequence such as, but not limited to, SEQ ID NOs: 4048-315235, 318727-584784, 589062-913309, 917532-1241079, 1245402-1559931, 1564373-1879188, and 1889208-2585259 of WO2016170348, the contents of which are incorporated herein by reference in their entirety. In one embodiment, the double-stranded saRNA may have a 3′ overhang on each strand.

In one embodiment, the saRNA of the present invention is an saRNA duplex. The saRNA duplex may be a pair of sense and antisense sequences such as, but not limited to, any of the sense sequence and corresponding antisense sequences described in the sequence listing referenced at the beginning of this application. The saRNA of the present invention may be the pair of the sense sequence and antisense sequence described in the sequence listing referenced at the beginning of this application.

The double-stranded saRNA of the present invention may be modified or unmodified.

### Bifunction Oligonucleotides

Bifunction or dual-functional oligonucleotides, e.g., saRNA may be designed to up-regulate the expression of a first gene and down-regulate the expression of at least one second gene. One strand of the dual-functional oligonucleotide activates the expression of the first gene and the other strand inhibits the expression of the second gene. Each strand might further comprise a Dicer substrate sequence.

Chemical Modifications of saRNA

Herein, in saRNA, the terms “modification” or, as appropriate, “modified” refer to structural and/or chemical modifications with respect to A, G, U or C ribonucleotides. Nucleotides in the saRNAs of the present invention may comprise non-standard nucleotides, such as non-naturally occurring nucleotides or chemically synthesized nucleotides or deoxynucleotides. The saRNA of the present invention may include any useful modification, such as to the sugar, the nucleobase, or the internucleoside linkage (e.g. to a linking phosphate/to a phosphodiester linkage/to the phosphodiester backbone). One or more atoms of a pyrimidine nucleobase may be replaced or substituted with optionally substituted amino, optionally substituted thiol, optionally substituted alkyl (e.g., methyl or ethyl), or halo (e.g., chloro or fluoro). In certain embodiments, modifications (e.g., one or more modifications) are present in each of the sugar and the internucleoside linkage. Modifications according to the present invention may be modifications of ribonucleic acids (RNAs) to deoxyribonucleic acids (DNAs), threose nucleic acids (TNAs), glycol nucleic acids (GNAs), peptide nucleic acids (PNAs), locked nucleic acids (LNAs) or hybrids thereof. In a non-limiting example, the 2′-OH of U is substituted with 2′-OMe.

In one embodiment, the saRNAs of the present invention may comprise at least one modification described herein.

In another embodiment, the saRNA is an saRNA duplex and the sense strand and antisense sequence may independently comprise at least one modification. As a non-limiting example, the sense sequence may comprise a modification and the antisense strand may be unmodified. As another non-limiting example, the antisense sequence may comprise a modification and the sense strand may be unmodified. As yet another non-limiting example, the sense sequence may comprise more than one modification and the antisense strand may comprise one modification. As a non-limiting example, the antisense sequence may comprise more than one modification and the sense strand may comprise one modification.

The saRNA of the present invention can include a combination of modifications to the sugar, the nucleobase, and/or the internucleoside linkage. These combinations can include any one or more modifications described herein or in International Application Publication WO2013/052523 filed Oct. 3, 2012, in particular Formulas (Ia)-(Ia-5), (Ib)-(If), (IIa)-(IIp), (IIb-1), (IIb-2), (IIc-1)-(IIc-2), (IIn-1), (IIn-2), (IVa)-(IV1), and (IXa)-(IXr)), the contents of which are incorporated herein by reference in their entirety.

The saRNA of the present invention may or may not be uniformly modified along the entire length of the molecule. For example, one or more or all types of nucleotide (e.g., purine or pyrimidine, or any one or more or all of A, G, U, C) may or may not be uniformly modified in the saRNA of the invention. In some embodiments, all nucleotides X in an saRNA of the invention are modified, wherein X may be any one of nucleotides A, G, U, C, or any one of the combinations A+G, A+U, A+C, G+U, G+C, U+C, A+G+U, A+G+C, G+U+C or A+G+C.

Different sugar modifications, nucleotide modifications, and/or internucleoside linkages (e.g., backbone structures) may exist at various positions in an saRNA. One of ordinary skill in the art will appreciate that the nucleotide analogs or other modification(s) may be located at any position(s) of an saRNA such that the function of saRNA is not substantially decreased. The saRNA of the present invention may contain from about 1% to about 100% modified nucleotides (either in relation to overall nucleotide content, or in relation to one or more types of nucleotide, i.e. any one or more of A, G, U or C) or any intervening percentage (e.g., from 1% to 20%, from 1% to 25%, from 1% to 50%, from 1% to 60%, from 1% to 70%, from 1% to 80%, from 1% to 90%, from 1% to 95%, from 10% to 20%, from 10% to 25%, from 10% to 50%, from 10% to 60%, from 10% to 70%, from 10% to 80%, from 10% to 90%, from 10% to 95%, from 10% to 100%, from 20% to 25%, from 20% to 50%, from 20% to 60%, from 20% to 70%, from 20% to 80%, from 20% to 90%, from 20% to 95%, from 20% to 100%, from 50% to 60%, from 50% to 70%, from 50% to 80%, from 50% to 90%, from 50% to 95%, from 50% to 100%, from 70% to 80%, from 70% to 90%, from 70% to 95%, from 70% to 100%, from 80% to 90%, from 80% to 95%, from 80% to 100%, from 90% to 95%, from 90% to 100%, and from 95% to 100%).

In some embodiments, the saRNA of the present invention may be modified to be a circular nucleic acid. The terminals of the saRNA of the present invention may be linked by chemical reagents or enzymes, producing circular saRNA that has no free ends. Circular saRNA is expected to be more stable than its linear counterpart and to be resistant to digestion with RNase R exonuclease. Circular saRNA may further comprise other structural and/or chemical modifications with respect to A, G, U or C ribonucleotides.

The saRNA of the present invention may be modified with any modifications of an oligonucleotide or polynucleotide disclosed in pages 136 to 247 of PCT Publication WO2013/151666 published Oct. 10, 2013, the contents of which are incorporated herein by reference in their entirety.

The saRNA of the present invention may comprise a combination of modifications. The saRNA may comprise at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 modifications for each strand.

In some embodiments, the saRNA is at least 50% modified, e.g., at least 50% of the nucleotides are modified. In some embodiments, the saRNA is at least 75% modified, e.g., at least 75% of the nucleotides are modified. In some embodiments, both strands of the saRNA may be modified across the whole length (100% modified). It is to be understood that since a nucleotide (sugar, base and phosphate moiety, e.g., linker) may each be modified, any modification to any portion of a nucleotide, or nucleoside, will constitute a modification.

In some embodiments, the saRNA is at least 10% modified in only one component of the nucleotide, with such component being selected from the nucleobase, sugar or linkage between nucleosides. For example, modifications of an saRNA may be made to at least 10%, 20%, 30%, 40%, 50%, 60%, 70%, 80%, 90% or 100% of the nucleobases, sugars or linkages of said saRNA.

In some embodiments, the saRNA comprises at least one sugar modification. Nonlimiting examples of the sugar modification may include the following:

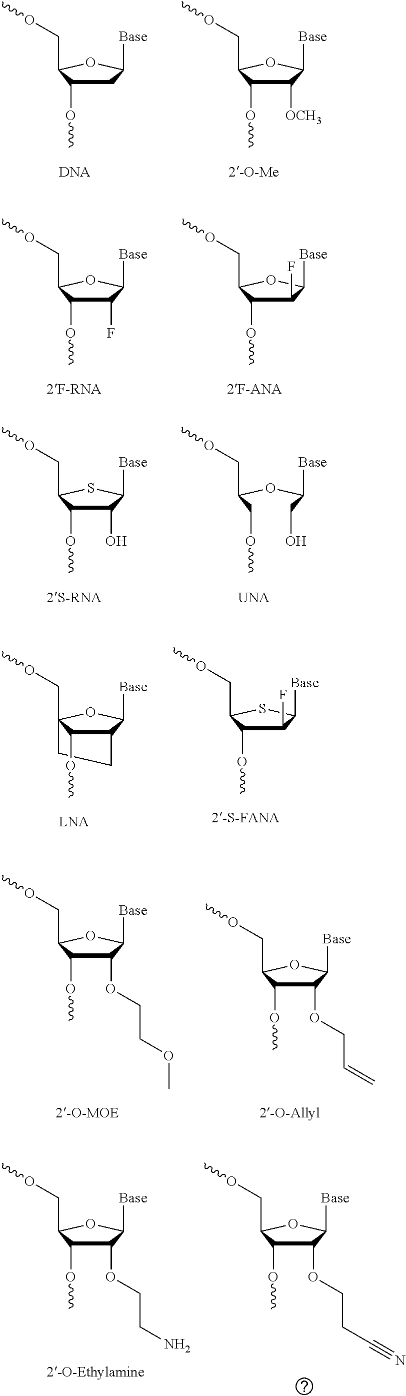

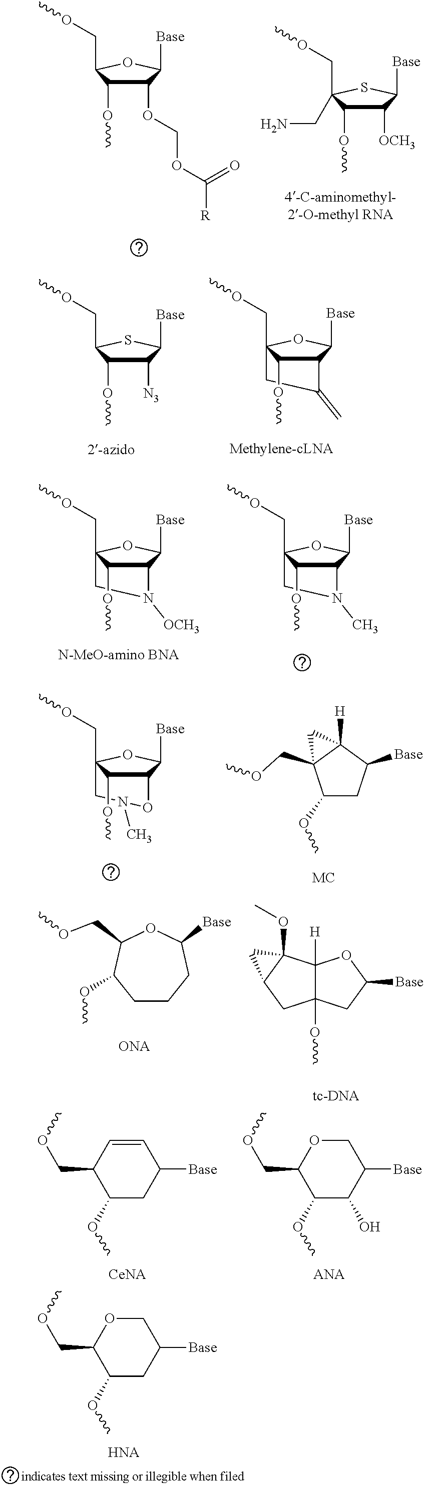

In some embodiments, at least one of the 2′ positions of the sugar (OH in RNA or H in DNA) of a nucleotide of the saRNA is substituted with —OMe, referred to as 2′-OMe.

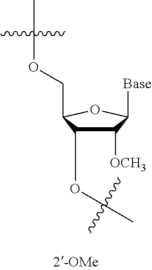

In some embodiments, at least one of the 2′ positions_of the sugar (OH in RNA or H in DNA) of a nucleotide of the saRNA is substituted with —F, referred to as 2′-F.

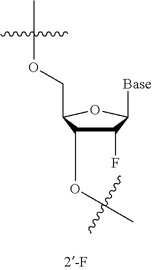

In some embodiments, the saRNA comprises at least one phosphorothioate linkage or methylphosphonate linkage between nucleotides.

In some embodiments, the saRNA comprises 3′ and/or 5′ capping or overhang. In some embodiments, the saRNA of the present invention may comprise at least one inverted deoxyribonucleoside overhang (e.g., dT). The inverted overhang, e.g., dT, may be at the 5′ terminus or 3′ terminus of the passenger (sense) strand. In some embodiments, the saRNA of the present invention may comprise inverted abasic modifications on the passenger strand. The at least one inverted abasic modification may be on 5′ end, or 3′ end, or both ends of the passenger strand. The inverted abasic modification may encourage preferential loading of the guide (antisense) strand.

In some embodiments, the saRNA comprises at least one 5′-(E)-vinylphosphonate (5′-E-VP) modification.

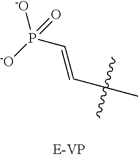

In some embodiments, the saRNA comprises at least one glycol nucleic acid (GNA), an acyclic nucleic acid analogue, as a modification.

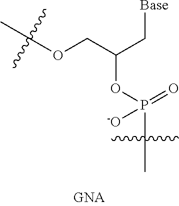

In some embodiments, the saRNA at least one motif of at least 2 consecutive nucleotides that have the same sugar modification. In one example, such a motif may comprise 2 or 3 consecutive nucleotides. In some embodiments, the consecutive nucleotides of the motif comprise 2′-F modifications. In some embodiments, the consecutive nucleotides of the motif comprise 2′-OMe modifications.

In some embodiments, when the saRNA is double-stranded, the passenger strand and the guide strand of the saRNA each comprises at least one motif of consecutive nucleotides that have the same sugar modification.

In some embodiments, the passenger strand and the guide strand of the saRNA each comprises at least two motifs of consecutive nucleotides that have the same sugar modification. In some embodiments, the at least two motifs on a given strand independently have different sugar modifications. For example, the passenger strand or the guide strand may have at least one motif of 2′-OMe modifications and at least one motif of 2′-F modifications. In some embodiments, the at least two motifs on a given strand are separated by at least one (such as 1, 2, 3, 4, 5, 6, 7, 8, 9, 10, 11, 12, 13, 14, 15, 16, 17, 18, 19, or 20) nucleotide. In some embodiments, the at least two motifs on a given strand are connected. In some embodiments, at least one motif on the passenger strand and its complementary motif on the guide strand have different sugar modifications. For example, the nucleotides of a motif on the passenger strand has 2′-F modifications and the nucleotides of a motif on the guide strand has 2′-OMe modifications, wherein the two motifs are complimentary to each other. In another example, the nucleotides of a motif on the passenger strand has 2′-OMe modifications and the nucleotides of a motif on the guide strand has 2′-F modifications, wherein the two motifs are complimentary to each other.

In some embodiments, the modification of a motif is different from the modifications of the immediately adjacent nucleotides on both sides of the motif

In some embodiments, the saRNA comprises at least one motif of alternating sugar modifications. In one example, the motif of alternating sugar modifications comprises 2 to 30 nucleotides. In some embodiments, the motif comprises alternating 2′-F and 2′-OMe modifications.

In some embodiments, when the saRNA is double stranded, the passenger strand and the guide strand each comprises at least one motif of alternating sugar modifications. In some embodiments, at least one nucleotide on the passenger strand and its complementary nucleotide on the guide strand have different sugar modifications. For example, one nucleotide of the base pair on the passenger strand has a 2′-F modification and the other nucleotide of the base pair on the guide strand has a 2′-OMe modification. In another example, one nucleotide of the base pair on the passenger strand has a 2′-OMe modification and the other nucleotide of the base pair on the guide strand has a 2′-F modification.

In some embodiments, the saRNA is double-stranded and has general formula of:

Passenger (Sense or SS): 5′ overhang1—NT1—(XXX-NT2)n—overhang2 3′,

Guide (Antisense or AS): 3′ overhang3—NT1′—(YYY-NT2′)n—overhang4 5′, (I)

wherein:

each strand is 14-30 nucleotides in length,

each of overhang1, overhang2, overhang3 and overhang4 independently represents an oligonucleotide sequence comprising 0-5 nucleotides,

NT1 and NT 1′ represent an oligonucleotide sequence comprising 0-20 nucleotides, and wherein NT1 is complementary to NT1′,

each of XXX-NT2 and YYY-NT2′ independently represents a motif of consecutive nucleotides, wherein the first 3 consecutive nucleotides have the same chemical modification, followed by an oligonucleotide sequence comprising 0-20 nucleotides, and wherein XXX is complementary to YYY, and NT2 is complementary to NT2′,

each of NT1, NT2, NT1′, and NT2′ comprises at least one chemical modification, and n is a number between 1 and 5.

The guide strand of the saRNA having formula (I) comprises a sequence that is at least 80% identical to the reverse complement of a targeted sequence located in the TSS core on the template strand of the target gene. In another word, the guide strand of the saRNA having formula (I) comprises a sequence that is at least 80% complementary to a targeted sequence located in the TSS core on the template strand of the target gene. “Targeted sequence” and “TSS core” are defined above.

The 3 consecutive nucleotides in XXX and YYY don't need to the same. They only need to have the same chemical modification.

In some cases, each strand comprises 14-17 nucleotides, 17-25 nucleotides, 17-23 nucleotides, 23-27 nucleotides, 19-21 nucleotides, 21-23 nucleotides, or 27-30 nucleotides.

In some cases, each strand comprises at least one sugar modification.

In some cases, at least one nucleotide on the passenger strand and its complementary nucleotide on the guide strand have different sugar modifications.

In some cases, NT1, NT2, NT1′, and NT2′ have alternating modifications, such as alternating 2′-OMe and 2′-F modifications.

In some cases, the 3 consecutive nucleotides of XXX have 2′-OMe modifications and the 3 consecutive nucleotides of YYY have 2′-F modifications.

In some cases, the 3 consecutive nucleotides of XXX have 2′-F modifications and the 3 consecutive nucleotides of YYY have 2′-OMe modifications.

In some cases, the modification of XXX or YYY is different from the modifications of the immediately adjacent nucleotides on both sides of XXX or YYY.

In some cases, the YYY motif may start at the 8^{th}, 9^{th}, 10^{th}, 11^{th}, 12^{th}, or 13^{th} position of the antisense strand from the 5′ end.

In some cases, the XXX motif may start at the 8^{th}, 9^{th}, 10^{th}, 11^{th}, 12^{th}, or 13^{th} position of the sense strand from the 3′ end.

In some cases, overhang1, overhang2, overhang3 and/or overhang4 comprises uu.

In some cases, overhang1, overhang2, and/or overhang3 comprises an inverted dT.

In some cases, overhang1, overhang2, and/or overhang3 comprises an inverted abasic nucleoside.

In some cases, the saRNA comprises at least one phosphorothioate linkage (

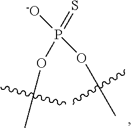

referred to as s in the sequences) or methylphosphonate linkage between the nucleotides. The phosphorothioate linkage or methylphosphonate linkage may be at the 3′ end of one strand, e.g., sense strand or antisense strand. For example, the overhang on the 3′ end of the antisense strand may be: usu.

In some cases, the passenger strand of the saRNA comprises a linker at its 3′ end or 5′ end, which enables a moiety to be attached to the 3′ end or 5′ end of the passenger strand. Overhang1 or overhang2 may comprise the linker. The linker may be any suitable linker, such as NH₂—(CH₂)₆— (referred to as NH2C6 in the sequences). There may be a phosphorothioate linkage between the linker and the passenger strand.

In some embodiments, the modified saRNA has improved stability compared with the non-modified version. The serum half-life of the modified saRNA may be longer than the non-modified version by about at least about 6 hours, about 12 hours, about 18 hours, about 24 hours, about 30 hours, about 36 hours, about 42 hours, about 48 hours, about 54 hours, 60 hours, 66 hours, 72 hours, 78 hours, 84 hours, 90 hours, or 96 hours. In some embodiments, the modified saRNA has a half-life of at least 48 hours, 60 hours, 72 hours, 84 hours, or 96 hours.

In some embodiments, the saRNA up-regulates CEBPA. Non-limiting examples of CEBPA-saRNA sequences with at least one modification include the saRNAs in Table 2. The parent sequence has no modification.

TABLE 2
Modified CEBPA-saRNA sequences

| | | | SEQ |
|---|---|---|---|
| Name | | Sequences | ID No. |
| Parent | SS 5′-3′ | GCGGUCAUUGUCACUGGUCUU | 2 |
| (XD- | | 21-mer | |
| 03302) | AS 5′-3′ | GACCAGUGACAAUGACCGCUU | 3 |
| | | 21-mer | |
| S1 (XD- | SS 5′-3′ | (NH2C6)sGCGGUCAUUGUCA | 4 |
| 06409) | | CUGGUCUU(invdT) | |
| | AS 5′-3′ | GACCAGUGACAAUGACCGCUs | 5 |
| | | U | |
| S2 (XD- | SS 5′-3′ | (NH2C6)sGfcGfgUfcAfuU | 6 |
| 06410) | | fgUfcAfcUfgGfuCfuu | |
| | | (invdT) | |
| | AS 5′-3′ | gAfcCfaGfuGfaCfaAfuGf | 7 |
| | | aCfcGfcusu | |
| S3 (XD- | SS 5′-3′ | (NH2C6)sGfcGfgUfcAfuU | 8 |
| 06411) | | fgUfcAfcUfgGfuCfuu | |
| | | (invdT) | |
| | AS 5′-3′ | gAfcCfaGfuGfaCfaAfuGf | 9 |
| | | aCfcGfcsusu | |
| S4 (XD- | SS 5′-3′ | (NH2C6)sGfcGfgUfcAfuU | 10 |
| 06412) | | fGfUfcAfcUfgGfuCfuu | |
| | | (invdT) | |
| | AS 5′-3′ | gAfcCfaGfuGfacaAfuGfa | 11 |
| | | CfcGfcsusu | |
| S5 (XD- | SS 5′-3′ | (NH2C6)sGfcGfgUfcAfuA | 12 |
| 06413) | | fCfAfcAfcUfgGfuCfuu | |
| | | (invdT) | |
| | AS 5′-3′ | gAfcCfaGfuGfuguAfuGfa | 13 |
| | | CfcGfcsusu | |
| S6 (XD- | SS 5′-3′ | (NH₂C6)sGfcGfgUfcAfUf | 14 |
| 06414) | | UfgUfcAfcUfgGfuCfuu | |
| | | (invdT) | |
| | AS 5′-3′ | gAfcCfaGfuGfaCfaauGfa | 15 |
| | | CfcGfcsusu | |
| S7 (XD- | SS 5′-3′ | (NH2C6)sgCfgGfuCfaUfu | 16 |
| 06415) | | GfuCfaCfuGfgUfcuu | |
| | | (invdT) | |
| | AS 5′-3′ | GfaCfcAfgUfgAfcAfaUfg | 17 |
| | | AfcCfgCfsusu | |
| S8 | SS 5′-3′ | GfcGfgUfcAfUfUfgUfcAf | 18 |
| | | cUfgGfuCfuu(invdT) | |
| | AS 5′-3′ | gAfcCfaGfuGfaCfaauGfa | 19 |
| | | CfcGfcsusu | |
| XD-07139 | SS 5′-3′ | (invdT)sGfcGfgUfcAfUf | 20 |
| | | UfgUfcAfcUfgGfuCfuu | |
| | | (invdT) | |
| | AS 5′-3′ | gAfcCfaGfuGfaCfaauGfa | 21 |
| | | CfcGfcsusu | |
| XD-03934 | SS 5′-3′ | (invabasic)gcGgUCAUUg | 22 |
| | | UCAcUGGUCuu | |
| | AS 5′-3′ | GACCAGUGACAAUGACCGCuu | 23 |
| XD- | SS 5′-3′ | GfscsGfgUfcAfUfUfgUfc | 24 |
| 14369K1 | | AfcUfgGfuCf | |
| | AS 5′-3′ | GfsAfscCfaGfuGfaCfaau | 25 |
| | | GfaCfcGfcsUfsu | |

Nf = the nucleotide N (N may be A, U, C, or G) has a 2′-Fluoro (2′-F) modification
lower case = the nucleotide has a 2′-O-Methyl (2′-OMe) modification
s: phosphorothioate linkage
invdT: inverted deoxy T (dT)
invabasic: inverted abasic nucleotide

The (NH2C6) linker on any modified saRNA may be replaced with another suitable linker.

In one embodiment, the CEBPA-saRNA comprises formula (I). The antisense strand of the CEBPA-saRNA is at least 80% identical to the reverse complement of a region on the CEBPA TSS core. Non-limiting examples of CEBPA-saRNA having general formula (I) include S6 (XD-06414, SEQ ID Nos. 14 and 15).

saRNA Conjugates and Combinations

Conjugation may result in increased stability and/or half-life and may be particularly useful in targeting the saRNA of the present invention to specific sites in the cell, tissue or organism. The saRNA of the present invention can be designed to be conjugated to other polynucleotides, dyes, intercalating agents (e.g. acridines), cross-linkers (e.g. psoralene, mitomycin C), porphyrins (TPPC4, texaphyrin, Sapphyrin), polycyclic aromatic hydrocarbons (e.g., phenazine, dihydrophenazine), artificial endonucleases (e.g. EDTA), alkylating agents, phosphate, amino, mercapto, PEG (e.g., PEG-40K), MPEG, [MPEG]₂, polyamino, alkyl, substituted alkyl, radiolabeled markers, enzymes, haptens (e.g. biotin), transport/absorption facilitators (e.g., aspirin, vitamin E, folic acid), synthetic ribonucleases, proteins, e.g., glycoproteins, or peptides, e.g., molecules having a specific affinity for a co-ligand, or antibodies e.g., an antibody, that binds to a specified cell type such as a cancer cell, endothelial cell, or bone cell, hormones and hormone receptors, non-peptidic species, such as lipids, lectins, carbohydrates, vitamins, cofactors, or a drug. Suitable conjugates for nucleic acid molecules are disclosed in International Publication WO 2013/090648 filed Dec. 14, 2012, the contents of which are incorporated herein by reference in their entirety.

According to the present invention, saRNA of the present invention may be administered with, or further include one or more of RNAi agents, small interfering RNAs (siRNAs), small hairpin RNAs (shRNAs), long non-coding RNAs (lncRNAs), enhancer RNAs, enhancer-derived RNAs or enhancer-driven RNAs (eRNAs), microRNAs (miRNAs), miRNA binding sites, antisense RNAs, ribozymes, catalytic DNA, tRNA, RNAs that induce triple helix formation, aptamers or vectors, and the like to achieve different functions. The one or more RNAi agents, small interfering RNAs (siRNAs), small hairpin RNAs (shRNAs), long non-coding RNAs (lncRNA), microRNAs (miRNAs), miRNA binding sites, antisense RNAs, ribozymes, catalytic DNA, tRNA, RNAs that induce triple helix formation, aptamers or vectors may comprise at least one modification or substitution.

In some embodiments, the modification is selected from a chemical substitution of the nucleic acid at a sugar position, a chemical substitution at a phosphate position and a chemical substitution at a base position. In other embodiments, the chemical modification is selected from incorporation of a modified nucleotide; 3′ capping; conjugation to a high molecular weight, non-immunogenic compound; conjugation to a lipophilic compound; and incorporation of phosphorothioate into the phosphate backbone. In one embodiment, the high molecular weight, non-immunogenic compound is polyalkylene glycol, or polyethylene glycol (PEG).

In one embodiment, saRNA comprising at least one modification may show efficacy in proliferating cells.

In one embodiment, saRNA of the present invention may be attached to a transgene so it can be co-expressed from an RNA polymerase II promoter. In a non-limiting example, saRNA of the present invention is attached to green fluorescent protein gene (GFP).

In one embodiment, saRNA of the present invention may be attached to a DNA or RNA aptamer, thereby producing saRNA-aptamer conjugate. Aptamers are oligonucleotides or peptides with high selectivity, affinity and stability. They assume specific and stable three-dimensional shapes, thereby providing highly specific, tight binding to target molecules. An aptamer may be a nucleic acid species that has been engineered through repeated rounds of in vitro selection or equivalently, SELEX (systematic evolution of ligands by exponential enrichment) to bind to various molecular targets such as small molecules, proteins, nucleic acids, and even cells, tissues and organisms. Nucleic acid aptamers have specific binding affinity to molecules through interactions other than classic Watson-Crick base pairing. Nucleic acid aptamers, like peptides generated by phage display or monoclonal antibodies (mAbs), are capable of specifically binding to selected targets and, through binding, block their targets' ability to function. In some cases, aptamers may also be peptide aptamers. For any specific molecular target, nucleic acid aptamers can be identified from combinatorial libraries of nucleic acids, e.g. by SELEX. Peptide aptamers may be identified using a yeast two hybrid system. A skilled person is therefore able to design suitable aptamers for delivering the saRNAs or cells of the present invention to target cells such as liver cells. DNA aptamers, RNA aptamers and peptide aptamers are contemplated. Administration of saRNA of the present invention to the liver using liver-specific aptamers is preferred.

As used herein, a typical nucleic acid aptamer is approximately 10-15 kDa in size (20-45 nucleotides), binds its target with at least nanomolar affinity, and discriminates against closely related targets. Nucleic acid aptamers may be ribonucleic acid, deoxyribonucleic acid, or mixed ribonucleic acid and deoxyribonucleic acid. Aptamers may be single-stranded ribonucleic acid, deoxyribonucleic acid or mixed ribonucleic acid and deoxyribonucleic acid. Aptamers may comprise at least one chemical modification.

A suitable nucleotide length for an aptamer ranges from about 15 to about 100 nucleotides (nt), and in various other embodiments, 15-30 nt, 20-25 nt, 30-100 nt, 30-60 nt, 25-70 nt, 25-60 nt, 40-60 nt, 25-40 nt, 30-40 nt, any of 22, 23, 24, 25, 26, 27, 28, 29, 30, 31, 32, 33, 34, 35, 36, 37, 38, 39 or 40 nt or 40-70 nt in length. However, the sequence can be designed with sufficient flexibility such that it can accommodate interactions of aptamers with two targets at the distances described herein. Aptamers may be further modified to provide protection from nuclease and other enzymatic activities. The aptamer sequence can be modified by any suitable methods known in the art.

The saRNA-aptamer conjugate may be formed using any known method for linking two moieties, such as direct chemical bond formation, linkage via a linker such as streptavidin and so on.

In one embodiment, saRNA of the present invention may be attached to an antibody. Methods of generating antibodies against a target cell surface receptor are well known. The saRNAs of the invention may be attached to such antibodies with known methods, for example using RNA carrier proteins. The resulting complex may then be administered to a subject and taken up by the target cells via receptor-mediated endocytosis.

In one embodiment, saRNA of the present invention may be conjugated with lipid moieties such as a cholesterol moiety (Letsinger et al., Proc. Natl. Acid. Sci. USA, 1989, 86: 6553-6556), cholic acid (Manoharan et al., Biorg. Med. Chem. Let., 1994, 4:1053-1060), a thioether, e.g., beryl-5-tritylthiol (Manoharan et al., Ann. N.Y. Acad. Sci., 1992, 660:306-309; Manoharan et al., Biorg. Med. Chem. Let., 1993, 3:2765-2770), a thiocholesterol (Oberhauser et al., Nucl. Acids Res., 1992, 20:533-538), an aliphatic chain, e.g., dodecandiol or undecyl residues (Saison-Behmoaras et al., EMBO J, 1991, 10:1111-1118; Kabanov et al., FEBS Lett., 1990, 259:327-330; Svinarchuk et al., Biochimie, 1993, 75:49-54), a phospholipid, e.g., di-hexadecyl-rac-glycerol or triethyl-ammonium 1,2-di-O-hexadecyl-rac-glycero-3-Hphosphonate (Manoharan et al., Tetrahedron Lett., 1995, 36:3651-3654; Shea et al., Nucl. Acids Res., 1990, 18:3777-3783), a polyamine or a polyethylene glycol chain (Manoharan et al., Nucleosides & Nucleotides, 1995, 14:969-973), or adamantane acetic acid (Manoharan et al., Tetrahedron Lett., 1995, 36:3651-3654), a palmityl moiety (Mishra et al., Biochim. Biophys. Acta, 1995, 1264:229-237), or an octadecylamine or hexylamino-carbonyloxycholesterol moiety (Crooke et al., J. Pharmacol. Exp. Ther., 1996, 277:923-937), the content of each of which is herein incorporated by reference in its entirety.

In one embodiment, the saRNA of the present invention is conjugated with a ligand. In one non-limiting example, the ligand may be any ligand disclosed in US 20130184328 to Manoharan et al., the contents of which are incorporated herein by reference in their entirety. The conjugate has a formula of Ligand-[linker]_{optical}-[tether]_{optical}-oligonucleotide agent. The oligonucleotide agent may comprise a subunit having formulae (I) disclosed by US 20130184328 to Manoharan et al., the contents of which are incorporated herein by reference in their entirety. In another non-limiting example, the ligand may be any ligand disclosed in US 20130317081 to Akinc et al., the contents of which are incorporated herein by reference in their entirety, such as a lipid, a protein, a hormone, or a carbohydrate ligand of Formula II-XXVI. The ligand may be coupled with the saRNA with a bivalent or trivalent branched linker in Formula XXXI-XXXV disclosed in Akinc.

Representative U.S. patents that teach the preparation of such nucleic acid/lipid conjugates include, but are not limited to, U.S. Pat. Nos. 4,828,979; 4,948,882; 5,218,105; 5,525,465; 5,541,313; 5,545,730; 5,552,538; 5,578,717, 5,580,731; 5,591,584; 5,109,124; 5,118,802; 5,138,045; 5,414,077; 5,486,603; 5,512,439; 5,578,718; 5,608,046; 4,587,044; 4,605,735; 4,667,025; 4,762,779; 4,789,737; 4,824,941; 4,835,263; 4,876,335; 4,904,582; 4,958,013; 5,082,830; 5,112,963; 5,214,136; 5,082,830; 5,112,963; 5,214,136; 5,245,022; 5,254,469; 5,258,506; 5,262,536; 5,272,250; 5,292,873; 5,317,098; 5,371,241, 5,391,723; 5,416,203, 5,451,463; 5,510,475; 5,512,667; 5,514,785; 5,565,552; 5,567,810; 5,574,142; 5,585,481; 5,587,371; 5,595,726; 5,597,696; 5,599,923; 5,599,928 and 5,688,941, the content of each of which is herein incorporated by reference in its entirety.

The saRNA of the present invention may be provided in combination with other active ingredients known to have an effect in the particular method being considered. The other active ingredients may be administered simultaneously, separately, or sequentially with the saRNA of the present invention. In one embodiment, saRNA of the present invention is administered with saRNA modulating a different target gene. Non-limiting examples include saRNA that modulates albumin, insulin or HNF4A genes. Modulating any gene may be achieved using a single saRNA or a combination of two or more different saRNAs. Non-limiting examples of saRNA that can be administered with saRNA of the present invention include saRNA modulating albumin or HNF4A disclosed in International Publication WO 2012/175958 filed Jun. 20, 2012, saRNA modulating insulin disclosed in International Publications WO 2012/046084 and WO 2012/046085 both filed Oct. 10, 2011, saRNA modulating human progesterone receptor, human major vault protein (hMVP), E-cadherin gene, p53 gene, or PTEN gene disclosed in U.S. Pat. No. 7,709,456 filed Nov. 13, 2006 and US Pat. Publication US 2010/0273863 filed Apr. 23, 2010, saRNAs targeting p21 gene disclosed in International Publication WO 2006/113246 filed Apr. 11, 2006, any nucleic acid disclosed in WO2012/065143 filed Nov. 12, 2011 that upregulates the expression of genes in Table 8 of WO 2012/065143 or increases the expression of a tumor suppressor, any oligonucleotide that activates target genes in Table 4 of WO2013/173635 filed May 16, 2013, any oligonucleotide that activates target genes in Table 4 of WO2013/173637 filed May 16, 2013, any oligonucleotide complementary to a sequence selected from the sequences in SEQ ID NOs: 1-1212 of WO2013/173652 filed May 16, 2013, any oligonucleotide modulating APOA1 and ABCA1 gene expressions disclosed in WO2013173647 filed May 16, 2013, any oligonucleotide modulating SMN family gene expressions disclosed in WO2013173638 filed May 16, 2013, any oligonucleotide modulating PTEN gene expression disclosed in WO2013173605 filed May 16, 2013, any oligonucleotide modulating MECP2 gene expression disclosed in WO2013173608 filed May 16, 2013, any oligonucleotide modulating ATP2A2 gene expression disclosed in WO2013173598 filed May 16, 2013, any oligonucleotide modulating UTRN gene expression disclosed in WO2013173645 filed May 16, 2013, any nucleic acid molecule that modulates the expression of CD97, TS-α, C/EBP delta, CDC23, PINK1, HIF1α, Gnbp3g, Adrenomedullin AM1 receptor, 3-oxoacid CoA transferase, Cathepsin W or BACE1 disclosed in U.S. Pat. No. 8,288,354 filed Dec. 28, 2006, antagoNAT with formula (I) disclosed in US 2013/0245099 filed Nov. 17, 2011, any antagoNAT that upregulates the expression of hemoglobin (HBF/HBG) polynucleotides disclosed in U.S. Pat. No. 8,318,690 filed Apr. 30, 2010, any antisense oligonucleotide that increases the expression of apolipoprotein (ApoA1) polynucleotide disclosed in U.S. Pat. No. 8,153,696 filed Oct. 2, 2009 (CURNA), the contents of each of which are incorporated herein by reference in their entirety.

In on embodiment, the saRNA is conjugated with a carbohydrate ligand, such as any carbohydrate ligand disclosed in U.S. Pat. No. 8,106,022 and 8,828,956 to Manoharan et al. (Alnylam Pharmaceuticals), the contents of which are incorporated herein by reference in their entirety. For example, the carbohydrate ligand may be monosaccharide, disaccharide, trisaccharide, tetrasaccharide, oligosaccharide, or polysaccharide. These carbohydrate-conjugated RNA agents may target the parenchymal cells of the liver. In one embodiment, the saRNA is conjugated with more than one carbohydrate ligand, preferably two or three. In one embodiment, the saRNA is conjugated with one or more galactose moiety. In another embodiment, the saRNA is conjugated at least one (e.g., two or three or more) lactose molecules (lactose is a glucose coupled to a galactose). In another embodiment, the saRNA is conjugated with at least one (e.g., two or three or more) N-Acetyl-Galactosamine (GalNAc), N-Ac-Glucosamine (GluNAc), or mannose (e.g., mannose-6-phosphate). In one embodiment, the saRNA is conjugated with at least one mannose ligand, and the conjugated saRNA targets macrophages.

GalNAc-nucleotide (GalNAc-saRNA/GalNAc-siRNA) Conjugates

In some embodiments, the saRNA is covalently connected to a carbohydrate moiety, wherein the moiety comprises at least one (e.g., two or three or more) N-Acetyl-Galactosamine (GalNAc) or derivative thereof, to form a GalNAc-saRNA conjugate. GalNAc is an amino sugar derivative of galactose comprising a structure of

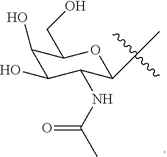 [SMILES: *C1OC(CO)C(O)C(O)C1NC(C)=O]

GalNAc is an effective moiety to carry nucleic acids construct into hepatocyte. It has been shown that discrete structure of ternary GalNAc were optimal for efficacious delivery of single stranded and double stranded oligonucleotides for gene silencing. The GalNAc-nucleotide conjugate may be delivered to cells expressing asialoglycoprotein receptor without any transfection agent. The nucleotide may be part of a saRNA, and the GalNAc-nucleotide conjugate is referred to as a GalNAc-saRNA conjugate. The nucleotide may also be part of a small inhibiting RNA (also known as small interfering RNA or siRNA) that inhibits the expression of a gene, and the GalNAc-nucleotide conjugate is referred to as a GalNAc-siRNA conjugate.

In some embodiments, the present disclosure provides a GalNAc-saRNA conjugate comprising a small activating RNA (saRNA) connected to a GalNAc moiety, wherein the saRNA comprises at least one modification, such modification which may optionally be independent of the connected GalNAc. The saRNA may comprise at least 2, 3, 4, 5, 6, 7, 8, 9, 10, 11, 12, 13, 14, 15, 16, 17, 18, 19, 20, 21, 22, 23, 24, 25, 26, 27, 28, 29 or 30 modifications for each strand.

In some embodiments, the saRNA of the conjugate is at least 50% modified, i.e., at least 50% of the nucleotides are modified. In some embodiments, the saRNA is at least 75% modified, i.e., at least 75% of the nucleotides are modified. In some embodiments, both strands of the saRNA may be modified across the whole length (100% modified). It is to be understood that since a nucleotide (sugar, base and phosphate moiety, e.g., linker) may each be modified, any modification to any portion of a nucleotide, or nucleoside, will constitute a modification.

In some embodiments, the saRNA is at least 10% modified in only one component of the nucleotide, with such component being selected from the nucleobase, sugar or linkage between nucleosides. For example, modifications of an saRNA may be made to at least 10%, 20%, 30%, 40%, 50%, 60%, 70%, 80%, 90% or 100% of the nucleobases, sugars or linkages of said saRNA.

In some embodiments, the saRNA of the conjugate comprises at least one sugar modification. In some embodiments, at least one of the 2′ positions of the sugar (OH in RNA or H in DNA) of a nucleotide of the saRNA is substituted with —OMe, referred to as 2′-OMe. In some embodiments, at least one of the 2′ positions of the sugar (OH in RNA or H in DNA) of a nucleotide of the saRNA is substituted with —F, referred to as 2′-F.

In some embodiments, the saRNA of the conjugate comprises 3′ and/or 5′ capping or overhang. In some embodiments, the saRNA of the present invention may comprise at least one inverted deoxyribonucleoside overhang. The inverted overhang, e.g., dT, may be at 5′ terminus or 3′ terminus of the passenger (sense) strand. In some embodiments, the saRNA of the present invention may comprise inverted abasic modifications on the passenger strand. The at least one inverted abasic modification may be on 5′ end, or 3′ end, or both ends of the passenger strand. The inverted abasic modification may encourage preferential loading of the guide strand.

In some embodiments, the saRNA at least one motif of at least 2 consecutive nucleotides that have the same sugar modification. In one example, such a motif may comprise 2 or 3 consecutive nucleotides. In some embodiments, the consecutive nucleotides of the motif comprise 2′-F modifications. In some embodiments, the consecutive nucleotides of the motif comprise 2′-OMe modifications.

In some embodiments, when the saRNA is double-stranded, the passenger strand and the guide strand of the saRNA each comprises at least one motif of consecutive nucleotides that have the same sugar modification.

In some embodiments, the passenger strand and the guide strand of the saRNA each comprises at least two motifs of consecutive nucleotides that have the same sugar modification. In some embodiments, the at least two motifs on a given strand independently have different sugar modifications. For example, the passenger strand or the guide strand may have at least one motif of 2′-OMe modifications and at least one motif of 2′-F modifications. In some embodiments, the at least two motifs on a given strand are separated by at least one (such as 1, 2, 3, 4, 5, 6, 7, 8, 9, 10, 11, 12, 13, 14, 15, 16, 17, 18, 19, or 20) nucleotide. In some embodiments, the at least two motifs on a given strand are connected. In some embodiments, at least one motif on the passenger strand and its complementary motif on the guide strand have different sugar modifications. For example, the nucleotides of a motif on the passenger strand has 2′-F modifications and the nucleotides of a motif on the guide strand has 2′-OMe modifications, wherein the two motifs are complimentary to each other. In another example, the nucleotides of a motif on the passenger strand has 2′-OMe modifications and the nucleotides of a motif on the guide strand has 2′-F modifications, wherein the two motifs are complimentary to each other.

In some embodiments, the modification of a motif is different from the modifications of the immediately adjacent nucleotides on both sides of the motif

In some embodiments, the saRNA comprises at least one motif of alternating sugar modifications. In one example, the motif of alternating sugar modifications comprises 2 to 30 nucleotides. In some embodiments, the motif comprises alternating 2′-F and 2′-OMe modifications.

In some embodiments, when the saRNA is double stranded, the passenger strand and the guide strand each comprises at least one motif of alternating sugar modifications. In some embodiments, at least one nucleotide on the passenger strand and its complementary nucleotide on the guide strand have different sugar modifications. For example, one nucleotide of the base pair on the passenger strand has a 2′-F modification and the other nucleotide of the base pair on the guide strand has a 2′-OMe modification. In another example, one nucleotide of the base pair on the passenger strand has a 2′-OMe modification and the other nucleotide of the base pair on the guide strand has a 2′-F modification.

In some embodiments, the saRNA comprises at least one phosphorothioate linkage or methylphosphonate linkage between nucleotides.

In some embodiments, the saRNA of the conjugate comprises a general formula of formula (I) described herein.

In some embodiments, the present disclosure provides a GalNAc-siRNA conjugate comprising a small inhibiting RNA (siRNA) connected to a GalNAc moiety.

In some embodiments, the GalNAc moiety is attached to the 2′- or 3′-position of the ribosugar, or to a nucleobase of a nucleotide of a saRNA or siRNA. A phosphodiester or phosphorothioate linkage may be between the GalNAc moiety and the nucleotide.

In some embodiments, the GalNAc moiety is attached to a nucleotide of a saRNA or siRNA via a linker. The linker may be attached to any appropriate position of a nucleotide of the saRNA or siRNA. The linker may bind to the GalNAc moiety covalently or non-covalently.

In some cases, the linker is connected to the terminal of a strand of the saRNA or siRNA. In some cases, the linker is connected to the 5′ end of the sense strand. In some cases, the linker is connected to the 3′ end of the sense strand.

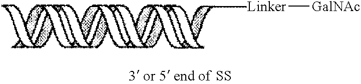

In some cases, the linker is connected to an internal nucleotide of a strand of the saRNA or siRNA. In some cases, the linker is connected to an internal nucleotide of the sense strand of the saRNA or siRNA. In some cases, the linker is connected to an internal nucleotide of the anti-sense strand of the saRNA or siRNA.

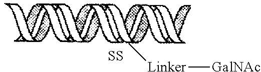

Any attachment method disclosed in Manoharan et al., Chemical Biology, vol. 10(5):1181, (2015) or Manoharan et al., ChemBioChem, vol.16(6):903, (2015), the contents of each of which are incorporated herein by reference in their entirety, may be used to attach the GalNAc moiety to the saRNA.

In some cases, the linker of the GalNAc-saRNA conjugate or GalNAc-siRNA conjugate is a direct bond or an atom such as oxygen or sulfur, a unit such as —NH—, —C(O)—, —C(O)NH—, —S(O)—, —SO2-, —SO2NH— or a chain of atoms, such as, but not limited to, alkyl, alkenyl, \ alkynyl, arylalkyl, arylalkenyl, arylalkynyl, heteroarylalkyl, heteroarylalkenyl, heteroarylalkynyl, heterocyclylalkyl, heterocyclylalkenyl, heterocyclylalkynyl, aryl, heteroaryl, heterocyclyl, cycloalkyl, cycloalkenyl, alkylarylalkyl, alkylarylalkenyl, alkylarylalkynyl, alkenylarylalkyl, alkenylarylalkenyl, alkenylarylalkynyl, alkynylarylalkyl, alkynylarylalkenyl, alkynylarylalkynyl, alkylheteroarylalkyl, alkylheteroarylalkenyl, alkylheteroarylalkynyl, alkenylheteroarylalkyl, alkenylheteroarylalkenyl, alkenylheteroarylalkynyl, alkynylheteroarylalkyl, alkynylheteroarylalkenyl, alkynylheteroarylalkynyl, alkylheterocyclylalkyl, alkylheterocyclylalkenyl, alkylhererocyclylalkynyl, alkenylheterocyclylalkyl, alkenylheterocyclylalkenyl, alkenylheterocyclylalkynyl, alkynylheterocyclylalkyl, alkynylheterocyclylalkenyl, alkynylheterocyclylalkynyl, alkylaryl, alkenylaryl, alkynylaryl, alkylheteroaryl, alkenylheteroaryl, alkynylhereroaryl, wherein each group may be substituted or unsub stituted.

In some cases, the linker is a cleavable linker. The cleavable linker may be cleaved at a certain pH, by a certain enzyme, or at a certain redox environment. The cleavable linker may comprise an ester bond, an acid-labile bond, a disulfide bond, or a phosphate bond by way of example.

In some cases, the linker is a non-cleavable linker.

In some cases, the linker comprises an amine group, such as —NH—(CH₂)₆— or NH₂—(CH₂)₆— (referred to as NH2C6, C6NH2, or C6). For GalNAc clusters that comprise a carboxylic acid at the terminus, the carboxylic acid reacts with the amine on the linker and the GalNAc cluster is directly attached to the saRNA-C6NH— or siRNA-C6NH.

In some cases, the linker comprises a carboxylic acid group, such as —O—CO—(CH₂)n-CO—NH—(CH₂)₆—, n=2, 3, 4, 5 or 6. For GalNAc clusters that comprise an amine at the terminus, the amine reacts with the carboxylic acid on the linker and the GalNAc cluster is directed attached to saRNA-(CH₂)₆—NH—CO—(CH₂)n-CO— or siRNA-(CH₂)₆—NH—CO—(CH₂)n-CO—.

In some cases, a phosphorothioate linkage is between the linker and the sense strand.

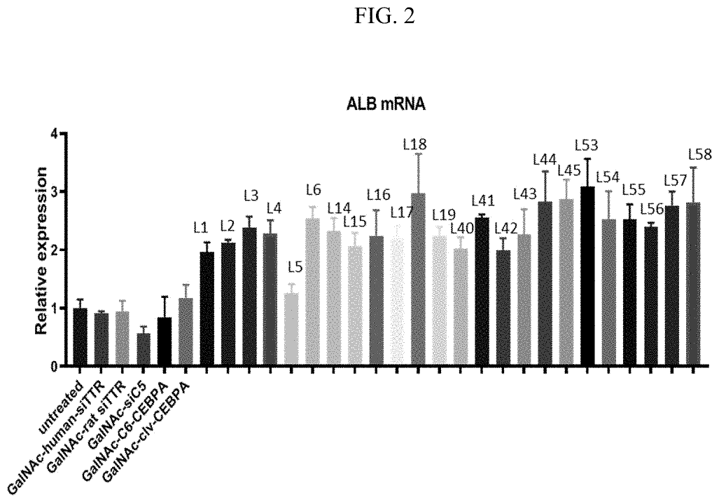

In some cases, the GalNAc moiety may be a triantennary GalNAc-cluster. Any GalNAc cluster disclosed in Prakash et al., Journal of Medicinal Chemistry, vol.59:2718-2733 (2016), the contents of which are incorporated herein by reference in their entirety, such as Tris based GalNAc clusters, Triacid based GalNAc clusters, Lys-Lys based GalNAc clusters, Lys-Gly based GalNAc clusters, Trebler based clusters, hydroxyprolinol based clusters in FIG. 2 of Prakash et al., may be used according to the current disclosure. The GalNAc cluster may have a structure of:

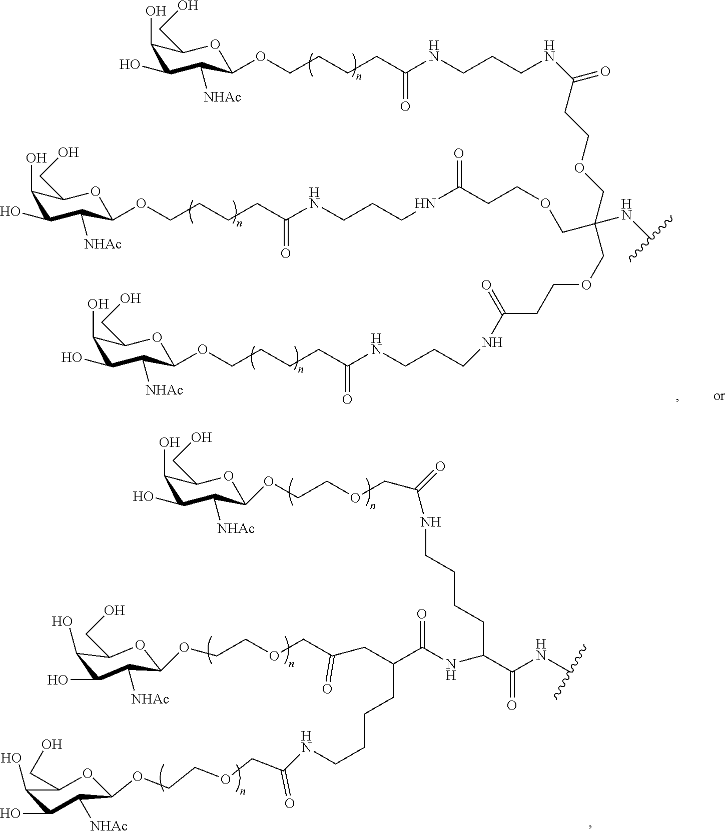

n=1, 2, 3, 4, 5, or 6.

When a linker is used to connect the 3′ or 5′ end of the sense strand to the GalNAc moiety, the GalNAc-saRNA conjugate or the GalNAc-siRNA conjugate comprises a structure of:

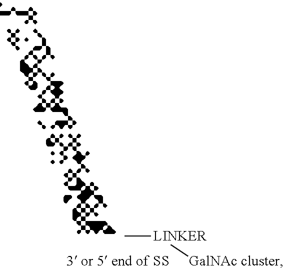

such as

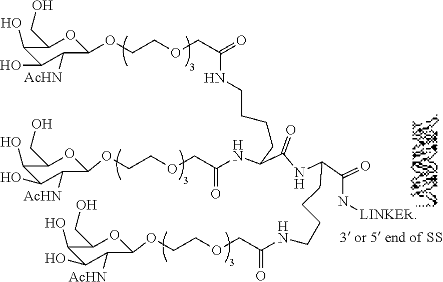

For example, the GalNAc-saRNA conjugate or the GalNAc-siRNA conjugate may have a structure of:

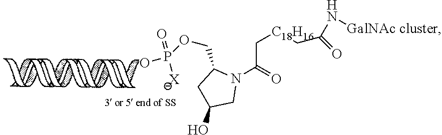

such as

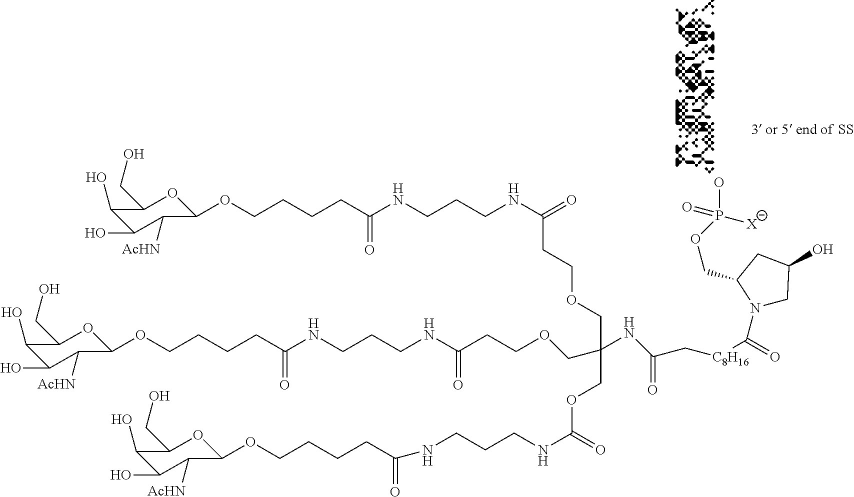

wherein X is O or S.

In some embodiments, the GalNAc-saRNA conjugate or the GalNAc-siRNA conjugate comprises at least one GalNAc monomer selected from M1, M1′, M2, M2′, M3, M3′, M4, M4′, M5, M5′, M6, M6′ or a derivative thereof. The GalNAc-saRNA conjugate may comprise one, two, three, four, five, six, seven, eight or nine GalNAc monomers selected from M1, M1′, M2, M2′, M3, M3′, M4, M4′, M5, M5′, M6, M6′ or a derivative thereof.

In one embodiment, the GalNAc-saRNA conjugate or the GalNAc-siRNA conjugate comprises at least one GalNAc monomer selected from M1, M1′, or a derivative thereof.

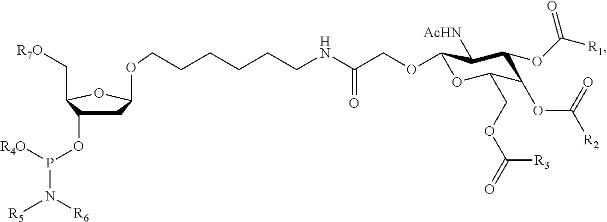

wherein R1, R2, and R3 can be the same or different, and wherein R1, R2, and R3 are independently selected from an alkyl, aryl, and alkenyl group. In some embodiments, at least one of R1, R2 and R3 is —CH₃. In some embodiments, R1, R2 and R3 are all —CH₃.
wherein R4 is a suitable protecting group or C1-6 straight or branched alkyl, which includes but not limited to methyl, ethyl, n-propyl, 2-propyl, n-butyl, isobutyl and other alkyl groups. In some embodiments R4 is —CH₃ or CH₂CH₃. In some embodiments, R4 is —CH₂CH₂CN. wherein R5, R6 are each independently C1-6 straight or branched alkyl, which includes but not limited to methyl, ethyl, n-propyl, 2-propyl, n-butyl, isobutyl and similar alkyl groups. In some embodiments R5 and R6 are both 2-propyl. and

wherein R7 is a suitable protecting group. In some embodiments, the protecting group is 4,4′-dimethoxytrityl.

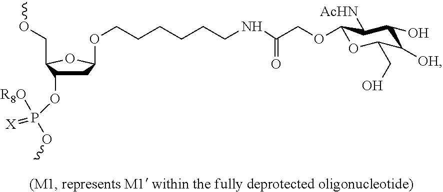

wherein R₈ is —H, or C1-6 straight or branched alkyl, which includes but not limited to methyl, ethyl, n-propyl, 2-propyl, n-butyl, isobutyl and similar alkyl groups; and wherein X is O or S. In some embodiments R8 is —CH₃ or —CH₂CH₃.

In another embodiment, the GalNAc-saRNA conjugate or the GalNAc-siRNA conjugate comprises at least one GalNAc monomer selected from M2, M2′, or a derivative thereof.

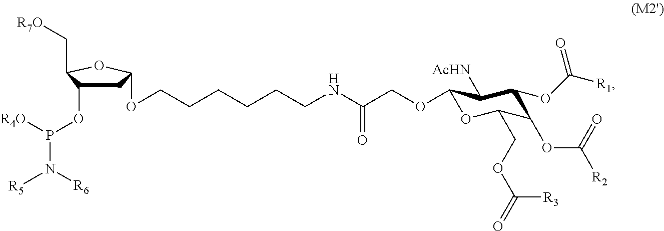

wherein R1, R2, and R3 can be the same or different, and wherein R1, R2, and R3 are independently selected from an alkyl, aryl, and alkenyl group. In some embodiments, at least one of R1, R2 and R3 is —CH₃. In some embodiments, R1, R2 and R3 are all —CH₃. wherein R4 is -a protecting group or C1-6 straight or branched alkyl, which includes but not limited to methyl, ethyl, n-propyl, 2-propyl, n-butyl, isobutyl and similar alkyl groups. In some embodiments, R4 is —CH₃ or CH₂CH₃. In some embodiments, R4 is —CH₂CH₂CN. wherein R5, R6 are each independently C1-6 straight or branched alkyl, which includes but not limited to methyl, ethyl, n-propyl, 2-propyl, n-butyl, isobutyl and other alkyl groups. In some embodiments, R5 and R6 are both 2-propyl. and wherein R7 is a suitable protecting group. In some embodiments the protecting group is 4,4′-dimethoxytrityl.

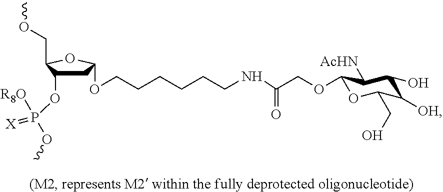

wherein R₈ is -H, or C1-6 straight or branched alkyl, which includes but not limited to methyl, ethyl, n-propyl, 2-propyl, n-butyl, isobutyl and similar alkyl groups; and wherein X is O or S. In some embodiments, R8 is —CH₃ or —CH₂CH₃.

In one embodiment, the GalNAc-saRNA conjugate or the GalNAc-siRNA conjugate comprises at least one GalNAc monomer selected from M3, M3′, or a derivative thereof.

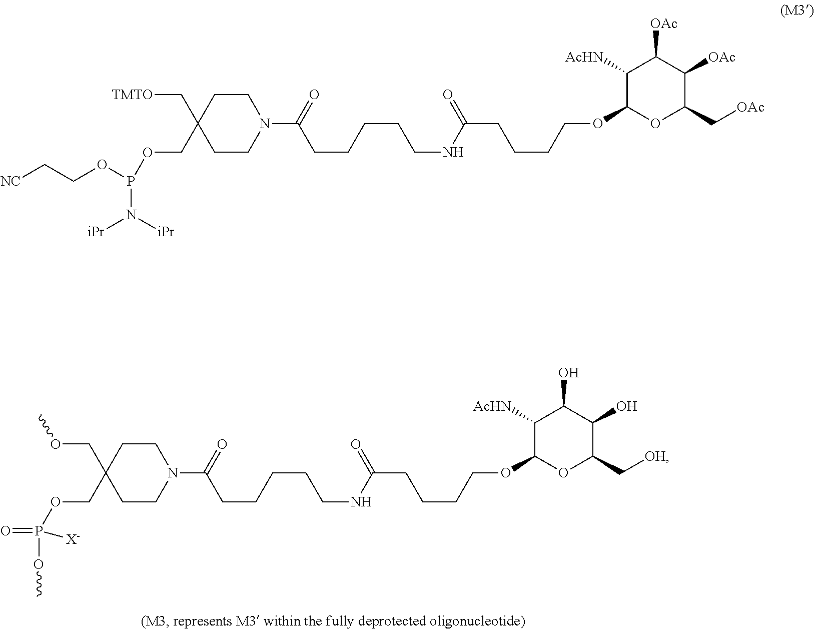

wherein X is O or S.

In one embodiment, the GalNAc-saRNA conjugate or the GalNAc-siRNA conjugate comprises at least one GalNAc monomer selected from M4, M4′, or a derivative thereof.

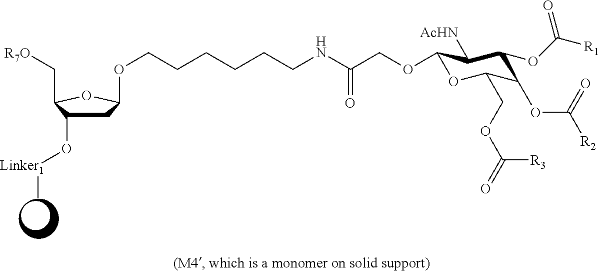

wherein R1, R2, and R3 can be the same or different, and wherein R1, R2, and R3 are independently selected from an alkyl, aryl, and alkenyl group. In some embodiments, at least one of R1, R2 and R3 is —CH₃. In some embodiments, R1, R2 and R3 are all —CH₃. wherein R7 is a protecting group. In some embodiments, the protecting group is 4,4′-dimethoxytrityl. and wherein Linker1 is a cleavable linker. In some embodiments, Linker1 is succinyl.

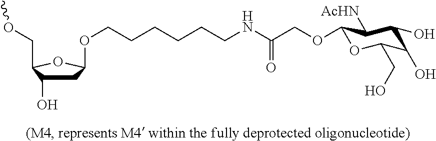

In one embodiment, the GalNAc-saRNA conjugate or the GalNAc-siRNA conjugate comprises at least one GalNAc monomer selected from M5, M5′, or a derivative thereof.

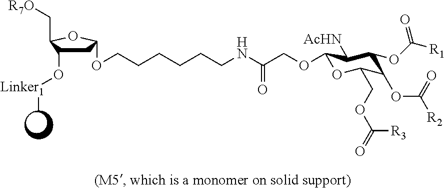

wherein R1, R2, and R3 can be the same or different, and wherein R1, R2, and R3 are independently selected from an alkyl, aryl, and alkenyl group. In some embodiments, at least one of R1, R2 and R3 is —CH₃. In some embodiments, R1, R2 and R3 are all —CH₃. wherein R7 is a suitable protecting group. In some embodiments, the protecting group is 4,4′-dimethoxytrityl. and wherein Linker1 is a cleavable linker. In some embodiments, Linker1 is succinyl.

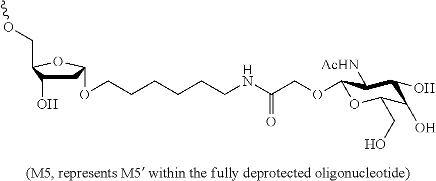

In one embodiment, the GalNAc-saRNA conjugate or the GalNAc-siRNA conjugate comprises at least one GalNAc monomer selected from M6, M6′, or a derivative thereof.

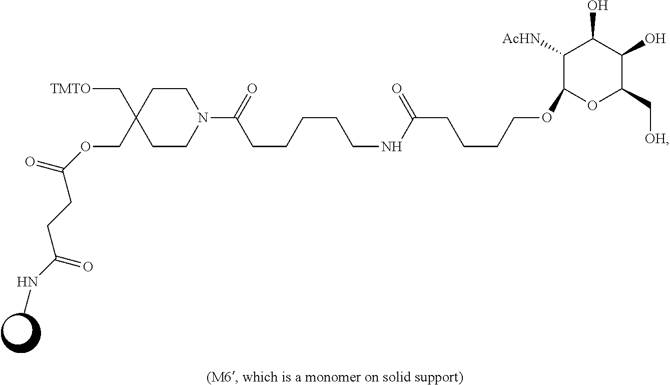

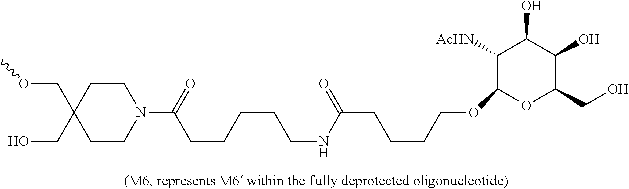

The GalNAc monomers are used to build GalNAc moieties which when conjugated to a saRNA achieve delivery of saRNA to a targeted organ, such as liver. The GalNAc moiety comprises at least one GalNAc monomer. In some embodiments, the GalNAc moiety may be a GalNAc cluster (or multimer) comprising at least two GalNAc monomers. In some embodiments, the GalNAc cluster may be a GalNAc dimer cluster comprising 2 GalNAc monomers. In some embodiments, the GalNAc cluster may be a triantennary GalNAc cluster comprising 3 GalNAc monomers. The GalNAc monomer building block is compatible with standard oligonucleotide synthesis by the phosphoramidite methods. The monomer can be used to provide functionalised support or added in-line during oligonucleotide synthesis. The GalNAc monomers may be attached in-line at the 5′ of an oligonucleotide linked to form a GalNAc conjugate. As used herein, ‘in-line’ refers to the automated process of elongation of oligonucleotide during synthesis. The GalNAc monomer can be added at any position of the oligonucleotide alone or in combination with other GalNAc monomers. They can be added sequentially without any linker or separated by nucleotides or other linkers.

In some embodiments, the GalNAc moiety comprises at least one GalNAc monomer and at least one spacer (may also be called a linker in some circumstances), wherein the GalNAc monomer is attached to the spacer via a bond (such as a phosphate bond or a phosphorothioate bond). In some embodiments, the GalNAc moieties comprise at least two GalNAc monomers (such as 2 monomers, 3 monomers, 4 monomers, 5 monomers, or 6 monomers) and optionally at least one spacer, wherein the monomers are attached to each other or to the spacer via a bond (such as a phosphate bond or a phosphorothioate bond). The spacer may be a non-cleavable linker, such as but not limited to hexaethylene glycol (HEG), C12, an abasic furan, a triethylene glycol (TEG), C3, or a derivative thereof (e.g., with a suitable protecting group):
1). HEG spacer (HEG) as shown within the fully deprotected oligonucleotide

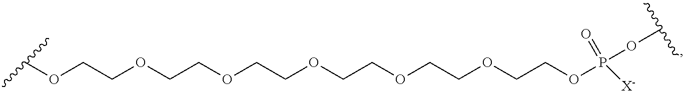

wherein X is O or S. In the present disclosure, when ‘HEG’ is used, X is O. When ‘S-HEG’ is used, X is S.
2). C12 spacer (C12) as shown within the fully deprotected oligonucleotide:

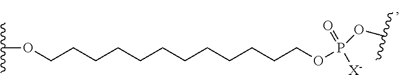

wherein X is O or S. In the present disclosure, when ‘C12 is used, X is O. When ‘S-C12 is used, X is S.
3). Abasic spacer (ab) as shown within the fully deprotected oligonucleotide:

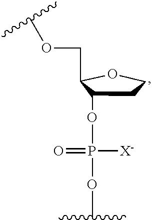

wherein X is O or S. In the present disclosure, when ‘ab is used, X is O. When ‘S-ab is used, X is S.
4). TEG spacer (TEG) as shown within the fully deprotected oligonucleotide:

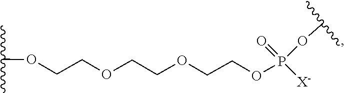

wherein X is O or S. In the present disclosure, when ‘TEG’ is used, X is O. When ‘S-TEG’ is used, X is S.
5). C3 spacer (C3) as shown within the fully deprotected oligonucleotide:

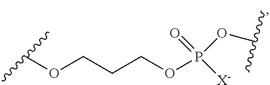

wherein X is O or S. In the present disclosure, when ‘C3 is used, X is O. When ‘S-C3’ is used, X is S.

A GalNAc moiety can be prepared by a process comprising the steps of:
1). providing at least one GalNAc monomer selected from the group consisting of M1′, M2′, M3′, M4′, M5′ and M6′; and
2). synthesizing a GalNAc moiety from the GalNAc monomer(s) in step 1), optionally adding at least one spacer and optionally removing the protecting groups.

In some embodiments, the GalNAc moiety comprises at least one M1 monomer (such as exactly one, exactly two, or exactly three M1 monomers) and at least one spacer. In some embodiments, the GalNAc moiety comprises: at least one M1 monomer; and at least one M2 or M3 monomer (such as three M3 monomers), or one M4, M5 or M6 monomer. In some embodiments, the GalNAc moiety comprises: at least one M1 monomer; at least one spacer; and at least one M2 or M3 monomer (such as three M3 monomers), or one M4, M5 or M6 monomer. In some embodiments, the GalNAc moiety comprises at least one M1 monomer (such as exactly one, exactly two, or exactly three M1 monomers) without any spacer.

In some embodiments, the GalNAc moiety comprises at least one M2 monomer (such as exactly one, exactly two, or exactly three M2 monomers) and at least one spacer. In some embodiments, the GalNAc moiety comprises: at least one M2 monomer; and at least one M1 or M3 monomer (such as three M3 monomers), or one M4, M5 or M6 monomer. In some embodiments, the GalNAc moiety comprises: at least one M2 monomer; at least one spacer; and at least one M1 or M3 monomer (such as three M3 monomers), or one M4, M5 or M6 monomer. In some embodiments, the GalNAc moiety comprises at least one M2 monomer (such as exactly one, exactly two, or exactly three M2 monomers) without any spacer.

In some embodiments, the GalNAc moiety comprises at least one M3 monomer and at least one spacer. In some embodiments, the GalNAc moiety comprises: at least one M3 monomer; and at least one M1 or M2 monomer, or one M4, M5 or M6 monomer. In some embodiments, the GalNAc moiety comprises: at least one M3 monomer; at least one spacer; and at least one M1 or M2 monomer, or one M4, M5 or M6 monomer. In some embodiments, the GalNAc moiety comprises three M3 monomers with at least one spacer. In some embodiments, the GalNAc moiety comprises three M3 monomers without any spacer. In some embodiments, the GalNAc moiety excludes a GalNAc moiety consisting of only one M3 monomer.

In some embodiments, the GalNAc moiety does not comprise more than one M4 monomers. In some embodiments, the GalNAc moiety comprises one M4 monomer. In some embodiments, the GalNAc moiety does not comprise any M4 monomer. In some embodiments, the GalNAc moiety comprises one M4 monomer; and at least one M1, M2, or M3 monomer, or one M5 or M6 monomer.

In some embodiments, the GalNAc moiety does not comprise more than one M5 monomers. In some embodiments, the GalNAc moiety comprises one M5 monomer. In some embodiments, the GalNAc moiety does not comprise any M5 monomer. In some embodiments, the GalNAc moiety comprises one M5 monomer; and at least one M1, M2, or M3 monomer, or one M4 or M6 monomer.

In some embodiments, the GalNAc moiety does not comprise more than one M6 monomers. In some embodiments, the GalNAc moiety comprises one M6 monomer. In some embodiments, the GalNAc moiety does not comprise any M6 monomer. In some embodiments, the GalNAc moiety comprises: one M6 monomer; and at least one M1, M2, or M3 monomer (such as three M3 monomers), or one M4 or M5 monomer. In some embodiments, the GalNAc moiety excludes a GalNAc moiety comprising one M6 monomer and two M3 monomers.

In some embodiments, the GalNAc moiety may be a triantennary GalNAc cluster having a structure of:

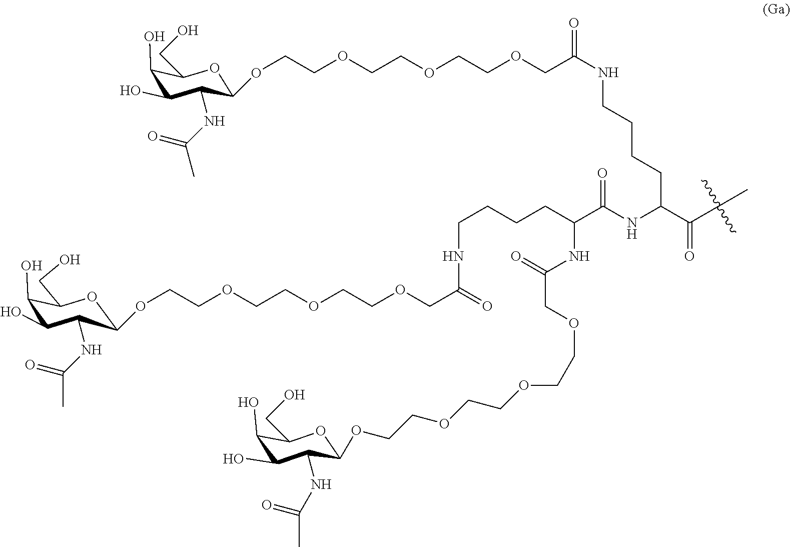

or any of the structures in Table 3. There GalNAc moieties are also referred to as GalNAc clusters.

TABLE 3
GalNAc moiety structures

| Cluster ID | Cluster Structure |
|---|---|
| G1 | (M3)-(M3)-(M3)- |
| G2 | (M3)-(M3)-(M3)-HEG |
| G3 | (M3)-HEG-(M3)-HEG-(M3)-HEG- |
| G4 | (M3)-(M3)-(M3)-C12- |
| G5 | (M3)-C12-(M3)-C12-(M3)-C12- |
| G6 | (M3)-ab-(M3)-ab-(M3)-HEG- |
| G7 | (M1)-(M1)-(M1)- |
| G8 | (M1)-(M1)-(M1)-HEG- |
| G9 | (M1)-HEG-(M1)-HEG-(M1)-HEG- |
| G10 | (M1)-(M1)-(M1)-C12- |
| G11 | (M1)-C12-(M1)-C12-(M1)-C12- |
| G12 | (M1)-ab-(M1)-ab-(M1)-HEG- |
| G13 | (M2)-TEG-(M2)-TEG-(M2)-TEG- |
| G14 | (M2)-C3-(M2)-C3-(M2)-C3- |
| G15 | (M2)-S-C3-S-(M2)-S-C3-S-(M2)-S-C3- |
| G16 | (M1)-TEG-(M1)-TEG-(M1)-TEG- |
| G17 | (M1)-C3-(M1)-C3-(M1)-C3- |
| G18 | (M1)-S-C3-S-(M1)-S-C3-S-(M1)-S-C3- |
| G19 | (M1)-C3-(M1)-C3-(M1)-C3-(M1)-C3- |
| G20 | (M1)-C3-(M1)-C3- |
| G21 | (M1)-C3- |
| G22 | -C3-(M1)-C3-(M1) |
| G23 | -C3-(M1)-C3-(M1)-C3-(M1) |
| G24 | -C3-(M2)-C3-(M2)-C3-(M2) |

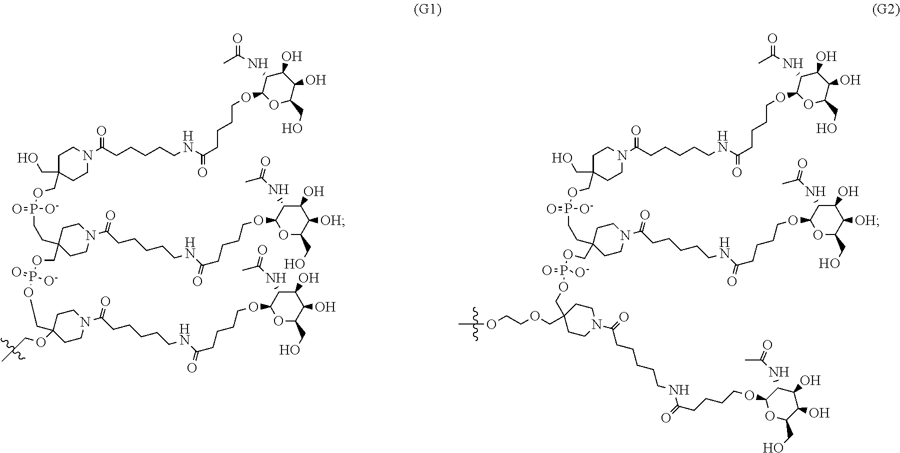

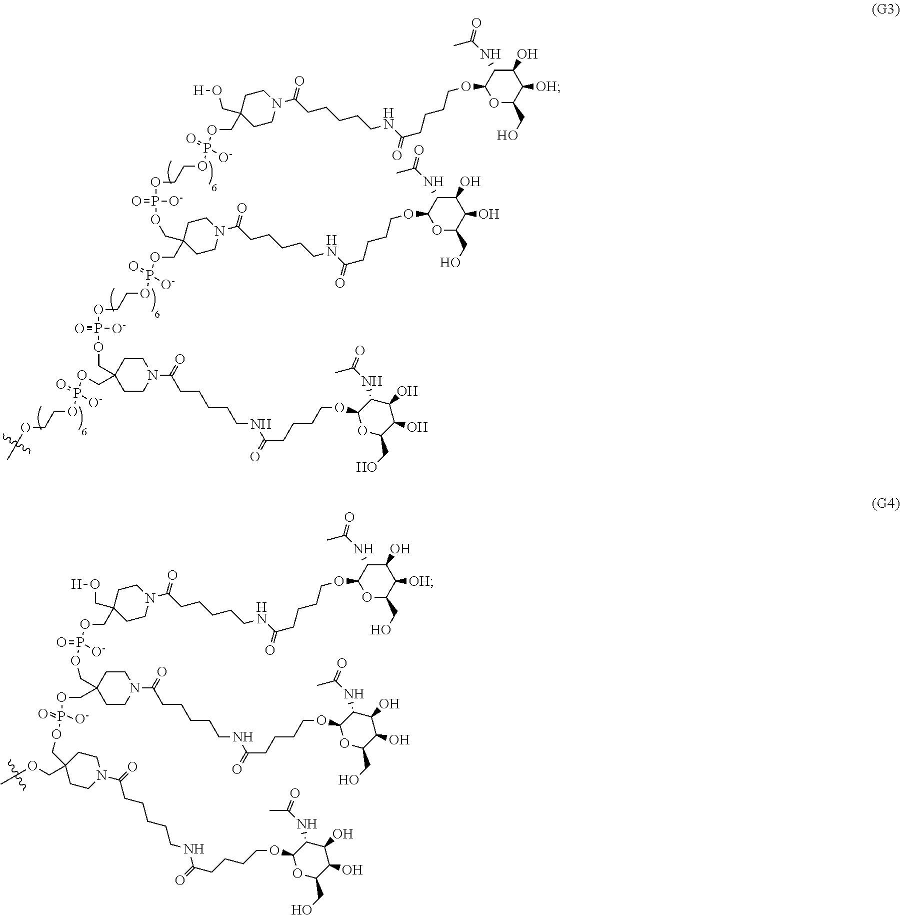

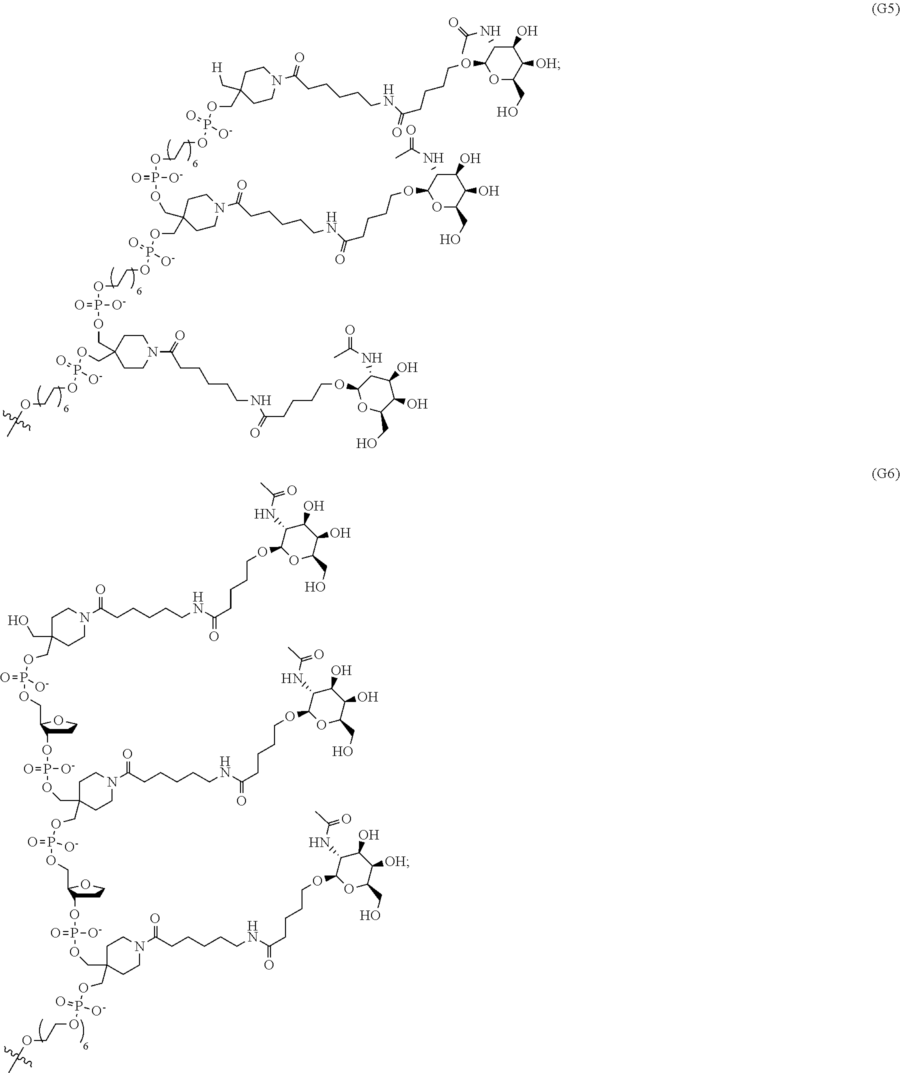

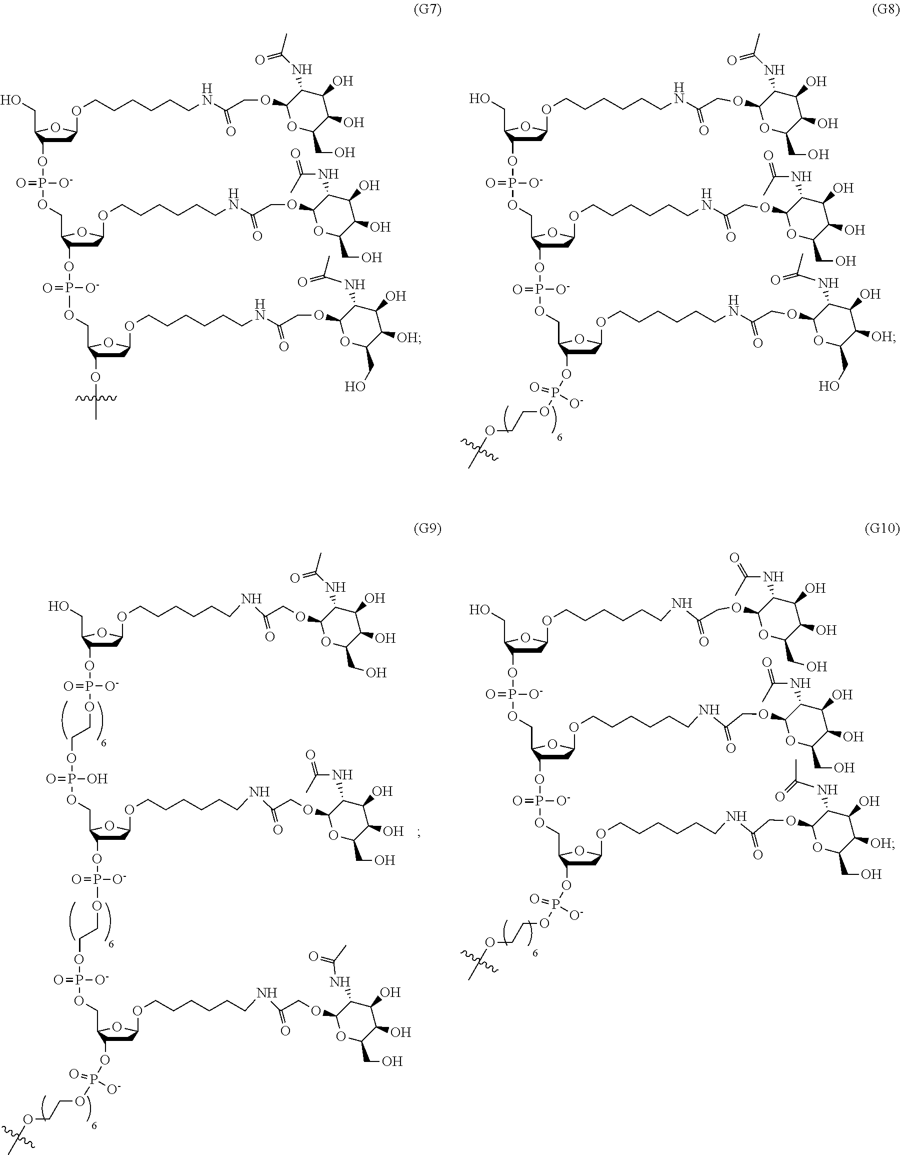

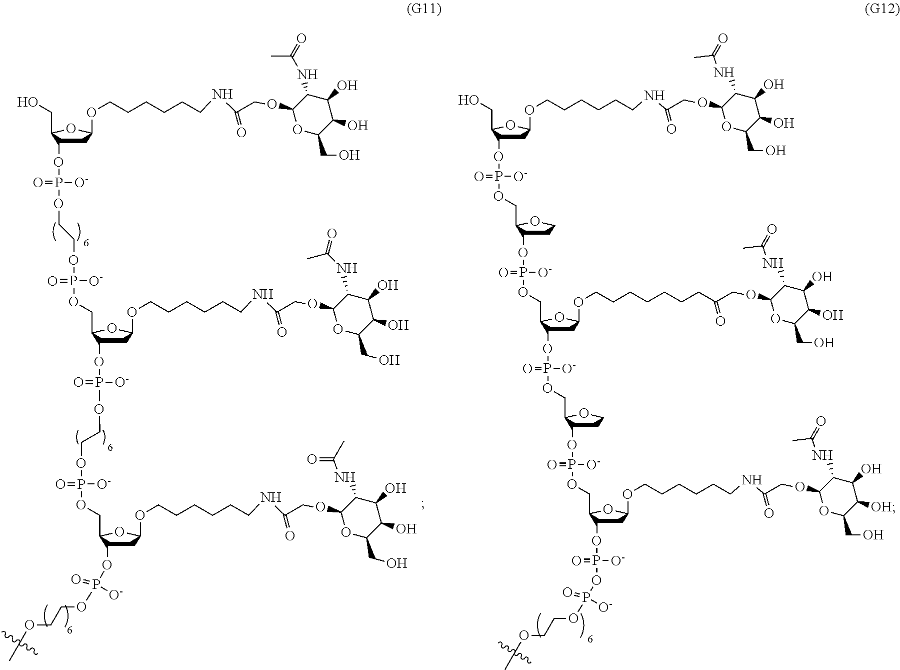

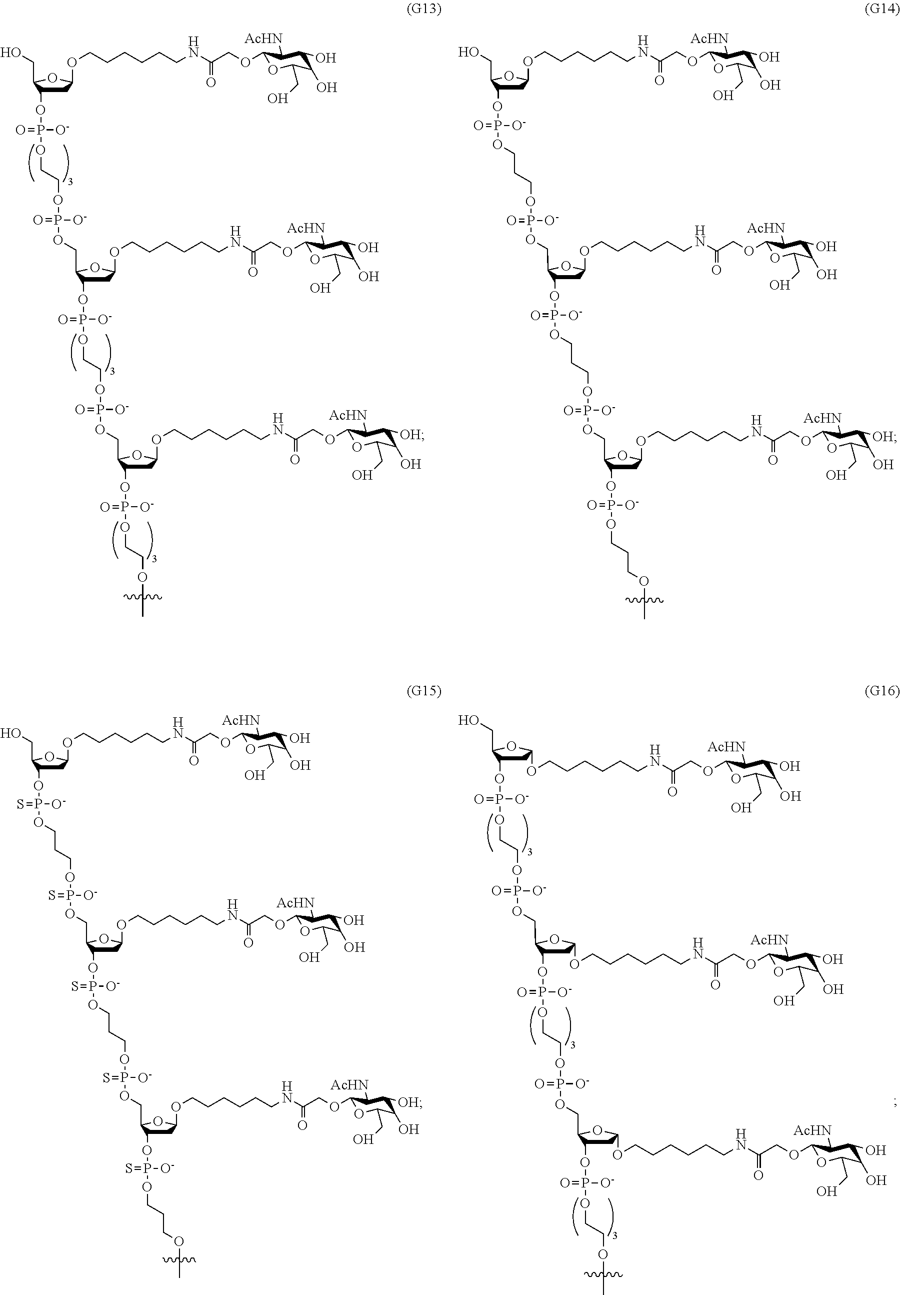

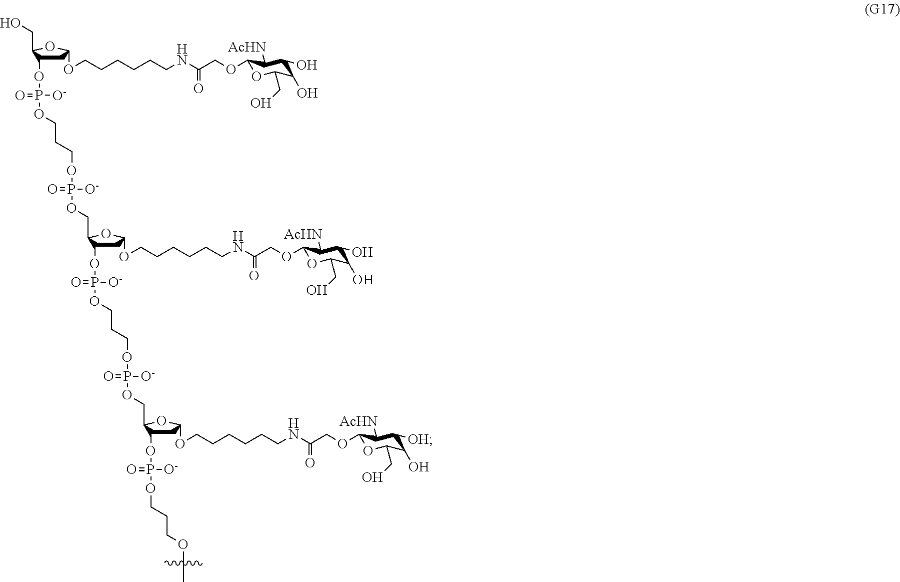

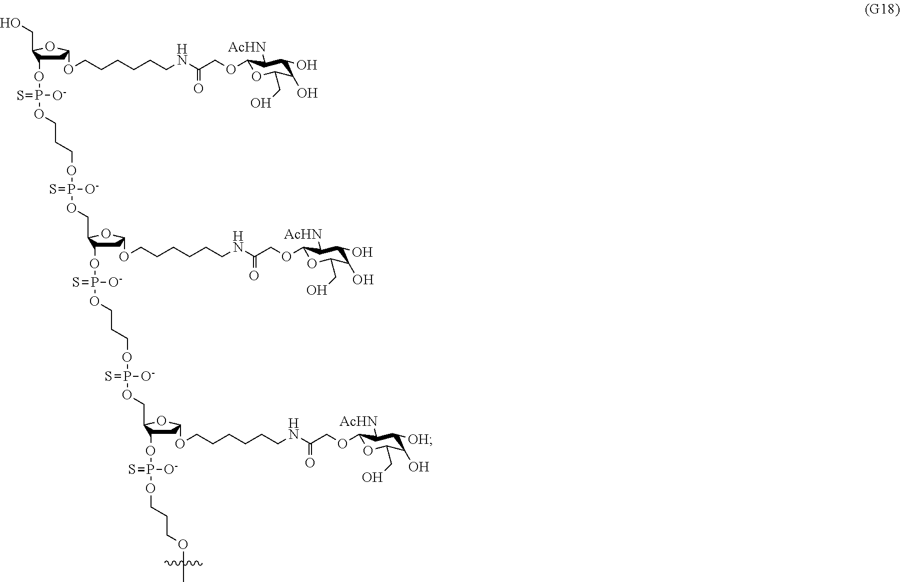

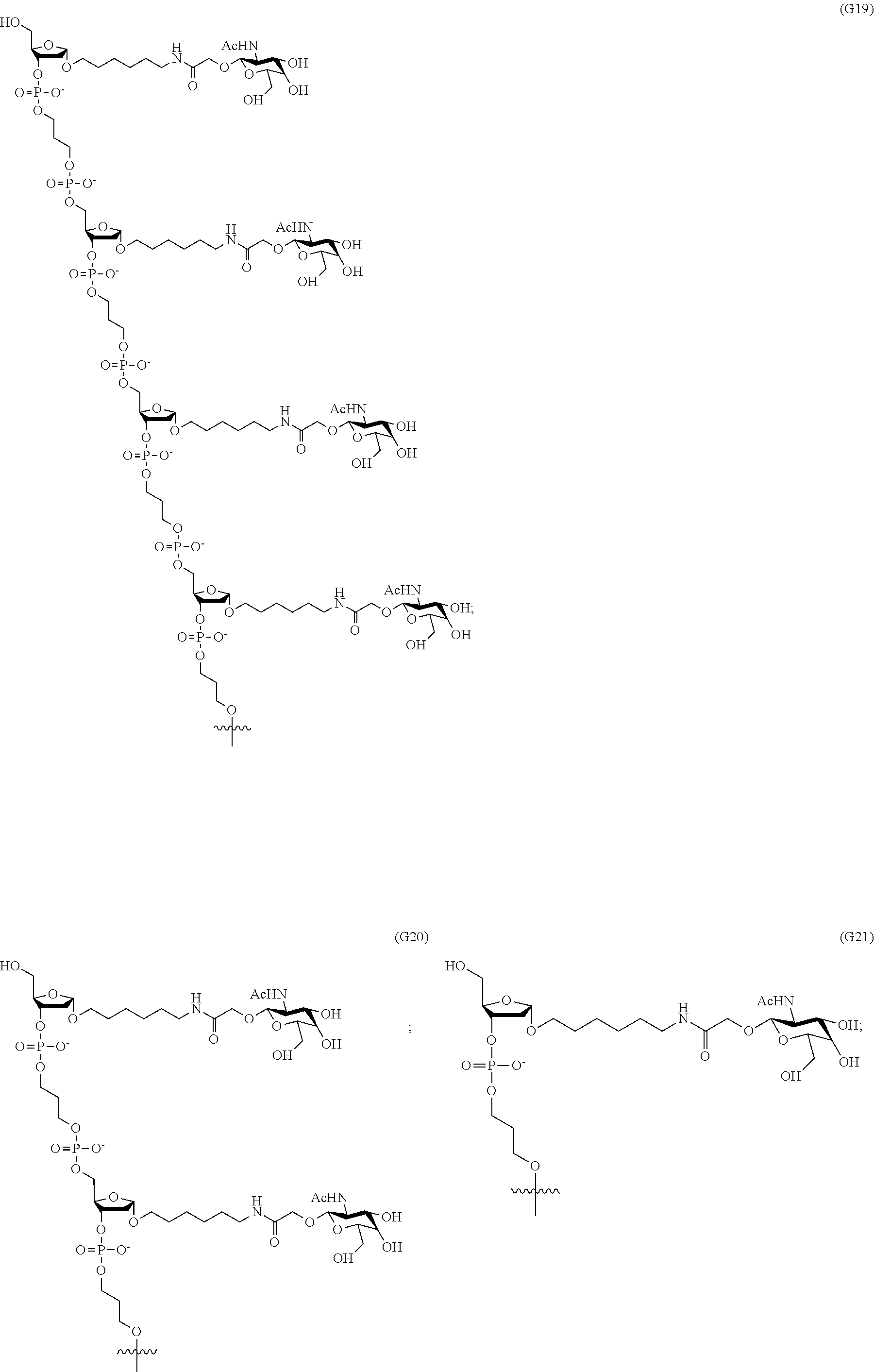

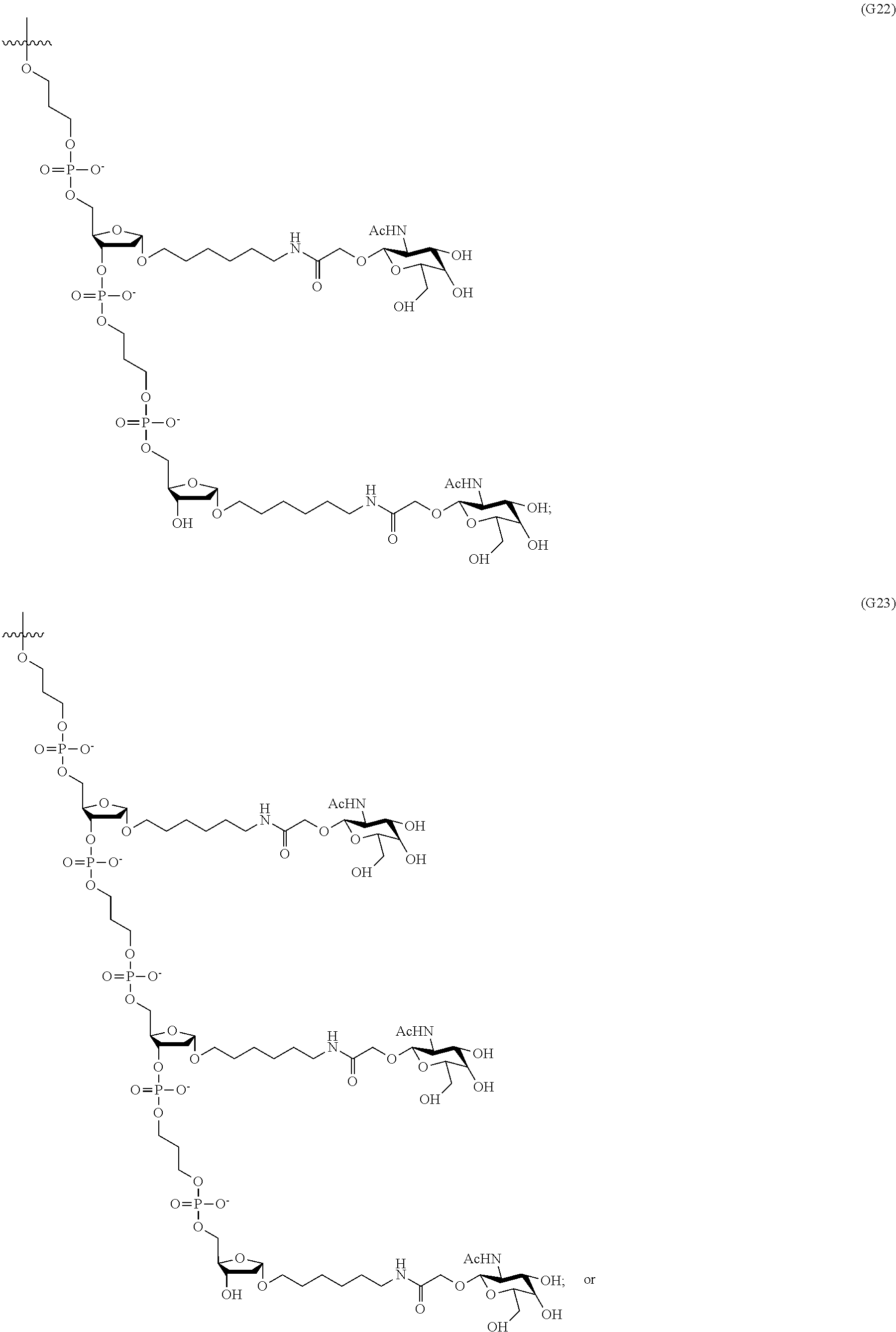

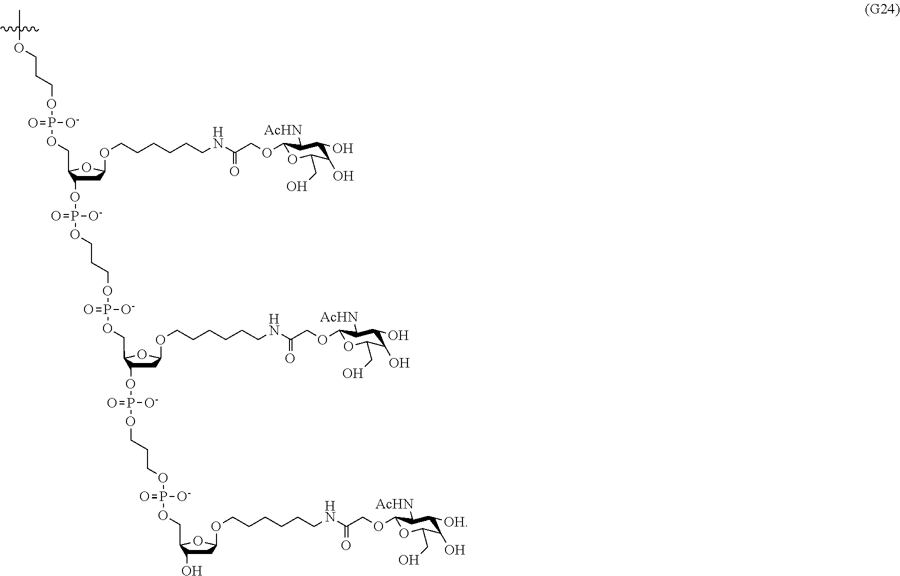

The GalNAc moiety may be attached to an oligonucleotide sequence (e.g., a sense strand of a double-stranded saRNA) by a bond (such as a phosphodiester or a phosphorothioate bond) with or without a cleavable linker to form a conjugate. The GalNAc moiety may be attached to the 5′ terminus O or 3′ terminus O of the oligonucleotide sequence. In some embodiments, the cleavable linker is a C6ssC6 linker with a structure of:

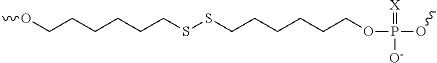

(C6ssC6 within the fully deprotected oligonucleotide), wherein X is O or S;

In some embodiments, the cleavable linker is a dT linker with a structure of:

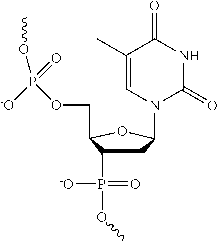

within the fully deprotected oligonucleotide.

A GalNAc-saRNA conjugate can be prepared by a process comprising the steps of:
1). providing at least one GalNAc monomer selected from the group consisting of M1′, M2′, M3′, M4′, M5′ and M6′; optionally adding at least one spacer;
2). providing at least one saRNA (such as any saRNA in Table 2); optionally adding at least one linker; and
3). synthesizing the GalNAc-saRNA conjugate from the GalNAc monomer(s) in step 1) and the saRNA(s) in step 2), optionally removing the protecting groups.

In some embodiments, the GalNAc moiety is attached to the 5′ end of the sense strand of a double-stranded saRNA (saRNA duplex) to form a conjugate, wherein the saRNA is a CEBPA-saRNA. In some embodiments, the GalNAc moiety is attached to the 3′ end of the sense strand of a double-stranded saRNA to form a conjugate, wherein the saRNA is a CEBPA-saRNA. The saRNA may be any saRNA in Table 2. In one embodiment, the saRNA has a sequence of:

XD-14369K1 duplex:

Antisense:
(SEQ ID NO: 25)
5′-GfsAfscCfaGfuGfaCfaauGfaCfcGfcsUfsu-3′
Sense:
(SEQ ID NO: 24)
5′-GfscsGfgUfcAfUfUfgUfcAfcUfgGfuCf-3′

or

XD-06414 duplex:

Antisense:
(SEQ ID NO: 15)
5′-gAfcCfaGfuGfaCfaauGfaCfcGfcsusu-3′
Sense:
(SEQ ID NO: 14)
5′-sGfcGfgUfcAfUfUfgUfcAfcUfgGfuCfuu(invdT)-3′
(Nf = the nucleotide N (N may be A, U, C, or
G) has a 2′-Fluoro (2′-F) modification; lower
case = the nucleotide has a 2′-O-Methyl (2′-
OMe) modification; s: phosphorothioate
linkage; and invdT: inverted deoxy T (dT).)

Non-limiting examples of GalNAc-saRNA conjugates or GalNAc-siRNA conjugates include the genus and species conjugates in Table 4. It is understood the sense strand of the saRNA or siRNA forms a duplex with the antisense strand of the saRNA or siRNA.

TABLE 4
GalNAc-saRNA conjugate or GalNAc-siRNA conjugate structures

| Con- | | |
|---|---|---|
| jugate | | |
| Genus | | |
| ID | Conjugate Genus Structure | Conjugate Species ID |
| CJ1 | G1-optional linker-sense strand of | L1, wherein linker is |
| | saRNA or siRNA | C6ssC6; L40, no linker. |
| CJ2 | G2-optional linker-sense strand of | L2, wherein linker is |
| | saRNA or siRNA | C6ssC6; L41, no linker. |
| CJ3 | G3-optional linker-sense strand of | L3, wherein linker is |
| | saRNA or siRNA | C6ssC6; L42, no linker. |
| CJ4 | G4-optional linker-sense strand of | L4, wherein linker is |
| | saRNA or siRNA | C6ssC6; L43, no linker. |
| CJ5 | G5-optional linker-sense strand of | L5, wherein linker is |
| | saRNA or siRNA | C6ssC6; L44, no linker. |
| CJ6 | G6-optional linker-sense strand of | L6, wherein linker is |
| | saRNA or siRNA | C6ssC6; L45, no linker. |
| CJ7 | G7-optional linker-sense strand of | L14, wherein linker |
| | saRNA or siRNA | is C6ssC6; |
| | | L53 and L80, no linker. |
| CJ8 | G8-optional linker-sense strand of | L15, wherein linker |
| | saRNA or siRNA | is C6ssC6; |
| | | L54, no linker. |
| CJ9 | G9-optional linker-sense strand of | L16, wherein linker |
| | saRNA or siRNA | is C6ssC6; |
| | | L55 and L81, no linker. |
| CJ10 | G10-optional linker-sense strand of | L17, wherein linker |
| | saRNA or siRNA | is C6ssC6; |
| | | L56, no linker. |
| CJ11 | G11-optional linker-sense strand of | L18, wherein linker |
| | saRNA or siRNA | is C6ssC6; |
| | | L57, no linker. |
| CJ12 | G12-optional linker-sense strand of | L19, wherein linker |
| | saRNA or siRNA | is C6ssC6; |
| | | L58, no linker. |
| CJ13 | G13-optional linker-sense strand of | L60, no linker, |
| | saRNA or siRNA | L66 wherein linker is dT |
| CJ14 | G14-optional linker-sense strand of | L61 no linker, |
| | saRNA or siRNA | L67 wherein linker is dT |
| CJ15 | G15-optional linker-sense strand of | L62 no linker, |
| | saRNA or siRNA | L68 wherein linker is dT |
| CJ16 | G16-optional linker-sense strand of | L63 no linker, |
| | saRNA or siRNA | L69 wherein linker is dT |
| CJ17 | G17-optional linker-sense strand of | L64 no linker, |
| | saRNA or siRNA | L70 wherein linker is dT |
| CJ18 | G18-optional linker-sense strand of | L65 no linker, |
| | saRNA or siRNA | L71 wherein linker is dT |
| CJ19 | G19-optional linker-sense strand of | L72 no linker |
| | saRNA or siRNA | |
| CJ20 | G20-optional linker-sense strand of | L73 no linker |
| | saRNA or siRNA-optional | |
| | linker-G22 | |
| CJ21 | G21-optional linker-sense strand of | L74 no linker |
| | saRNA or siRNA-optional | |
| | linker-G22 | |
| CJ22 | sense strand of saRNA or siRNA- | L75 no linker |
| | optional linker-G23 | |
| CJ23 | G17-optional linker-sense strand of | L76 no linker, |
| | saRNA or siRNA-optional | L77 wherein linker is dT |
| | linker-G23 | |
| CJ24 | G14-optional linker-sense strand of | L78 wherein linker is dT |
| | saRNA or siRNA-optional | |
| | linker-G24 | |
| CJ25 | sense strand of saRNA or siRNA- | L79 no linker |
| | optional linker-G24 | |

In some embodiments, the GalNAc moiety is attached to the 5′ end of the sense strand of XD-06414 duplex to form a conjugate. Non-limiting examples of the conjugates include any conjugate in Table 5. Conjugates L1 to L19 each comprises a cleavable linker. Conjugates L40 to L58 do not comprise any cleavable linker.

TABLE 5
Conjugates comprising a GalNAc Cluster and XD-06414 duplex

| | GalNAc | | | |
|---|---|---|---|---|
| Conjugate | Cluster | | | |
| No. | Structure | Linker | saRNA Sequences | |
| L1 | / | / | Antisense (3′-5′) | SEQ ID No. 15* |
| | G1 | C6ssC6 | Sense (5′-3′) | SEQ ID No. 14 |
| L2 | / | / | Antisense (3′-5′) | SEQ ID No. 15 |
| | G2 | C6ssC6 | Sense (5′-3′) | SEQ ID No. 14 |
| L3 | / | / | Antisense (3′-5′) | SEQ ID No. 15 |
| | G3 | C6ssC6 | Sense (5′-3′) | SEQ ID No. 14 |
| L4 | / | / | Antisense (3′-5′) | SEQ ID No. 15 |
| | G4 | C6ssC6 | Sense (5′-3′) | SEQ ID No. 14 |
| L5 | / | / | Antisense (3′-5′) | SEQ ID No. 15 |
| | G5 | C6ssC6 | Sense (5′-3′) | SEQ ID No. 14 |
| L6 | / | / | Antisense (3′-5′) | SEQ ID No. 15 |
| | G6 | C6ssC6 | Sense (5′-3′) | SEQ ID No. 14 |
| L14 | / | / | Antisense (3′-5′) | SEQ ID No. 15 |
| | G7 | C6ssC6 | Sense (5′-3′) | SEQ ID No. 14 |
| L15 | / | / | Antisense (3′-5′) | SEQ ID No. 15 |
| | G8 | C6ssC6 | Sense (5′-3′) | SEQ ID No. 14 |
| L16 | / | / | Antisense (3′-5′) | SEQ ID No. 15 |
| | G9 | C6ssC6 | Sense (5′-3′) | SEQ ID No. 14 |
| L17 | / | / | Antisense (3′-5′) | SEQ ID No. 15 |
| | G10 | C6ssC6 | Sense (5′-3′) | SEQ ID No. 14 |
| L18 | / | / | Antisense (3′-5′) | SEQ ID No. 15 |
| | G11 | C6ssC6 | Sense (5′-3′) | SEQ ID No. 14 |
| L19 | / | / | Antisense (3′-5′) | SEQ ID No. 15 |
| | G12 | C6ssC6 | Sense (5′-3′) | SEQ ID No. 14 |
| L40 | / | / | Antisense (3′-5′) | SEQ ID No. 15 |
| | G1 | / | Sense (5′-3′) | SEQ ID No. 14 |
| L41 | / | / | Antisense (3′-5′) | SEQ ID No. 15 |
| | G2 | / | Sense (5′-3′) | SEQ ID No. 14 |
| L42 | / | / | Antisense (3′-5′) | SEQ ID No. 15 |
| | G3 | / | Sense (5′-3′) | SEQ ID No. 14 |
| L43 | / | / | Antisense (3′-5′) | SEQ ID No. 15 |
| | G4 | / | Sense (5′-3′) | SEQ ID No. 14 |
| L44 | / | / | Antisense (3′-5′) | SEQ ID No. 15 |
| | G5 | / | Sense (5′-3′) | SEQ ID No. 14 |
| L45 | / | / | Antisense (3′-5′) | SEQ ID No. 15 |
| | G6 | / | Sense (5′-3′) | SEQ ID No. 14 |
| L53 | / | / | Antisense (3′-5′) | SEQ ID No. 15 |
| | G7 | / | Sense (5′-3′) | SEQ ID No. 14 |
| L54 | / | / | Antisense (3′-5′) | SEQ ID No. 15 |
| | G8 | / | Sense (5′-3′) | SEQ ID No. 14 |
| L55 | / | / | Antisense (3′-5′) | SEQ ID No. 15 |
| | G9 | / | Sense (5′-3′) | SEQ ID No. 14 |
| L56 | / | / | Antisense (3′-5′) | SEQ ID No. 15 |
| | G10 | / | Sense (5′-3′) | SEQ ID No. 14 |
| L57 | / | / | Antisense (3′-5′) | SEQ ID No. 15 |
| | G11 | / | Sense (5′-3′) | SEQ ID No. 14 |
| L58 | / | / | Antisense (3′-5′) | SEQ ID No. 15 |
| | G12 | / | Sense (5′-3′) | SEQ ID No. 14 |

*Although the sequence of the antisense strand in the present disclosure, such as SEQ ID No. 15 in Table 5, is presented in 5′-3′ direction, it is understood that the antisense strand hybridizes to the sense strand in the 3′-5′ direction.

TABLE 6
Conjugates comprising a GalNAc Cluster and XD-14369K1 duplex

| | | Linker | | | Linker | |
|---|---|---|---|---|---|---|
| | GalNAc | (attached to | | | (attached to | GalNAc |
| Conjugate | Cluster | 5′ of the | | | the 3′ of the | Cluster |
| No. | Structure | sense strand) | saRNA Sequences | | sense strand) | Structure |
| L60 | / | / | Antisense | SEQ ID | / | / |
| | | | (3′-5′) | No. 25* | | |
| | G13 | / | Sense | SEQ ID | / | / |
| | | | (5′-3′) | No. 24 | | |
| L66 | / | / | Antisense | SEQ ID | / | / |
| | | | (3′-5′) | No. 25 | | |
| | G13 | dT | Sense | SEQ ID | / | / |
| | | | (5′-3′) | No. 24 | | |
| L61 | / | / | Antisense | SEQ ID | / | / |
| | | | (3′-5′) | No. 25 | | |
| | G14 | / | Sense | SEQ ID | / | / |
| | | | (5′-3′) | No. 24 | | |
| L67 | / | / | Antisense | SEQ ID | / | / |
| | | | (3′-5′) | No. 25 | | |
| | G14 | dT | Sense | SEQ ID | / | / |
| | | | (5′-3′) | No. 24 | | |
| L62 | / | / | Antisense | SEQ ID | / | / |
| | | | (3′-5′) | No. 25 | | |
| | G15 | / | Sense | SEQ ID | / | / |
| | | | (5′-3′) | No. 24 | | |
| L68 | / | / | Antisense | SEQ ID | / | / |
| | | | (3′-5′) | No. 25 | | |
| | G15 | dT | Sense | SEQ ID | / | / |
| | | | (5′-3′) | No. 24 | | |
| L63 | / | / | Antisense | SEQ ID | / | / |
| | | | (3′-5′) | No. 25 | | |
| | G16 | / | Sense | SEQ ID | / | / |
| | | | (5′-3′) | No. 24 | | |
| L69 | / | / | Antisense | SEQ ID | / | / |
| | | | (3′-5′) | No. 25 | | |
| | G16 | dT | Sense | SEQ ID | / | / |
| | | | (5′-3′) | No. 24 | | |
| L64 | / | / | Antisense | SEQ ID | / | / |
| | | | (3′-5′) | No. 25 | | |
| | G17 | / | Sense | SEQ ID | / | / |
| | | | (5′-3′) | No. 24 | | |
| L70 | / | / | Antisense | SEQ ID | / | / |
| | | | (3′-5′) | No. 25 | | |
| | G17 | dT | Sense | SEQ ID | / | / |
| | | | (5′-3′) | No. 24 | | |
| L65 | / | / | Antisense | SEQ ID | / | / |
| | | | (3′-5′) | No. 25 | | |
| | G18 | / | Sense | SEQ ID | / | / |
| | | | (5′-3′) | No. 24 | | |
| L71 | / | / | Antisense | SEQ ID | / | / |
| | | | (3′-5′) | No. 25 | | |
| | G18 | dT | Sense | SEQ ID | / | / |
| | | | (5′-3′) | No. 24 | | |
| L72 | / | / | Antisense | SEQ ID | / | / |
| | | | (3′-5′) | No. 25 | | |
| | G19 | / | Sense | SEQ ID | / | / |
| | | | (5′-3′) | No. 24 | | |
| L73 | / | / | Antisense | SEQ ID | / | / |
| | | | (3′-5′) | No. 25 | | |
| | G20 | / | Sense | SEQ ID | / | G22 |
| | | | (5′-3′) | No. 24 | | |
| L74 | / | / | Antisense | SEQ ID | / | / |
| | | | (3′-5′) | No. 25 | | |
| | G21 | / | Sense | SEQ ID | / | G22 |
| | | | (5′-3′) | No. 24 | | |
| L75 | / | / | Antisense | SEQ ID | / | / |
| | | | (3′-5′) | No. 25 | | |
| | / | / | Sense | SEQ ID | / | G23 |
| | | | (5′-3′) | No. 24 | | |
| L76 | / | / | Antisense | SEQ ID | / | / |
| | | | (3′-5′) | No. 25 | | |
| | G17 | / | Sense | SEQ ID | / | G23 |
| | | | (5′-3′) | No. 24 | | |
| L77 | / | / | Antisense | SEQ ID | / | / |
| | | | (3′-5′) | No. 25 | | |
| | G17 | dT | Sense | SEQ ID | dT | G23 |
| | | | (5′-3′) | No. 24 | | |
| L78 | / | / | Antisense | SEQ ID | / | / |
| | | | (3′-5′) | No. 25 | | |
| | G14 | dT | Sense | SEQ ID | dT | G24 |
| | | | (5′-3′) | No. 24 | | |
| L79 | / | / | Antisense | SEQ ID | / | / |
| | | | (3′-5′) | No. 25 | | |
| | / | / | Sense | SEQ ID | | G24 |
| | | | (5′-3′) | No. 24 | | |
| L80 | / | / | Antisense | SEQ ID | / | / |
| | | | (3′-5′) | No. 25 | | |
| | G7 | / | Sense | SEQ ID | / | / |
| | | | (5′-3′) | No. 24 | | |
| L81 | / | / | Antisense | SEQ ID | / | / |
| | | | (3′-5′) | No. 25 | | |
| | G9 | / | Sense | SEQ ID | / | / |
| | | | (5′-3′) | No. 24 | | |

*Although the sequence of the antisense strand in the present disclosure, such as SEQ ID No. 25 in Table 6, is presented in 5′-3′ direction, it is understood that the antisense strand hybridizes to the sense strand in the 3′-5′direction.

In some embodiments, the GalNAc-nucleotide conjugate (such as GalNAc-saRNA conjugate or GalNAc-siRNA conjugate) has a structure of any of the following:
(L=an optional linker, such as C6ssC6 for Conjugates L1 to L19 or dT for L66 to L71; L is not present for Conjugates L40 to L58, L60-65, and L72-75;
Po=Phosphodiester bond;
Ps=Phosphorothioate bond; and
Nuc=nucleotide or oligonucleotide, such as a sense strand of a double-stranded saRNA (e.g., XD-06414)) or a double-stranded siRNA
1). C6-GalNAc (encompassing the GalNAc-saRNA conjugate disclosed in Example 2 of PCT/EP2018/074211 filed Sep. 7, 2018):

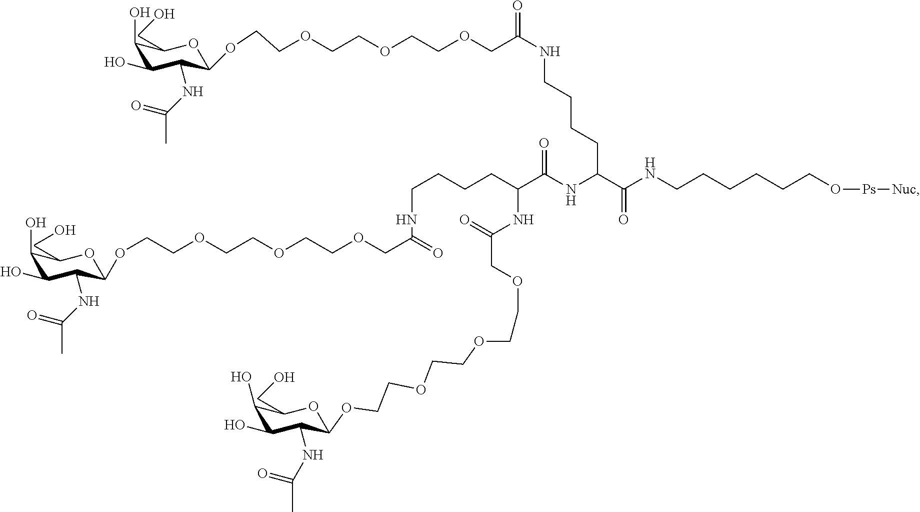

2). GalNAc-Clv (encompassing a GalNAc-saRNA conjugate disclosed in PCT/EP2018/074211 filed Sep. 7, 2018:

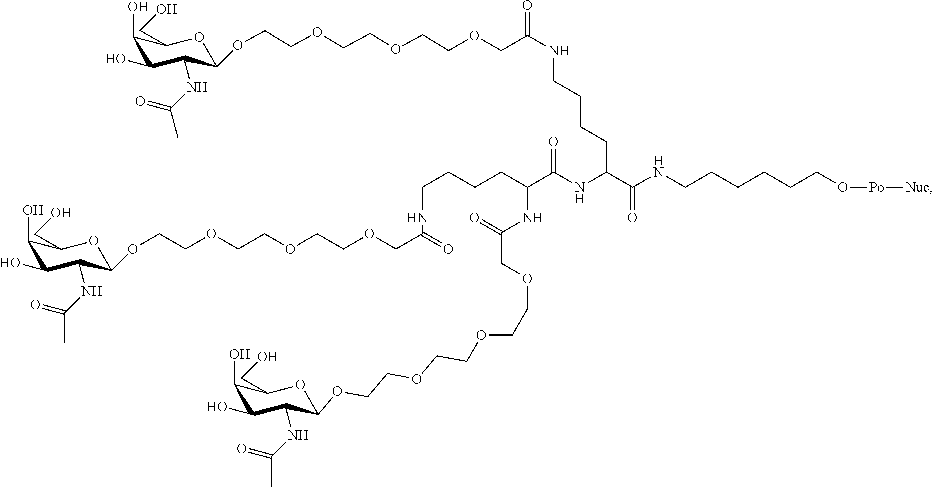

3). CJ1 (encompassing L1 and L40 in Table 5):

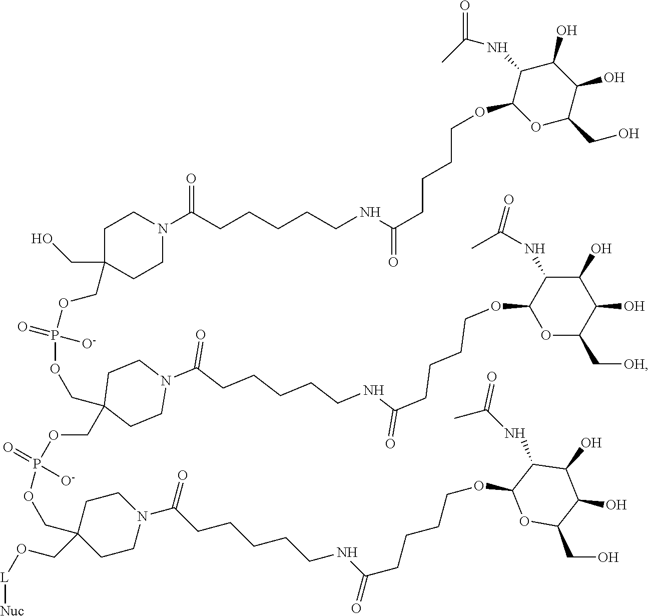

4). CJ2 (encompassing L2 and L41 in Table 5):

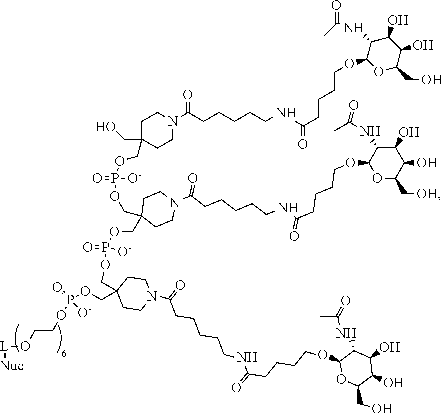

5). CJ3 (encompassing L3 and L42 in Table 5):

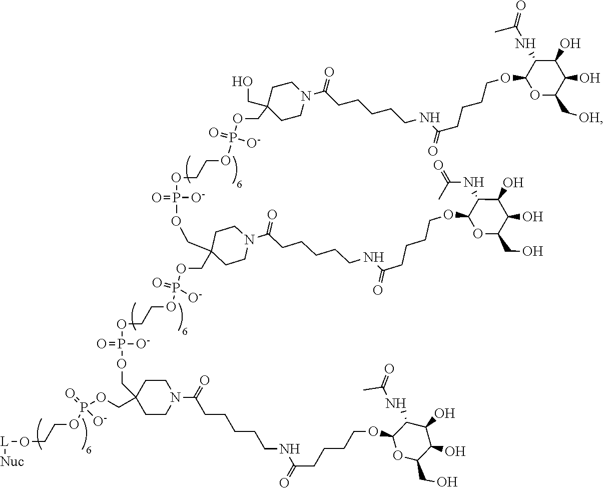

6). CJ4 (encompassing L4 and L43 in Table 5):

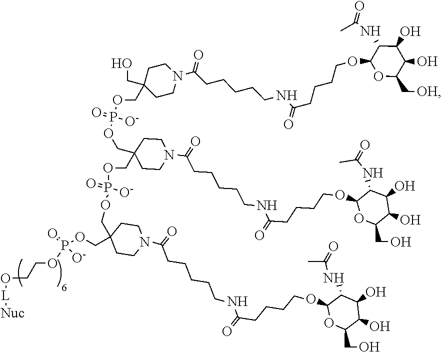

7). CJ5 (encompassing L5 and L44 in Table 5):

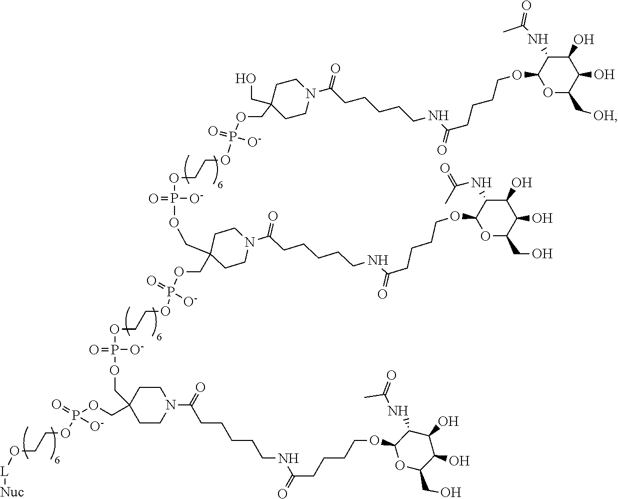

8). CJ6 (encompassing L6 and L45 in Table 5):

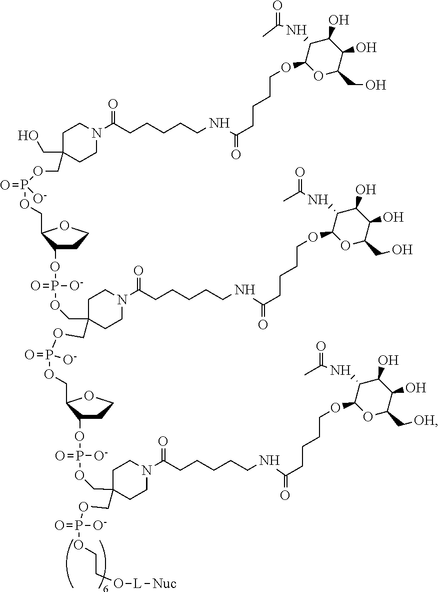

9). CJ7 (encompassing L14 and L53 in Table 5 and L80 in Table 6):

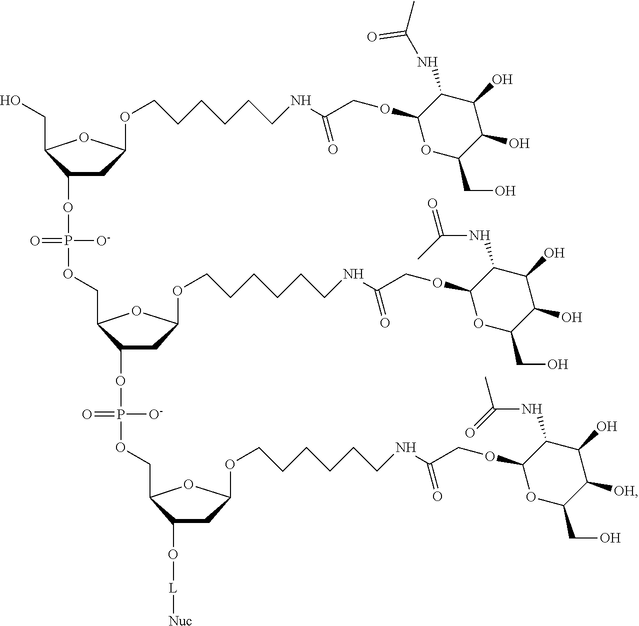

10). CJ8 (encompassing L15 and L54 in Table 5):

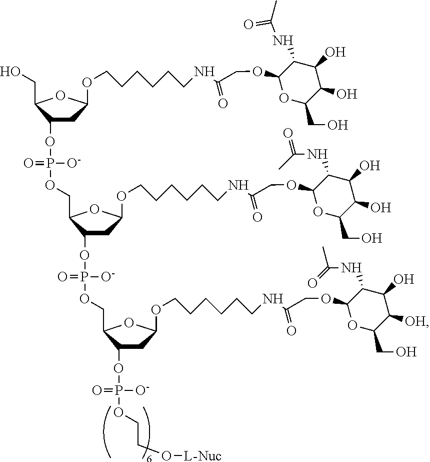

11). CJ9 (encompassing L16 and L55 in Table 5 and L81 in Table 6):

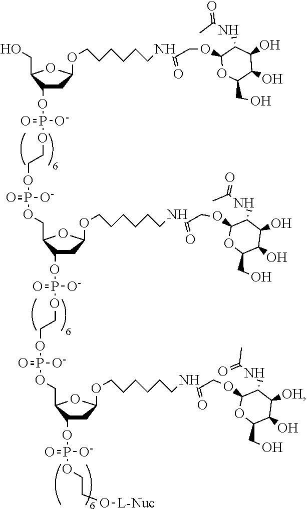

12). CJ10 (encompassing L17 and L56 in Table 5):

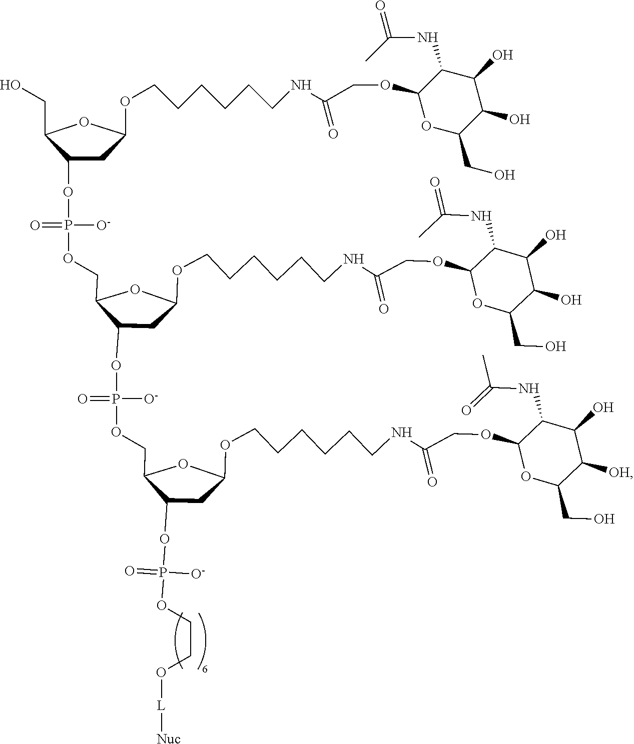

13). CJ11 (encompassing L18 and L57 in Table 5):

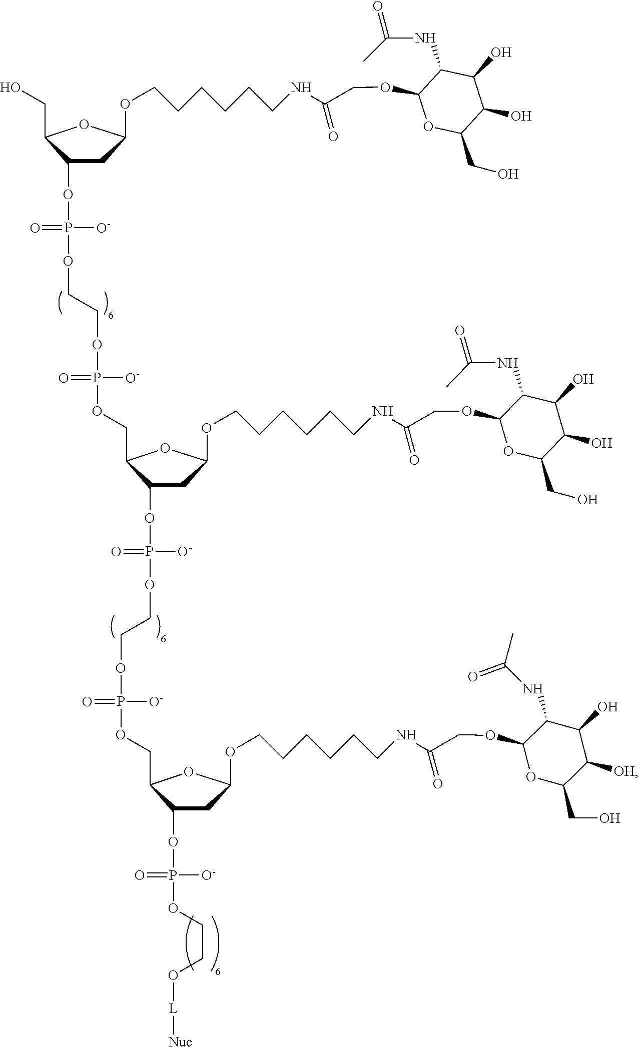

14). CJ12 (encompassing L19 and L58 in Table 5):

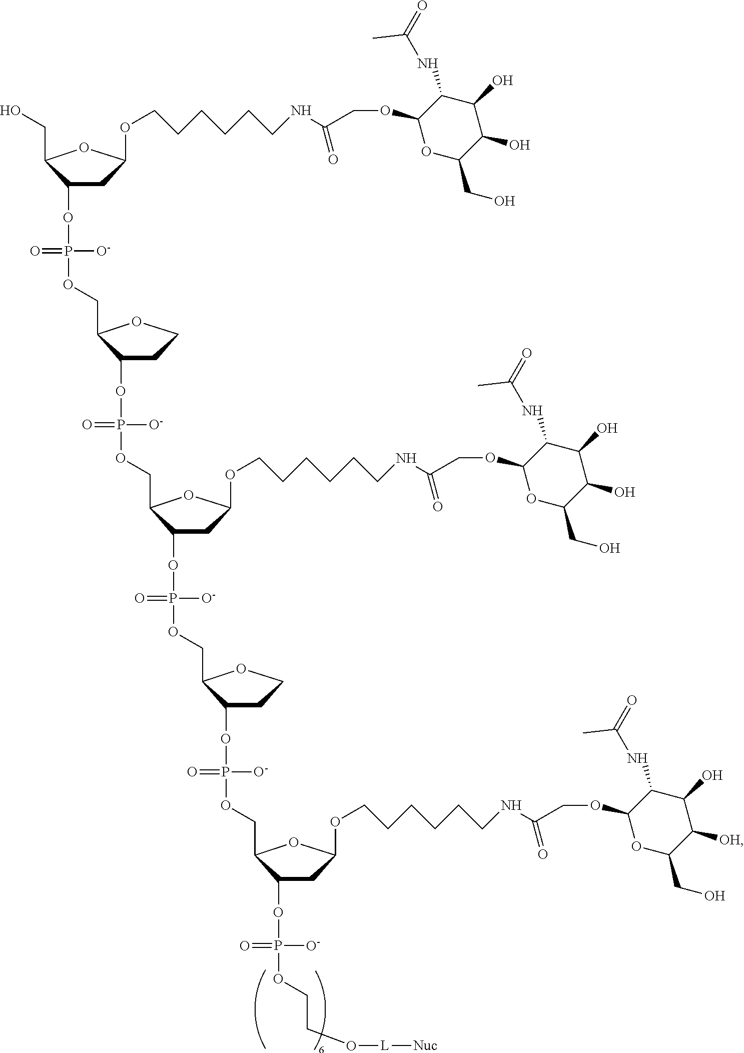

15). CJ13 (encompassing L60 and L66 in Table 6):

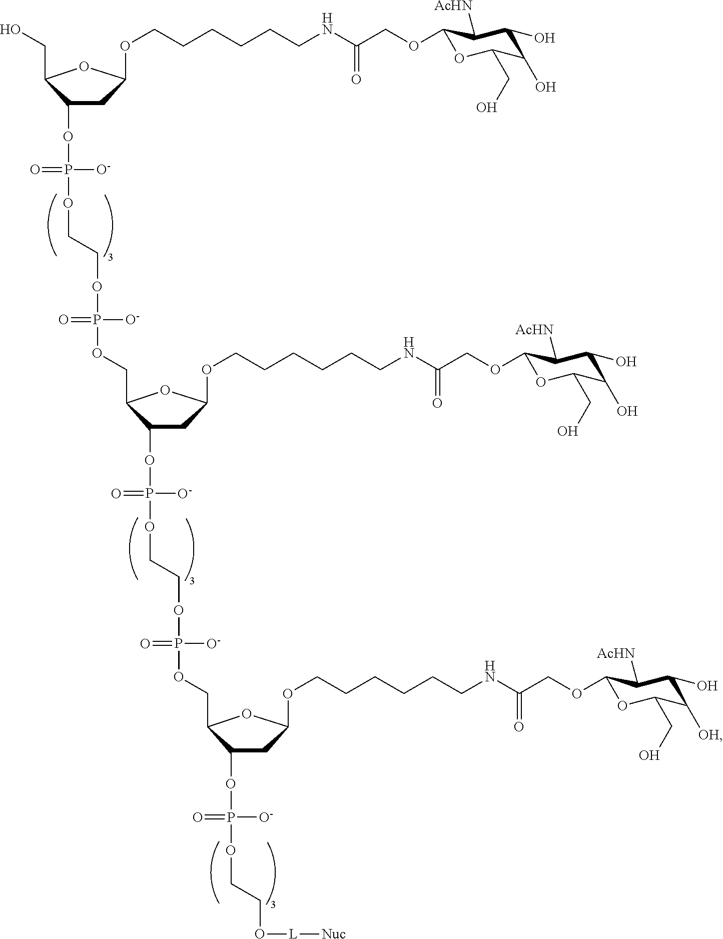

16). CJ14 (encompassing L61 and L67 in Table 6):

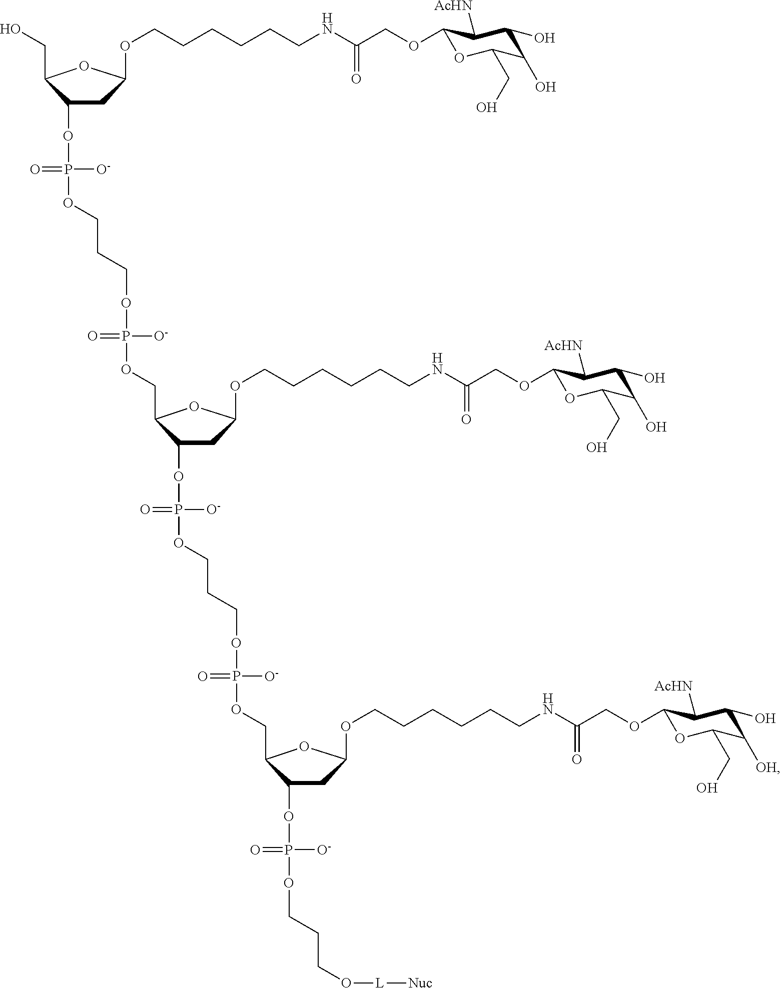

17). CJ15 (encompassing L62 and L68 in Table 6):

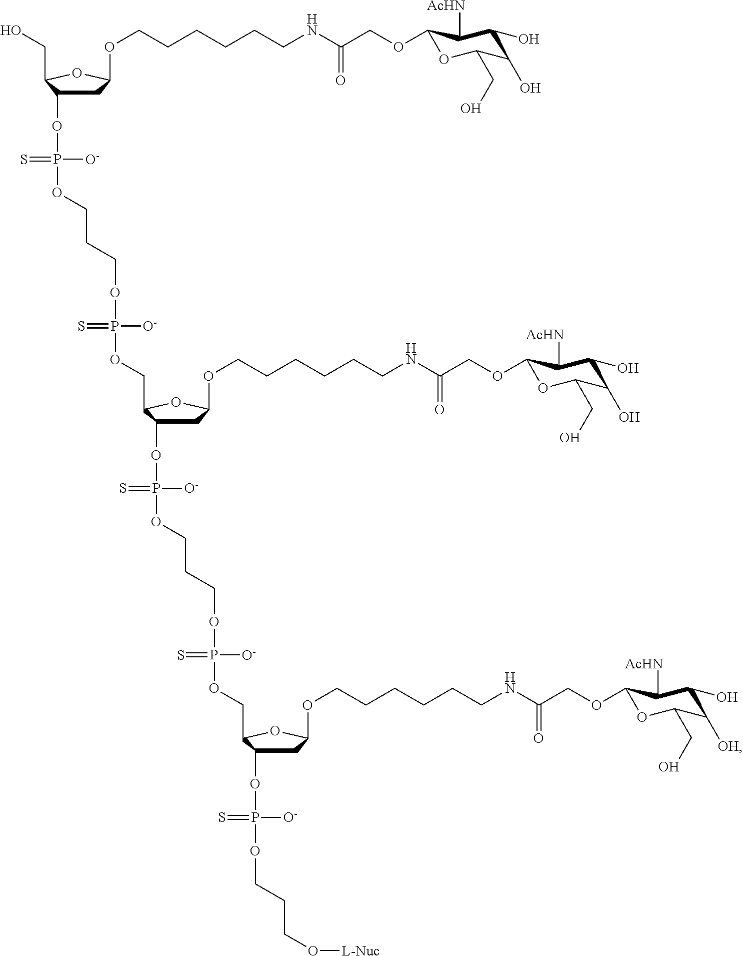

18). CJ16 (encompassing L63 and L69 in Table 6):

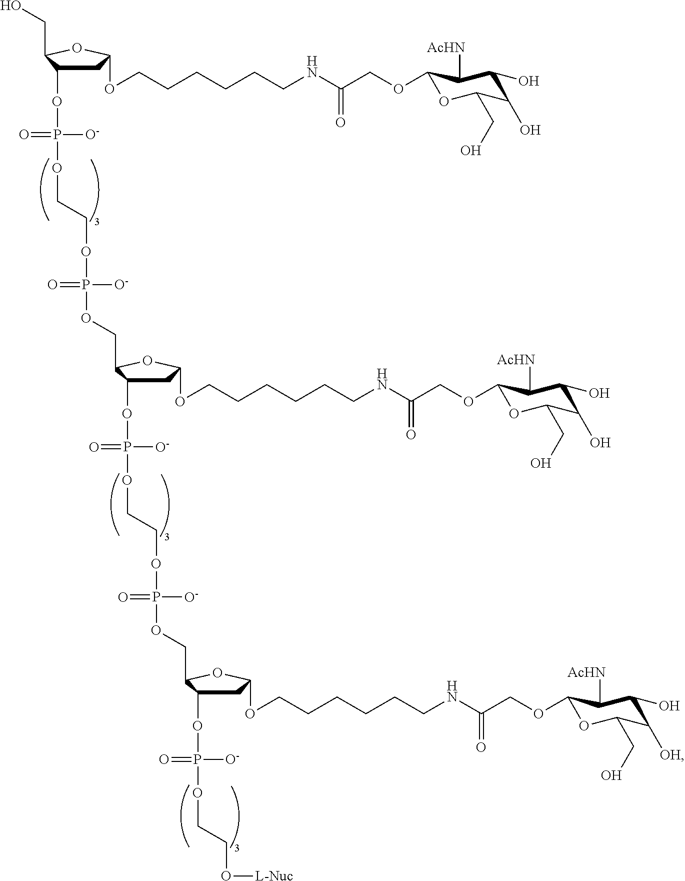

19). CJ17 (encompassing L64 and L70 in Table 6):

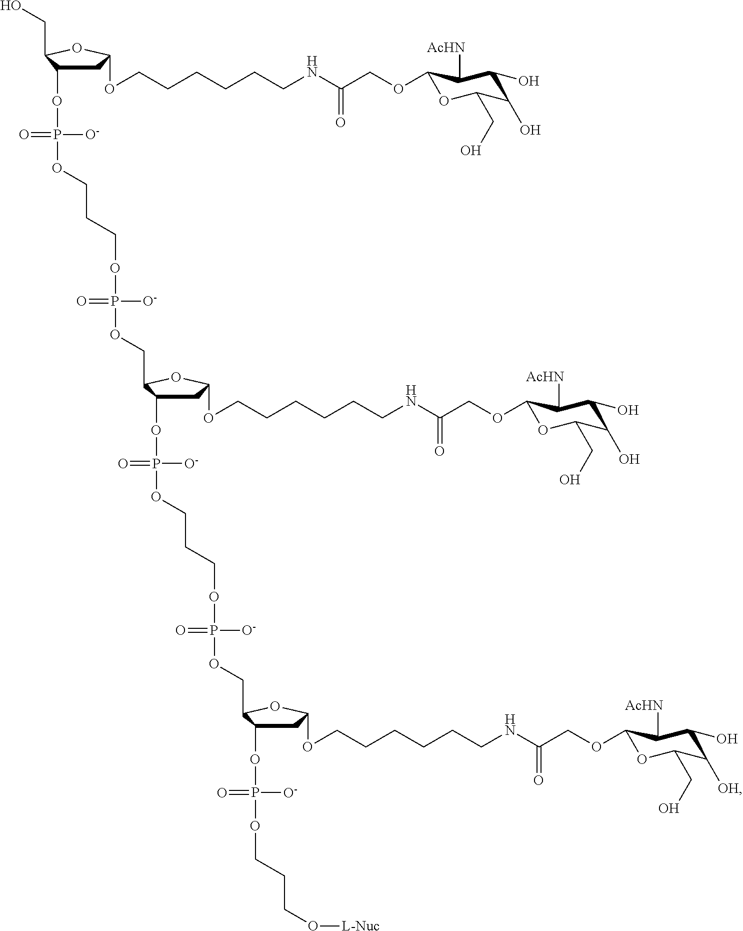

20). CJ18 (encompassing L65 and L71 in Table 6):

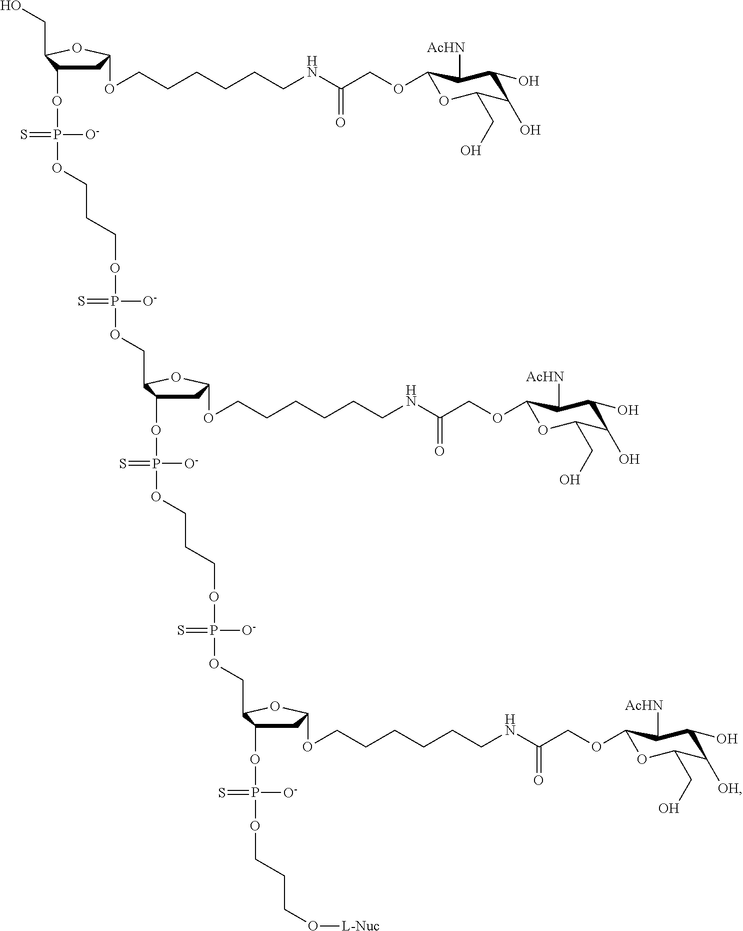

21). CJ19 (encompassing L72 and L73 in Table 6):

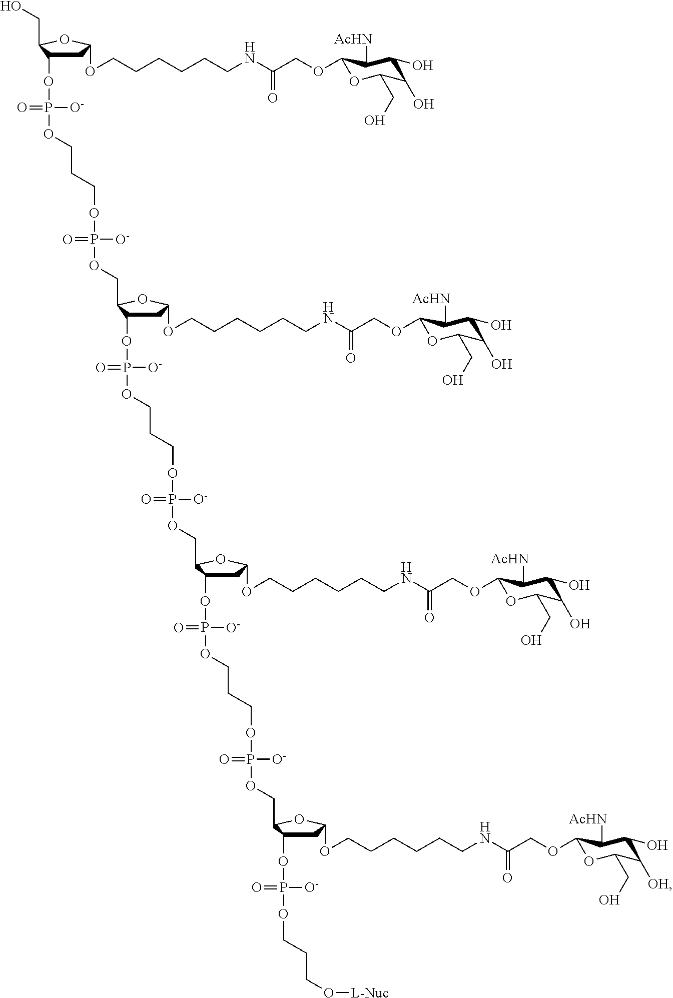

22). CJ20 (encompassing L73 in Table 6):

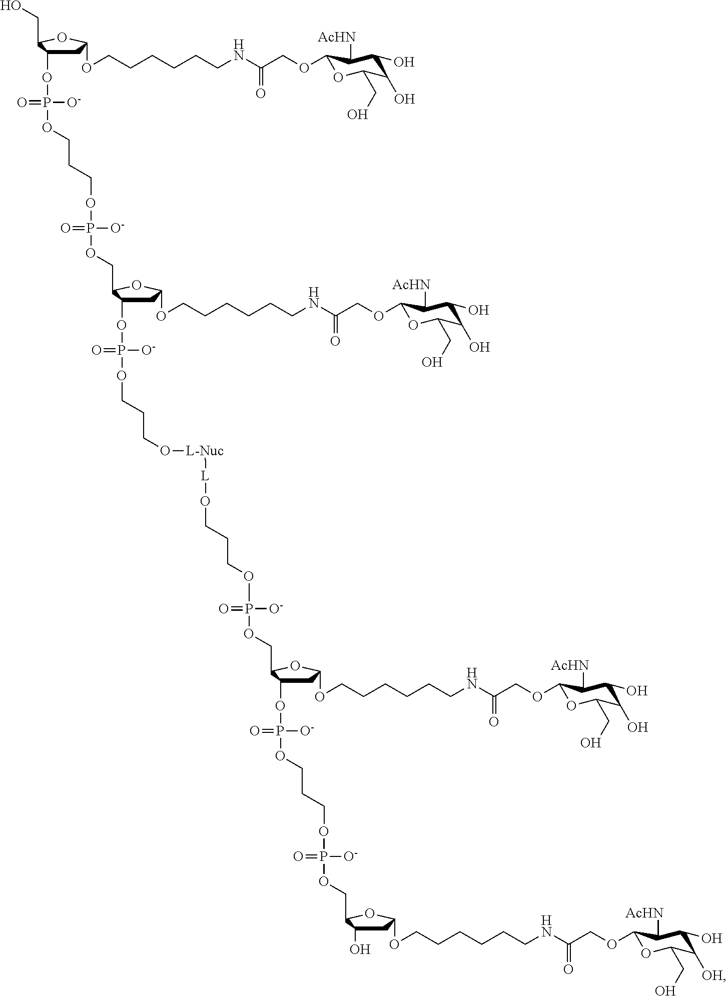

23). CJ21 (encompassing L74 in Table 6):

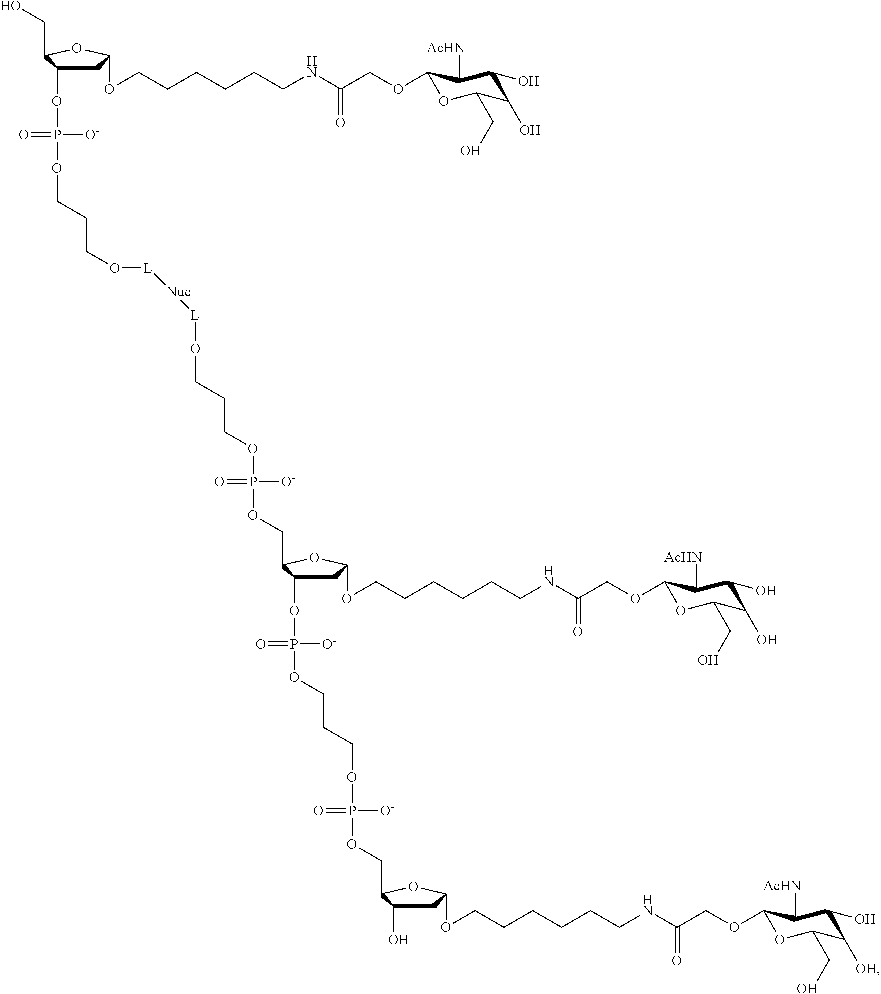

24). CJ22 (encompassing L75 in Table 6):

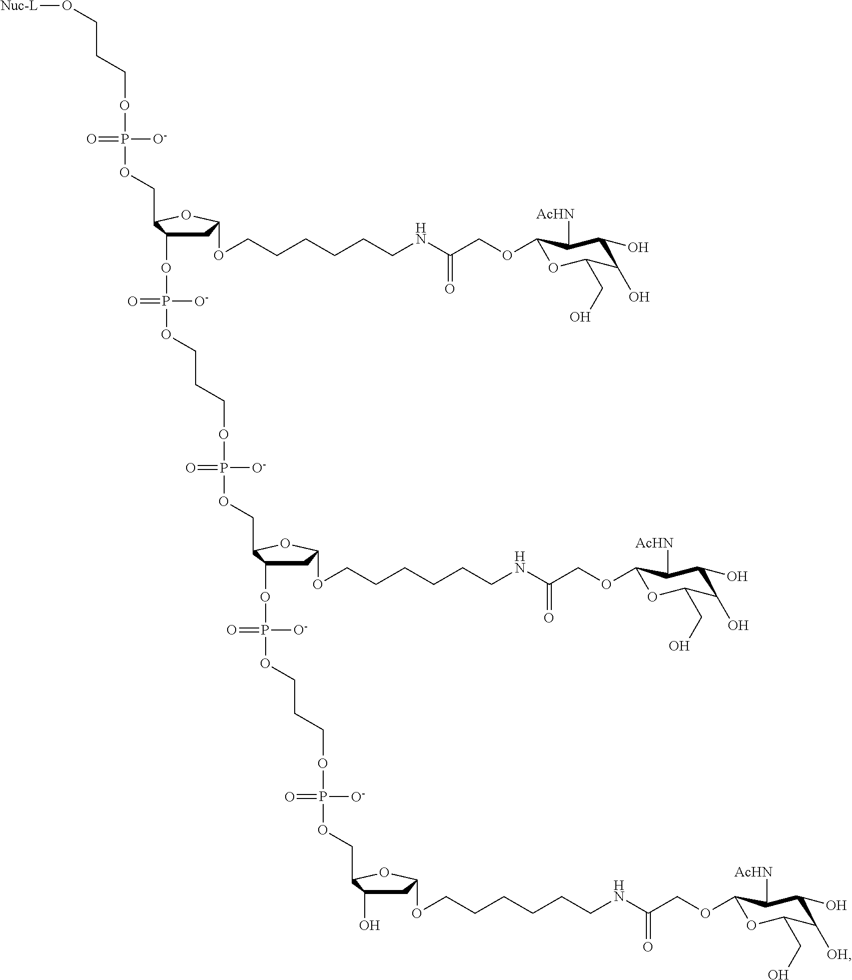

25). CJ23 (encompassing L76 and L77 in Table 6):
26). CJ24 (encompassing L78 in Table 6):
27). CJ25 (encompassing L79 in Table 6):

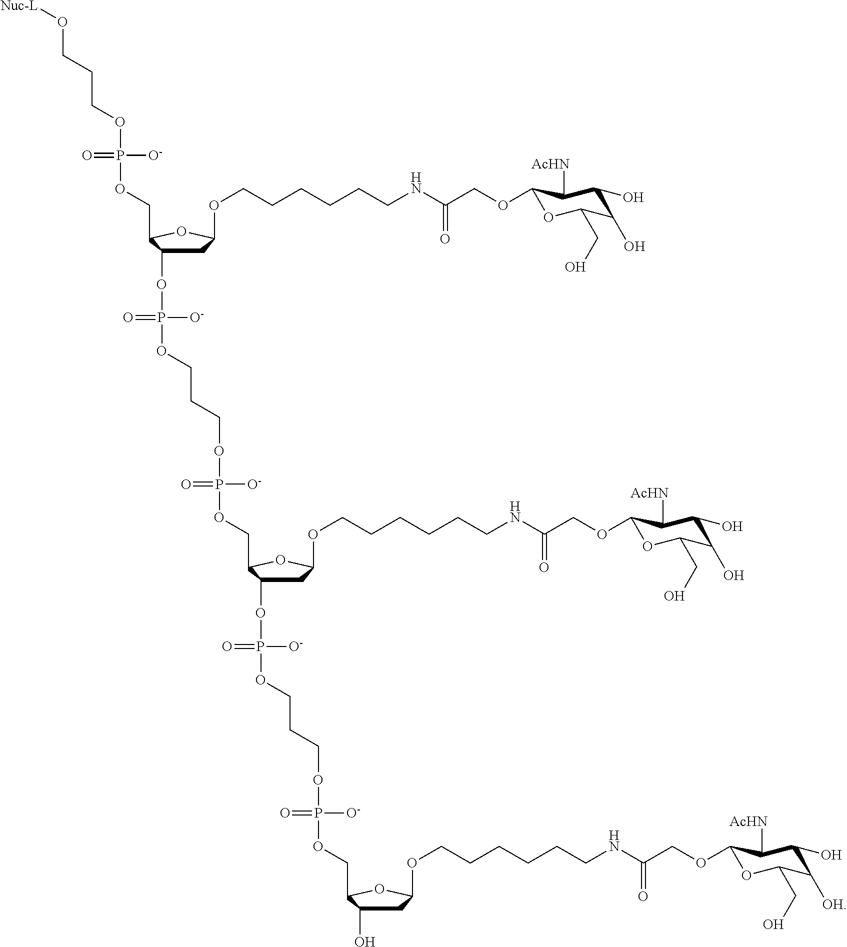

In some cases, the GalNAc-saRNA conjugate up-regulates the expression of CEBPA, wherein the saRNA is a CEBPA-saRNA. For example, the CEBPA-saRNA may be any saRNA in Table 2, such as XD-06414 (SEQ ID Nos. 14 and 15).

In some embodiments, the GalNAc-saRNA conjugate or the GalNAc-siRNA conjugate is delivered to a liver cell of a subject. The liver cell may be liver cancer cell.

The GalNAc-saRNA conjugate may be synthesized by any suitable method known in the art. For example, the GalNAc-saRNA conjugate may be synthesized according to the methods described in the experimental section of Prakash et al., Journal of Medicinal Chemistry, vol.59:2718-2733 (2016), the contents of which are incorporated herein by reference in their entirety.

In some embodiments, the GalNAc moieties are conjugated to an siRNA in order to form a GalNAc-siRNA conjugate.

In some cases, the GalNAc-siRNA conjugate down-regulates the expression of a targeted gene.

In some embodiments, the GalNAc-siRNA conjugate is delivered to a liver cell of a subject. The liver cell may be liver cancer cell.

The GalNAc-siRNA conjugate may be synthesized by any suitable method known in the art. For example, the GalNAc-siRNA conjugate may be synthesized according to the methods described in the experimental section of Prakash et al., Journal of Medicinal Chemistry, vol.59:2718-2733 (2016), the contents of which are incorporated herein by reference in their entirety.

### II. Pharmaceutical Composition

One aspect of the present invention provides pharmaceutical compositions comprising a small activating RNA (saRNA) that upregulates a target gene, and at least one pharmaceutically acceptable carrier.

### Formulation, Delivery, Administration, and Dosing

Pharmaceutical formulations may additionally comprise a pharmaceutically acceptable excipient, which, as used herein, includes, but is not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21^{st} Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

In some embodiments, compositions are administered to humans, human patients or subjects. For the purposes of the present disclosure, the phrase “active ingredient” generally refers to saRNA to be delivered as described herein.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, and/or rats; and/or birds, including commercially relevant birds such as poultry, chickens, ducks, geese, and/or turkeys.

In one embodiment, the efficacy of the formulated saRNA described herein may be determined in proliferating cells.

Formulations of the pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the invention may be prepared, packaged, and/or sold in bulk, as a single unit dose, and/or as a plurality of single unit doses. As used herein, a “unit dose” is discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition in accordance with the invention will vary, depending upon the identity, size, and/or condition of the subject treated and further depending upon the route by which the composition is to be administered. By way of example, the composition may comprise between 0.1% and 100%, e.g., between .5 and 50%, between 1-30%, between 5-80%, at least 80% (w/w) active ingredient.

In some embodiments, the formulations described herein may contain at least one saRNA. As a non-limiting example, the formulations may contain 1, 2, 3, 4 or 5 saRNAs with different sequences. In one embodiment, the formulation contains at least three saRNAs with different sequences. In one embodiment, the formulation contains at least five saRNAs with different sequences.

The saRNA of the present invention can be formulated using one or more excipients to: (1) increase stability; (2) increase cell transfection; (3) permit the sustained or delayed release (e.g., from a depot formulation of the saRNA); (4) alter the biodistribution (e.g., target the saRNA to specific tissues or cell types); (5) increase the translation of encoded protein in vivo; and/or (6) alter the release profile of encoded protein in vivo.

In addition to traditional excipients such as any and all solvents, dispersion media, diluents, or other liquid vehicles, dispersion or suspension aids, surface active agents, isotonic agents, thickening or emulsifying agents, preservatives, excipients of the present invention can include, without limitation, lipidoids, liposomes, lipid nanoparticles, polymers, lipoplexes, core-shell nanoparticles, peptides, proteins, cells transfected with saRNA (e.g., for transplantation into a subject), hyaluronidase, nanoparticle mimics and combinations thereof. Accordingly, the formulations of the invention can include one or more excipients, each in an amount that together increases the stability of the saRNA and/or increases cell transfection by the saRNA. Further, the saRNA of the present invention may be formulated using self-assembled nucleic acid nanoparticles. Pharmaceutically acceptable carriers, excipients, and delivery agents for nucleic acids that may be used in the formulation with the saRNA of the present invention are disclosed in International Publication WO 2013/090648 filed Dec. 14, 2012, the contents of which are incorporated herein by reference in their entirety.

### Delivery

The present disclosure encompasses the delivery of saRNA for any of therapeutic, prophylactic, pharmaceutical, diagnostic or imaging by any appropriate route taking into consideration likely advances in the sciences of drug delivery. Delivery may be naked or formulated.

The saRNA of the present invention may be delivered to a cell naked. As used herein in, “naked” refers to delivering saRNA free from agents which promote transfection. For example, the saRNA delivered to the cell may contain no modifications. The naked saRNA may be delivered to the cell using routes of administration known in the art and described herein.

The saRNA of the present invention may be formulated, using the methods described herein. The formulations may contain saRNA which may be modified and/or unmodified. The formulations may further include, but are not limited to, cell penetration agents, a pharmaceutically acceptable carrier, a delivery agent, a bioerodible or biocompatible polymer, a solvent, and a sustained-release delivery depot. The formulated saRNA may be delivered to the cell using routes of administration known in the art and described herein.

In some embodiments, the saRNA of the present invention is delivered with non-encapsulation technology, such as an agent comprising an N-acetylgalactosamine (GalNAc) group or derivatives thereof, or a cluster comprising more than one GalNAc groups or derivatives thereof connected through a bivalent or trivalent branched linker.

The compositions may also be formulated for direct delivery to an organ or tissue in any of several ways in the art including, but not limited to, direct soaking or bathing, via a catheter, by gels, powder, ointments, creams, gels, lotions, and/or drops, by using substrates such as fabric or biodegradable materials coated or impregnated with the compositions, and the like. The saRNA of the present invention may also be cloned into a retroviral replicating vector (RRV) and transduced to cells.

### Administration

The saRNA of the present invention may be administered by any route which results in a therapeutically effective outcome. These include, but are not limited to enteral, gastroenteral, epidural, oral, transdermal, epidural (peridural), intracerebral (into the cerebrum), intracerebroventricular (into the cerebral ventricles), epicutaneous (application onto the skin), intradermal, (into the skin itself), subcutaneous (under the skin), nasal administration (through the nose), intravenous (into a vein), intraarterial (into an artery), intramuscular (into a muscle), intracardiac (into the heart), intraosseous infusion (into the bone marrow), intrathecal (into the spinal canal), intraperitoneal, (infusion or injection into the peritoneum), intravesical infusion, intravitreal, (through the eye), intracavernous injection, (into the base of the penis), intravaginal administration, intrauterine, extra-amniotic administration, transdermal (diffusion through the intact skin for systemic distribution), transmucosal (diffusion through a mucous membrane), insufflation (snorting), sublingual, sublabial, enema, eye drops (onto the conjunctiva), or in ear drops. In specific embodiments, compositions may be administered in a way which allows them cross the blood-brain barrier, vascular barrier, or other epithelial barrier. Routes of administration disclosed in International Publication WO 2013/090648 filed Dec. 14, 2012, the contents of which are incorporated herein by reference in their entirety, may be used to administer the saRNA of the present invention.

### Dosage Forms

A pharmaceutical composition described herein can be formulated into a dosage form described herein, such as a topical, intranasal, intratracheal, or injectable (e.g., intravenous, intraocular, intravitreal, intramuscular, intracardiac, intraperitoneal, subcutaneous). Liquid dosage forms, injectable preparations, pulmonary forms, and solid dosage forms described in International Publication WO 2013/090648 filed Dec. 14, 2012, the contents of which are incorporated herein by reference in their entirety may be used as dosage forms for the saRNA of the present invention.

### III. Methods of Use

One aspect of the present invention provides methods of delivering saRNAs into cells by a GalNAc-saRNA conjugate without any transfection agent. The cells express asialoglycoprotein receptors. In some embodiments, targeted delivery of saRNAs into cells are achieved with GalNAc-saRNA conjugates of the present invention. In some cases, the cells are liver cells. In some cases, the cells are liver cancer cells.

Another aspect of the present invention provides methods of using saRNA or a GalNAc-saRNA conjugate of the present invention and pharmaceutical compositions comprising the saRNA or the GalNAc-saRNA conjugate and at least one pharmaceutically acceptable carrier. The saRNA or the GalNAc-saRNA conjugate of the present invention modulates the expression of its target gene. In one embodiment is provided a method of regulating the expression of a target gene in vitro and/or in vivo comprising administering the saRNA of the present invention. In one embodiment, the expression of the target gene is increased by at least 5, 10, 20, 30, 40%, or at least 45, 50, 55, 60, 65, 70, 75%, or at least 80% in the presence of the saRNA of the present invention compared to the expression of the target gene in the absence of the saRNA of the present invention. In a further embodiment, the expression of the target gene is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, in the presence of the saRNA of the present invention compared to the expression of the target gene in the absence of the saRNA of the present invention.

In one embodiment, the increase in gene expression of the saRNA descried herein is shown in proliferating cells.

In one embodiment, the saRNA described herein may be used as a spacer in a CRISPR (clustered regularly interspaced palindromic repeats) system, such as a CRISPR/Cas9 system. The CRISPR system comprising saRNA described herein may be used to cleave and edit a target gene.

In one embodiment, the increase in gene expression of the saRNA or the GalNAc-saRNA conjugate treatment descried herein is shown in proliferating cells.

### Hyperproliferation Disorders

In one embodiment of the invention, the saRNA or the GalNAc-saRNA conjugate of the present invention is used to reduce cell proliferation of hyperproliferative cells. Examples of hyperproliferative cells include cancerous cells, e.g., carcinomas, sarcomas, lymphomas and blastomas. Such cancerous cells may be benign or malignant. Hyperproliferative cells may result from an autoimmune condition such as rheumatoid arthritis, inflammatory bowel disease, or psoriasis. Hyperproliferative cells may also result within patients with an oversensitive immune system coming into contact with an allergen. Such conditions involving an oversensitive immune system include, but are not limited to, asthma, allergic rhinitis, eczema, and allergic reactions, such as allergic anaphylaxis. In one embodiment, tumor cell development and/or growth is inhibited. In a preferred embodiment, solid tumor cell proliferation is inhibited. In another preferred embodiment, metastasis of tumor cells is prevented. In another preferred example, undifferentiated tumor cell proliferation is inhibited.

Inhibition of cell proliferation or reducing proliferation means that proliferation is reduced or stops altogether. Thus, “reducing proliferation” is an embodiment of “inhibiting proliferation”. Proliferation of a cell is reduced by at least 20%, 30% or 40%, or preferably at least 45, 50, 55, 60, 65, 70 or 75%, even more preferably at least 80, 90 or 95% in the presence of the saRNA or the GalNAc-saRNA conjugate of the invention compared to the proliferation of said cell prior to treatment with the saRNA or the GalNAc-saRNA conjugate of the invention, or compared to the proliferation of an equivalent untreated cell. In embodiments wherein cell proliferation is inhibited in hyperproliferative cells, the “equivalent” cell is also a hyperproliferative cell. In preferred embodiments, proliferation is reduced to a rate comparable to the proliferative rate of the equivalent healthy (non-hyperproliferative) cell. Alternatively viewed, a preferred embodiment of “inhibiting cell proliferation” is the inhibition of hyperproliferation or modulating cell proliferation to reach a normal, healthy level of proliferation.

In one non-limiting example, the saRNA or the GalNAc-saRNA conjugate of the present invention is used to reduce the proliferation of leukemia and lymphoma cells. Preferably, the cells include Jurkat cells (acute T cell lymphoma cell line), K562 cells (erythroleukemia cell line), U373 cells (glioblastoma cell line), and 32Dp210 cells (myeloid leukemia cell line).

In another non-limiting example, the saRNA or the GalNAc-saRNA conjugate of the present invention is used to reduce the proliferation of ovarian cancer cells, liver cancer cells, pancreatic cancer cells, breast cancer cells, prostate cancer cells, rat liver cancer cells, and insulinoma cells. Preferably, the cells include PEO1 and PEO4 (ovarian cancer cell line), HepG2 (hepatocellular carcinoma cell line), Pancl (human pancreatic carcinoma cell line), MCF7 (human breast adenocarcinoma cell line), DU145 (human metastatic prostate cancer cell line), rat liver cancer cells, and MING (rat insulinoma cell line).

In one embodiment, the saRNA or the GalNAc-saRNA conjugate of the present invention is used to treat hyperproliferative disorders. Tumors and cancers represent a hyperproliferative disorder of particular interest, and all types of tumors and cancers, e.g. solid tumors and haematological cancers are included. Examples of cancer include, but not limited to, cervical cancer, uterine cancer, ovarian cancer, kidney cancer, gallbladder cancer, liver cancer, head and neck cancer, squamous cell carcinoma, gastrointestinal cancer, breast cancer, prostate cancer, testicular cancer, lung cancer, non-small cell lung cancer, non-Hodgkin's lymphoma, multiple myeloma, leukemia (such as acute lymphocytic leukemia, chronic lymphocytic leukemia, acute myelogenous leukemia, and chronic myelogenous leukemia), brain cancer (e.g. astrocytoma, glioblastoma, medulloblastoma), neuroblastoma, sarcomas, colon cancer, rectum cancer, stomach cancer, anal cancer, bladder cancer, endometrial cancer, plasmacytoma, lymphomas, retinoblastoma, Wilm's tumor, Ewing sarcoma, melanoma and other skin cancers. The liver cancer may include, but not limited to, cholangiocarcinoma, hepatoblastoma, haemangio sarcoma, or hepatocellular carcinoma (HCC). HCC is of particular interest.

Primary liver cancer is the fifth most frequent cancer worldwide and the third most common cause of cancer-related mortality. HCC represents the vast majority of primary liver cancers [El-Serag et al., Gastroenterology, vol. 132(7), 2557-2576 (2007), the contents of which are disclosed herein in their entirety]. HCC is influenced by the interaction of several factors involving cancer cell biology, immune system, and different aetiologies (viral, toxic and generic). The majority of patients with HCC develop malignant tumors from a background of liver cirrhosis. Currently most patients are diagnosed at an advanced stage and therefore the 5 year survival for the majority of HCC patients remains dismal. Surgical resection, loco-regional ablation and liver transplantation are currently the only therapeutic options which have the potential to cure HCC. However, based on the evaluation of individual liver function and tumor burden only about 5-15% of patients are eligible for surgical intervention. The present invention utilizes saRNA or the GalNAc-saRNA conjugate to modulate the expression of a target gene and treat liver cirrhosis and HCC.

The method of the present invention may reduce tumor volume by at least 10, 20, 30, 40, 50, 60, 70, 80 or 90%. Preferably, the development of one or more new tumors is inhibited, e.g. a subject treated according to the invention develops fewer and/or smaller tumors. Fewer tumors means that he develops a smaller number of tumors than an equivalent subject over a set period of time. For example, he develops at least 1, 2, 3, 4 or 5 fewer tumors than an equivalent control (untreated) subject. Smaller tumor means that the tumors are at least 10, 20, 30, 40, 50, 60, 70, 80 or 90% smaller in weight and/or volume than tumors of an equivalent subject. The method of the present invention reduces tumor burden by at least 10, 20, 30, 40, 50, 60, 70, 80 or 90%.

The set period of time may be any suitable period, e.g. 1, 2, 3, 4, 5, 6, 7, 8, 9 or 10 months or years.

In one non-limiting example, provided is a method of treating an undifferentiated tumor, comprising contacting a cell, tissue, organ or subject with the saRNA or the GalNAc-saRNA conjugate of the present invention. Undifferentiated tumors generally have a poorer prognosis compared to differentiated ones. As the degree of differentiation in tumors has a bearing on prognosis, it is hypothesized that the use of a differentiating biological agent could be a beneficial anti-proliferative drug. Undifferentiated tumors that may be treated with the saRNA or the GalNAc-saRNA conjugate include undifferentiated small cell lung carcinomas, undifferentiated pancreatic adenocarcinomas, undifferentiated human pancreatic carcinoma, undifferentiated human metastatic prostate cancer, and undifferentiated human breast cancer.

In one embodiment, the saRNA or the GalNAc-saRNA conjugate of the present invention is used to regulate oncogenes and tumor suppressor genes. Preferably, the expression of the oncogenes may be down-regulated. The expression of the oncogenes reduces by at least 20, 30, 40%, more preferably at least 45, 50, 55, 60, 65, 70, 75, 80, 85, 90, 95% in the presence of the saRNA or the GalNAc-saRNA conjugate of the invention compared to the expression in the absence of the saRNA or the GalNAc-saRNA conjugate of the invention. In a further preferable embodiment, the expression of the oncogenes is reduced by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100, in the presence of the saRNA or the GalNAc-saRNA conjugate of the invention compared to the expression in the absence of the saRNA or the GalNAc-saRNA conjugate of the invention. Preferably, the expressions of tumor suppressor genes may be inhibited. The expression of the tumor suppressor genes increase by at least 20, 30, 40%, more preferably at least 45, 50, 55, 60, 65, 70, 75, 80, 85,90, 95%, even more preferably at least 100% in the presence of the saRNA or the GalNAc-saRNA conjugate of the invention compared to the expression in the absence of the saRNA or the GalNAc-saRNA conjugate of the invention. In a further preferable embodiment, the expression of tumor suppressor genes is increased by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100 in the presence of the saRNA or the GalNAc-saRNA conjugate of the invention compared to the expression in the absence of the saRNA or the GalNAc-saRNA conjugate of the invention.

In one embodiment, the saRNA or the GalNAc-saRNA conjugate of the present invention is used to regulate micro RNAs (miRNA or miR) in the treatment of hepatocellular carcinoma. MicroRNAs are small non-coding RNAs that regulate gene expression. They are implicated in important physiological functions and they may be involved in every single step of carcinogenesis. They typically have 21 nucleotides and regulate gene expression at the post transcriptional level via blockage of mRNA translation or induction of mRNA degradation by binding to the 3′-untranslated regions (3′-UTR) of said mRNA.

In tumors, regulation of miRNA expression affects tumor development. In HCC, as in other cancers, miRNAs function either as oncogenes or tumor suppressor genes influencing cell growth and proliferation, cell metabolism and differentiation, apoptosis, angiogenesis, metastasis and eventually prognosis. [Lin et al., Biochemical and Biophysical Research Communications, vol. 375, 315-320 (2008); Kutay et al., J. Cell. Biochem., vol. 99, 671-678 (2006); Meng et al., Gastroenterology, vol. 133(2), 647-658 (2007), the contents of each of which are incorporated herein by reference in their entirety] The saRNA or the GalNAc-saRNA conjugate of the present invention modulates a target gene expression and/or function and also regulates miRNA levels in HCC cells. Non-limiting examples of miRNAs that may be regulated by the saRNA or the GalNAc-saRNA conjugate of the present invention include hsa-let-7a-5p, hsa-miR-133b, hsa-miR-122-5p, hsa-miR-335-5p, hsa-miR-196a-5p, hsa-miR-142-5p, hsa-miR-96-5p, hsa-miR-184, hsa-miR-214-3p, hsa-miR-15a-5p, hsa-let-7b-5p, hsa-miR-205-5p, hsa-miR-181a-5p, hsa-miR-140-5p, hsa-miR-146b-5p, hsa-miR-34c-5p, hsa-miR-134, hsa-let-7g-5p, hsa-let-7c, hsa-miR-218-5p, hsa-miR-206, hsa-miR-124-3p, hsa-miR-100-5p, hsa-miR-10b-5p, hsa-miR-155-5p, hsa-miR-1, hsa-miR-150-5p, hsa-let-7i-5p, hsa-miR-27b-3p, hsa-miR-127-5p, hsa-miR-191-5p, hsa-let-7f-5p, hsa-miR-10a-5p, hsa-miR-15b-5p, hsa-miR-16-5p, hsa-miR-34a-5p, hsa-miR-144-3p, hsa-miR-128, hsa-miR-215, hsa-miR-193a-5p, hsa-miR-23b-3p, hsa-miR-203a, hsa-miR-30c-5p, hsa-let-7e-5p, hsa-miR-146a-5p, hsa-let-7d-5p, hsa-miR-9-5p, hsa-miR-181b-5p, hsa-miR-181c-5p, hsa-miR-20b-5p, hsa-miR-125a-5p, hsa-miR-148b-3p, hsa-miR-92a-3p, hsa-miR-378a-3p, hsa-miR-130a-3p, hsa-miR-20a-5p, hsa-miR-132-3p, hsa-miR-193b-3p, hsa-miR-183-5p, hsa-miR-148a-3p, hsa-miR-138-5p, hsa-miR-3′73-3p, hsa-miR-29b-3p, hsa-miR-135b-5p, hsa-miR-21-5p, hsa-miR-181d, hsa-miR-301a-3p, hsa-miR-200c-3p, hsa-miR-7-5p, hsa-miR-29a-3p, hsa-miR-210, hsa-miR-17-5p, hsa-miR-98-5p, hsa-miR-25-3p, hsa-miR-143-3p, hsa-miR-19a-3p, hsa-miR-18a-5p, hsa-miR-125b-5p, hsa-miR-126-3p, hsa-miR-27a-3p, hsa-miR-372, hsa-miR-149-5p, and hsa-miR-32-5p.

In one non-limiting example, the miRNAs are oncogenic miRNAs and are downregulated by a factor of at least 0.01, 0.02, 0.05, 0.1, 0.2, 0.3, 0.5, 1, 1.5, 2, 2.5, and 3, in the presence of the saRNA or the GalNAc-saRNA conjugate of the invention compared to in the absence of the saRNA or the GalNAc-saRNA conjugate. In another non-limiting example, the miRNAs are tumor suppressing miRNAs and are upregulated by a factor of at least 0.01, 0.02, 0.05, 0.1, 0.2, 0.3, 0.5, 1, more preferably by a factor of at least 2, 3, 4, 5, 6, 7, 8, 9, 10, more preferably by a factor of at least 15, 20, 25, 30, 35, 40, 45, 50, even more preferably by a factor of at least 60, 70, 80, 90, 100, in the presence of the saRNA or the GalNAc-saRNA conjugate of the invention compared to in the absence of the saRNA or the GalNAc-saRNA conjugate.

### IV. Kits and Devices

### Kits

The invention provides a variety of kits for conveniently and/or effectively carrying out methods of the present invention. Typically kits will comprise sufficient amounts and/or numbers of components to allow a user to perform multiple treatments of a subject(s) and/or to perform multiple experiments.

In one embodiment, the present invention provides kits for regulate the expression of genes in vitro or in vivo, comprising saRNA or the GalNAc-saRNA conjugate of the present invention or a combination of saRNA or the GalNAc-saRNA conjugate of the present invention, saRNAs modulating other genes, siRNAs, miRNAs or other oligonucleotide molecules.

The kit may further comprise packaging and instructions and/or a delivery agent to form a formulation composition. The delivery agent may comprise a saline, a buffered solution, a lipidoid, a dendrimer or any delivery agent disclosed herein.

Non-limiting examples of genes are described herein in Table 1.

In one embodiment, the kits comprising saRNA or the GalNAc-saRNA conjugate described herein may be used with proliferating cells to show efficacy.

In one non-limiting example, the buffer solution may include sodium chloride, calcium chloride, phosphate and/or EDTA. In another non-limiting example, the buffer solution may include, but is not limited to, saline, saline with 2 mM calcium, 5% sucrose, 5% sucrose with 2 mM calcium, 5% Mannitol, 5% Mannitol with 2 mM calcium, Ringer's lactate, sodium chloride, sodium chloride with 2 mM calcium and mannose (See U.S. Pub. No. 20120258046; herein incorporated by reference in its entirety). In yet another non-limiting example, the buffer solutions may be precipitated or it may be lyophilized. The amount of each component may be varied to enable consistent, reproducible higher concentration saline or simple buffer formulations. The components may also be varied in order to increase the stability of saRNA or the GalNAc-saRNA conjugate in the buffer solution over a period of time and/or under a variety of conditions.

### Devices

The present invention provides for devices which may incorporate saRNA or the GalNAc-saRNA conjugate of the present invention. These devices contain in a stable formulation available to be immediately delivered to a subject in need thereof, such as a human patient.

Non-limiting examples of the devices include a pump, a catheter, a needle, a transdermal patch, a pressurized olfactory delivery device, iontophoresis devices, multi-layered microfluidic devices. The devices may be employed to deliver saRNA or the GalNAc-saRNA conjugate of the present invention according to single, multi- or split-dosing regiments. The devices may be employed to deliver saRNA or the GalNAc-saRNA conjugate of the present invention across biological tissue, intradermal, subcutaneously, or intramuscularly. More examples of devices suitable for delivering oligonucleotides are disclosed in International Publication WO 2013/090648 filed Dec. 14, 2012, the contents of which are incorporated herein by reference in their entirety.

### Definitions

For convenience, the meaning of certain terms and phrases used in the specification, examples, and appended claims, are provided below. If there is an apparent discrepancy between the usage of a term in other parts of this specification and its definition provided in this section, the definition in this section shall prevail.

About: As used herein, the term “about” means +/−10% of the recited value.

Administered in combination: As used herein, the term “administered in combination” or “combined administration” means that two or more agents are administered to a subject at the same time or within an interval such that there may be an overlap of an effect of each agent on the patient. In some embodiments, they are administered within about 60, 30, 15, 10, 5, or 1 minute of one another. In some embodiments, the administrations of the agents are spaced sufficiently close together such that a combinatorial (e.g., a synergistic) effect is achieved.

Amino acid: As used herein, the terms “amino acid” and “amino acids” refer to all naturally occurring L-alpha-amino acids. The amino acids are identified by either the one-letter or three-letter designations as follows: aspartic acid (Asp:D), isoleucine threonine (Thr:T), leucine (Leu:L), serine (Ser:S), tyrosine (Tyr:Y), glutamic acid (Glu:E), phenylalanine (Phe:F), proline (Pro:P), histidine (His:H), glycine (Gly:G), lysine (Lys:K), alanine (Ala:A), arginine (Arg:R), cysteine (Cys:C), tryptophan (Trp:W), valine (Val:V), glutamine (Gln:Q) methionine (Met:M), asparagines (Asn:N), where the amino acid is listed first followed parenthetically by the three and one letter codes, respectively.

Animal: As used herein, the term “animal” refers to any member of the animal kingdom. In some embodiments, “animal” refers to humans at any stage of development. In some embodiments, “animal” refers to non-human animals at any stage of development. In certain embodiments, the non-human animal is a mammal (e.g., a rodent, a mouse, a rat, a rabbit, a monkey, a dog, a cat, a sheep, cattle, a primate, or a pig). In some embodiments, animals include, but are not limited to, mammals, birds, reptiles, amphibians, fish, and worms. In some embodiments, the animal is a transgenic animal, genetically-engineered animal, or a clone.

Approximately: As used herein, the term “approximately” or “about,” as applied to one or more values of interest, refers to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Associated with: As used herein, the terms “associated with,” “conjugated,” “linked,” “attached,” and “tethered,” when used with respect to two or more moieties, means that the moieties are physically associated or connected with one another, either directly or via one or more additional moieties that serves as a linking agent, to form a structure that is sufficiently stable so that the moieties remain physically associated under the conditions in which the structure is used, e.g., physiological conditions. An “association” need not be strictly through direct covalent chemical bonding. It may also suggest ionic or hydrogen bonding or a hybridization based connectivity sufficiently stable such that the “associated” entities remain physically associated.

Bifunction or Bifunctional: As used herein, the terms “bifunction” and “bifunctional” refers to any substance, molecule or moiety which is capable of or maintains at least two functions. The functions may affect the same outcome or a different outcome. The structure that produces the function may be the same or different. For example, bifunctional saRNA of the present invention may comprise a cytotoxic peptide (a first function) while those nucleosides which comprise the saRNA are, in and of themselves, cytotoxic (second function).

Biocompatible: As used herein, the term “biocompatible” means compatible with living cells, tissues, organs or systems posing little to no risk of injury, toxicity or rejection by the immune system.

Biodegradable: As used herein, the term “biodegradable” means capable of being broken down into innocuous products by the action of living things.

Biologically active: As used herein, the phrase “biologically active” refers to a characteristic of any substance that has activity in a biological system and/or organism. For instance, a substance that, when administered to an organism, has a biological effect on that organism, is considered to be biologically active. In particular embodiments, the saRNA of the present invention may be considered biologically active if even a portion of the saRNA is biologically active or mimics an activity considered biologically relevant.

Cancer: As used herein, the term “cancer” in an individual refers to the presence of cells possessing characteristics typical of cancer-causing cells, such as uncontrolled proliferation, immortality, metastatic potential, rapid growth and proliferation rate, and certain characteristic morphological features. Often, cancer cells will be in the form of a tumor, but such cells may exist alone within an individual, or may circulate in the blood stream as independent cells, such as leukemic cells.

Cell growth: As used herein, the term “cell growth” is principally associated with growth in cell numbers, which occurs by means of cell reproduction (i.e. proliferation) when the rate of the latter is greater than the rate of cell death (e.g. by apoptosis or necrosis), to produce an increase in the size of a population of cells, although a small component of that growth may in certain circumstances be due also to an increase in cell size or cytoplasmic volume of individual cells. An agent that inhibits cell growth can thus do so by either inhibiting proliferation or stimulating cell death, or both, such that the equilibrium between these two opposing processes is altered.

Cell type: As used herein, the term “cell type” refers to a cell from a given source (e.g., a tissue, organ) or a cell in a given state of differentiation, or a cell associated with a given pathology or genetic makeup.

Chromosome: As used herein, the term “chromosome” refers to an organized structure of DNA and protein found in cells.

Complementary: As used herein, the term “complementary” as it relates to nucleic acids refers to hybridization or base pairing between nucleotides or nucleic acids, such as, for example, between the two strands of a double-stranded DNA molecule or between an oligonucleotide probe and a target are complementary.

Condition: As used herein, the term “condition” refers to the status of any cell, organ, organ system or organism. Conditions may reflect a disease state or simply the physiologic presentation or situation of an entity. Conditions may be characterized as phenotypic conditions such as the macroscopic presentation of a disease or genotypic conditions such as the underlying gene or protein expression profiles associated with the condition. Conditions may be benign or malignant.

Controlled Release: As used herein, the term “controlled release” refers to a pharmaceutical composition or compound release profile that conforms to a particular pattern of release to effect a therapeutic outcome.

Cytostatic: As used herein, “cytostatic” refers to inhibiting, reducing, suppressing the growth, division, or multiplication of a cell (e.g., a mammalian cell (e.g., a human cell)), bacterium, virus, fungus, protozoan, parasite, prion, or a combination thereof.

Cytotoxic: As used herein, “cytotoxic” refers to killing or causing injurious, toxic, or deadly effect on a cell (e.g., a mammalian cell (e.g., a human cell)), bacterium, virus, fungus, protozoan, parasite, prion, or a combination thereof.

Delivery: As used herein, “delivery” refers to the act or manner of delivering a compound, substance, entity, moiety, cargo or payload.

Delivery Agent: As used herein, “delivery agent” refers to any substance which facilitates, at least in part, the in vivo delivery of an saRNA of the present invention to targeted cells.

Destabilized: As used herein, the term “destable,” “destabilize,” or “destabilizing region” means a region or molecule that is less stable than a starting, wild-type or native form of the same region or molecule.

Detectable label: As used herein, “detectable label” refers to one or more markers, signals, or moieties which are attached, incorporated or associated with another entity that is readily detected by methods known in the art including radiography, fluorescence, chemiluminescence, enzymatic activity, absorbance and the like. Detectable labels include radioisotopes, fluorophores, chromophores, enzymes, dyes, metal ions, ligands such as biotin, avidin, streptavidin and haptens, quantum dots, and the like. Detectable labels may be located at any position in the oligonucleotides disclosed herein. They may be within the nucleotides or located at the 5′ or 3′ terminus.

Encapsulate: As used herein, the term “encapsulate” means to enclose, surround or encase.

Engineered: As used herein, embodiments of the invention are “engineered” when they are designed to have a feature or property, whether structural or chemical, that varies from a starting point, wild type or native molecule.

Equivalent subject: As used herein, “equivalent subject” may be e.g. a subject of similar age, sex and health such as liver health or cancer stage, or the same subject prior to treatment according to the invention. The equivalent subject is “untreated” in that he does not receive treatment with an saRNA according to the invention. However, he may receive a conventional anti-cancer treatment, provided that the subject who is treated with the saRNA of the invention receives the same or equivalent conventional anti-cancer treatment.

Exosome: As used herein, “exosome” is a vesicle secreted by mammalian cells.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end processing); (3) translation of an RNA into a polypeptide or protein; and (4) post-translational modification of a polypeptide or protein.

Feature: As used herein, a “feature” refers to a characteristic, a property, or a distinctive element.

Formulation: As used herein, a “formulation” includes at least one saRNA of the present invention and a delivery agent.

Fragment: A “fragment,” as used herein, refers to a portion. For example, fragments of proteins may comprise polypeptides obtained by digesting full-length protein isolated from cultured cells. Fragments of oligonucleotides may comprise nucleotides, or regions of nucleotides.

Functional: As used herein, a “functional” biological molecule is a biological molecule in a form in which it exhibits a property and/or activity by which it is characterized.

Gene: As used herein, the term “gene” refers to a nucleic acid sequence that comprises control and most often coding sequences necessary for producing a polypeptide or precursor. Genes, however, may not be translated and instead code for regulatory or structural RNA molecules.

A gene may be derived in whole or in part from any source known to the art, including a plant, a fungus, an animal, a bacterial genome or episome, eukaryotic, nuclear or plasmid DNA, cDNA, viral DNA, or chemically synthesized DNA. A gene may contain one or more modifications in either the coding or the untranslated regions that could affect the biological activity or the chemical structure of the expression product, the rate of expression, or the manner of expression control. Such modifications include, but are not limited to, mutations, insertions, deletions, and substitutions of one or more nucleotides. The gene may constitute an uninterrupted coding sequence or it may include one or more introns, bound by the appropriate splice junctions.

Gene expression: As used herein, the term “gene expression” refers to the process by which a nucleic acid sequence undergoes successful transcription and in most instances translation to produce a protein or peptide. For clarity, when reference is made to measurement of “gene expression”, this should be understood to mean that measurements may be of the nucleic acid product of transcription, e.g., RNA or mRNA or of the amino acid product of translation, e.g., polypeptides or peptides. Methods of measuring the amount or levels of RNA, mRNA, polypeptides and peptides are well known in the art.

Genome: The term “genome” is intended to include the entire DNA complement of an organism, including the nuclear DNA component, chromosomal or extrachromosomal DNA, as well as the cytoplasmic domain (e.g., mitochondrial DNA).

Homology: As used herein, the term “homology” refers to the overall relatedness between polymeric molecules, e.g. between nucleic acid molecules (e.g. DNA molecules and/or RNA molecules) and/or between polypeptide molecules. In some embodiments, polymeric molecules are considered to be “homologous” to one another if their sequences are at least 25%, 30%, 35%, 40%, 45%, 50%, 55%, 60%, 65%, 70%, 75%, 80%, 85%, 90%, 95%, or 99% identical or similar. The term “homologous” necessarily refers to a comparison between at least two sequences (polynucleotide or polypeptide sequences). In accordance with the invention, two polynucleotide sequences are considered to be homologous if the polypeptides they encode are at least about 50%, 60%, 70%, 80%, 90%, 95%, or even 99% for at least one stretch of at least about 20 amino acids. In some embodiments, homologous polynucleotide sequences are characterized by the ability to encode a stretch of at least 4-5 uniquely specified amino acids. For polynucleotide sequences less than 60 nucleotides in length, homology is determined by the ability to encode a stretch of at least 4-5 uniquely specified amino acids. In accordance with the invention, two protein sequences are considered to be homologous if the proteins are at least about 50%, 60%, 70%, 80%, or 90% identical for at least one stretch of at least about 20 amino acids.

The term “hyperproliferative cell” may refer to any cell that is proliferating at a rate that is abnormally high in comparison to the proliferating rate of an equivalent healthy cell (which may be referred to as a “control”). An “equivalent healthy” cell is the normal, healthy counterpart of a cell. Thus, it is a cell of the same type, e.g. from the same organ, which performs the same functions(s) as the comparator cell. For example, proliferation of a hyperproliferative hepatocyte should be assessed by reference to a healthy hepatocyte, whereas proliferation of a hyperproliferative prostate cell should be assessed by reference to a healthy prostate cell.

By an “abnormally high” rate of proliferation, it is meant that the rate of proliferation of the hyperproliferative cells is increased by at least 20, 30, 40%, or at least 45, 50, 55, 60, 65, 70, 75%, or at least 80%, as compared to the proliferative rate of equivalent, healthy (non-hyperproliferative) cells. The “abnormally high” rate of proliferation may also refer to a rate that is increased by a factor of at least 2, 3, 4, 5, 6, 7, 8, 9, 10, or by a factor of at least 15, 20, 25, 30, 35, 40, 45, 50, or by a factor of at least 60, 70, 80, 90, 100, compared to the proliferative rate of equivalent, healthy cells.

Hyperproliferative disorder: As used herein, a “hyperproliferative disorder” may be any disorder which involves hyperproliferative cells as defined above. Examples of hyperproliferative disorders include neoplastic disorders such as cancer, psoriatic arthritis, rheumatoid arthritis, gastric hyperproliferative disorders such as inflammatory bowel disease, skin disorders including psoriasis, Reiter's syndrome, pityriasis rubra pilaris, and hyperproliferative variants of the disorders of keratinization.

The skilled person is fully aware of how to identify a hyperproliferative cell. The presence of hyperproliferative cells within an animal may be identifiable using scans such as X-rays, MM or CT scans. The hyperproliferative cell may also be identified, or the proliferation of cells may be assayed, through the culturing of a sample in vitro using cell proliferation assays, such as MTT, XTT, MTS or WST-1 assays. Cell proliferation in vitro can also be determined using flow cytometry.

Identity: As used herein, the term “identity” refers to the overall relatedness between polymeric molecules, e.g., between oligonucleotide molecules (e.g. DNA molecules and/or RNA molecules) and/or between polypeptide molecules. Calculation of the percent identity of two polynucleotide sequences, for example, can be performed by aligning the two sequences for optimal comparison purposes (e.g., gaps can be introduced in one or both of a first and a second nucleic acid sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or 100% of the length of the reference sequence. The nucleotides at corresponding nucleotide positions are then compared. When a position in the first sequence is occupied by the same nucleotide as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using methods such as those described in Computational Molecular Biology, Lesk, A. M., ed., Oxford University Press, New York, 1988; Biocomputing: Informatics and Genome Projects, Smith, D. W., ed., Academic Press, New York, 1993; Sequence Analysis in Molecular Biology, von Heinje, G., Academic Press, 1987; Computer Analysis of Sequence Data, Part I, Griffin, A. M., and Griffin, H. G., eds., Humana Press, New Jersey, 1994; and Sequence Analysis Primer, Gribskov, M. and Devereux, J., eds., M Stockton Press, New York, 1991; each of which is incorporated herein by reference. For example, the percent identity between two nucleotide sequences can be determined using the algorithm of Meyers and Miller (CABIOS, 1989, 4:11-17), which has been incorporated into the ALIGN program (version 2.0) using a PAM120 weight residue table, a gap length penalty of 12 and a gap penalty of 4. The percent identity between two nucleotide sequences can, alternatively, be determined using the GAP program in the GCG software package using an NWSgapdna.CMP matrix. Methods commonly employed to determine percent identity between sequences include, but are not limited to those disclosed in Carillo, H., and Lipman, D., SIAM J Applied Math., 48:1073 (1988); incorporated herein by reference. Techniques for determining identity are codified in publicly available computer programs. Exemplary computer software to determine homology between two sequences include, but are not limited to, GCG program package, Devereux, J., et al., Nucleic Acids Research, 12(1), 387 (1984)), BLASTP, BLASTN, and FASTA Altschul, S. F. et al., J. Molec. Biol., 215, 403 (1990)).

Inhibit expression of a gene: As used herein, the phrase “inhibit expression of a gene” means to cause a reduction in the amount of an expression product of the gene. The expression product can be an RNA transcribed from the gene (e.g., an mRNA) or a polypeptide translated from an mRNA transcribed from the gene. Typically a reduction in the level of an mRNA results in a reduction in the level of a polypeptide translated therefrom. The level of expression may be determined using standard techniques for measuring mRNA or protein.

In vitro: As used herein, the term “in vitro” refers to events that occur in an artificial environment, e.g., in a test tube or reaction vessel, in cell culture, in a Petri dish, etc., rather than within an organism (e.g., animal, plant, or microbe).

In vivo: As used herein, the term “in vivo” refers to events that occur within an organism (e.g., animal, plant, or microbe or cell or tissue thereof).

Isolated: As used herein, the term “isolated” refers to a substance or entity that has been separated from at least some of the components with which it was associated (whether in nature or in an experimental setting). Isolated substances may have varying levels of purity in reference to the substances from which they have been associated. Isolated substances and/or entities may be separated from at least about 10%, about 20%, about 30%, about 40%, about 50%, about 60%, about 70%, about 80%, about 90%, or more of the other components with which they were initially associated. In some embodiments, isolated agents are more than about 80%, about 85%, about 90%, about 91%, about 92%, about 93%, about 94%, about 95%, about 96%, about 97%, about 98%, about 99%, or more than about 99% pure. As used herein, a substance is “pure” if it is substantially free of other components. Substantially isolated: By “substantially isolated” is meant that the compound is substantially separated from the environment in which it was formed or detected. Partial separation can include, for example, a composition enriched in the compound of the present disclosure. Substantial separation can include compositions containing at least about 50%, at least about 60%, at least about 70%, at least about 80%, at least about 90%, at least about 95%, at least about 97%, or at least about 99% by weight of the compound of the present disclosure, or salt thereof. Methods for isolating compounds and their salts are routine in the art.

Label: The term “label” refers to a substance or a compound which is incorporated into an object so that the substance, compound or object may be detectable.

Linker: As used herein, a linker refers to a group of atoms, e.g., 10-1,000 atoms, and can be comprised of the atoms or groups such as, but not limited to, carbon, amino, alkylamino, oxygen, sulfur, sulfoxide, sulfonyl, carbonyl, and imine. The linker can be attached to a modified nucleoside or nucleotide on the nucleobase or sugar moiety at a first end, and to a payload, e.g., a detectable or therapeutic agent, at a second end. The linker may be of sufficient length as to not interfere with incorporation into a nucleic acid sequence. The linker can be used for any useful purpose, such as to form saRNA conjugates, as well as to administer a payload, as described herein.

Examples of chemical groups that can be incorporated into the linker and/or spacer include, but are not limited to, alkyl, alkenyl, alkynyl, amido, amino, ether, thioether, ester, alkylene, heteroalkylene, aryl, or heterocyclyl, each of which can be optionally substituted, as described herein. Examples of spacer include, but are not limited to, unsaturated alkanes, polyethylene glycols (e.g., ethylene or propylene glycol monomeric units, e.g., diethylene glycol, dipropylene glycol, triethylene glycol, tripropylene glycol, tetraethylene glycol, or tetraethylene glycol), and dextran polymers and derivatives thereof. Examples of linkers include, but are not limited to, those with cleavable moieties within the linker, such as, for example, a disulfide bond (—S—S—) or an azo bond (—N═N—), which can be cleaved using a reducing agent or photolysis. Non-limiting examples of a selectively cleavable bond include a disulphide bond which can be cleaved for example by the use of tris(2-carboxyethyl)phosphine (TCEP), or other reducing agents.

Metastasis: As used herein, the term “metastasis” means the process by which cancer spreads from the place at which it first arose as a primary tumor to distant locations in the body. Metastasis also refers to cancers resulting from the spread of the primary tumor. For example, someone with breast cancer may show metastases in their lymph system, liver, bones or lungs.

Modified: As used herein “modified” refers to a changed state or structure of a molecule of the invention. Molecules may be modified in many ways including chemically, structurally, and functionally. In one embodiment, the saRNAs of the present invention are modified by the introduction of non-natural nucleosides and/or nucleotides.

Naturally occurring: As used herein, “naturally occurring” means existing in nature without artificial aid.

Nucleic acid: The term “nucleic acid” as used herein, refers to a molecule comprised of one or more nucleotides, i.e., ribonucleotides, deoxyribonucleotides, or both. The term includes monomers and polymers of ribonucleotides and deoxyribonucleotides, with the ribonucleotides and/or deoxyribonucleotides being bound together, in the case of the polymers, via 5′ to 3′ linkages. The ribonucleotide and deoxyribonucleotide polymers may be single or double-stranded. However, linkages may include any of the linkages known in the art including, for example, nucleic acids comprising 5′ to 3′ linkages. The nucleotides may be naturally occurring or may be synthetically produced analogs that are capable of forming base-pair relationships with naturally occurring base pairs. Examples of non-naturally occurring bases that are capable of forming base-pairing relationships include, but are not limited to, aza and deaza pyrimidine analogs, aza and deaza purine analogs, and other heterocyclic base analogs, wherein one or more of the carbon and nitrogen atoms of the pyrimidine rings have been substituted by heteroatoms, e.g., oxygen, sulfur, selenium, phosphorus, and the like.

Patient: As used herein, “patient” refers to a subject who may seek or be in need of treatment, requires treatment, is receiving treatment, will receive treatment, or a subject who is under care by a trained professional for a particular disease or condition.

Peptide: As used herein, “peptide” is less than or equal to 50 amino acids long, e.g., about 5, 10, 15, 20, 25, 30, 35, 40, 45, or 50 amino acids long.

Pharmaceutically acceptable: The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Pharmaceutically acceptable excipients: The phrase “pharmaceutically acceptable excipient,” as used herein, refers any ingredient other than the compounds described herein (for example, a vehicle capable of suspending or dissolving the active compound) and having the properties of being substantially nontoxic and non-inflammatory in a patient. Excipients may include, for example: antiadherents, antioxidants, binders, coatings, compression aids, disintegrants, dyes (colors), emollients, emulsifiers, fillers (diluents), film formers or coatings, flavors, fragrances, glidants (flow enhancers), lubricants, preservatives, printing inks, sorbents, suspensing or dispersing agents, sweeteners, and waters of hydration. Exemplary excipients include, but are not limited to: butylated hydroxytoluene (BHT), calcium carbonate, calcium phosphate (dibasic), calcium stearate, croscarmellose, crosslinked polyvinyl pyrrolidone, citric acid, crospovidone, cysteine, ethylcellulose, gelatin, hydroxypropyl cellulose, hydroxypropyl methylcellulose, lactose, magnesium stearate, maltitol, mannitol, methionine, methylcellulose, methyl paraben, microcrystalline cellulose, polyethylene glycol, polyvinyl pyrrolidone, povidone, pregelatinized starch, propyl paraben, retinyl palmitate, shellac, silicon dioxide, sodium carboxymethyl cellulose, sodium citrate, sodium starch glycolate, sorbitol, starch (corn), stearic acid, sucrose, talc, titanium dioxide, vitamin A, vitamin E, vitamin C, and xylitol.

Pharmaceutically acceptable salts: The present disclosure also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form (e.g., by reacting the free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

Pharmaceutically acceptable solvate: The term “pharmaceutically acceptable solvate,” as used herein, means a compound of the invention wherein molecules of a suitable solvent are incorporated in the crystal lattice. A suitable solvent is physiologically tolerable at the dosage administered. For example, solvates may be prepared by crystallization, recrystallization, or precipitation from a solution that includes organic solvents, water, or a mixture thereof. Examples of suitable solvents are ethanol, water (for example, mono-, di-, and tri-hydrates), N-methylpyrrolidinone (NMP), dimethyl sulfoxide (DMSO), N,N′-dimethylformamide (DMF), N,N′-dimethylacetamide (DMAC), 1,3-dimethyl-2-imidazolidinone (DMEU), 1,3-dimethyl-3,4,5,6-tetrahydro-2-(1H)-pyrimidinone (DMPU), acetonitrile (ACN), propylene glycol, ethyl acetate, benzyl alcohol, 2-pyrrolidone, benzyl benzoate, and the like. When water is the solvent, the solvate is referred to as a “hydrate.”

Pharmacologic effect: As used herein, a “pharmacologic effect” is a measurable biologic phenomenon in an organism or system which occurs after the organism or system has been contacted with or exposed to an exogenous agent. Pharmacologic effects may result in therapeutically effective outcomes such as the treatment, improvement of one or more symptoms, diagnosis, prevention, and delay of onset of disease, disorder, condition or infection. Measurement of such biologic phenomena may be quantitative, qualitative or relative to another biologic phenomenon. Quantitative measurements may be statistically significant. Qualitative measurements may be by degree or kind and may be at least 10%, 20%, 30%, 40%, 50%, 60%, 70%, 80%, 90% or more different. They may be observable as present or absent, better or worse, greater or less. Exogenous agents, when referring to pharmacologic effects are those agents which are, in whole or in part, foreign to the organism or system. For example, modifications to a wild type biomolecule, whether structural or chemical, would produce an exogenous agent. Likewise, incorporation or combination of a wild type molecule into or with a compound, molecule or substance not found naturally in the organism or system would also produce an exogenous agent.

The saRNA of the present invention, comprises exogenous agents. Examples of pharmacologic effects include, but are not limited to, alteration in cell count such as an increase or decrease in neutrophils, reticulocytes, granulocytes, erythrocytes (red blood cells), megakaryocytes, platelets, monocytes, connective tissue macrophages, epidermal langerhans cells, osteoclasts, dendritic cells, microglial cells, neutrophils, eosinophils, basophils, mast cells, helper T cells, suppressor T cells, cytotoxic T cells, natural killer T cells, B cells, natural killer cells, or reticulocytes. Pharmacologic effects also include alterations in blood chemistry, pH, hemoglobin, hematocrit, changes in levels of enzymes such as, but not limited to, liver enzymes AST and ALT, changes in lipid profiles, electrolytes, metabolic markers, hormones or other marker or profile known to those of skill in the art.

Physicochemical: As used herein, “physicochemical” means of or relating to a physical and/or chemical property.

Preventing: As used herein, the term “preventing” refers to partially or completely delaying onset of an infection, disease, disorder and/or condition; partially or completely delaying onset of one or more symptoms, features, or clinical manifestations of a particular infection, disease, disorder, and/or condition; partially or completely delaying onset of one or more symptoms, features, or manifestations of a particular infection, disease, disorder, and/or condition; partially or completely delaying progression from an infection, a particular disease, disorder and/or condition; and/or decreasing the risk of developing pathology associated with the infection, the disease, disorder, and/or condition.

Prodrug: The present disclosure also includes prodrugs of the compounds described herein. As used herein, “prodrugs” refer to any substance, molecule or entity which is in a form predicate for that substance, molecule or entity to act as a therapeutic upon chemical or physical alteration. Prodrugs may by covalently bonded or sequestered in some way and which release or are converted into the active drug moiety prior to, upon or after administered to a mammalian subject. Prodrugs can be prepared by modifying functional groups present in the compounds in such a way that the modifications are cleaved, either in routine manipulation or in vivo, to the parent compounds. Prodrugs include compounds wherein hydroxyl, amino, sulfhydryl, or carboxyl groups are bonded to any group that, when administered to a mammalian subject, cleaves to form a free hydroxyl, amino, sulfhydryl, or carboxyl group respectively. Preparation and use of prodrugs is discussed in T. Higuchi and V. Stella, “Pro-drugs as Novel Delivery Systems,” Vol. 14 of the A.C.S. Symposium Series, and in Bioreversible Carriers in Drug Design, ed. Edward B. Roche, American Pharmaceutical Association and Pergamon Press, 1987, both of which are hereby incorporated by reference in their entirety.

Prognosing: As used herein, the term “prognosing” means a statement or claim that a particular biologic event will, or is very likely to, occur in the future.

Progression: As used herein, the term “progression” or “cancer progression” means the advancement or worsening of or toward a disease or condition.

Proliferate: As used herein, the term “proliferate” means to grow, expand or increase or cause to grow, expand or increase rapidly. “Proliferative” means having the ability to proliferate. “Anti-proliferative” means having properties counter to or inapposite to proliferative properties.

Protein: A “protein” means a polymer of amino acid residues linked together by peptide bonds. The term, as used herein, refers to proteins, polypeptides, and peptides of any size, structure, or function. Typically, however, a protein will be at least 50 amino acids long. In some instances the protein encoded is smaller than about 50 amino acids. In this case, the polypeptide is termed a peptide. If the protein is a short peptide, it will be at least about 10 amino acid residues long. A protein may be naturally occurring, recombinant, or synthetic, or any combination of these. A protein may also comprise a fragment of a naturally occurring protein or peptide. A protein may be a single molecule or may be a multi-molecular complex. The term protein may also apply to amino acid polymers in which one or more amino acid residues are an artificial chemical analogue of a corresponding naturally occurring amino acid.

Protein expression: The term “protein expression” refers to the process by which a nucleic acid sequence undergoes translation such that detectable levels of the amino acid sequence or protein are expressed.

Purified: As used herein, “purify,” “purified,” “purification” means to make substantially pure or clear from unwanted components, material defilement, admixture or imperfection.

Regression: As used herein, the term “regression” or “degree of regression” refers to the reversal, either phenotypically or genotypically, of a cancer progression. Slowing or stopping cancer progression may be considered regression.

Sample: As used herein, the term “sample” or “biological sample” refers to a subset of its tissues, cells or component parts (e.g. body fluids, including but not limited to blood, mucus, lymphatic fluid, synovial fluid, cerebrospinal fluid, saliva, amniotic fluid, amniotic cord blood, urine, vaginal fluid and semen). A sample further may include a homogenate, lysate or extract prepared from a whole organism or a subset of its tissues, cells or component parts, or a fraction or portion thereof, including but not limited to, for example, plasma, serum, spinal fluid, lymph fluid, the external sections of the skin, respiratory, intestinal, and genitourinary tracts, tears, saliva, milk, blood cells, tumors, organs. A sample further refers to a medium, such as a nutrient broth or gel, which may contain cellular components, such as proteins or nucleic acid molecule.

Signal Sequences: As used herein, the phrase “signal sequences” refers to a sequence which can direct the transport or localization of a protein.

Single unit dose: As used herein, a “single unit dose” is a dose of any therapeutic administered in one dose/at one time/single route/single point of contact, i.e., single administration event.

Similarity: As used herein, the term “similarity” refers to the overall relatedness between polymeric molecules, e.g. between polynucleotide molecules (e.g. DNA molecules and/or RNA molecules) and/or between polypeptide molecules. Calculation of percent similarity of polymeric molecules to one another can be performed in the same manner as a calculation of percent identity, except that calculation of percent similarity takes into account conservative substitutions as is understood in the art.

Split dose: As used herein, a “split dose” is the division of single unit dose or total daily dose into two or more doses.

Stable: As used herein “stable” refers to a compound that is sufficiently robust to survive isolation to a useful degree of purity from a reaction mixture, and in one embodiment, capable of formulation into an efficacious therapeutic agent.

Stabilized: As used herein, the term “stabilize”, “stabilized,” “stabilized region” means to make or become stable.

Subject: As used herein, the term “subject” or “patient” refers to any organism to which a composition in accordance with the invention may be administered, e.g., for experimental, diagnostic, prophylactic, and/or therapeutic purposes. Typical subjects include animals (e.g., mammals such as mice, rats, rabbits, non-human primates, and humans) and/or plants.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Substantially equal: As used herein as it relates to time differences between doses, the term means plus/minus 2%.

Substantially simultaneously: As used herein and as it relates to plurality of doses, the term means within 2 seconds.

Suffering from: An individual who is “suffering from” a disease, disorder, and/or condition has been diagnosed with or displays one or more symptoms of a disease, disorder, and/or condition.

Susceptible to: An individual who is “susceptible to” a disease, disorder, and/or condition has not been diagnosed with and/or may not exhibit symptoms of the disease, disorder, and/or condition but harbors a propensity to develop a disease or its symptoms. In some embodiments, an individual who is susceptible to a disease, disorder, and/or condition (for example, cancer) may be characterized by one or more of the following: (1) a genetic mutation associated with development of the disease, disorder, and/or condition; (2) a genetic polymorphism associated with development of the disease, disorder, and/or condition; (3) increased and/or decreased expression and/or activity of a protein and/or nucleic acid associated with the disease, disorder, and/or condition; (4) habits and/or lifestyles associated with development of the disease, disorder, and/or condition; (5) a family history of the disease, disorder, and/or condition; and (6) exposure to and/or infection with a microbe associated with development of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Sustained release: As used herein, the term “sustained release” refers to a pharmaceutical composition or compound release profile that conforms to a release rate over a specific period of time.

Synthetic: The term “synthetic” means produced, prepared, and/or manufactured by the hand of man. Synthesis of polynucleotides or polypeptides or other molecules of the present invention may be chemical or enzymatic.

Targeted Cells: As used herein, “targeted cells” refers to any one or more cells of interest. The cells may be found in vitro, in vivo, in situ or in the tissue or organ of an organism. The organism may be an animal, in one embodiment, a mammal, or a human and most In one embodiment, a patient.

Therapeutic Agent: The term “therapeutic agent” refers to any agent that, when administered to a subject, has a therapeutic, diagnostic, and/or prophylactic effect and/or elicits a desired biological and/or pharmacological effect.

Therapeutically effective amount: As used herein, the term “therapeutically effective amount” means an amount of an agent to be delivered (e.g., nucleic acid, drug, therapeutic agent, diagnostic agent, prophylactic agent, etc.) that is sufficient, when administered to a subject suffering from or susceptible to an infection, disease, disorder, and/or condition, to treat, improve symptoms of, diagnose, prevent, and/or delay the onset of the infection, disease, disorder, and/or condition.

Therapeutically effective outcome: As used herein, the term “therapeutically effective outcome” means an outcome that is sufficient in a subject suffering from or susceptible to an infection, disease, disorder, and/or condition, to treat, improve symptoms of, diagnose, prevent, and/or delay the onset of the infection, disease, disorder, and/or condition.

Total daily dose: As used herein, a “total daily dose” is an amount given or prescribed in 24 hour period. It may be administered as a single unit dose.

Transcription factor: As used herein, the term “transcription factor” refers to a DNA-binding protein that regulates transcription of DNA into RNA, for example, by activation or repression of transcription. Some transcription factors effect regulation of transcription alone, while others act in concert with other proteins. Some transcription factor can both activate and repress transcription under certain conditions. In general, transcription factors bind a specific target sequence or sequences highly similar to a specific consensus sequence in a regulatory region of a target gene. Transcription factors may regulate transcription of a target gene alone or in a complex with other molecules.

Treating: As used herein, the term “treating” refers to partially or completely alleviating, ameliorating, improving, relieving, delaying onset of, inhibiting progression of, reducing severity of, and/or reducing incidence of one or more symptoms or features of a particular infection, disease, disorder, and/or condition. For example, “treating” cancer may refer to inhibiting survival, growth, and/or spread of a tumor. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

The phrase “a method of treating” or its equivalent, when applied to, for example, cancer refers to a procedure or course of action that is designed to reduce, eliminate or prevent the number of cancer cells in an individual, or to alleviate the symptoms of a cancer. “A method of treating” cancer or another proliferative disorder does not necessarily mean that the cancer cells or other disorder will, in fact, be completely eliminated, that the number of cells or disorder will, in fact, be reduced, or that the symptoms of a cancer or other disorder will, in fact, be alleviated. Often, a method of treating cancer will be performed even with a low likelihood of success, but which, given the medical history and estimated survival expectancy of an individual, is nevertheless deemed an overall beneficial course of action.

Tumor growth: As used herein, the term “tumor growth” or “tumor metastases growth”, unless otherwise indicated, is used as commonly used in oncology, where the term is principally associated with an increased mass or volume of the tumor or tumor metastases, primarily as a result of tumor cell growth.

Tumor Burden: As used herein, the term “tumor burden” refers to the total Tumor Volume of all tumor nodules with a diameter in excess of 3 mm carried by a subject.

Tumor Volume: As used herein, the term “tumor volume” refers to the size of a tumor. The tumor volume in mm³ is calculated by the formula: volume=(width)²×length/2.

Unmodified: As used herein, “unmodified” refers to any substance, compound or molecule prior to being changed in any way. Unmodified may, but does not always, refer to the wild type or native form of a biomolecule. Molecules may undergo a series of modifications whereby each modified molecule may serve as the “unmodified” starting molecule for a subsequent modification.

### Equivalents and Scope

Those skilled in the art will recognize, or be able to ascertain using no more than routine experimentation, many equivalents to the specific embodiments in accordance with the invention described herein. The scope of the present invention is not intended to be limited to the above Description, but rather is as set forth in the appended claims.

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention includes embodiments in which more than one, or all of the group members are present in, employed in, or otherwise relevant to a given product or process.

It is also noted that the term “comprising” is intended to be open and permits the inclusion of additional elements or steps.

Where ranges are given, endpoints are included. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise.

In addition, it is to be understood that any particular embodiment of the present invention that falls within the prior art may be explicitly excluded from any one or more of the claims. Since such embodiments are deemed to be known to one of ordinary skill in the art, they may be excluded even if the exclusion is not set forth explicitly herein. Any particular embodiment of the compositions of the invention (e.g., any nucleic acid or protein encoded thereby; any method of production; any method of use; etc.) can be excluded from any one or more claims, for any reason, whether or not related to the existence of prior art.

All cited sources, for example, references, publications, databases, database entries, and art cited herein, are incorporated into this application by reference, even if not expressly stated in the citation. In case of conflicting statements of a cited source and the instant application, the statement in the instant application shall control.

The invention is further illustrated by the following non-limiting examples.

### EXAMPLES

### Example 1. Synthesis of GalNAc Monomers

### (3aS,5R,6R,7R,7aR)-5-(acetoxymethyl)-2-methyl-3a,6,7,7a-tetrahydro-5H-pyrano[3,2-d]oxazole-6,7-diyl diacetate 2

To a stirred suspension of GalNAc (50 g, 129 mmol) in dichloromethane (580 mL) at RT is added trimethylsilyl trifluoromethanesulfonate (47 mL, 316 mmol, 2.46 eq) and reaction mixture heated to reflux. Reaction stirred for 24 h then cooled to 0° C. Reaction quenched with triethylamine, washed with saturated aqueous NaHCO₃, dried over Na₂SO₄, filtered and concentrated in vacuo to give 2 as a crude brown gum used directly in the next step.

### (2R,3R,4R,5R,6R)-5-acetamido-2-(acetoxymethyl)-6-(allyloxy)tetrahydro-2H-pyran-3,4-diyl diacetate 3

A solution of 2 (42 g, 128 mmol) in dichloromethane (1000 mL) is stirred at RT over activated 4A molecular sieves (160 g) and allyl alcohol (9.6 mL, 141 mmol, 1.1 eq) is added. Reaction mixture stirred for 30 mins before adding trimethylsilyl trifluoromethanesulfonate (20.5 mL, 138 mmol, 1.0 eq). Reaction mixture stirred for a further 3 h 15 mins before filtering through celite and washing with saturated aqueous NaHCO₃. Mixture is dried over Na₂SO₄, filtered and concentrated in vacuo. Crude product recrystallised from ethyl acetate/diethyl ether then ethyl acetate, washed with ethyl acetate (x4), diethyl ether (x2) and dried under high vacuum to give 3 as a brown solid in 35% yield from GalNAc.

### 2-(((2R,3R,4R, 5R, 6R)-3 -acetamido-4, 5-diacetoxy-6-(acetoxymethyl)tetrahydro-2H-pyran-2-yl)oxy)acetic acid 4

To a stirred solution of 3 (19.2 g, 49.6 mmol) in 1:1 dichloromethane/acetonitrile (192 mL) at RT was added sodium periodate (40.3 g, 189 mmol, 3.8 eq) and water (45 mL). The mixture was cooled to 5° C. and ruthenium chloride (1.03 g, 4.96 mmol, 0.1 eq) was added in a single portion. The reaction was warmed to RT and stirred for 16 h. The organic solvent was removed in vacuo and the aqueous phase extracted with dichloromethane (x9). The organic phases were combined, dried over Na₂SO₄, filtered and concentrated in vacuo. The crude product was recrystallised from ethyl acetate, washed with ethyl acetate (x2) then diethyl ether (x2) and dried under high vacuum to give 4 as an off-white solid in 70% yield.

### (2R,3R,4R,5R,6R)-5-acetamido-2-(acetoxymethyl)-6-(2-((6-(((2R,4S,5R)-5-((bis(4-methoxyphenyl)(phenyl)methoxy)methyl)-4-hydroxytetrahydrofuran-2-yl)oxy)hexyl)amino)-2-oxoethoxy)tetrahydro-2H-pyran-3,4-diyl diacetate 6

To a stirred suspension of 4 (4.65 g, 11.5 mmol) and 5 (6.15 g, 11.5 mmol) in tetrahydrofuran (100 mL) at RT was added hydroxybenzotriazole (1.86 g, 13.8 mmol, 1.2 eq) then 1-ethyl-3-(3-dimethylaminopropyl) carbodiimide (2.65 g, 13.8 mmol, 1.2 eq) and the reaction mixture stirred for 16 h. The mixture was concentrated in vacuo, dissolved in ethyl acetate, washed with 10:3 water/brine, back-extracted with ethyl acetate, dried over Na₂SO₄, filtered and concentrated in vacuo. The crude oil was purified by flash column chromatography (silica, dichloromethane/acetone gradient) to give 6 as a yellow solid in 68% yield.

### (2R,3R,4R,5R,6R)-5-acetamido-2-(acetoxymethyl)-6-(2-((6-(((2R,4S,5R)-5-((bis(4-methoxyphenyl)(phenyl)methoxy)methyl)-4-(((2-cyanoethoxy)(diisopropylamino)phosphaneyl)oxy)tetrahydrofuran-2-yl)oxy)hexyl)amino)-2-oxoethoxy)tetrahydro-2H-pyran-3,4-diyl diacetate M1′ (where R₁=R₂=R₃=Ac and R₄=OCH₂CH₂CN, R₅=R₆=2-propyl).

6 (6.88 g, 7.38 mmol) is azeotroped with dichloromethane (x3) then dissolved in dichloromethane (70 mL) and stirred at RT. To the mixture is added a solution of 2-cyanoethoxy-bis(N,N-diisopropylamino)phosphine (2.45 g, 8.12 mmol, 1.1 eq) in dichloromethane followed by diisopropylammonium tetrazolide (0.63 g, 3.69 mmol, 0.5 eq) and the mixture stirred at RT for 16 h. The reaction mixture is washed with water then brine, dried over Na₂SO₄, filtered and concentrated in vacuo. The crude oil is precipitated with pentane (x5) then purified by flash column chromatography (silica, ethyl acetate) to give a yellow gum which is dissolved in acetonitrile, filtered and concentrated in vacuo to give 7 as a yellow solid in 63% yield.

### Triethylammonium 4-(((2R,3S,5R)-5-((6-(2-(((2R,3R,4R,5R,6R)-3-acetamido-4,5-diacetoxy-6-(acetoxymethyl)tetrahydro-2H-pyran-2-yl)oxy)acetamido)hexyl)oxy)-2-((bis(4-methoxyphenyl)(phenyl)methoxy)methyl)tetrahydrofuran-3-yl)oxy)-4-oxobutanoate 8

To a stirred suspension of 6 (2 g, 2.17 mmol) in dichloromethane (6 mL) at RT is added succinic anhydride (0.54 g, 5.42 mmol, 2.5 eq) and triethylamine (0.76 mL, 5.42 mmol, 2.5 eq)and the mixture stirred at RT for 16 h. The mixture was diluted with dichloromethane and washed with saturated aqueous NaHCO₃ then brine. The aqueous phases were combined, back-extracted with dichloromethane, dried over NaSO₄, filtered and concentrated in vacuo. The crude material was purified by flash column chromatography (silica, dichloromethane/methanol gradient) to give 8 in 29% yield.

### β-dR-GalNAc-succinyl-LCAA-CPG (1000 Å) M4′ (where R₁=R₂=R₃=Ac, L₁=succinyl and the support is 1000 Å LCAA-CPG)

To a stirred suspension of 8 (2.38 g, 2.11 mmol) in 2% triethylamine/dichloromethane (8 mL) is added 2-(1H-benzotriazole-1-yl)-1,1,3,3,-tetramethylaminium tetrafluoroborate (1.02 g, 3.18 mmol, 1.5 eq) and the mixture stirred at RT for 15 mins. The reaction mixture is added to pre-washed amino SynBase™ LCAA CPG 1000/100 (49 g) and mixed by bubbling with a stream of nitrogen for 2 h. The CPG is filtered, washed with dichloromethane (x3) then suspended in a solution of dimethylaminopyridine (0.25 g) and acetic anhydride (3.8 mL) in pyridine (150 mL). The mixture is allowed to sit for 30 mins with occasional gentle agitation before filtering, washing with methanol (x3), dichloromethane (x3) and diethyl ether (x3) and air drying to give a free-flowing white solid.

### Example 2. Synthesis of saRNA-GalNAc Conjugates

The monomeric GalNAc building blocks are compatible with standard oligonucleotide synthesis by the phosphoramidite methods. The phosphoramidites and functionalised solid supports are used during the synthesis process. The GalNAc phosphoramidites are added at any position of the oligonucleotide alone or in combination with other GalNAc monomers. They are added sequentially without any spacer or linker or separated by nucleotides, spacers or linkers. GalNAc solid supports are used to incorporate GalNAc modifications at the 3′-end of the oligonucleotide.

saRNA-GalNAc conjugates were prepared using typical oligonucleotide synthesis, deprotection, purification and annealing protocols for this type of modified oligonucleotide.

### Example 3. In-Vitro and In-Vivo Studies with CEBPA-saRNA-GalNAc Conjugates

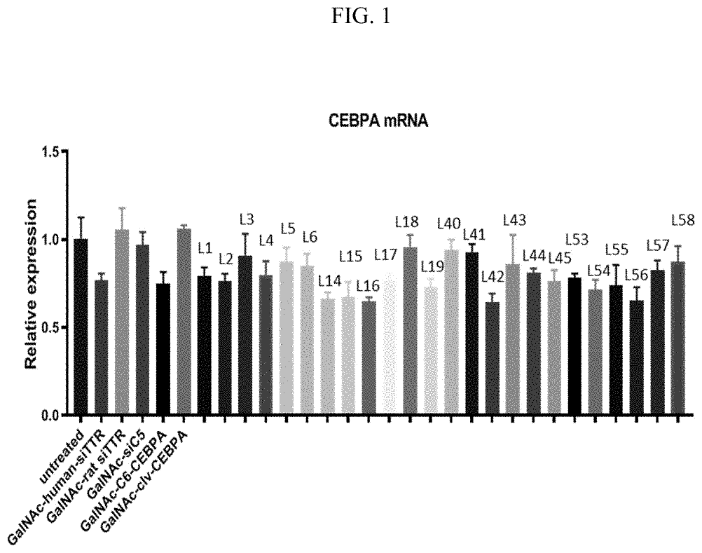

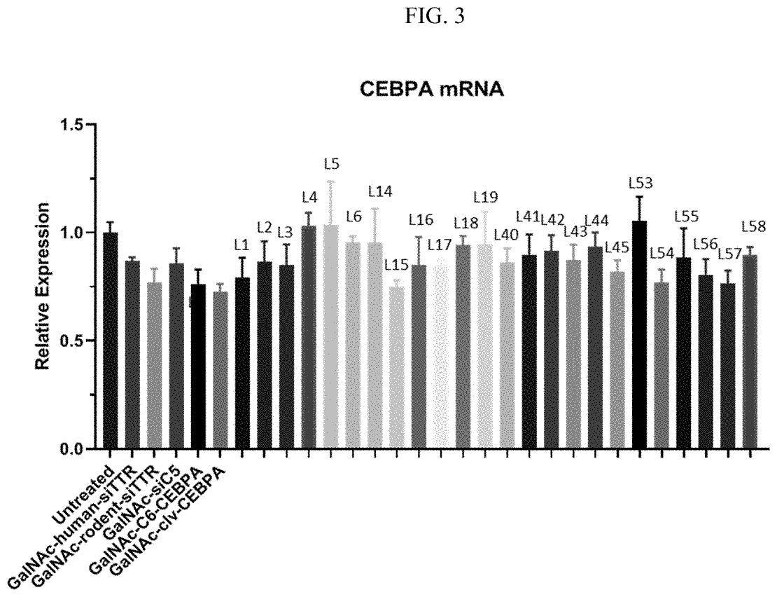

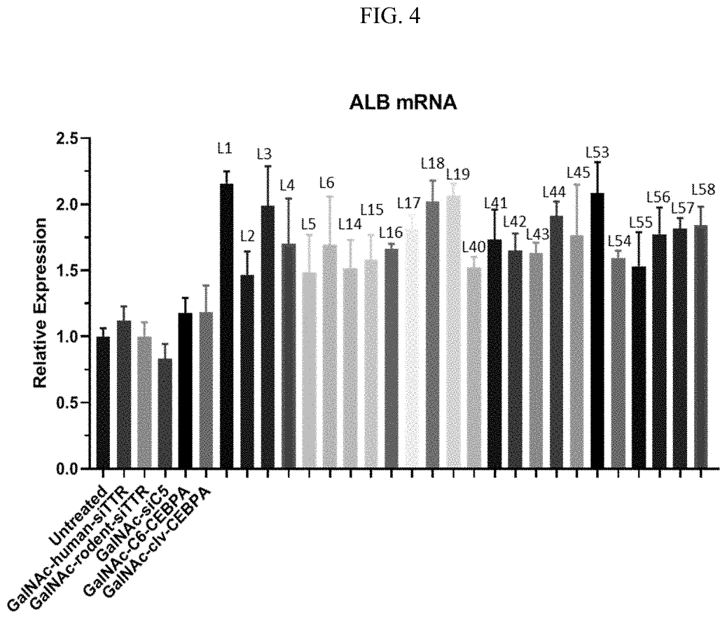

The 24 GalNAc-CEBPA-saRNA conjugates in Table 5 were synthesised and tested in vitro in primary hepatocyte for activity by passive transfection against the previously described fully modified GalNAc-C6-CEBPA saRNA conjugate (Example 2 of PCT/EP2018/074211 filed Sep. 7, 2018) encompassed by the C6-GalNAc structure described herein. All new designs gave equivalent or better upregulation of CEBPA and albumin mRNAs than GalNAc-C6-CEBPA by passive transfection in primary rat hepatocyte at 500 nM (FIG. 1 and FIG. 2) and 104 (FIG. 3 and FIG. 4).

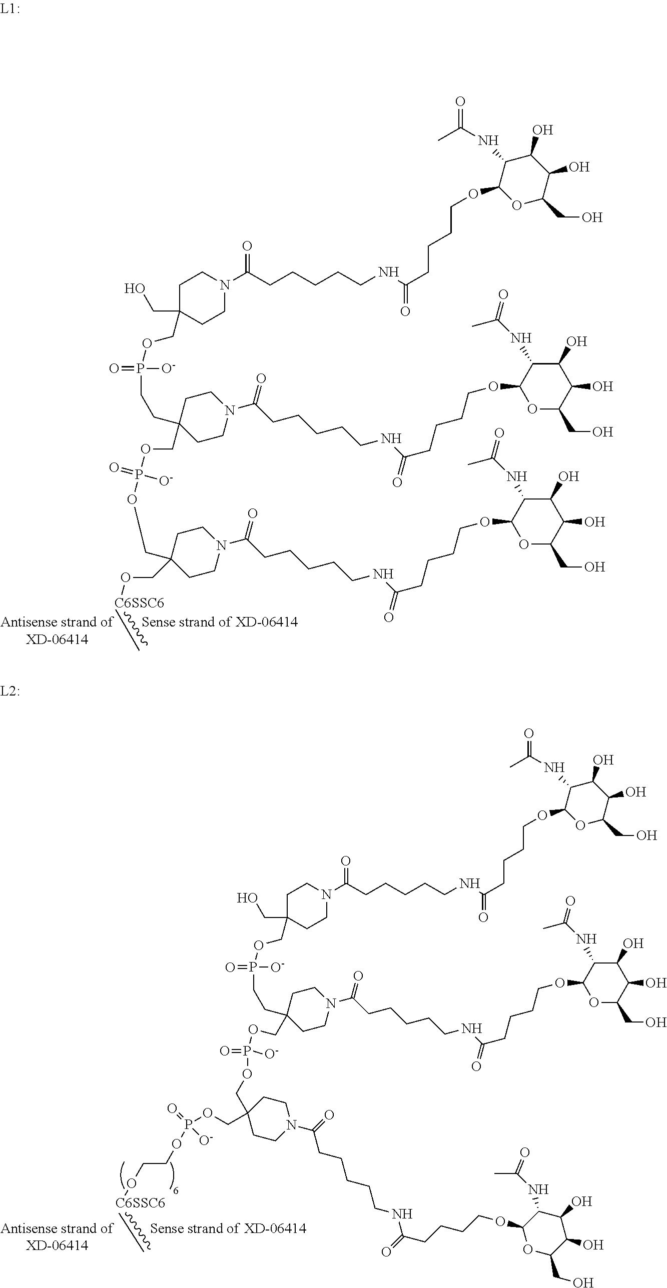

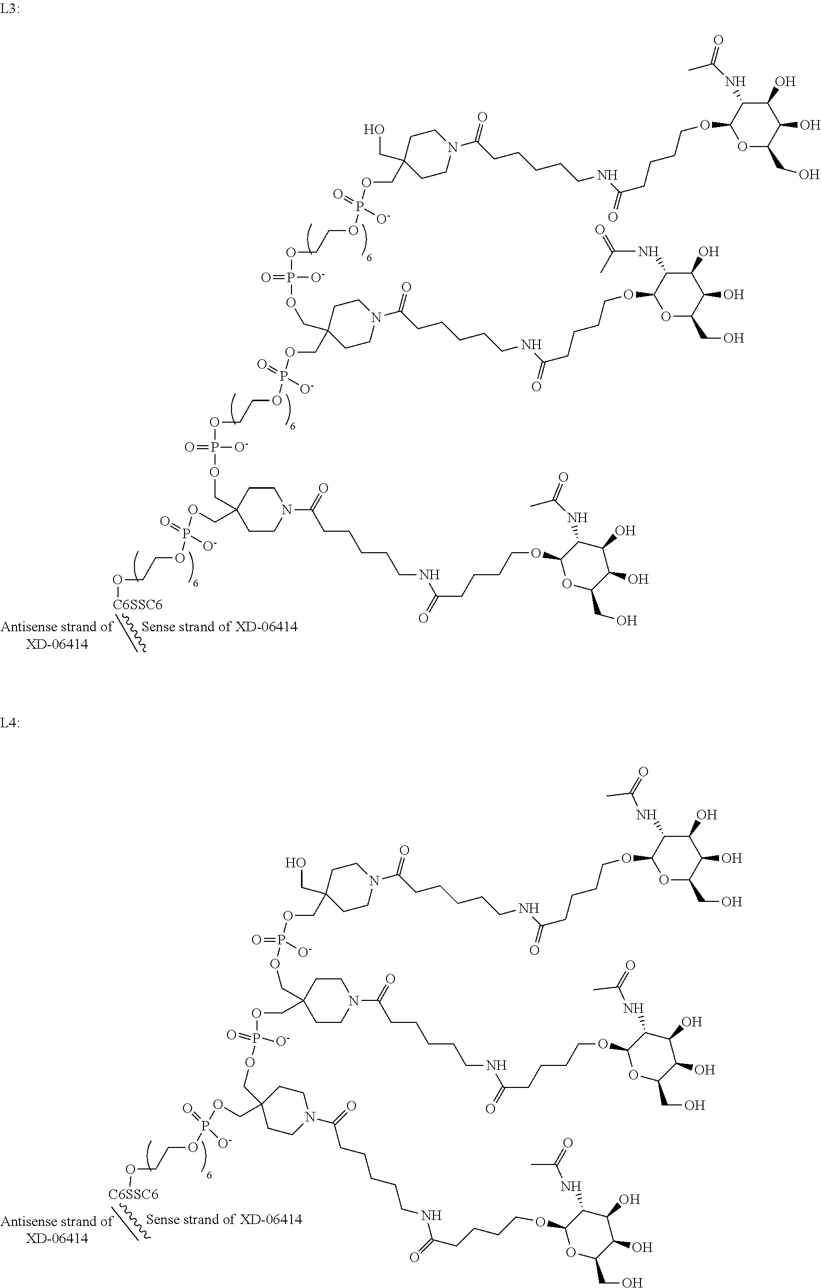

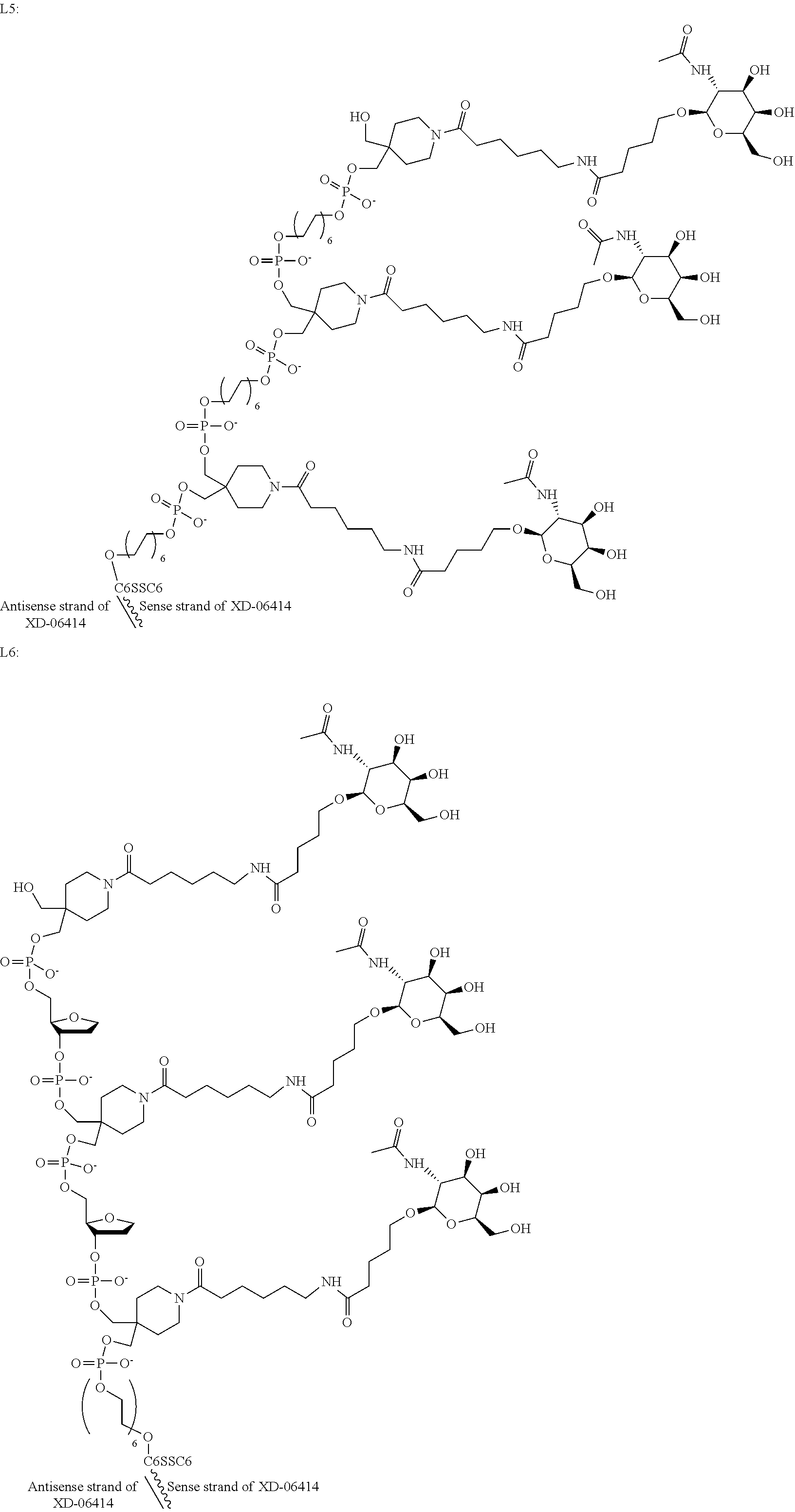

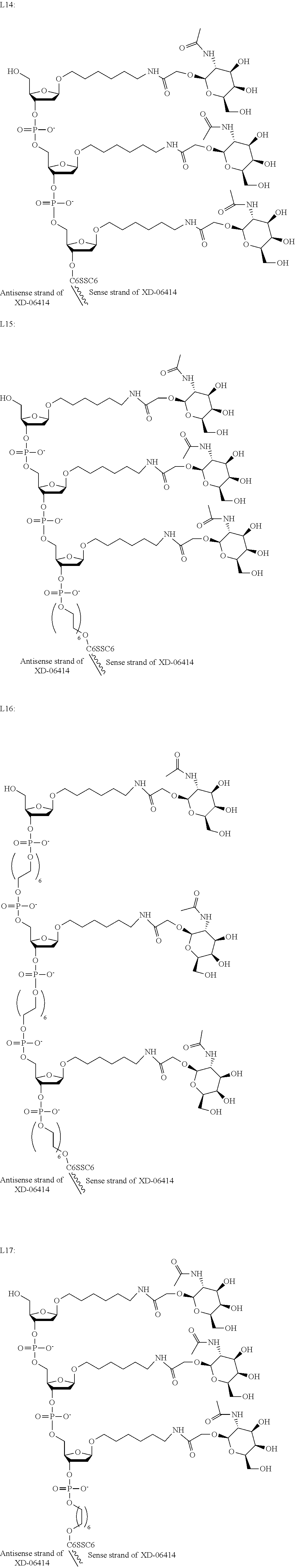

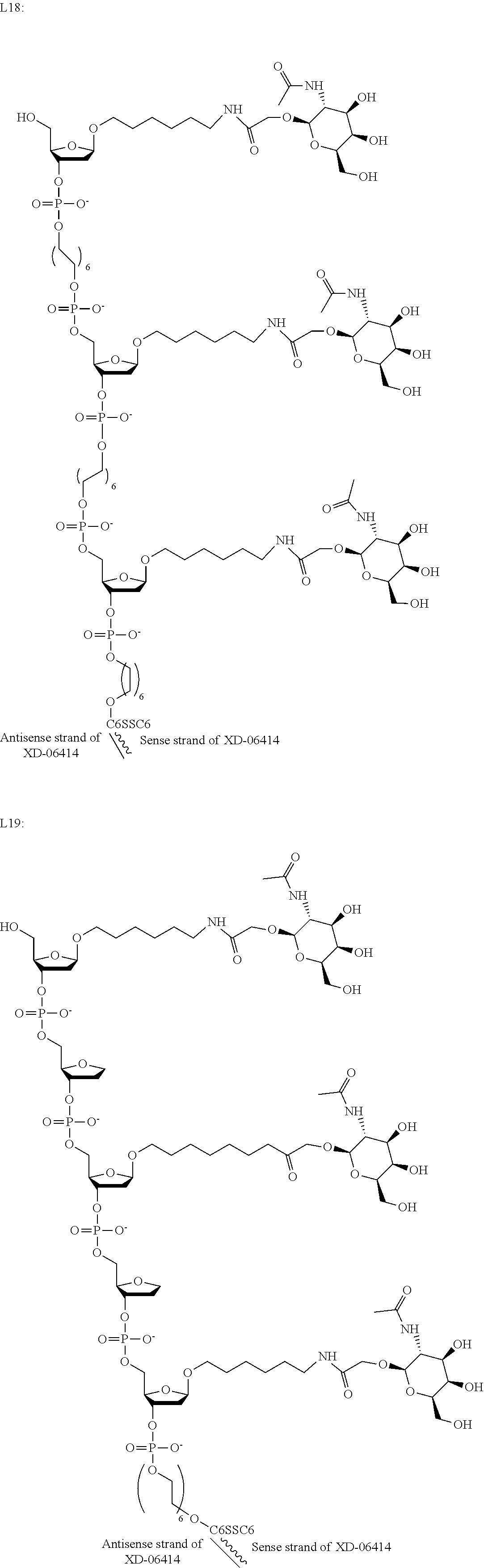

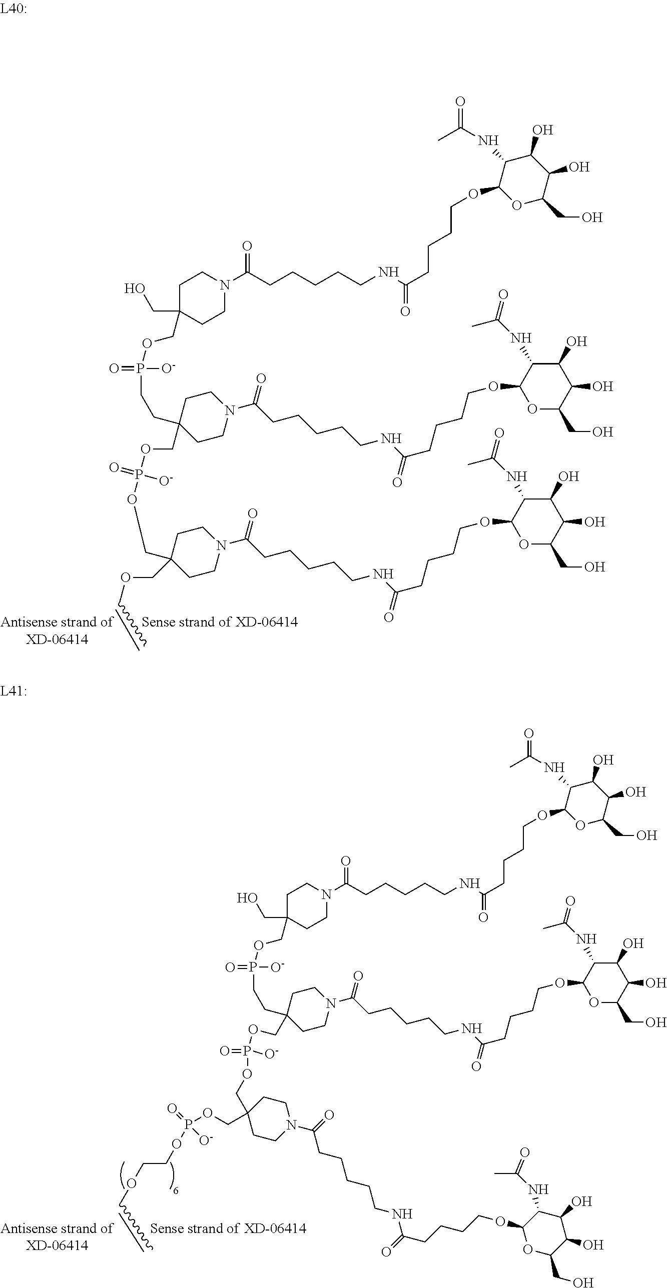

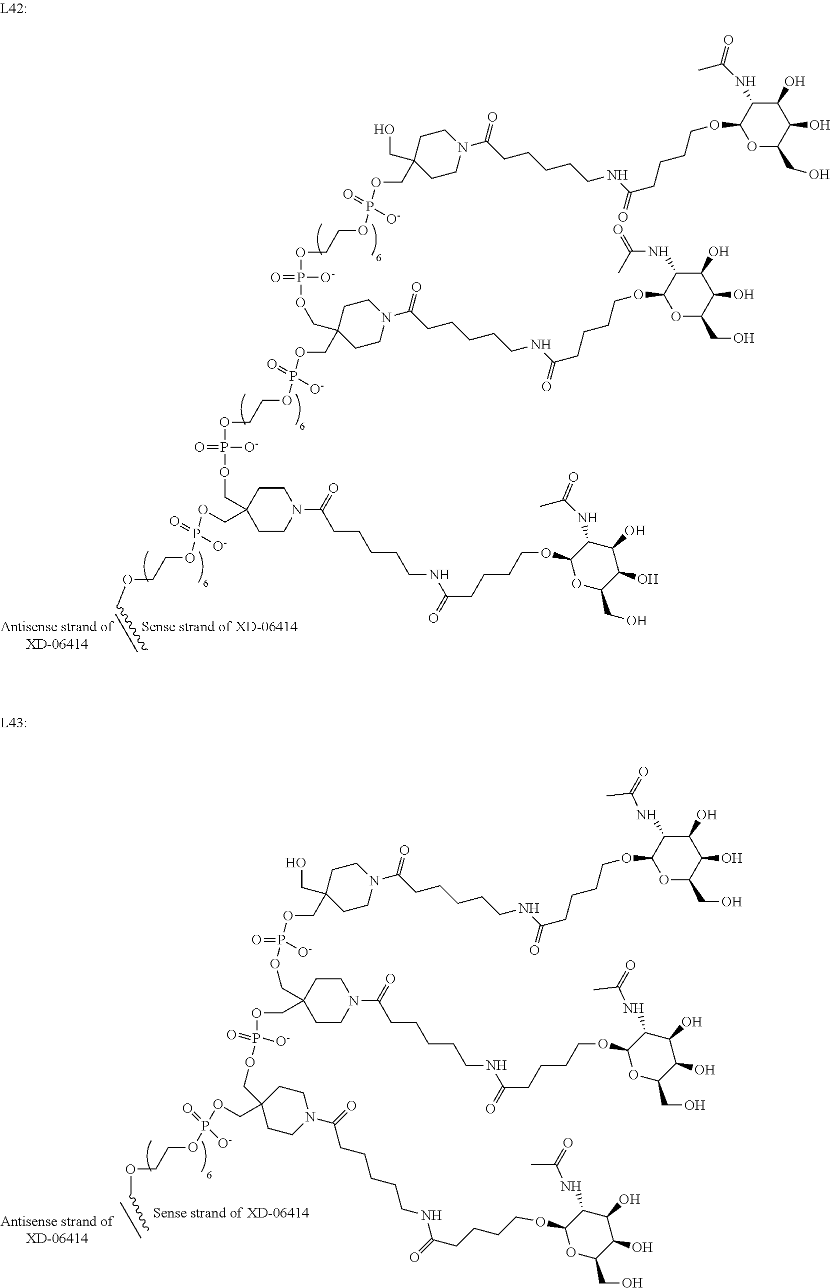

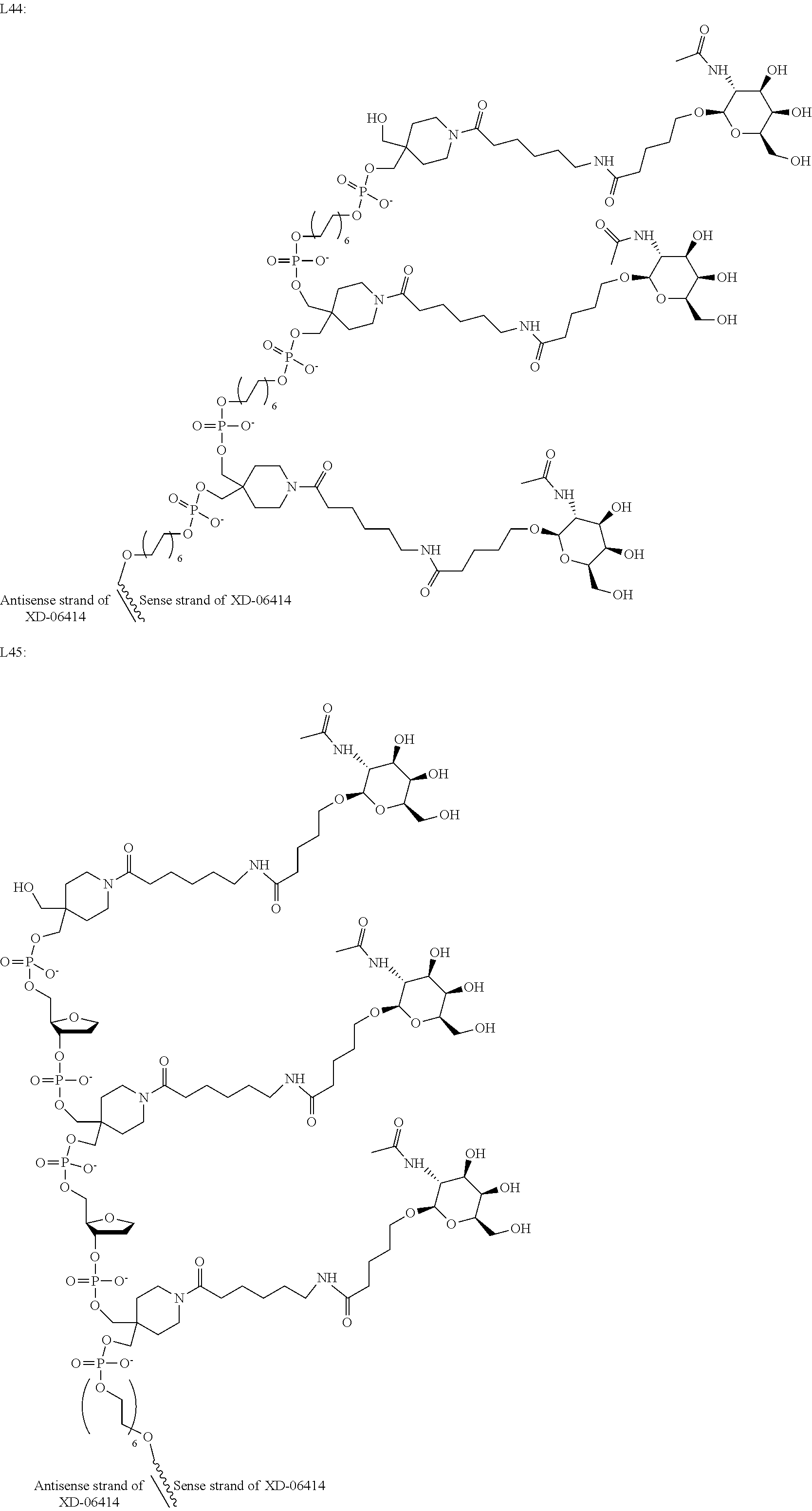

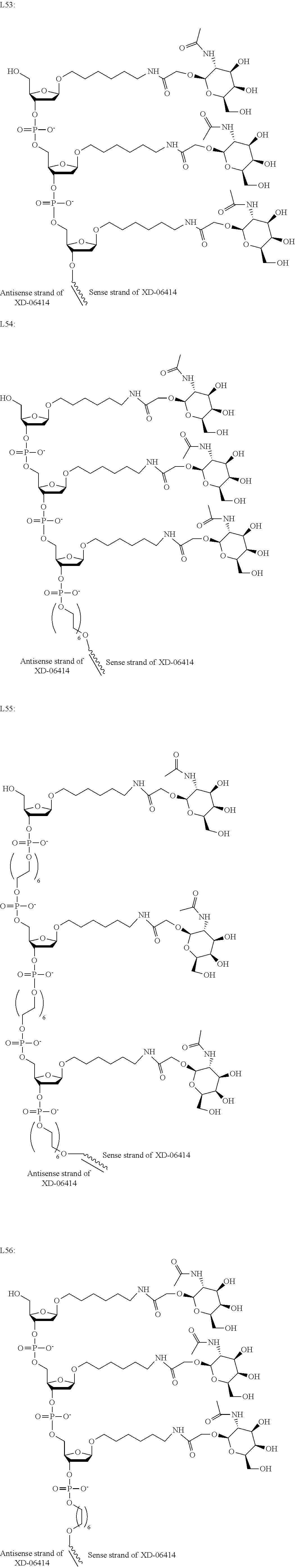

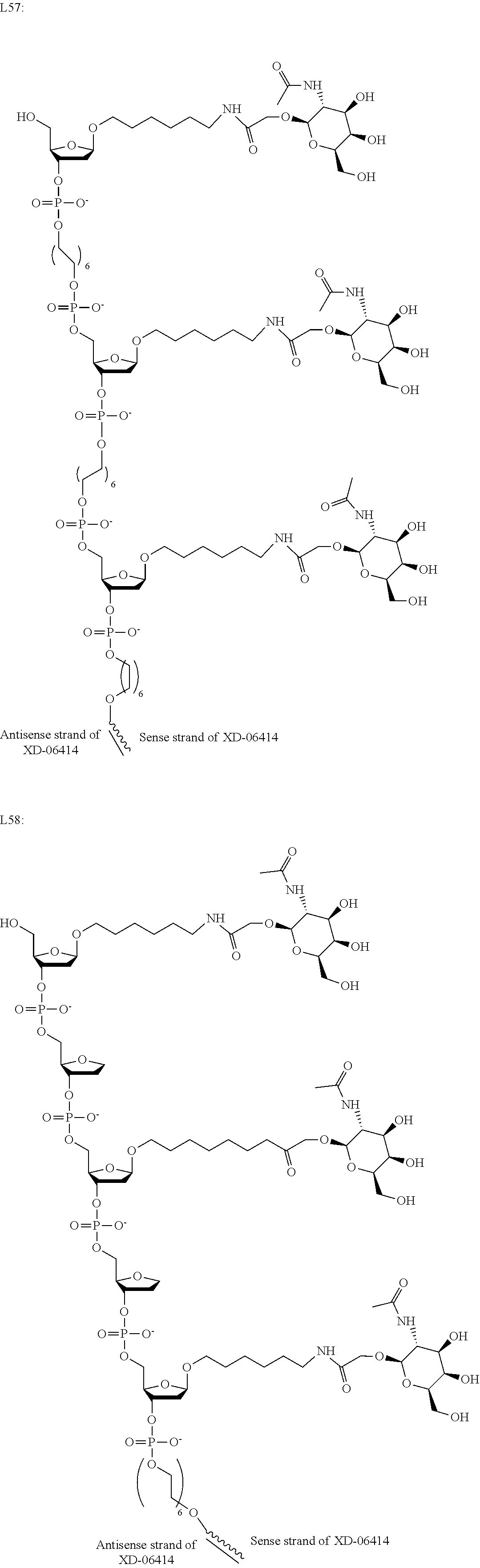

Following the in-vitro experiments the most promising compounds were taken in-vivo in normal mice.

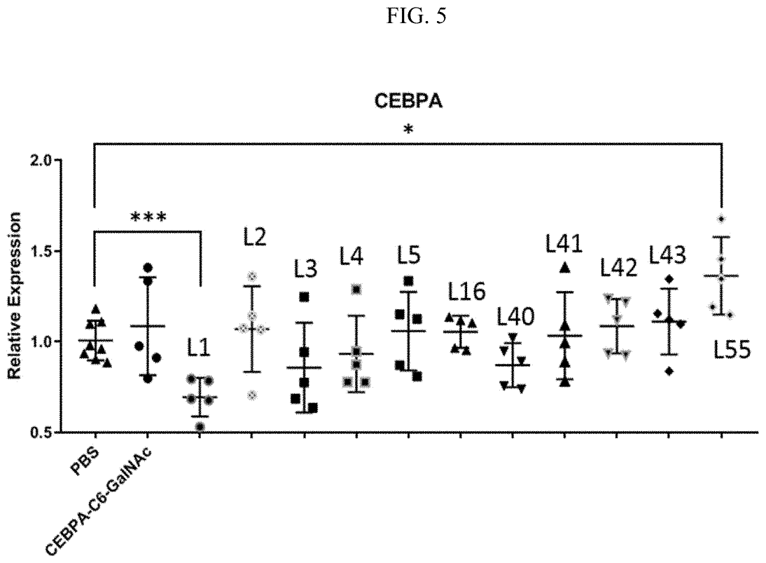

Conjugates L1, L2, L3, L4, L5, L16, L40, L41, L42, L43 and L55 were injected intravenously (IV) at 30 mg/Kg on day 1 and day 3 and the liver was harvested at day 5 to look at CEBPA mRNA upregulation (FIG. 5). Only L55 showed upregulation of CEBPA in the liver, while GalNAc-C6-CEBPA conjugate didn't show any by this mode of administration. Unexpectedly L1 showed downregulation of CEBPA mRNA.

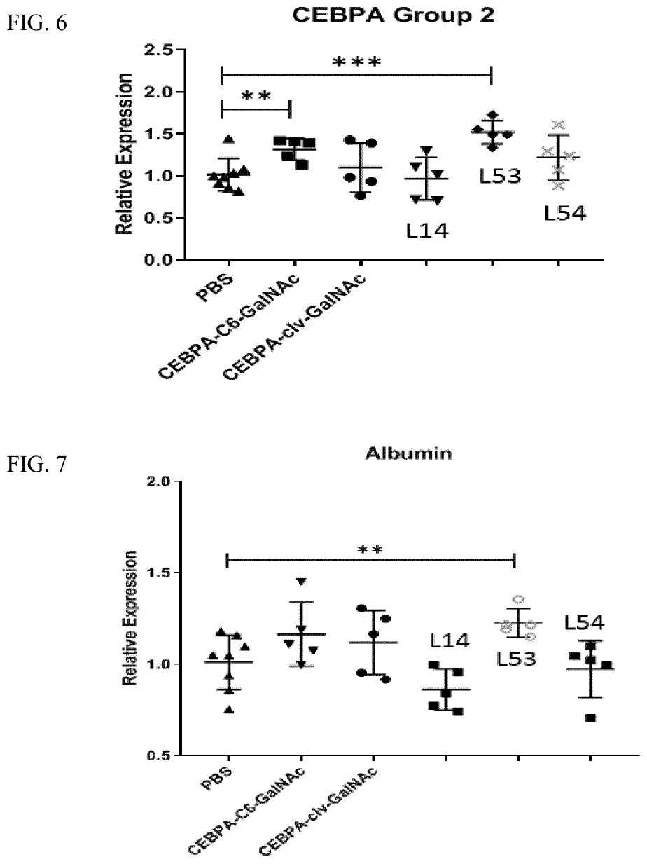

Later L14, L53, and L54 were injected intravenously following the same protocol as the previous experiment (FIG. 6 and FIG. 7). The original GalNAc-C6-CEBPA conjugate showed significant upregulation of CEBPA mRNA and L53 showed significant upregulation of both CEBPA and albumin mRNA. L53 worked better than GalNAc-C6-CEBPA in this experiment.

Finally, L6, L18, L19, L56, L57, and L58 were injected in normal mice subcutaneously (SC) at 30 mg/kg day 1 and day 3 and the liver harvested at day 5. None of the conjugate tested show upregulation in these conditions.

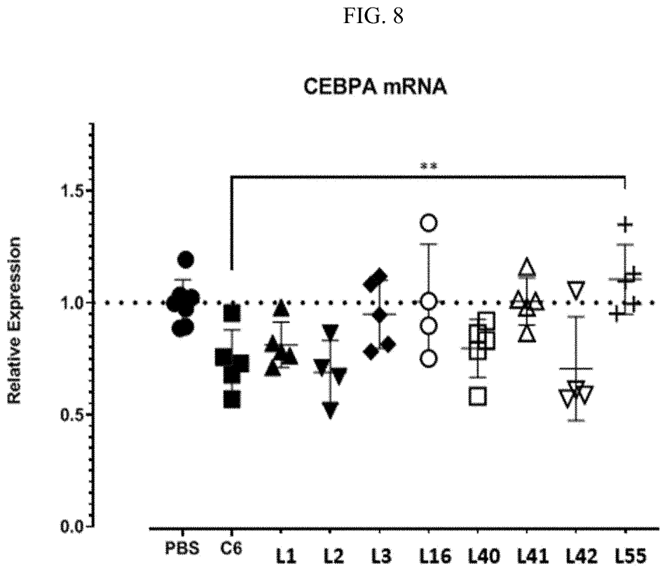

In a further study, L1, L2, L3, L16, L40, L41, L42, and L55 are synthesized again and injected subcutaneously (SC). The GalNAc saRNA conjugates were injected via SC in normal mice at 30 mg/kg day 1 and day 3 and the liver harvested at day 5. As shown in FIG. 8, L55 showed significant better upregulation of CEBPA mRNA than the original GalNAc-C6-CEBPA conjugate via SC administration as well.

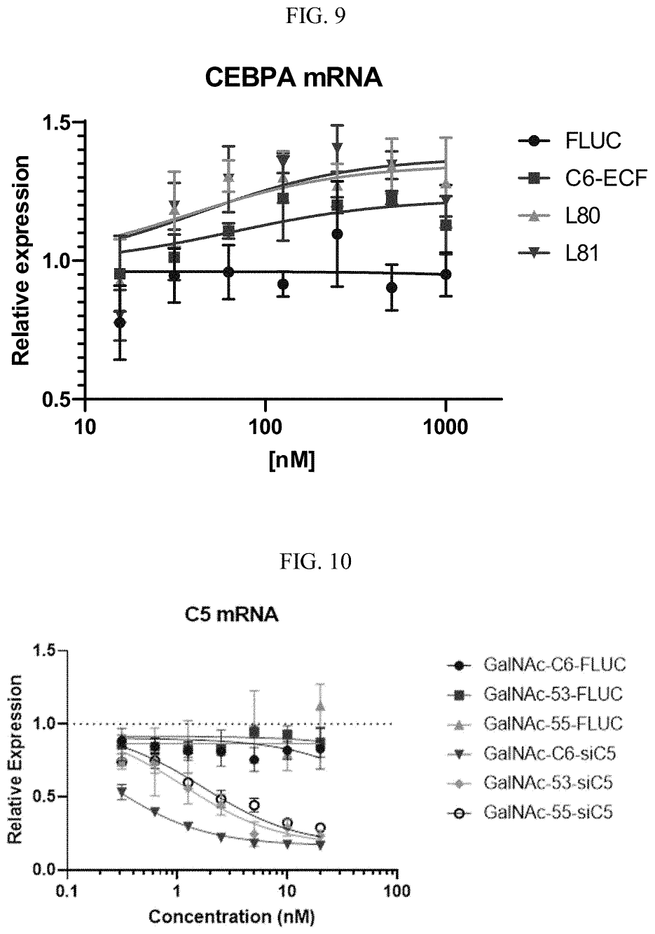

In yet another study, CEBPa-saRNA-GalNAc conjugates L80 (XD-14369K1 conjugated to GalNac cluster G7) and L81 (XD-14369K1 conjugated to GalNac cluster G8) were administered to cells at various doses up to 1000 nM. CEBPA mRNA levels were measured. FIG. 9 shows in-vitro dose response of L80 and L81.

### Example 4. In-Vitro Studies with C5-siRNA-GalNAc Conjugates

In this in vitro study, siRNAs that target the complement C5 gene (C5-siRNAs) were conjugated to GalNAc clusters. The C5-siRNAs were delivered to cells with passive transfection and the C5 mRNA levels were later measured. The sequence of the siRNA is:

| | Original | Modified |
|---|---|---|
| | Sequence | Sequence |
| Sense/ | AAGCAAGAUAUUU | AfsasGfcAfaGfaUfAf |
| Passenger | UUAUAAUA | UfnUfnUfaUfaAfuAf- |
| Strand | | C6-GalNAc |
| Antisense/ | UAUUAUAAAAAUA | usAfsuUfaUfaAfaAfa |
| Guide | UCUUGCUUUU | uaUfcUfuGfcUfususu |
| Strand | | |

Nf = the nucleotide N (N may be A, U, C, or G) has a 2′-Fluoro (2′-F) modification
lower case = the nucleotide has a 2′-O-Methyl (2′-OMe) modification
s: phosphorothioate linkage

The C5-siRNA-GalNAc conjugates tested in this study included:
1. C5-siRNA-C6-GalNAc (GalNAc-C6-siC5 in FIG. 10) having a structure of

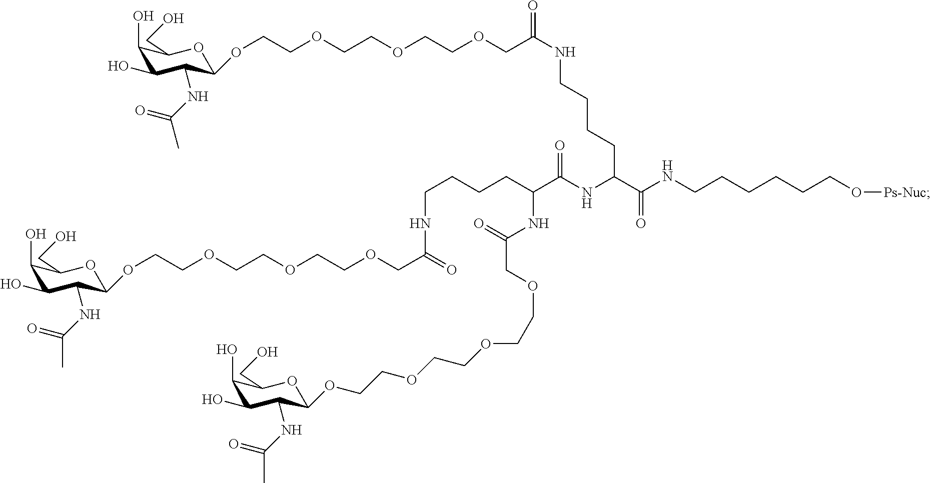

2. C5-siRNA-G7 (GalNAc-53-siC5 in FIG. 10) having a structure of

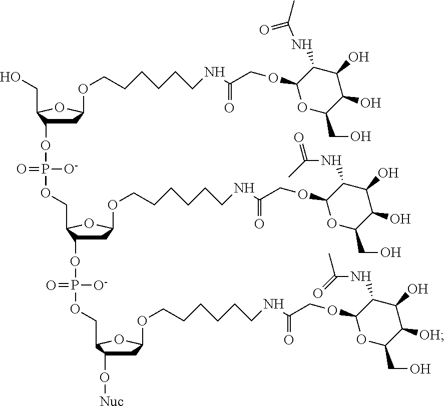

and
3. C5-siRNA-G9 (GalNAc-55-siC5 in FIG. 10) having a structure of

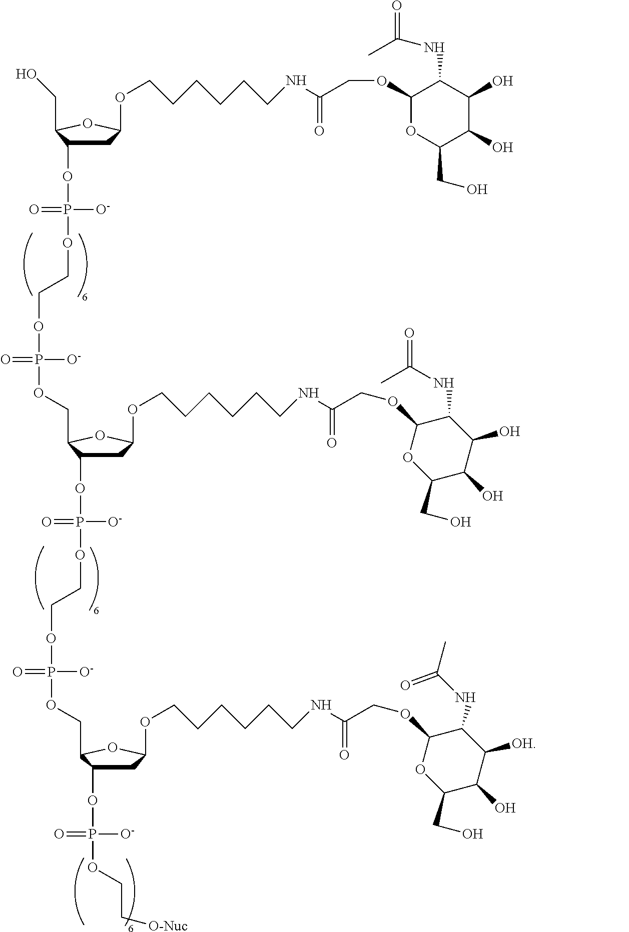

GalNAc-C6-siC5, GalNAc-53-siC5, GalNAc-55-siC5, and the controls (GalNAc-C6-FLUC, GalNAc-53-FLUC and GalNAc-55-FLUC) were administered to primary rat hepatocyte cells at a dose between 0.3125 nM to 20 nM. Then C5 mRNA levels in the cells were measured by qPCR. As shown in FIG. 10, C5-siRNA conjugated to GalNAc cluster G7 (GalNAc-53-siC5), C5-siRNA conjugated to GalNAc cluster G9 (GalNAc-55-siC5) and GalNAc-C6-siC5 all reduced the C5 mRNA levels.

### OTHER EMBODIMENTS

It is to be understood that while the present disclosure has been described in conjunction with the detailed description thereof, the foregoing description is intended to illustrate and not limit the scope of the present disclosure, which is defined by the scope of the appended claims. Other aspects, advantages, and modifications are within the scope of the following claims.

1. A N-Acetyl-Galactosamine (GalNAc) monomer comprising a structure selected from the group consisting of 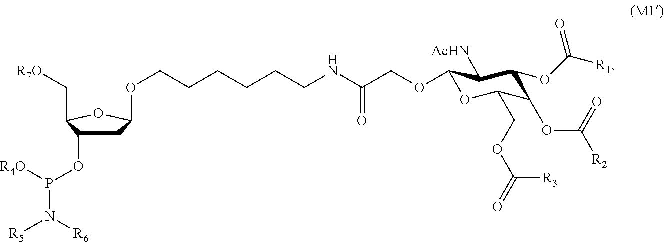
wherein R1, R2, and R3 can be the same or different, and wherein R1, R2, and R3 are independently selected from an alkyl, aryl, and alkenyl group,
wherein R4 is a suitable protecting group or a C1-6 straight or branched alkyl group,
wherein R5 and R6 are each independently a C1-6 straight or branched alkyl group, and
wherein R7 is a suitable protecting group; 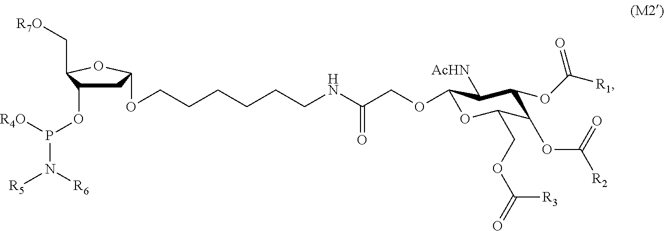
wherein R1, R2, and R3 can be the same or different, and wherein R1, R2, and R3 are independently selected from an alkyl, aryl, and alkenyl group;
wherein R4 is a protecting group or a C1-6 straight or branched alkyl group,
wherein R5 and R6 are each independently C1-6 straight or branched alkyl; and
wherein R7 is a suitable protecting group; 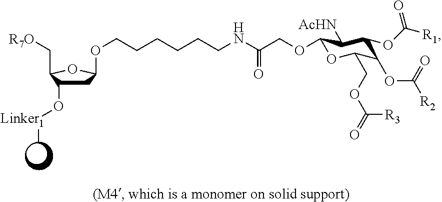
wherein R1, R2, and R3 can be the same or different, and wherein R1, R2, and R3 are independently selected from an alkyl, aryl, and alkenyl group,
wherein R7 is a suitable protecting group, and
wherein Linker1 is a cleavable linker; and 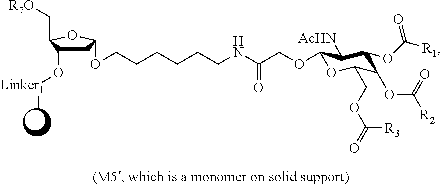
wherein R1, R2, and R3 can be the same or different, and wherein R1, R2, and R3 are independently selected from the group consisting of an alkyl, aryl, and alkenyl group,
wherein R7 is a suitable protecting group, and
wherein Linker1 is a cleavable linker.

2. A GalNAc moiety comprising at least one GalNAc monomer, wherein the GalNAc monomers are selected from the group consisting of 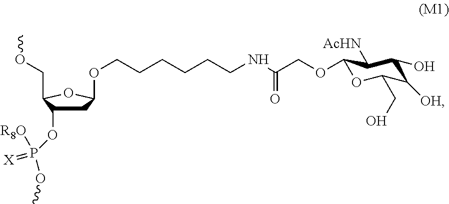
wherein R₈ is —H or a C1-6 straight or branched alkyl group; 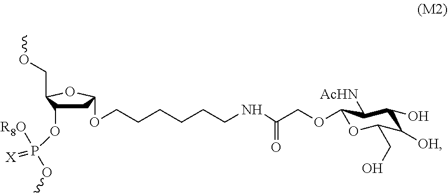
wherein R₈ is —H or a C1-6 straight or branched alkyl group, and wherein X is O or S; 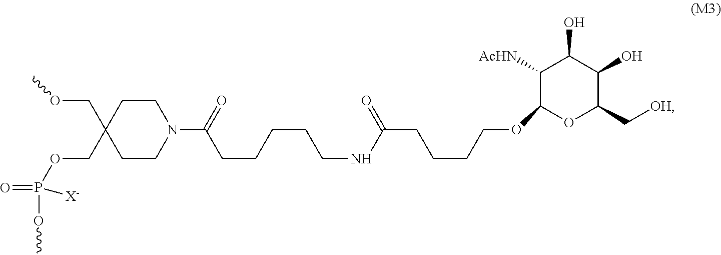
wherein X is O or S; 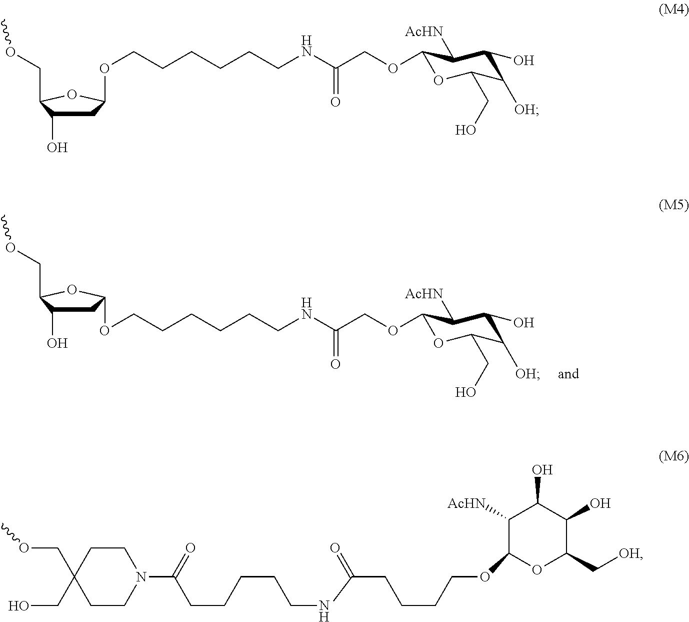
wherein the GalNAc moiety does not comprise more than one M4, M5 or M6 monomer, and
wherein the GalNAc moiety is not a GalNAc moiety consisting of only one M3 monomer,
only one M6 monomer, or one M6 monomer and two M3 monomers.

3. The GalNAc moiety of claim 2, wherein the GalNAc moiety comprises a spacer comprising a structure of HEG, C12, ab, TEG, or C3.

4. The GalNAc moiety of claim 2, wherein the GalNAc moiety comprises two or three GalNAc monomers.

5. The GalNAc moiety of claim 2, wherein the GalNAc moiety comprises three M1, M2 or M3 monomers.

6. The GalNAc moiety of claim 5, wherein the GalNAc moiety comprises three M1 monomers.

7. The GalNAc moiety of claim 2, wherein the GalNAc cluster comprises a structure of

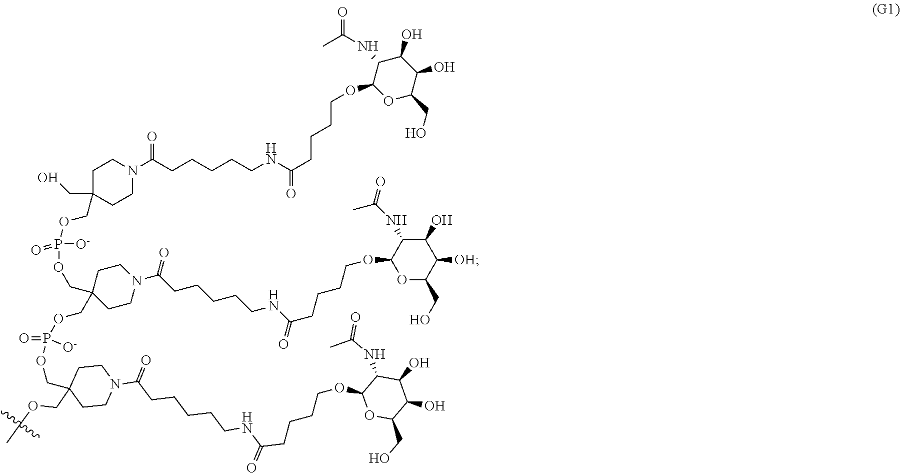 [SMILES: *OCC1(COP(=O)([O-])OCC2(COP(=O)([O-])OCC3(CO)CCN(C(=O)CCCCCNC(=O)CCCCO[C@@H]4O[C@H](CO)[C@H](O)[C@H](O)[C@H]4NC(C)=O)CC3)CCN(C(=O)CCCCCNC(=O)CCCCO[C@@H]3O[C@H](CO)[C@H](O)[C@H](O)[C@H]3NC(C)=O)CC2)CCN(C(=O)CCCCCNC(=O)CCCCO[C@@H]2O[C@H](CO)[C@H](O)[C@H](O)[C@H]2NC(C)=O)CC1]

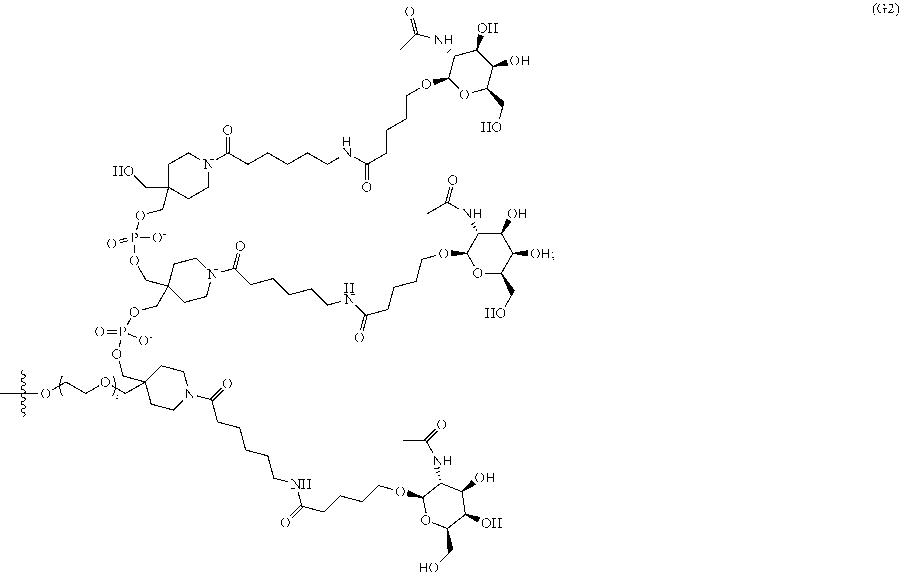

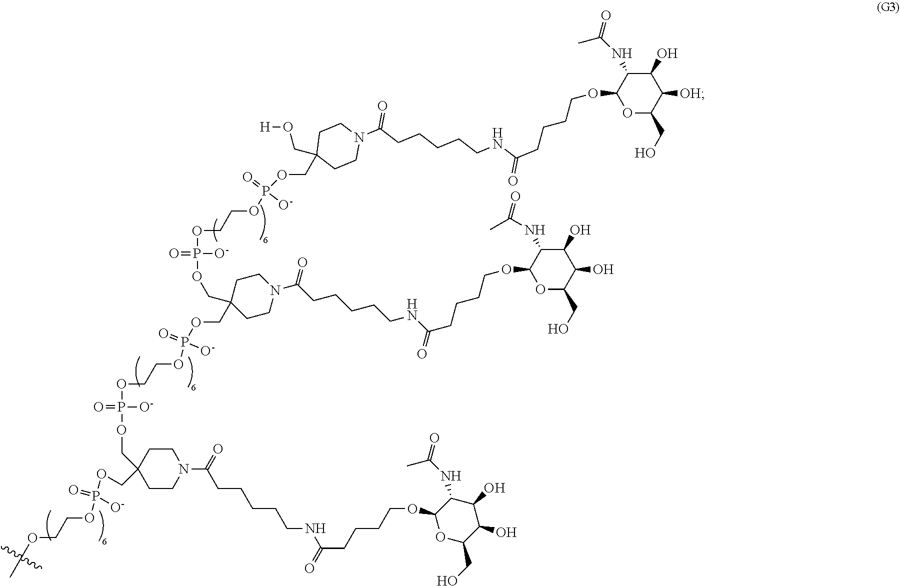

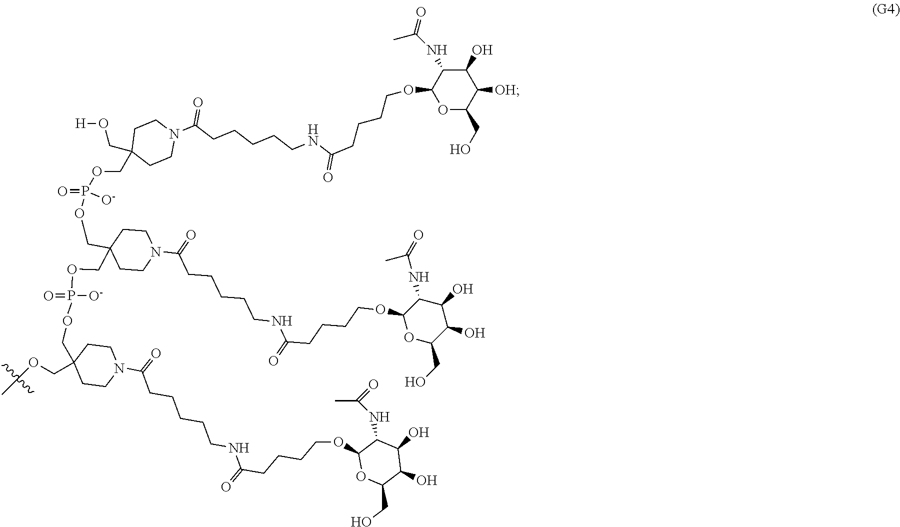 [SMILES: *OCC1(COP(=O)([O-])OCC2(COP(=O)([O-])OCC3(CO)CCN(C(=O)CCCCCNC(=O)CCCCO[C@@H]4O[C@H](CO)[C@H](O)[C@H](O)[C@H]4NC(C)=O)CC3)CCN(C(=O)CCCCCNC(=O)CCCCO[C@@H]3O[C@H](CO)[C@H](O)[C@H](O)[C@H]3NC(C)=O)CC2)CCN(C(=O)CCCCCNC(=O)CCCCO[C@@H]2O[C@H](CO)[C@H](O)[C@H](O)[C@H]2NC(C)=O)CC1]

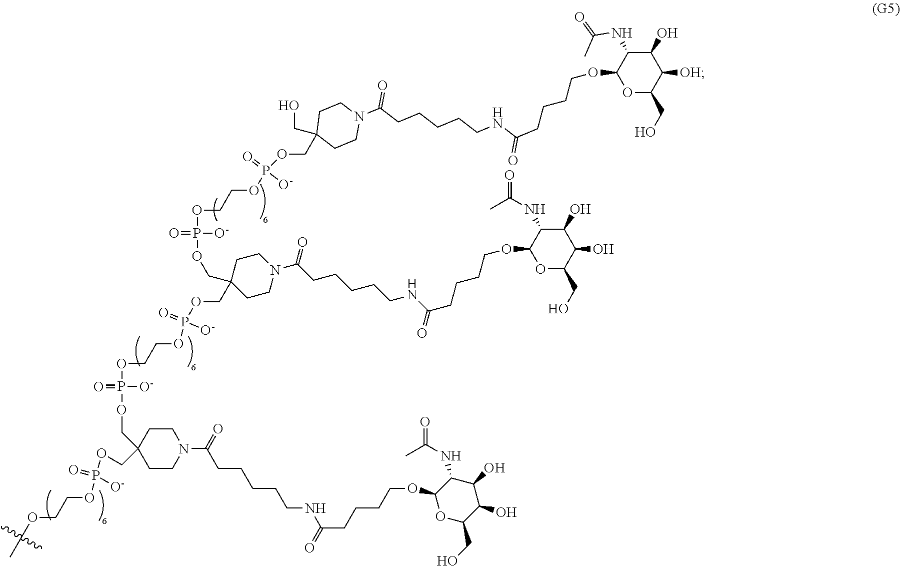

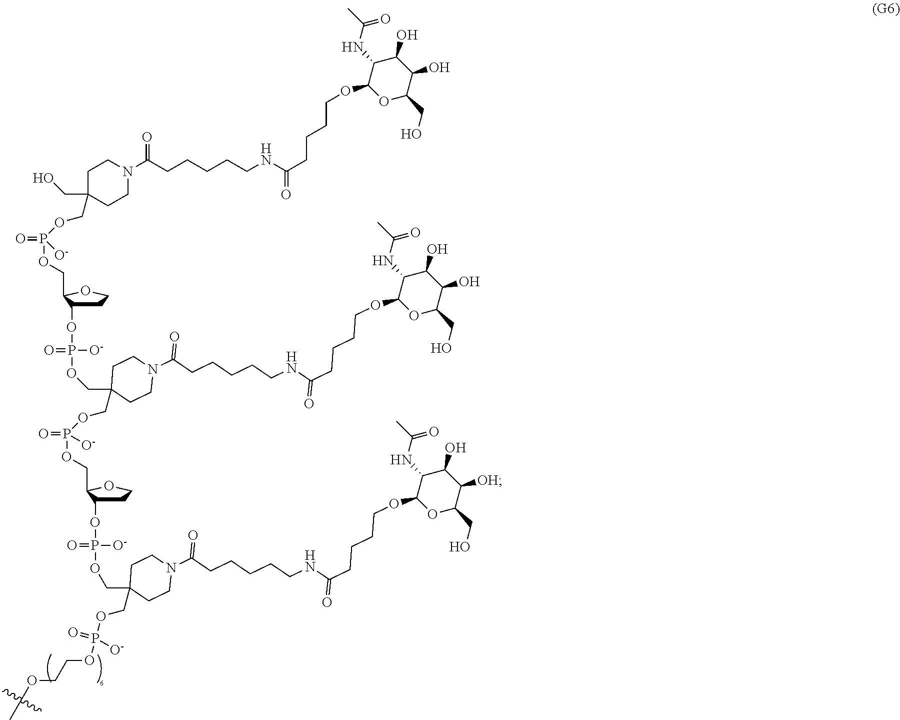

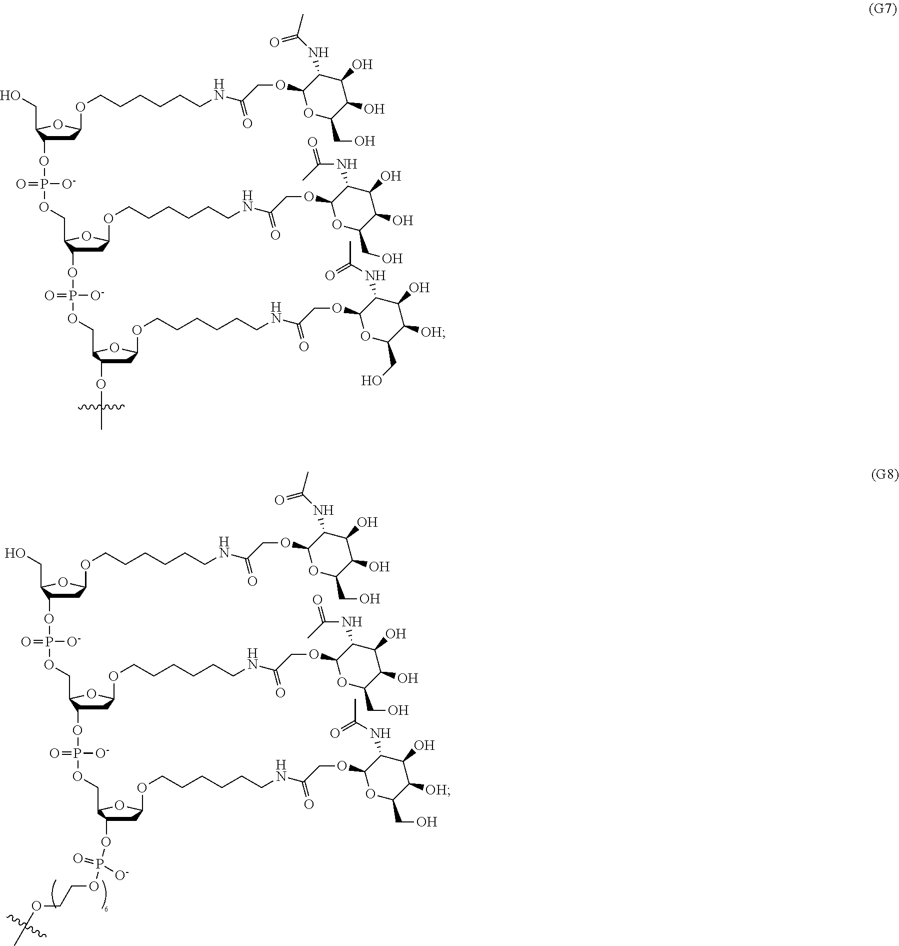

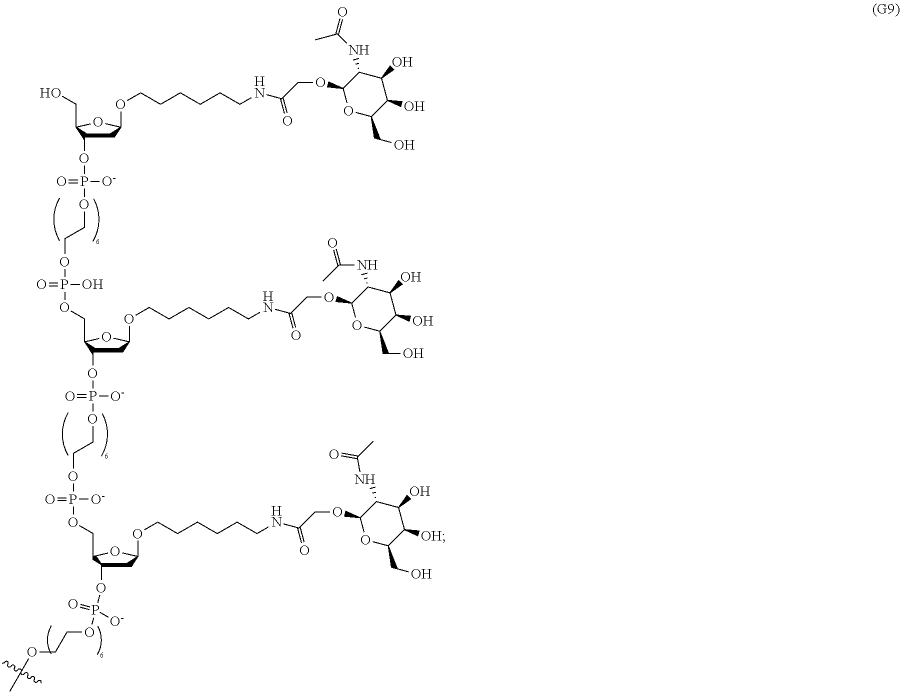

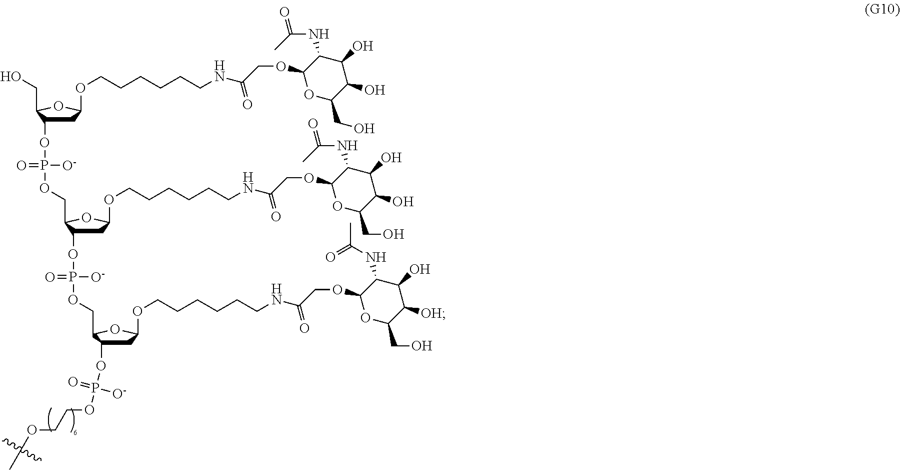

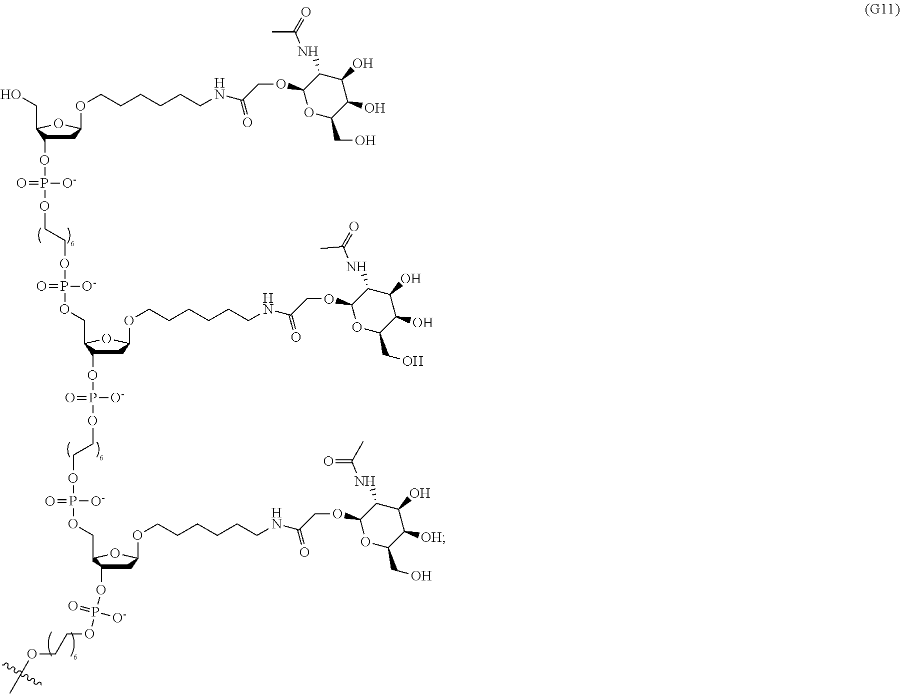

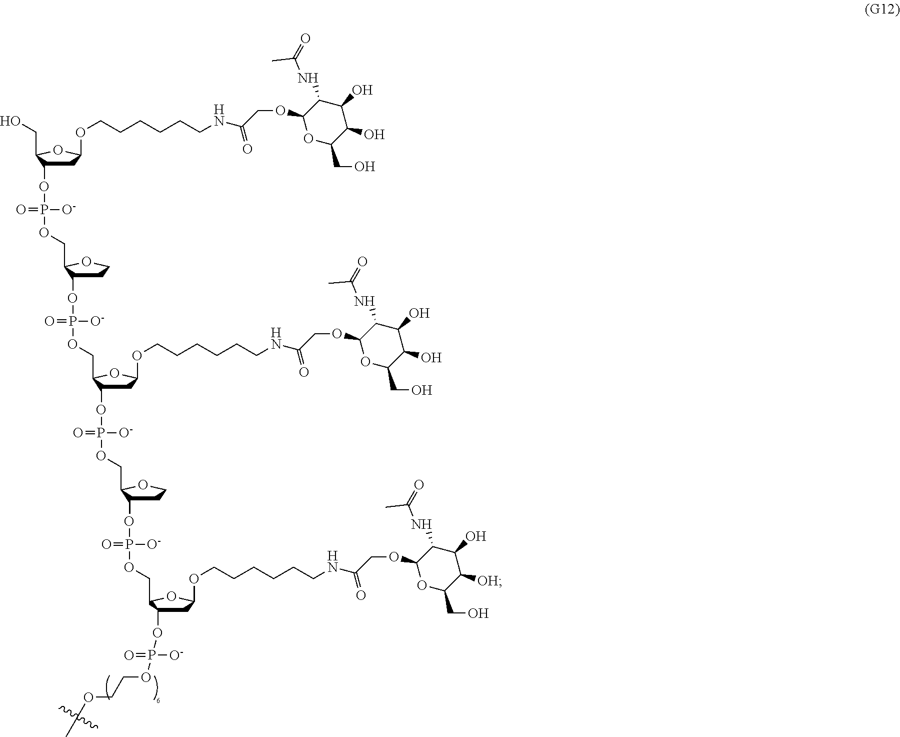

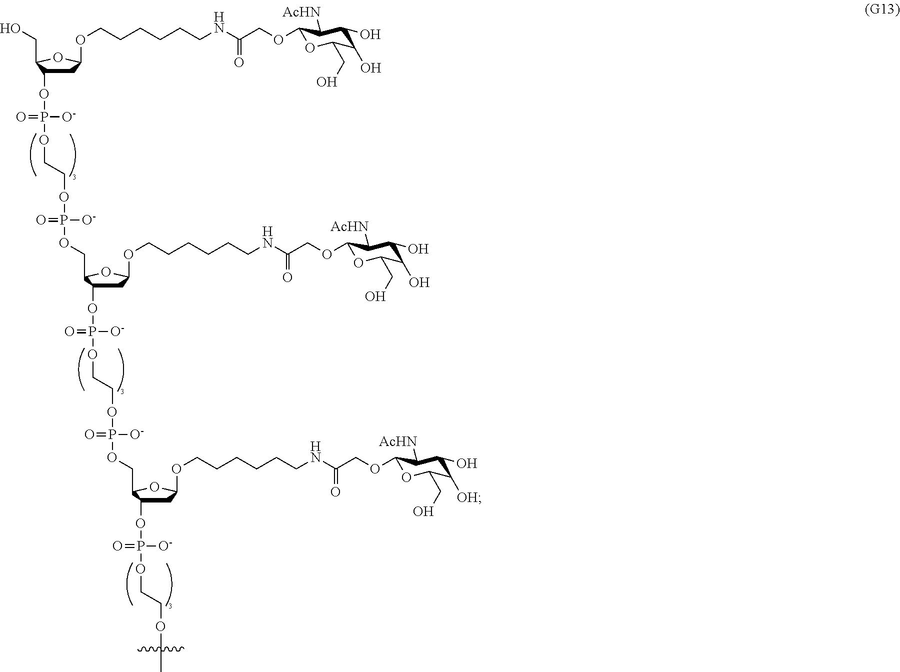

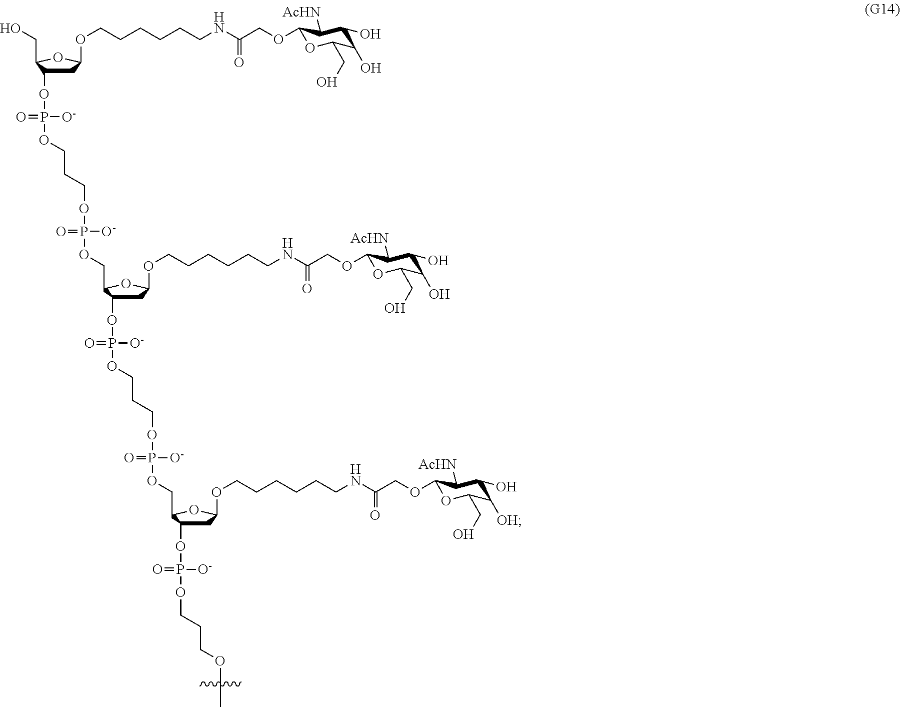

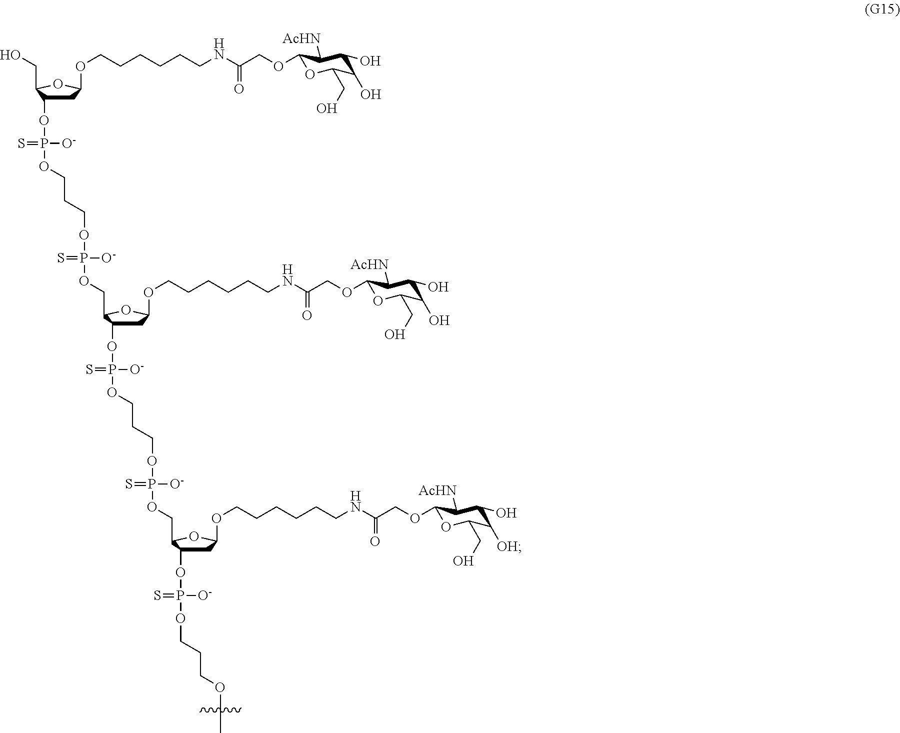

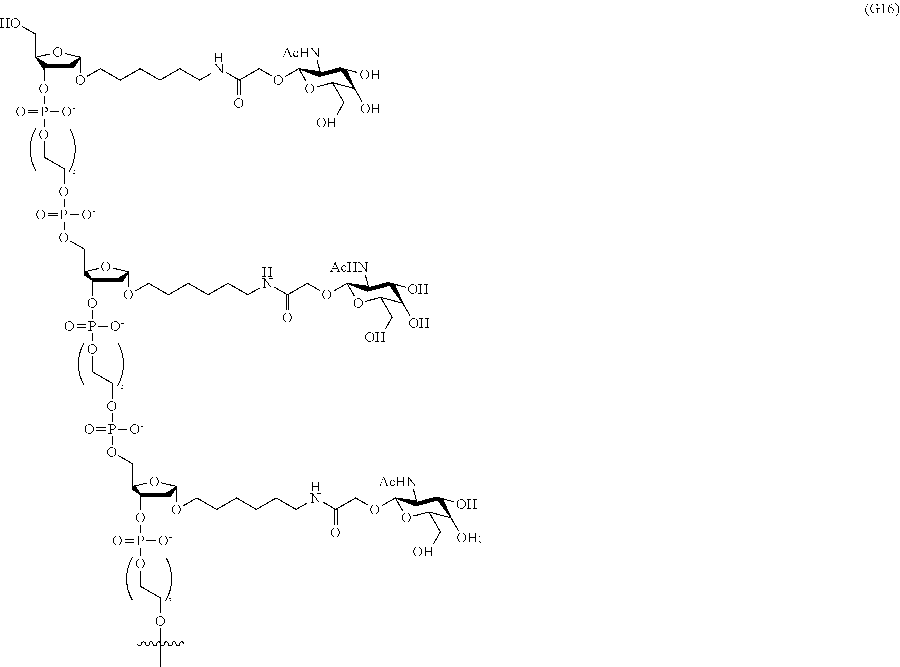

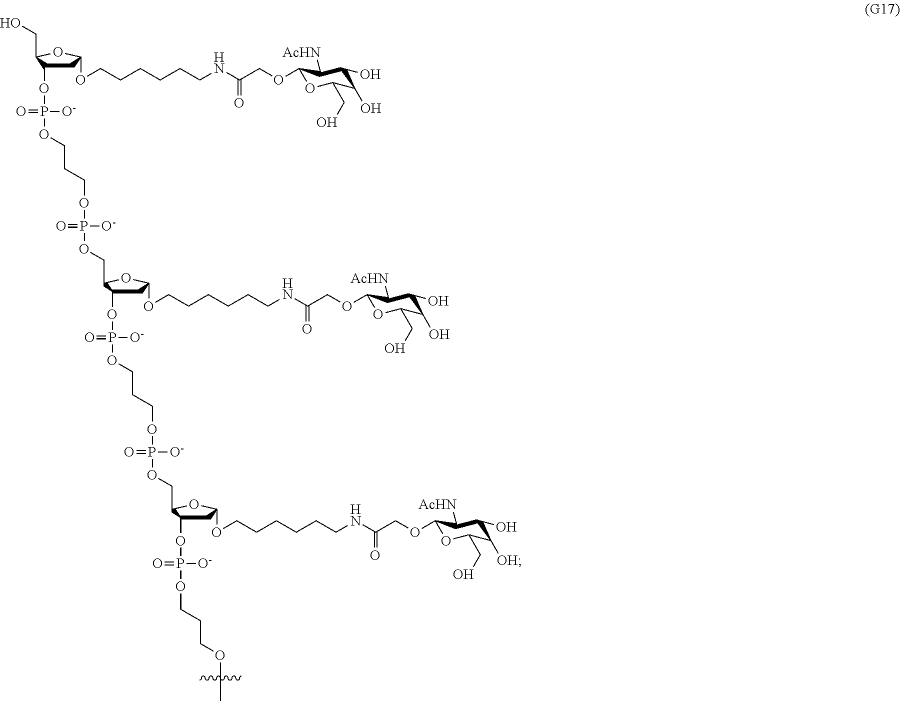

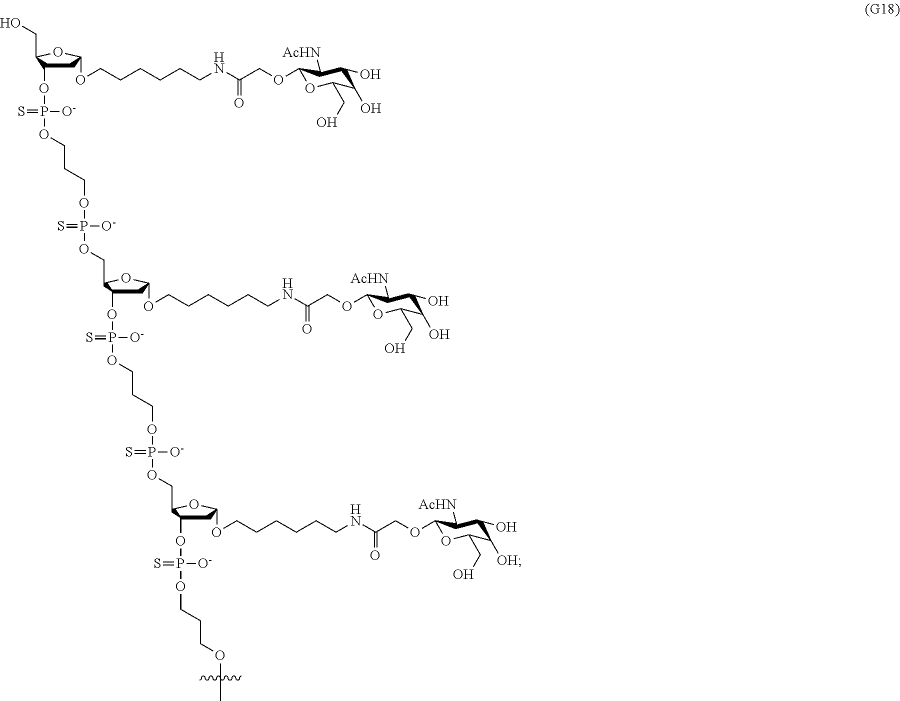

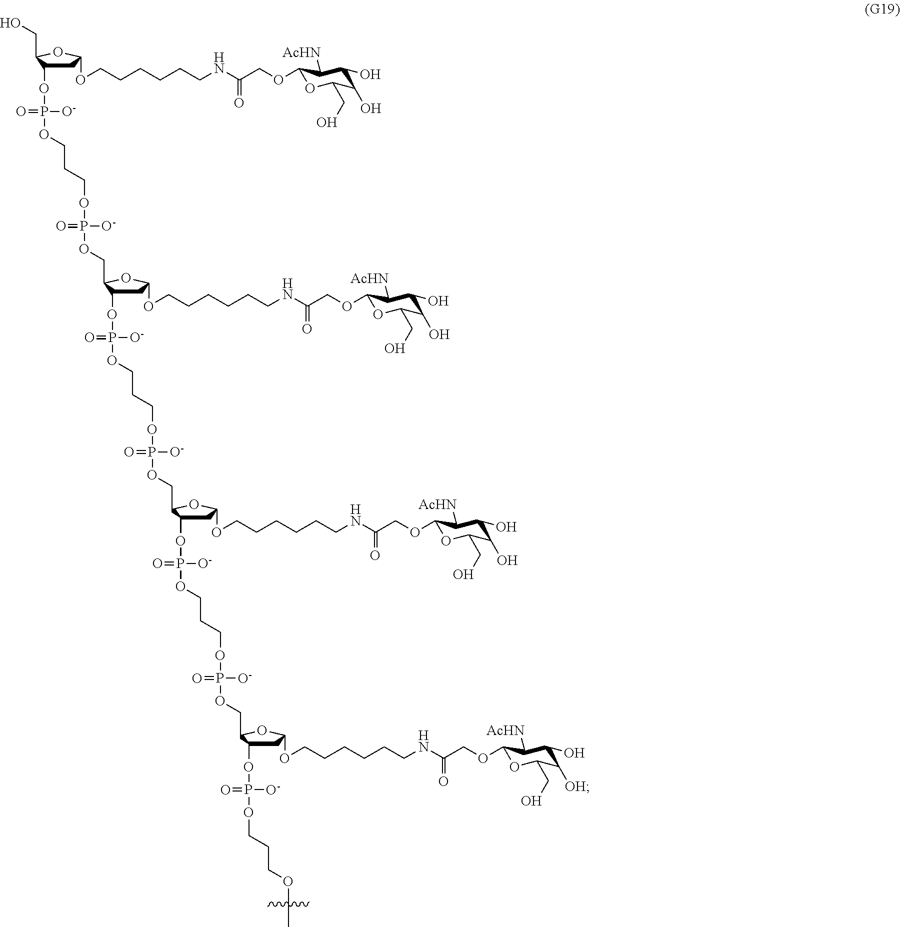

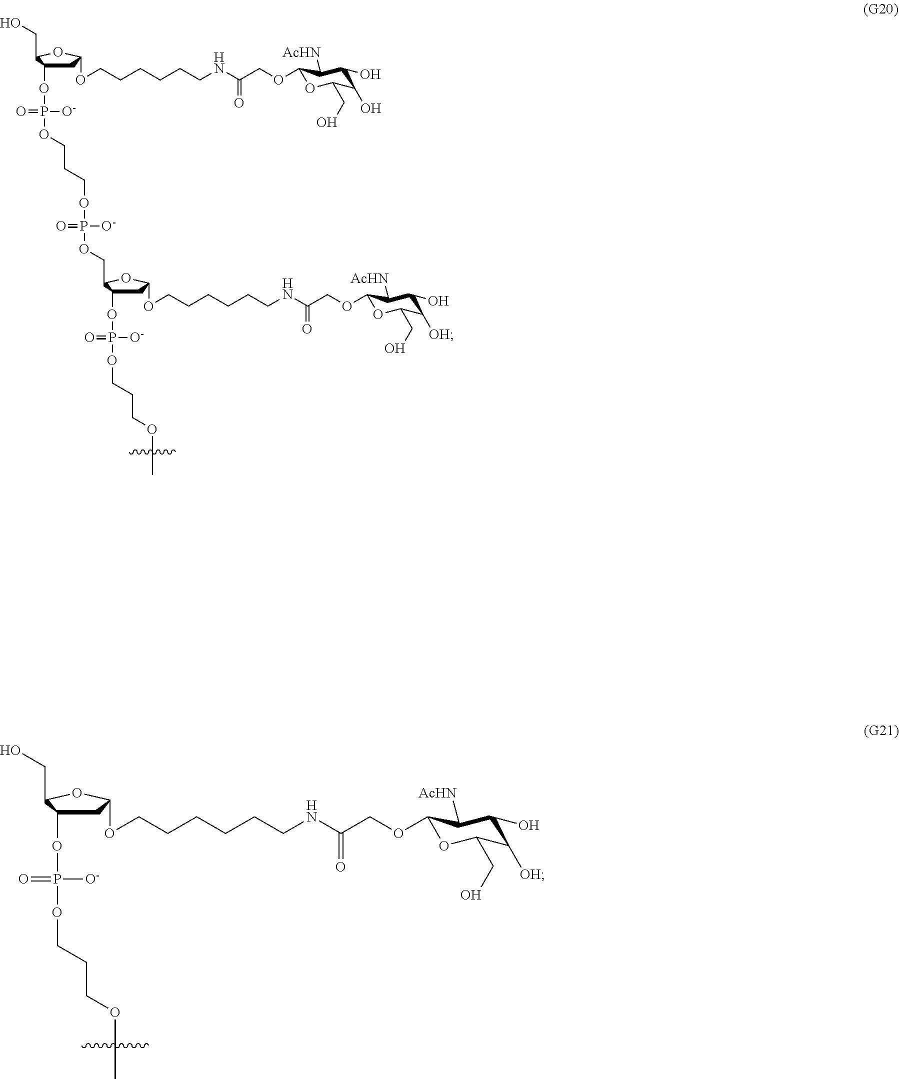

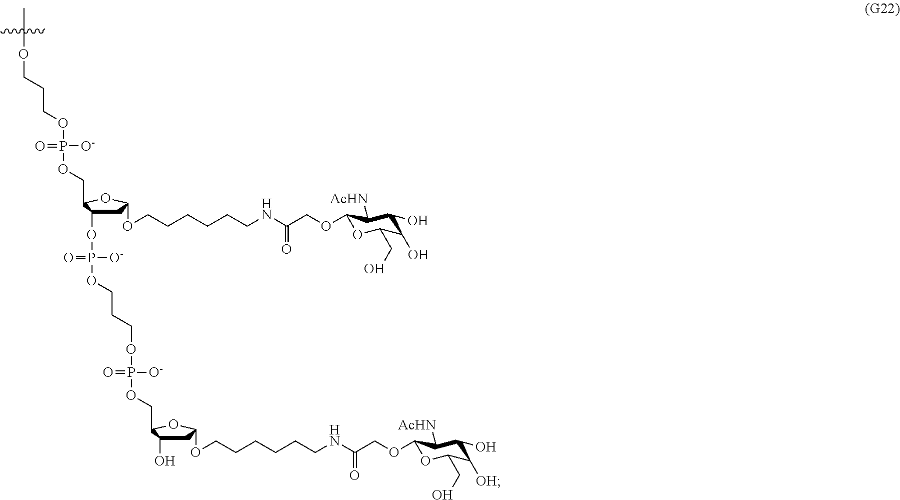

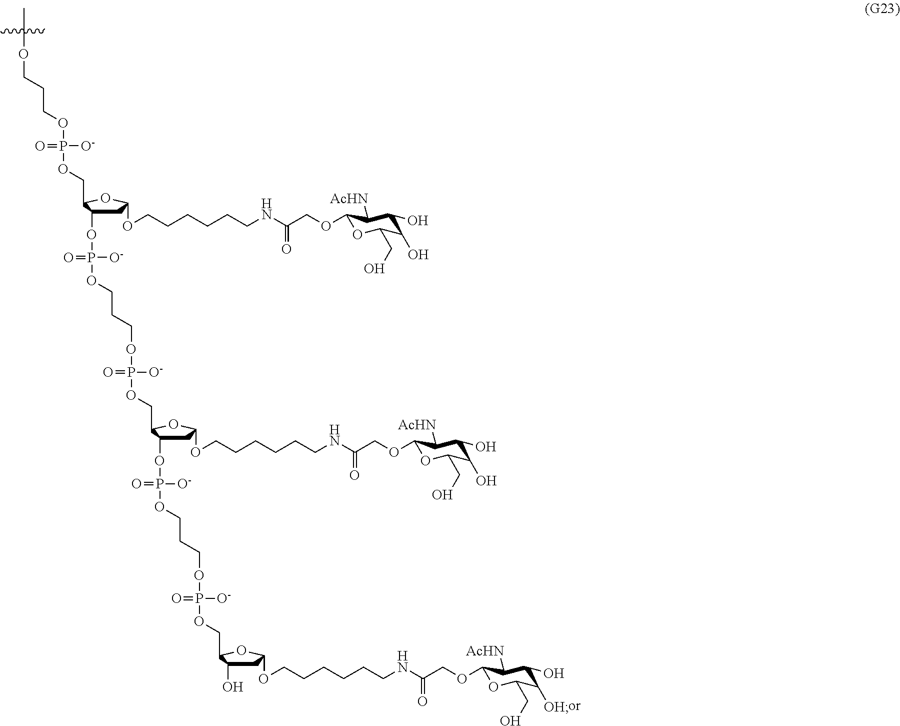

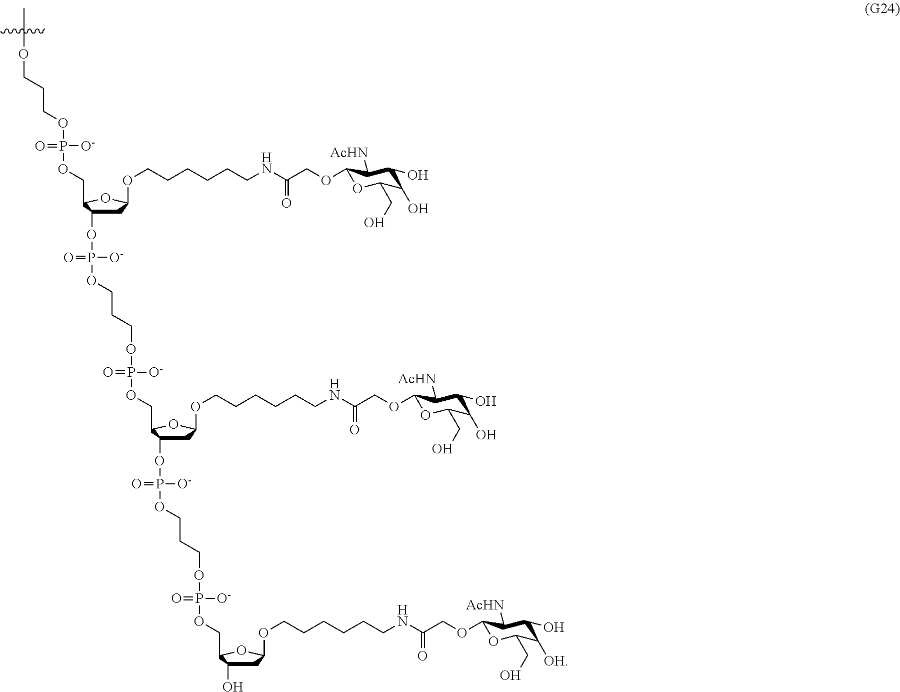

8. A GalNAc moiety prepared by a process comprising the steps of:
1). Providing at least one GalNAc monomer selected from the group consisting of any monomer of claim 1, 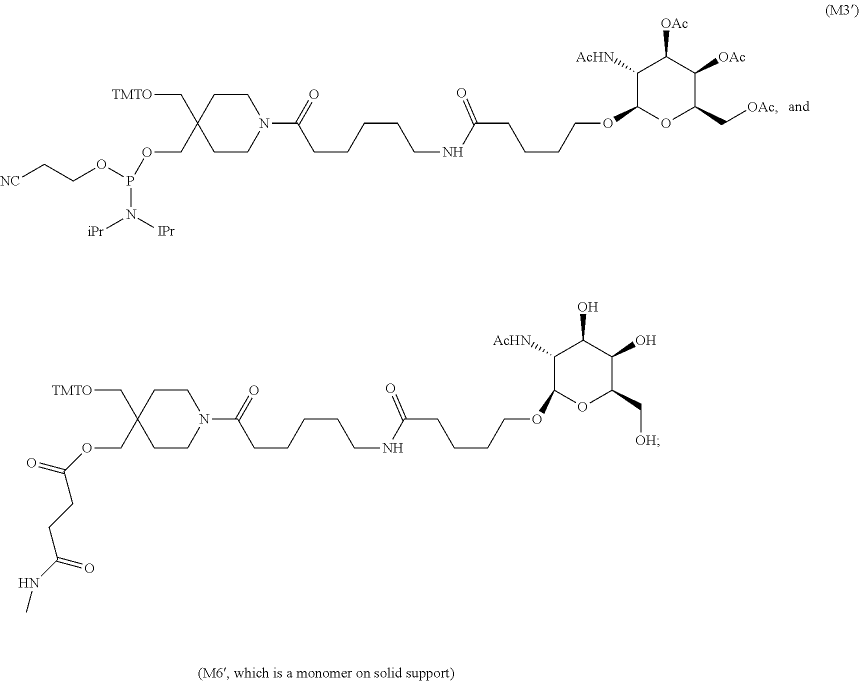
and
2). synthesizing a GalNAc moiety from the GalNAc monomers in step 1); optionally adding at least one spacer and optionally removing the protecting groups.

9. A conjugate comprising an oligonucleotide and the GalNAc moiety of claim 2, wherein the oligonucleotide regulates the expression of a target gene, and wherein the oligonucleotide and the GalNAc moiety are connected by a bond or a cleavable linker.

10. The conjugate of claim 9, wherein the oligonucleotide and the GalNAc moiety are connected by a cleavable linker.

11. The conjugate of claim 10, wherein the linker is C6ssC6 or dT.

12. The conjugate of claim 9, wherein the oligonucleotide and the GalNAc moiety are connected by a bond, wherein the bond is a phosphodiester bond or a phosphorothioate bond.

13. The conjugate of claim 9, wherein the conjugate comprises a structure of CJ1, CJ2, CJ3, CJ4, CJ5, CJ6, CJ7, CJ8, CJ9, CJ10, CJ11, CJ12, CJ13, CJ14, CJ15, CJ16, CJ17, CJ18, CJ19, CJ20, CJ21, CJ22, CJ23, CJ24, or CJ25.

14. The conjugate of claim 9, wherein the oligonucleotide is a synthetic isolated small activating RNA (saRNA).

15. The conjugate of claim 14, wherein the target gene is CEBPA.

16. The conjugate of claim 14, wherein saRNA is a double-stranded saRNA.

17. The conjugate claim 16, wherein the GalNAc cluster is connected to the 5′ or 3′ end of the sense strand.

18. The conjugate of claim 16, wherein the double-stranded saRNA is selected from the group consisting of XD-03302, S1 (XD-06409), S2 (XD-06410), S3 (XD-06411), S4 (XD-06412), S5 (XD-06413), S6 (XD-06414), S7 (XD-06415), S8, XD-07139, XD-03934, and XD-14369K1.

19. The conjugate of claim 16, wherein the saRNA is XD-06414 having an antisense strand comprising SEQ ID NO: 15 and a sense strand comprising SEQ ID NO: 14.

20. The conjugate of claim 19, wherein the conjugate is selected from the group consising of

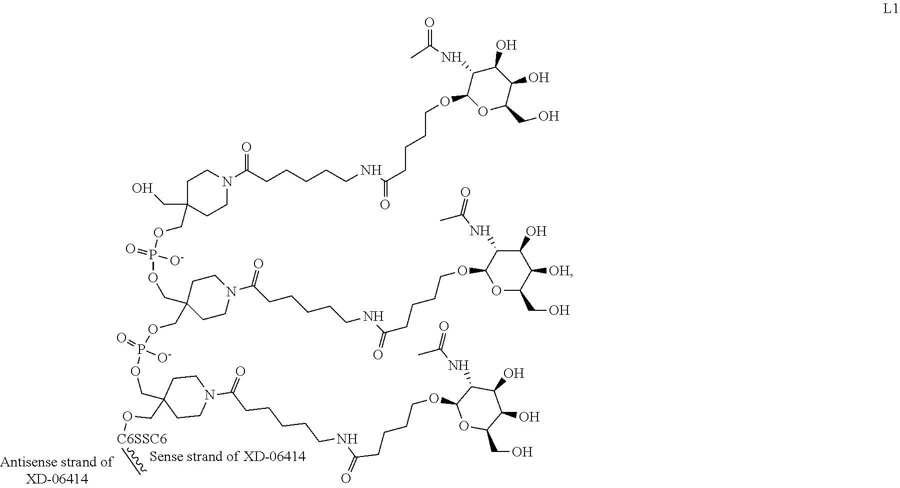

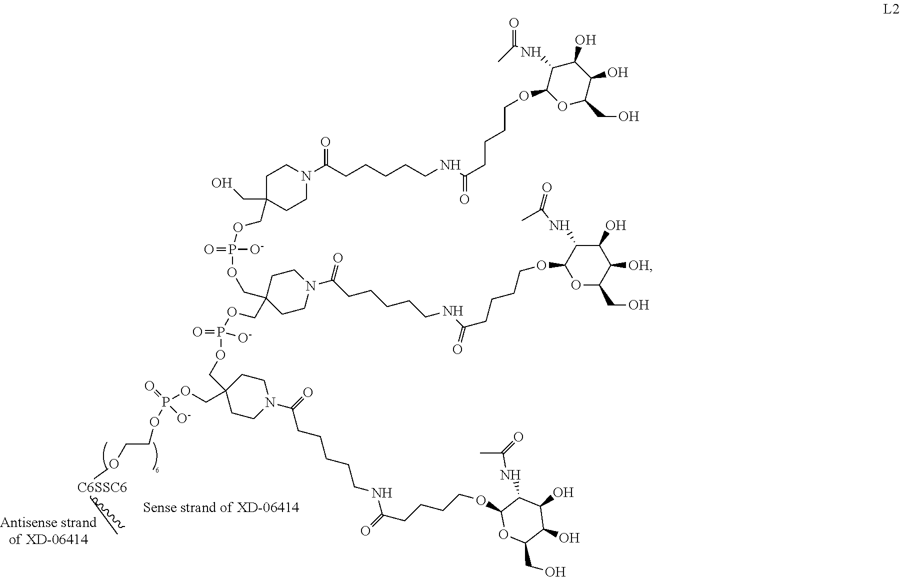

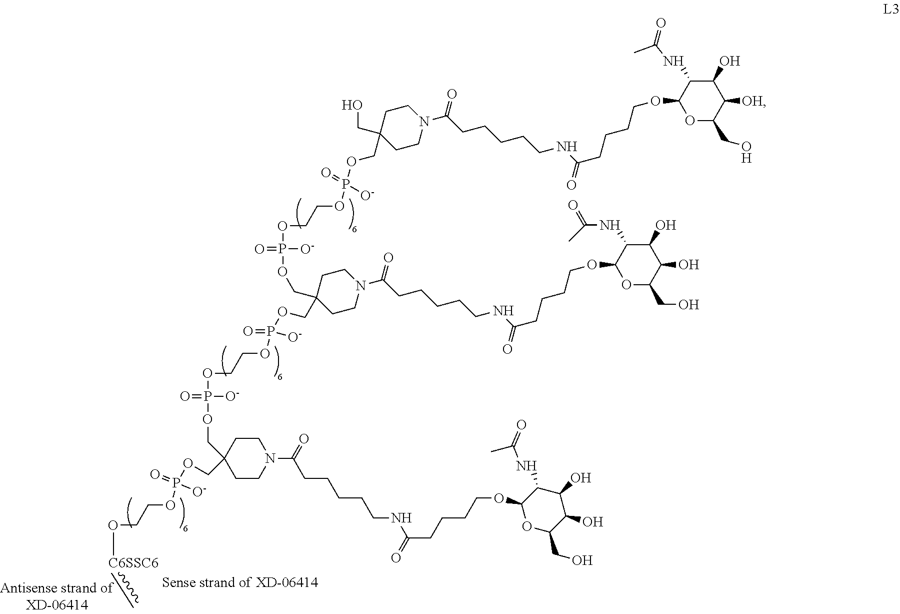

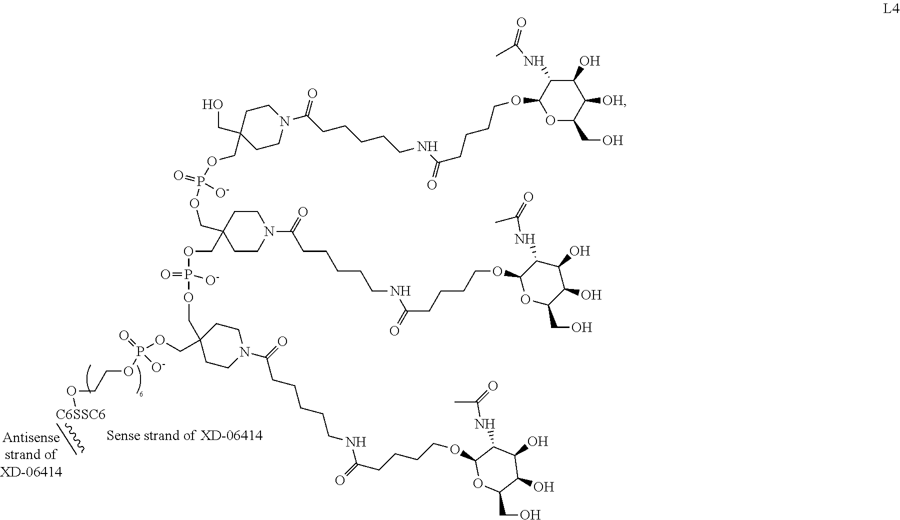

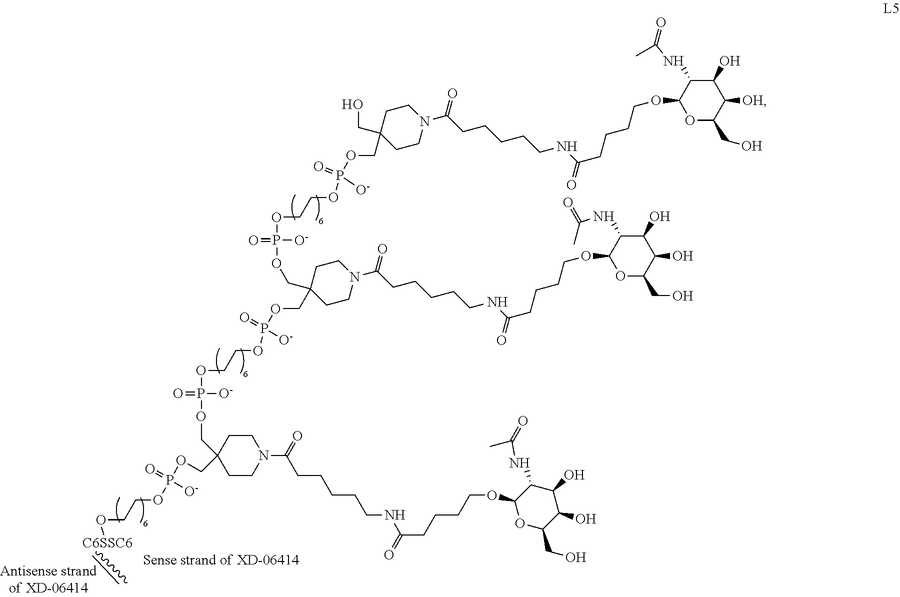

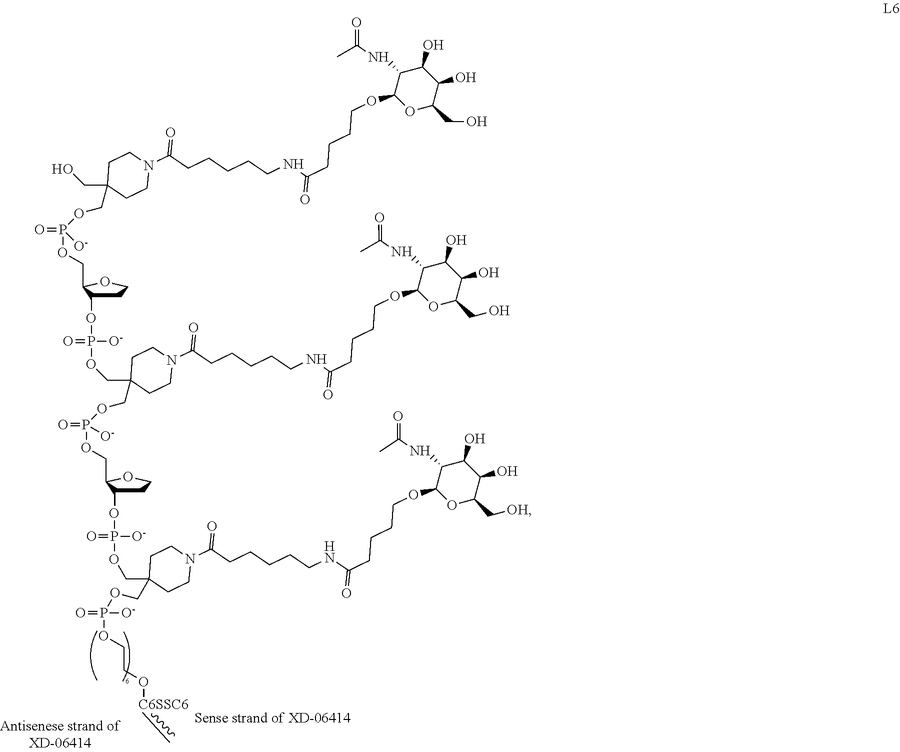

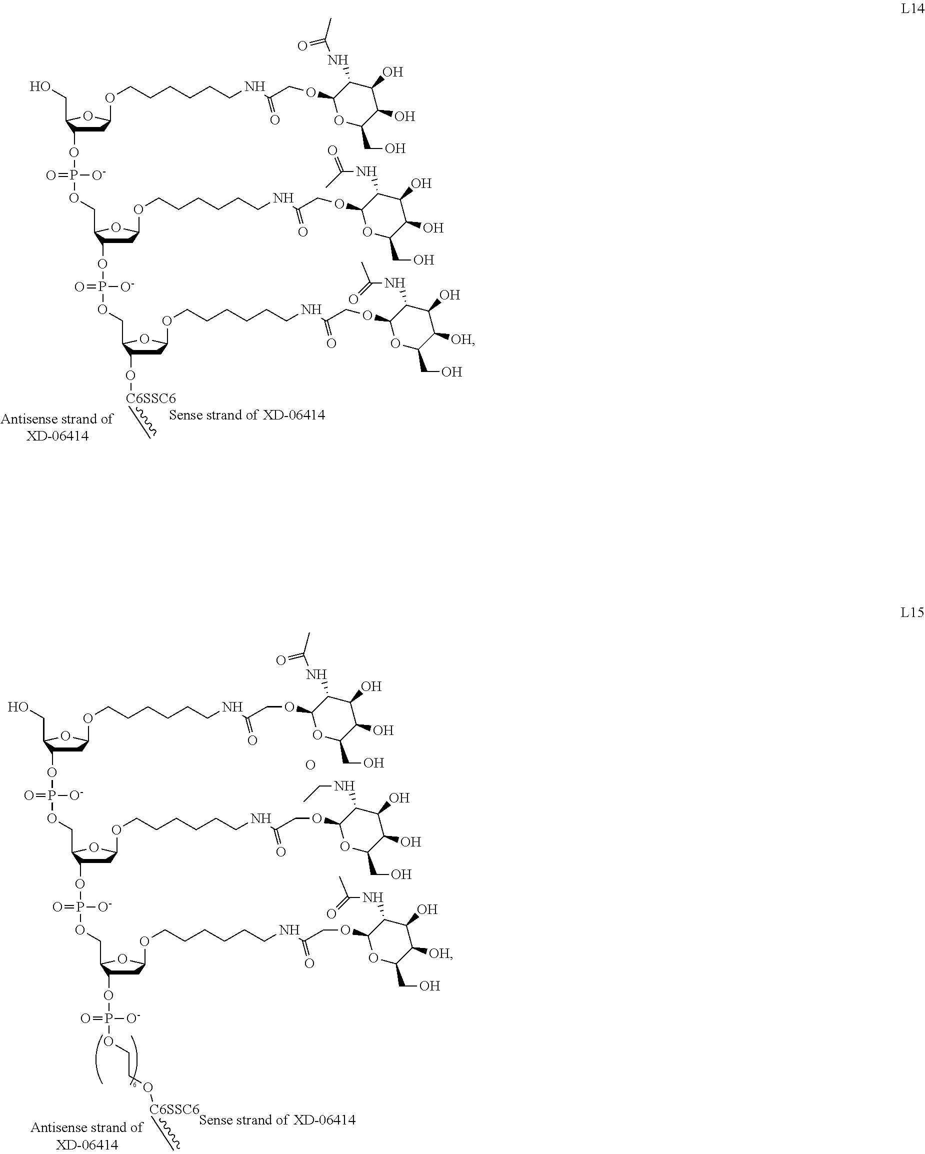

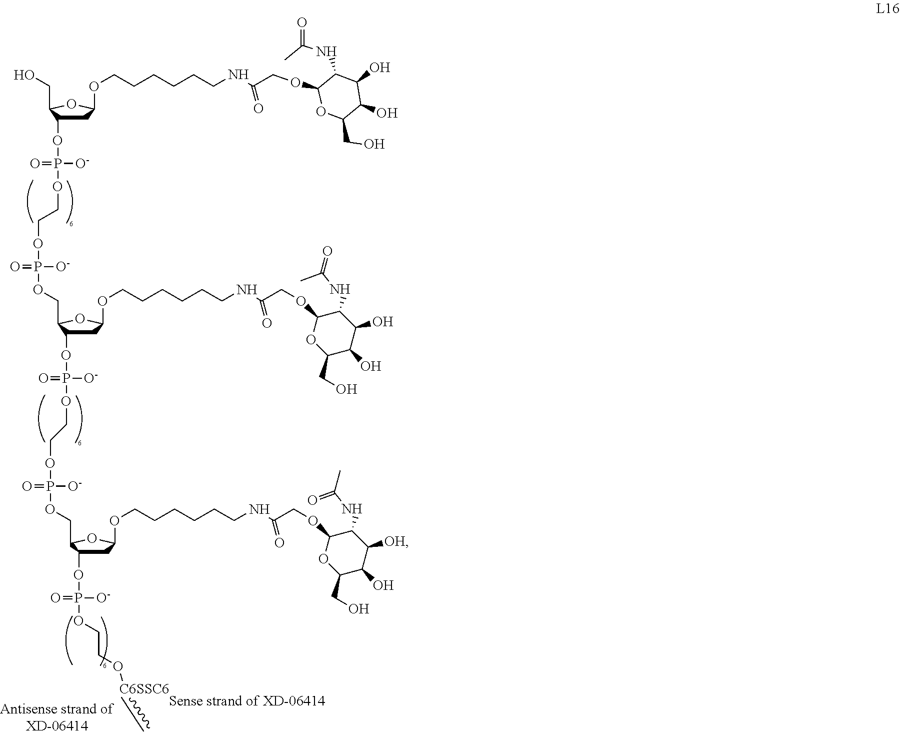

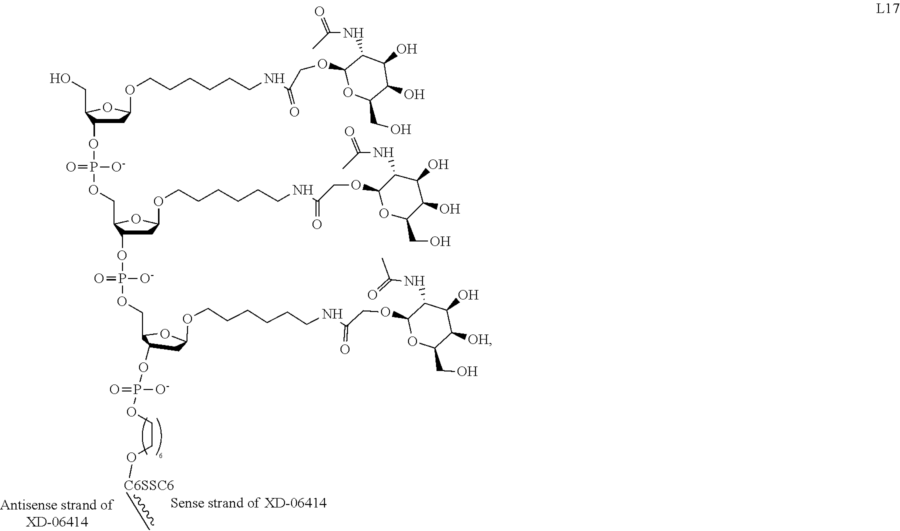

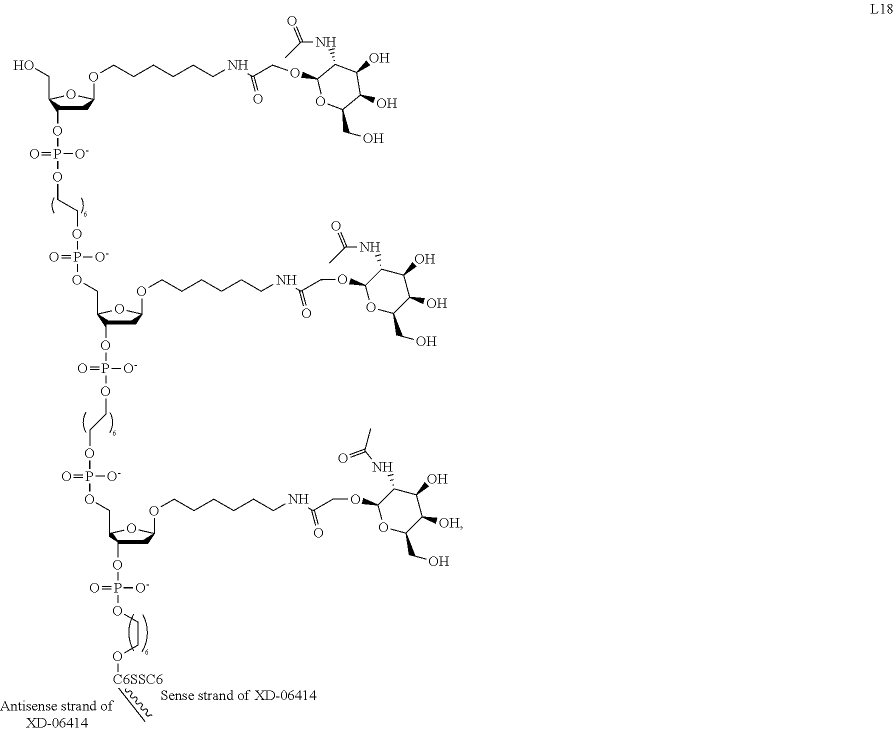

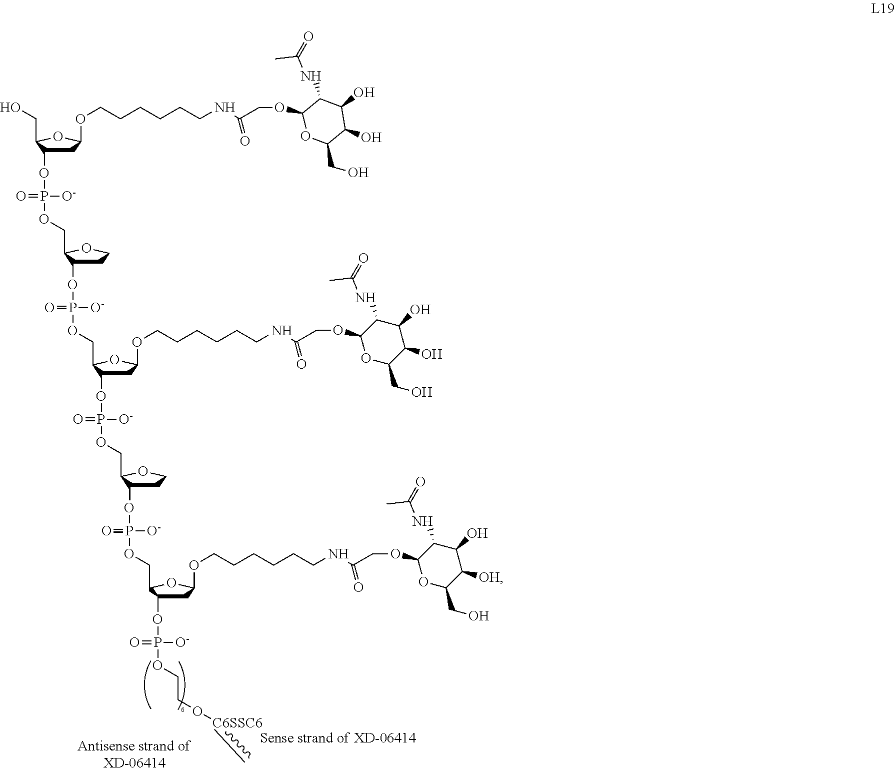

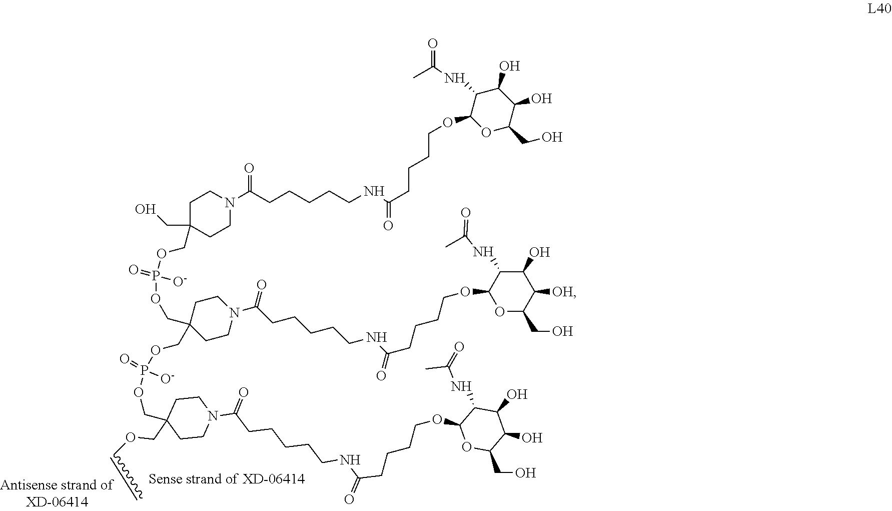

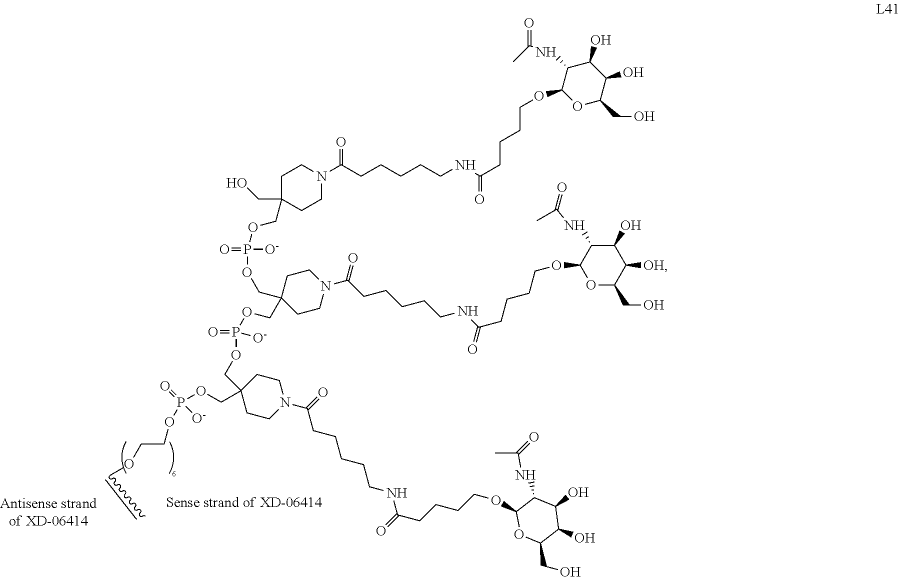

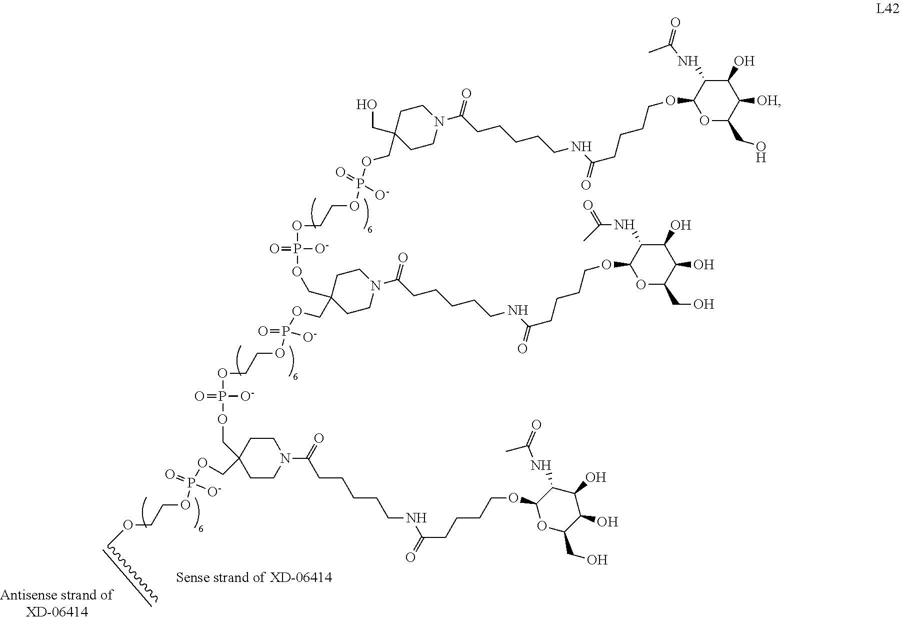

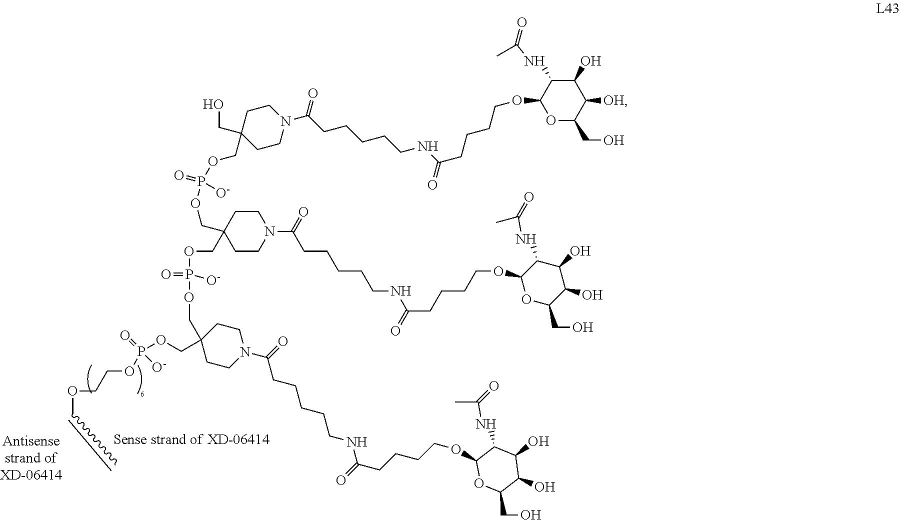

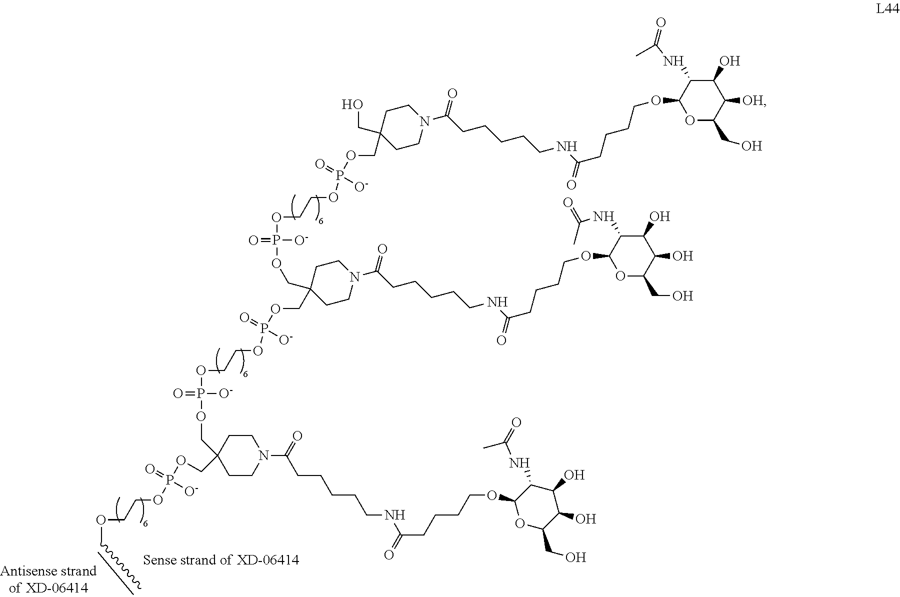

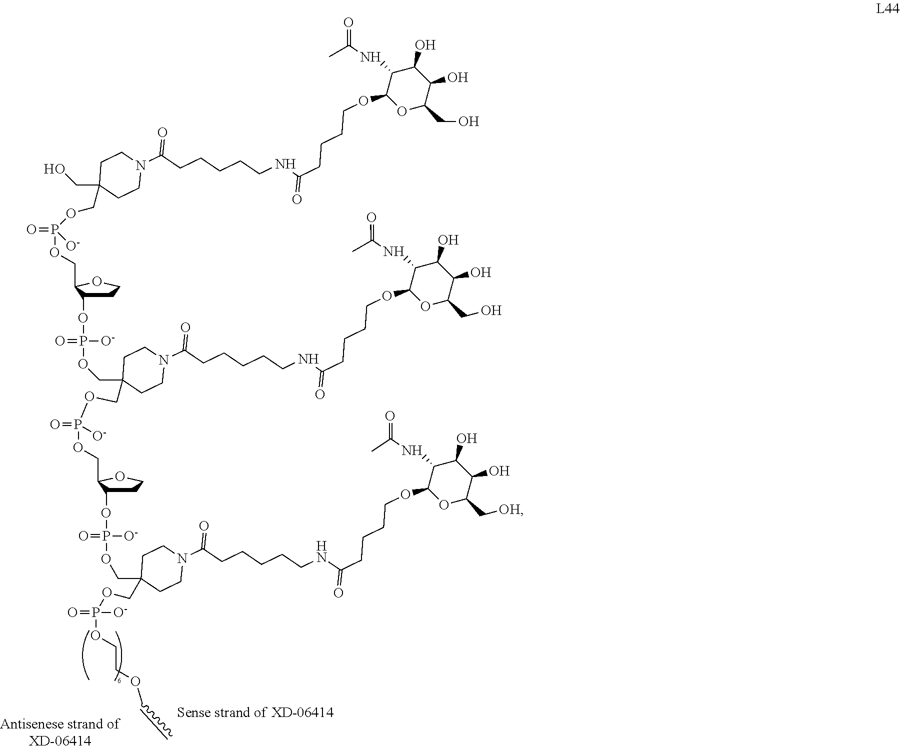

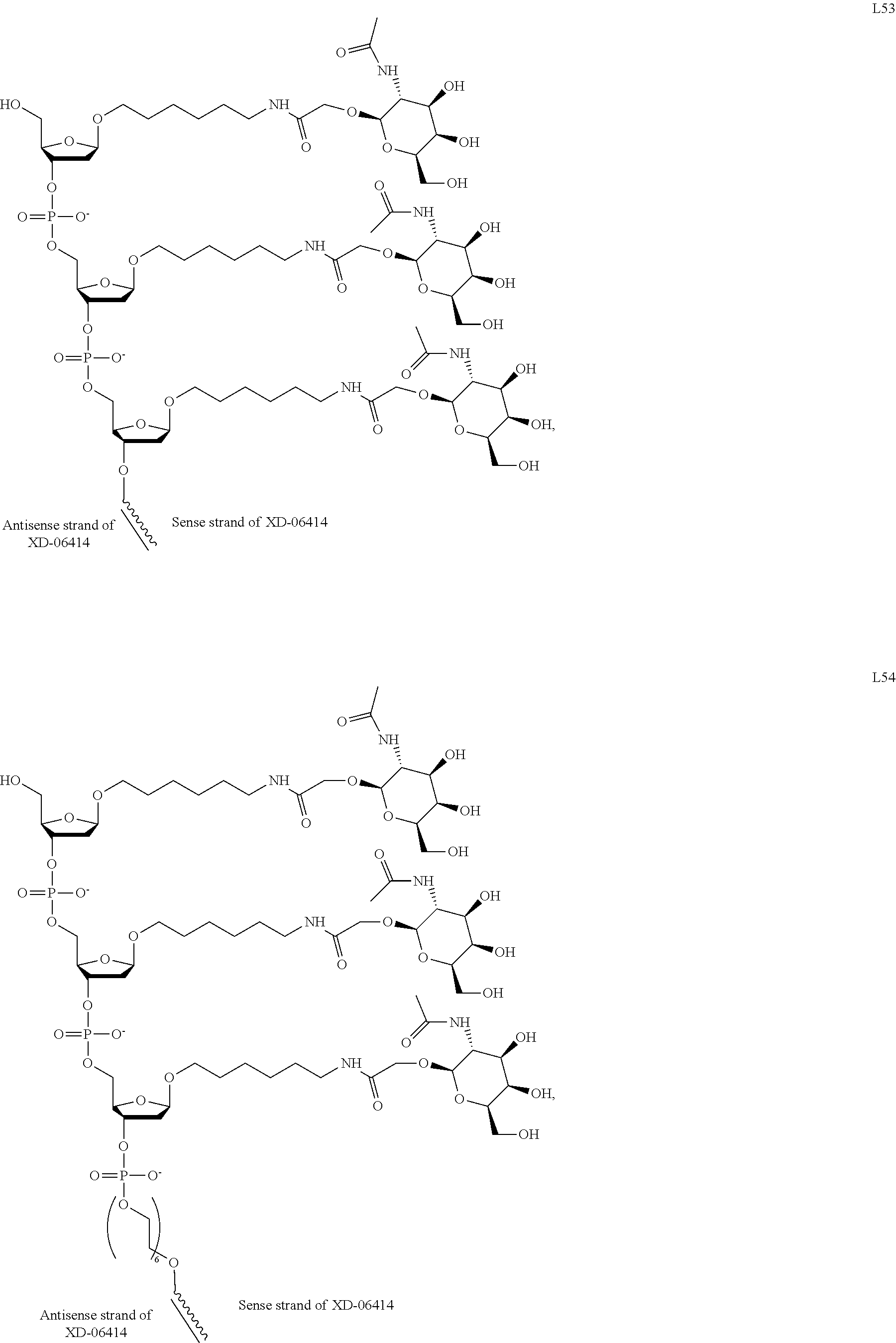

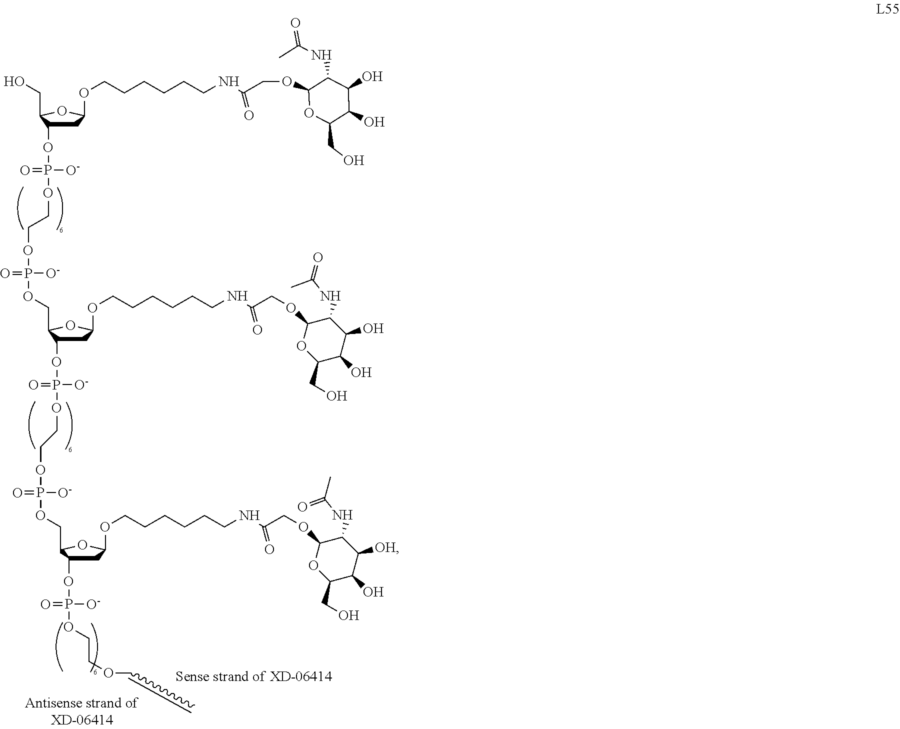

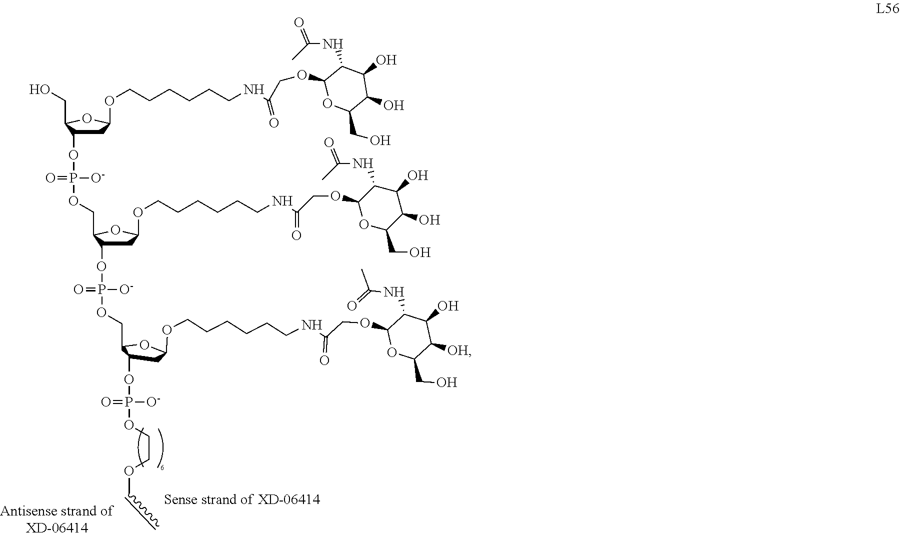

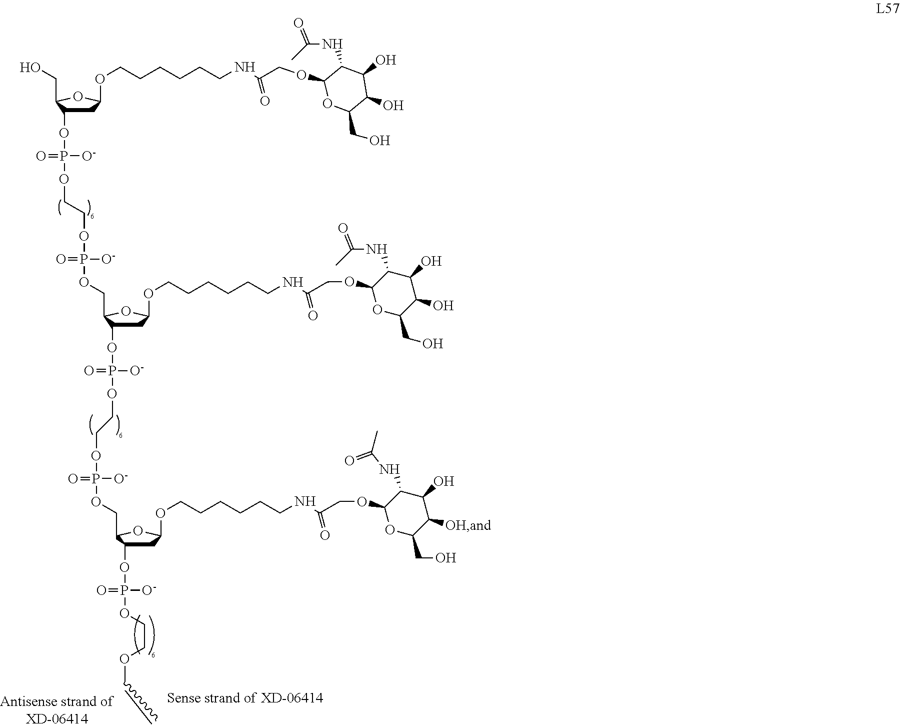

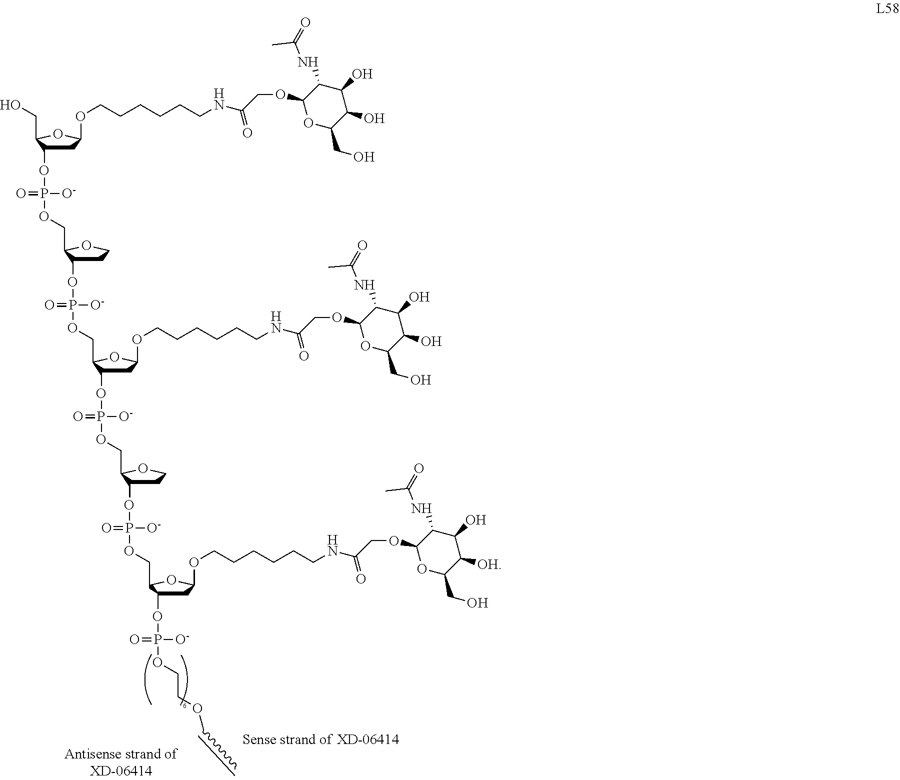

21. The conjugate of claim 19, wherein the conjugate is L53.

22. The conjugate of claim 19, wherein the conjugate is L55.

23. The conjugate of claim 9, wherein the oligonucleotide is a synthetic isolated small inhibiting RNA (siRNA).

24. The conjugate of claim 23, wherein siRNA is a double-stranded siRNA.

25. The conjugate claim 24, wherein the GalNAc cluster is connected to the 5′ or 3′ end of the sense strand.

26. A conjugate prepared by a process comprising the steps of:
1). providing at least one GalNAc monomer selected from the group consisting of M1′, M2′, M3′, M4′, M5′ and M6′; optionally adding at least one spacer;
2). providing at least one oligonucleotide; optionally adding at least one linker; and
3). synthesizing the conjugate from the GalNAc monomer(s) in step 1) and the oligonucleotide(s) in step 2), optionally removing the protecting groups.

27. The conjugate of claim 26, wherein the oligonucleotide is an saRNA.

28. The conjugate of claim 27, wherein the saRNA is selected from the group consisting of XD-03302, S1 (XD-06409), S2 (XD-06410), S3 (XD-06411), S4 (XD-06412), S5 (XD-06413), S6 (XD-06414), S7 (XD-06415), S8, XD-07139, XD-03934, and XD-14369K1.

29. The conjugate of claim 26, wherein the oligonucleotide is an siRNA.

30. A pharmaceutical composition comprising the conjugate of claim 9 and at least one pharmaceutically acceptable excipient.

31. The pharmaceutical composition of claim 30, wherein the conjugate comprises an saRNA.

32. The pharmaceutical composition of claim 30, wherein the conjugate comprises an siRNA.

33. A method of delivering an oligonucleotide to cells comprising administering the conjugate of claim 9 to cells, wherein no transfection agent is used.

34. The method of claim 33, wherein the oligonucleotide comprises saRNA.

35. The method of claim 33, wherein the oligonucleotide comprises siRNA.

36. A method of regulating the expression of a target gene in a patient in need thereof, comprising administering the conjugate of claim 9.

37. The method of claim 36, wherein the conjugate comprises an saRNA.

38. The method of claim 36, wherein the expression of the target gene is increased.

39. The method of claim 37, wherein the target gene is CEBPA.

40. The method of claim 39, wherein the expression of albumin is increased in the patient.

41. The method of claim 36, wherein the conjugate comprises an siRNA.

42. The method of claim 36, wherein the expression of the target gene is reduced.

43. The method of claim 42, wherein the target gene is C5.


SEQUENCE LISTING (29 sequences)

SEQ ID NO: 1 - DNA, 4001 bp, Homo sapiens
tccctctcccaccaggggtatacatcctcagagctgacccacgacctagctttctggtgtgactcggggtgggggctcccactggtcacctggtgacccccatcgcagtgagttccgccccaaggggaagcccagcctatagcaggctggggtggggtgtgtgcggagggaggtgggagaggcgtggaactagagaccctccaccttcatgtagaactaggggaacaaccttaggttccaagccccaagtccctatgtttccacccctttctaaggacaggcgtggaggagcggctggggctggcgggcttgtcgggatctcagctccctgagccctcctcctgccacgggcctgctcccctccttctctcatgggggtctgctgtagcctcgggaaggaggcaggaaacctccaaataaaatgacaaggcacgatttgctccccctactcagtaggcattggagcggtgagtttgcatttccaaggcacaaggttatcctaaatactagagttgccgggctcccagctcagccccaagaattctcccctcctcgcagggagaagccaccgcctggccccctcatcttagacgcaccaagtccggcgcagaggaagggaggggacacgcggagcaggccaggctttcaggaggcaccggaatctcctagtcctggctcgcacggctcgggcaagcctcgagatccggcgaccccaaaccactccctgggtccccgccggaggctggcccagggcggtcccacagccgcgcgcctcacgcgcagttgcccatggccttgaccaaggagctctctggcagctggcggaagatgccccgcagcgtgtccagttcgcggctcagctgttccacccgcttgcgcaggcggtcattgtcactggtcagctccagcaccttctgctgcgtctccacgttgcgctgcttggccttgtcgcggctcttgcgcaccgcgatgttgttgcgctcgcgccgcacccggtactcgttgctgttcttgtccaccgacttcttggccttgcccgcgccgctgccgccactcgcgcggaggtcggggtgcgcggcgcccagccccttgagcgcgctgccagggcccggcaggccggcggcaccgagcgcgggcgcggggtgcgggctgggcacgggcgtgggcggcggcgtggggtgaccgggctgcaggtgcatggtggtctggccgcagtgcgcgatctggaactgcaggtgcggggcggccaggtgcgcgggcggcgggtgcgggtgcgggtgcgagggcggcggcggcggcggcggctggtaagggaagaggccggccagcgccagctgcttggcttcatcctcctcgcggggctcctgcttgatcaccagcggccgcagcgccggcgccccgacgcgctcgtacaggggctccagcctgccgtccaggtagccggcggccgcgcagccgtagccgggcgggggcccgtgcgctcccccgggcatgacggcgccgccggggcccgcgggcgcgcccgggtagtcaaagtcgccgccgccgccgccgcccgtggggcccacggccgccttggccttctcctgctgccggctgtgctggaacaggtcggccaggaactcgtcgttgaaggcggccgggtcgatgtaggcgctgatgtcgatggacgtctcgtgctcgcagatgccgcccagcggctccggggcggcaggtggggcgggaggctgcgcggggcccgcgccccggggaaagccgaaggcggcgctgctgggcgcgtgcggggggctctgcaggtggctgctcatcgggggccgcggctccgcctcgtagaagtcggccgactccatgggggagttagagttctcccggcatggcgagcctcggcggcctccagcctgcgcggggcgtcgccgccgcccacccggagaccctgctcgcccgcgcccgcgcacctccgggtcgcgaatggcccggcccgcgccggcccagcttttatacccggcaggccgcgtcgccccctagagtccgaggcggcctctgtccccgggctgcggcggcgcggcgcctgctgggtcctagcgcgcggccggcatggggcggcgaaccagcgcggcacagcgccgcgctccccaggcaggccgcggcgcaacgcccaccgcctccagcgcgcccagcagagccgcggcgctcgctccaagctccgcccccggcccggccgtcgcccccgcgcccacgtggtcggtagcgggggccccctcctcctgcctgccctaggcgcccgtatccagccacggccgggagcccaggagtatcccgaggctgcacggggtaggggtggggggcggagggcgagtcttggtcttgagctgctggggcgcggattctctttcaaagccagaaccaggcctgtcccggacccgcgtcccggggaggctgcagcgcagagcagcggggctggggccggtggggggccgtttgggacgcgcggagaggtcctgagcgcggtggctctgcgtctcctagctctgatctccaggctacccctgtgattccgcgcagaggtacctctcggaggacgccggggtcccatgggcggcgccgcgcagggcgctaggaccccgcggggagcggaggcggcctcggcccgggagcctggaggacctggccggtcgatccgcccgggctggaaaactttctttataattacttctccaggtcggagcgcgcggcttgctaggcgcgcggggccggcgctgttacccggcgtggagtcgccgattttttttcctgcgggaccgcggggccccccagactagcggagctggacgccggggcgagcacggggaggggcgcaccgagggaggagacaaacttaactctggggccgggattccgaggcgggggccgcagccctcgaggcccgaagccaccgcttcctcccccgcctccccattcaggtgggcgccaacggcgggagcgagggtgtccaggccgccgggctgccaggtccgagcacgcacagggagaactctgcccagtggttcgccgggcgctgtagtccccgggatcctagggaccgaggcggccaggccctggggccccttgagtgcggcagctaatgctctcaccgcggcgggggaaggagcttgccaccgagacccccagccacgtgcgtccctcgcattctttaccggggccggggtggcggctacggaccgtcagctgggcccagatggagtcttgggagccctcaagtgtctcctgtccttgcccgcgccgcccctcgccactggcgctgaggcctgacgccgcctgcgtcccggctagaggcgcgcttgcctacaggtgagggaagacccccttcaccgacagtggccttaggcctggcaaggcgccacgacccgcccaggagccccggagggggcacagctaaaaacaccgctggagagccccgagcttccacgacgatcgcagtaaagaagcagtttcatctgggcaacgcacactgcgctttaatcaagttcctattcaacatagtcccagtgattaatagcccaactgcttcgttttcggtccagagctcataaacaagatatttttagcttgacgcttttggacgggagggagtaaaaaccagatacgttaaataaatatcccgatgtgagccggagagctgcttgctgagccaaatgcaggacccattcatatagcattcacctgtggagggagacctggacggaaatcaaaaagcaccaagagcgatttgcgtttttttctgcggtgctaaaactaatggcttttcctacctaggaacaaagaaacgccactgtacatgcacggttcccggcctgtggagttgtgggaggaaggcgatgtctggccttttttgcacagctgctgttgcctgcccagagatcgggaactctgccccgtaggactggaagaaacctcagtaatgggaataagactttgtccaatagggggctgatgaatgtgtg

SEQ ID NO: 2 - RNA, 21 nt, Artificial Sequence
  Description of Artificial Sequence Synthetic oligonucleotide
gcggucauugucacuggucuu

SEQ ID NO: 3 - RNA, 21 nt, Artificial Sequence
  Description of Artificial Sequence Synthetic oligonucleotide
gaccagugacaaugaccgcuu

SEQ ID NO: 4 - DNA, 22 bp, Artificial Sequence
  Description of Artificial Sequence Synthetic oligonucleotide
  Description of Combined DNA/RNA Molecule Synthetic oligonucleotide
  5′ NH2-(C6H12)-linker
  modified_base (22)..(22): Inverted nucleotide
gcggucauugucacuggucuut

SEQ ID NO: 5 - RNA, 21 nt, Artificial Sequence
  Description of Artificial Sequence Synthetic oligonucleotide
  misc_feature (20)..(21): Phosphorothioate linkage
gaccagugacaaugaccgcuu

SEQ ID NO: 6 - DNA, 22 bp, Artificial Sequence
  Description of Artificial Sequence Synthetic oligonucleotide
  Description of Combined DNA/RNA Molecule Synthetic oligonucleotide
  5′ NH2-(C6H12)-linker
  modified_base (1)..(1): 2′-Fluoro nucleotide
  modified_base (2)..(2): 2′-O-Methyl nucleotide
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-Fluoro nucleotide
  modified_base (14)..(14): 2′-O-Methyl nucleotide
  modified_base (15)..(15): 2′-Fluoro nucleotide
  modified_base (16)..(16): 2′-O-Methyl nucleotide
  modified_base (17)..(17): 2′-Fluoro nucleotide
  modified_base (18)..(18): 2′-O-Methyl nucleotide
  modified_base (19)..(19): 2′-Fluoro nucleotide
  modified_base (20)..(21): 2′-O-Methyl nucleotide
  modified_base (22)..(22): Inverted nucleotide
gcggucauugucacuggucuut

SEQ ID NO: 7 - RNA, 21 nt, Artificial Sequence
  Description of Artificial Sequence Synthetic oligonucleotide
  modified_base (1)..(1): 2′-O-Methyl nucleotide
  modified_base (2)..(2): 2′-Fluoro nucleotide
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-Fluoro nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(21): 2′-O-Methyl nucleotide
  misc_feature (20)..(21): Phosphorothioate linkage
gaccagugacaaugaccgcuu

SEQ ID NO: 8 - DNA, 22 bp, Artificial Sequence
  Description of Artificial Sequence Synthetic oligonucleotide
  Description of Combined DNA/RNA Molecule Synthetic oligonucleotide
  5′ NH2-(C6H12)-linker
  modified_base (1)..(1): 2′-Fluoro nucleotide
  modified_base (2)..(2): 2′-O-Methyl nucleotide
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-Fluoro nucleotide
  modified_base (14)..(14): 2′-O-Methyl nucleotide
  modified_base (15)..(15): 2′-Fluoro nucleotide
  modified_base (16)..(16): 2′-O-Methyl nucleotide
  modified_base (17)..(17): 2′-Fluoro nucleotide
  modified_base (18)..(18): 2′-O-Methyl nucleotide
  modified_base (19)..(19): 2′-Fluoro nucleotide
  modified_base (20)..(21): 2′-O-Methyl nucleotide
  modified_base (22)..(22): Inverted nucleotide
gcggucauugucacuggucuut

SEQ ID NO: 9 - RNA, 21 nt, Artificial Sequence
  Description of Artificial Sequence Synthetic oligonucleotide
  modified_base (1)..(1): 2′-O-Methyl nucleotide
  modified_base (2)..(2): 2′-Fluoro nucleotide
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-Fluoro nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(21): 2′-O-Methyl nucleotide
  misc_feature (19)..(21): Phosphorothioate linkage
gaccagugacaaugaccgcuu

SEQ ID NO: 10 - DNA, 22 bp, Artificial Sequence
  Description of Artificial Sequence Synthetic oligonucleotide
  Description of Combined DNA/RNA Molecule Synthetic oligonucleotide
  5′ NH2-(C6H12)-linker
  modified_base (1)..(1): 2′-Fluoro nucleotide
  modified_base (2)..(2): 2′-O-Methyl nucleotide
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(11): 2′-Fluoro nucleotide
  modified_base (12)..(12): 2′-O-Methyl nucleotide
  modified_base (13)..(13): 2′-Fluoro nucleotide
  modified_base (14)..(14): 2′-O-Methyl nucleotide
  modified_base (15)..(15): 2′-Fluoro nucleotide
  modified_base (16)..(16): 2′-O-Methyl nucleotide
  modified_base (17)..(17): 2′-Fluoro nucleotide
  modified_base (18)..(18): 2′-O-Methyl nucleotide
  modified_base (19)..(19): 2′-Fluoro nucleotide
  modified_base (20)..(21): 2′-O-Methyl nucleotide
  modified_base (22)..(22): Inverted nucleotide
gcggucauugucacuggucuut

SEQ ID NO: 11 - RNA, 21 nt, Artificial Sequence
  Description of Artificial Sequence Synthetic oligonucleotide
  modified_base (1)..(1): 2′-O-Methyl nucleotide
  modified_base (2)..(2): 2′-Fluoro nucleotide
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(11): 2′-O-Methyl nucleotide
  modified_base (12)..(12): 2′-Fluoro nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(21): 2′-O-Methyl nucleotide
  misc_feature (19)..(21): Phosphorothioate linkage
gaccagugacaaugaccgcuu

SEQ ID NO: 12 - DNA, 22 bp, Artificial Sequence
  Description of Artificial Sequence Synthetic oligonucleotide
  Description of Combined DNA/RNA Molecule Synthetic oligonucleotide
  5′ NH2-(C6H12)-linker
  modified_base (1)..(1): 2′-Fluoro nucleotide
  modified_base (2)..(2): 2′-O-Methyl nucleotide
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(11): 2′-Fluoro nucleotide
  modified_base (12)..(12): 2′-O-Methyl nucleotide
  modified_base (13)..(13): 2′-Fluoro nucleotide
  modified_base (14)..(14): 2′-O-Methyl nucleotide
  modified_base (15)..(15): 2′-Fluoro nucleotide
  modified_base (16)..(16): 2′-O-Methyl nucleotide
  modified_base (17)..(17): 2′-Fluoro nucleotide
  modified_base (18)..(18): 2′-O-Methyl nucleotide
  modified_base (19)..(19): 2′-Fluoro nucleotide
  modified_base (20)..(21): 2′-O-Methyl nucleotide
  modified_base (22)..(22): Inverted nucleotide
gcggucauacacacuggucuut

SEQ ID NO: 13 - RNA, 21 nt, Artificial Sequence
  Description of Artificial Sequence Synthetic oligonucleotide
  modified_base (1)..(1): 2′-O-Methyl nucleotide
  modified_base (2)..(2): 2′-Fluoro nucleotide
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(11): 2′-O-Methyl nucleotide
  modified_base (12)..(12): 2′-Fluoro nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(21): 2′-O-Methyl nucleotide
  misc_feature (19)..(21): Phosphorothioate linkage
gaccaguguguaugaccgcuu

SEQ ID NO: 14 - DNA, 22 bp, Artificial Sequence
  Description of Artificial Sequence Synthetic oligonucleotide
  Description of Combined DNA/RNA Molecule Synthetic oligonucleotide
  5′ NH2-(C6H12)-linker or absent
  modified_base (1)..(1): 2′-Fluoro nucleotide
  modified_base (2)..(2): 2′-O-Methyl nucleotide
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-Fluoro nucleotide
  modified_base (14)..(14): 2′-O-Methyl nucleotide
  modified_base (15)..(15): 2′-Fluoro nucleotide
  modified_base (16)..(16): 2′-O-Methyl nucleotide
  modified_base (17)..(17): 2′-Fluoro nucleotide
  modified_base (18)..(18): 2′-O-Methyl nucleotide
  modified_base (19)..(19): 2′-Fluoro nucleotide
  modified_base (20)..(21): 2′-O-Methyl nucleotide
  modified_base (22)..(22): Inverted nucleotide
gcggucauugucacuggucuut

SEQ ID NO: 15 - RNA, 21 nt, Artificial Sequence
  Description of Artificial Sequence Synthetic oligonucleotide
  modified_base (1)..(1): 2′-O-Methyl nucleotide
  modified_base (2)..(2): 2′-Fluoro nucleotide
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-Fluoro nucleotide
  modified_base (11)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(21): 2′-O-Methyl nucleotide
  misc_feature (19)..(21): Phosphorothioate linkage
gaccagugacaaugaccgcuu

SEQ ID NO: 16 - DNA, 22 bp, Artificial Sequence
  Description of Artificial Sequence Synthetic oligonucleotide
  Description of Combined DNA/RNA Molecule Synthetic oligonucleotide
  5′ NH2-(C6H12)-linker
  modified_base (1)..(1): 2′-O-Methyl nucleotide
  modified_base (2)..(2): 2′-Fluoro nucleotide
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-Fluoro nucleotide
  modified_base (11)..(11): 2′-O-Methyl nucleotide
  modified_base (12)..(12): 2′-Fluoro nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(21): 2′-O-Methyl nucleotide
  modified_base (22)..(22): Inverted nucleotide
gcggucauugucacuggucuut

SEQ ID NO: 17 - RNA, 21 nt, Artificial Sequence
  Description of Artificial Sequence Synthetic oligonucleotide
  modified_base (1)..(1): 2′-Fluoro nucleotide
  modified_base (2)..(2): 2′-O-Methyl nucleotide
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-Fluoro nucleotide
  modified_base (14)..(14): 2′-O-Methyl nucleotide
  modified_base (15)..(15): 2′-Fluoro nucleotide
  modified_base (16)..(16): 2′-O-Methyl nucleotide
  modified_base (17)..(17): 2′-Fluoro nucleotide
  modified_base (18)..(18): 2′-O-Methyl nucleotide
  modified_base (19)..(19): 2′-Fluoro nucleotide
  misc_feature (19)..(21): Phosphorothioate linkage
  modified_base (20)..(21): 2′-O-Methyl nucleotide
gaccagugacaaugaccgcuu

SEQ ID NO: 18 - DNA, 22 bp, Artificial Sequence
  Description of Artificial Sequence Synthetic oligonucleotide
  Description of Combined DNA/RNA Molecule Synthetic oligonucleotide
  modified_base (1)..(1): 2′-Fluoro nucleotide
  modified_base (2)..(2): 2′-O-Methyl nucleotide
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-Fluoro nucleotide
  modified_base (14)..(14): 2′-O-Methyl nucleotide
  modified_base (15)..(15): 2′-Fluoro nucleotide
  modified_base (16)..(16): 2′-O-Methyl nucleotide
  modified_base (17)..(17): 2′-Fluoro nucleotide
  modified_base (18)..(18): 2′-O-Methyl nucleotide
  modified_base (19)..(19): 2′-Fluoro nucleotide
  modified_base (20)..(21): 2′-O-Methyl nucleotide
  modified_base (22)..(22): Inverted nucleotide
gcggucauugucacuggucuut

SEQ ID NO: 19 - RNA, 21 nt, Artificial Sequence
  Description of Artificial Sequence Synthetic oligonucleotide
  modified_base (1)..(1): 2′-O-Methyl nucleotide
  modified_base (2)..(2): 2′-Fluoro nucleotide
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-Fluoro nucleotide
  modified_base (11)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(21): 2′-O-Methyl nucleotide
  misc_feature (19)..(21): Phosphorothioate linkage
gaccagugacaaugaccgcuu

SEQ ID NO: 20 - DNA, 23 bp, Artificial Sequence
  Description of Artificial Sequence Synthetic oligonucleotide
  Description of Combined DNA/RNA Molecule Synthetic oligonucleotide
  modified_base (1)..(1): Inverted nucleotide
  modified_base (2)..(2): 2′-Fluoro nucleotide
  misc_feature (1)..(2): Phosphorothioate linkage
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(10): 2′-Fluoro nucleotide
  modified_base (11)..(11): 2′-O-Methyl nucleotide
  modified_base (12)..(12): 2′-Fluoro nucleotide
  modified_base (13)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-Fluoro nucleotide
  modified_base (21)..(22): 2′-O-Methyl nucleotide
  modified_base (23)..(23): Inverted nucleotide
tgcggucauugucacuggucuut

SEQ ID NO: 21 - RNA, 21 nt, Artificial Sequence
  Description of Artificial Sequence Synthetic oligonucleotide
  modified_base (1)..(1): 2′-O-Methyl nucleotide
  modified_base (2)..(2): 2′-Fluoro nucleotide
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-Fluoro nucleotide
  modified_base (11)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(21): 2′-O-Methyl nucleotide
  misc_feature (19)..(21): Phosphorothioate linkage
gaccagugacaaugaccgcuu

SEQ ID NO: 22 - RNA, 21 nt, Artificial Sequence
  Description of Artificial Sequence Synthetic oligonucleotide
  modified_base (1)..(1): Inverted abasic capped nucleotide
  modified_base (1)..(2): 2′-O-Methyl nucleotide
  modified_base (4)..(4): 2′-O-Methyl nucleotide
  modified_base (10)..(10): 2′-O-Methyl nucleotide
  modified_base (14)..(14): 2′-O-Methyl nucleotide
  modified_base (20)..(21): 2′-O-Methyl nucleotide
gcggucauugucacuggucuu

SEQ ID NO: 23 - RNA, 21 nt, Artificial Sequence
  Description of Artificial Sequence Synthetic oligonucleotide
  modified_base (20)..(21): 2′-O-Methyl nucleotide
gaccagugacaaugaccgcuu

SEQ ID NO: 24 - RNA, 19 nt, Artificial Sequence
  Description of Artificial Sequence Synthetic oligonucleotide
  modified_base (1)..(1): 2′-Fluoro nucleotide
  misc_feature (1)..(2): Phosphorothioate linkage
  modified_base (2)..(2): 2′-O-Methyl nucleotide
  misc_feature (2)..(3): Phosphorothioate linkage
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(9): 2′-Fluoro nucleotide
  modified_base (10)..(10): 2′-O-Methyl nucleotide
  modified_base (11)..(11): 2′-Fluoro nucleotide
  modified_base (12)..(12): 2′-O-Methyl nucleotide
  modified_base (13)..(13): 2′-Fluoro nucleotide
  modified_base (14)..(14): 2′-O-Methyl nucleotide
  modified_base (15)..(15): 2′-Fluoro nucleotide
  modified_base (16)..(16): 2′-O-Methyl nucleotide
  modified_base (17)..(17): 2′-Fluoro nucleotide
  modified_base (18)..(18): 2′-O-Methyl nucleotide
  modified_base (19)..(19): 2′-Fluoro nucleotide
gcggucauugucacugguc

SEQ ID NO: 25 - RNA, 21 nt, Artificial Sequence
  Description of Artificial Sequence Synthetic oligonucleotide
  modified_base (1)..(1): 2′-Fluoro nucleotide
  misc_feature (1)..(2): Phosphorothioate linkage
  modified_base (2)..(2): 2′-Fluoro nucleotide
  misc_feature (2)..(3): Phosphorothioate linkage
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-Fluoro nucleotide
  modified_base (11)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  misc_feature (19)..(20): Phosphorothioate linkage
  modified_base (20)..(20): 2′-Fluoro nucleotide
  misc_feature (20)..(21): Phosphorothioate linkage
  modified_base (21)..(21): 2′-O-Methyl nucleotide
gaccagugacaaugaccgcuu

SEQ ID NO: 26 - RNA, 21 nt, Artificial Sequence
  Description of Artificial Sequence Synthetic oligonucleotide
aagcaagauauuuuuauaaua

SEQ ID NO: 27 - RNA, 23 nt, Artificial Sequence
  Description of Artificial Sequence Synthetic oligonucleotide
uauuauaaaaauaucuugcuuuu

SEQ ID NO: 28 - RNA, 21 nt, Artificial Sequence
  Description of Artificial Sequence Synthetic oligonucleotide
  modified_base (1)..(1): 2′-Fluoro nucleotide
  misc_feature (1)..(2): Phosphorothioate linkage
  modified_base (2)..(2): 2′-O-Methyl nucleotide
  misc_feature (2)..(3): Phosphorothioate linkage
  modified_base (3)..(3): 2′-Fluoro nucleotide
  modified_base (4)..(4): 2′-O-Methyl nucleotide
  modified_base (5)..(5): 2′-Fluoro nucleotide
  modified_base (6)..(6): 2′-O-Methyl nucleotide
  modified_base (7)..(7): 2′-Fluoro nucleotide
  modified_base (8)..(8): 2′-O-Methyl nucleotide
  modified_base (9)..(11): 2′-Fluoro nucleotide
  modified_base (12)..(12): 2′-O-Methyl nucleotide
  modified_base (13)..(13): 2′-Fluoro nucleotide
  modified_base (14)..(14): 2′-O-Methyl nucleotide
  modified_base (15)..(15): 2′-Fluoro nucleotide
  modified_base (16)..(16): 2′-O-Methyl nucleotide
  modified_base (17)..(17): 2′-Fluoro nucleotide
  modified_base (18)..(18): 2′-O-Methyl nucleotide
  modified_base (19)..(19): 2′-Fluoro nucleotide
  modified_base (20)..(20): 2′-O-Methyl nucleotide
  modified_base (21)..(21): 2′-Fluoro nucleotide
aagcaagauauuuuuauaaua

SEQ ID NO: 29 - RNA, 23 nt, Artificial Sequence
  Description of Artificial Sequence Synthetic oligonucleotide
  modified_base (1)..(1): 2′-O-Methyl nucleotide
  misc_feature (1)..(2): Phosphorothioate linkage
  modified_base (2)..(2): 2′-Fluoro nucleotide
  misc_feature (2)..(3): Phosphorothioate linkage
  modified_base (3)..(3): 2′-O-Methyl nucleotide
  modified_base (4)..(4): 2′-Fluoro nucleotide
  modified_base (5)..(5): 2′-O-Methyl nucleotide
  modified_base (6)..(6): 2′-Fluoro nucleotide
  modified_base (7)..(7): 2′-O-Methyl nucleotide
  modified_base (8)..(8): 2′-Fluoro nucleotide
  modified_base (9)..(9): 2′-O-Methyl nucleotide
  modified_base (10)..(10): 2′-Fluoro nucleotide
  modified_base (11)..(13): 2′-O-Methyl nucleotide
  modified_base (14)..(14): 2′-Fluoro nucleotide
  modified_base (15)..(15): 2′-O-Methyl nucleotide
  modified_base (16)..(16): 2′-Fluoro nucleotide
  modified_base (17)..(17): 2′-O-Methyl nucleotide
  modified_base (18)..(18): 2′-Fluoro nucleotide
  modified_base (19)..(19): 2′-O-Methyl nucleotide
  modified_base (20)..(20): 2′-Fluoro nucleotide
  modified_base (21)..(23): 2′-O-Methyl nucleotide
  misc_feature (21)..(22): Phosphorothioate linkage
  misc_feature (22)..(23): Phosphorothioate linkage
uauuauaaaaauaucuugcuuuu